SEQUENCE LISTING (542 sequences)

SEQ ID NO: 1 - protein, 16 aa, Homo sapiens
MAEPRQEFEVMEDHAG

SEQ ID NO: 2 - protein, 16 aa, Homo sapiens
EVMEDHAGTYGLGDRK

SEQ ID NO: 3 - protein, 16 aa, Homo sapiens
TYGLGDRKDQGGYTMH

SEQ ID NO: 4 - protein, 16 aa, Homo sapiens
DQGGYTMHQDQEGDTD

SEQ ID NO: 5 - protein, 16 aa, Homo sapiens
QDQEGDTDAGLKESPL

SEQ ID NO: 6 - protein, 16 aa, Homo sapiens
AGLKESPLQTPTEDGS

SEQ ID NO: 7 - protein, 16 aa, Homo sapiens
QTPTEDGSEEPGSETS

SEQ ID NO: 8 - protein, 16 aa, Homo sapiens
EEPGSETSDAKSTPTA

SEQ ID NO: 9 - protein, 16 aa, Homo sapiens
DAKSTPTAEDVTAPLV

SEQ ID NO: 10 - protein, 16 aa, Homo sapiens
EDVTAPLVDEGAPGKQ

SEQ ID NO: 11 - protein, 16 aa, Homo sapiens
DEGAPGKQAAAQPHTE

SEQ ID NO: 12 - protein, 16 aa, Homo sapiens
AAAQPHTEIPEGTTAE

SEQ ID NO: 13 - protein, 16 aa, Homo sapiens
IPEGTTAEEAGIGDTP

SEQ ID NO: 14 - protein, 16 aa, Homo sapiens
EAGIGDTPSLEDEAAG

SEQ ID NO: 15 - protein, 16 aa, Homo sapiens
SLEDEAAGHVTQARMV

SEQ ID NO: 16 - protein, 16 aa, Homo sapiens
HVTQARMVSKSKDGTG

SEQ ID NO: 17 - protein, 16 aa, Homo sapiens
SKSKDGTGSDDKKAKG

SEQ ID NO: 18 - protein, 16 aa, Homo sapiens
SDDKKAKGADGKTKIA

SEQ ID NO: 19 - protein, 16 aa, Homo sapiens
ADGKTKIATPRGAAPP

SEQ ID NO: 20 - protein, 16 aa, Homo sapiens
TPRGAAPPGQKGQANA

SEQ ID NO: 21 - protein, 16 aa, Homo sapiens
GQKGQANATRIPAKTP

SEQ ID NO: 22 - protein, 16 aa, Homo sapiens
TRIPAKTPPAPKTPPS

SEQ ID NO: 23 - protein, 16 aa, Homo sapiens
  MOD_RES (5)..(5): PHOSPHORYLATION
PAPKTPPSSGEPPKSG

SEQ ID NO: 24 - protein, 16 aa, Homo sapiens
SGEPPKSGDRSGYSSP

SEQ ID NO: 25 - protein, 16 aa, Homo sapiens
  MOD_RES (10)..(10): PHOSPHORYLATION
  MOD_RES (13)..(13): PHOSPHORYLATION
DRSGYSSPGSPGTPGS

SEQ ID NO: 26 - protein, 16 aa, Homo sapiens
GSPGTPGSRSRTPSLP

SEQ ID NO: 27 - protein, 16 aa, Homo sapiens
RSRTPSLPTPPTREPK

SEQ ID NO: 28 - protein, 16 aa, Homo sapiens
TPPTREPKKVAVVRTP

SEQ ID NO: 29 - protein, 16 aa, Homo sapiens
  MOD_RES (7)..(7): PHOSPHORYLATION
KVAVVRTPPKSPSSAK

SEQ ID NO: 30 - protein, 16 aa, Homo sapiens
PKSPSSAKSRLQTAPV

SEQ ID NO: 31 - protein, 16 aa, Homo sapiens
SRLQTAPVPMPDLKNV

SEQ ID NO: 32 - protein, 16 aa, Homo sapiens
  MOD_RES (14)..(14): PHOSPHORYLATION
PMPDLKNVKSKIGSTE

SEQ ID NO: 33 - protein, 16 aa, Homo sapiens
KSKIGSTENLKHQPGG

SEQ ID NO: 34 - protein, 16 aa, Homo sapiens
NLKHQPGGGKVQIINK

SEQ ID NO: 35 - protein, 16 aa, Homo sapiens
GKVQIINKKLDLSNVQ

SEQ ID NO: 36 - protein, 16 aa, Homo sapiens
KLDLSNVQSKCGSKDN

SEQ ID NO: 37 - protein, 16 aa, Homo sapiens
SKCGSKDNIKHVPGGG

SEQ ID NO: 38 - protein, 16 aa, Homo sapiens
IKHVPGGGSVQIVYKP

SEQ ID NO: 39 - protein, 16 aa, Homo sapiens
SVQIVYKPVDLSKVTS

SEQ ID NO: 40 - protein, 16 aa, Homo sapiens
VDLSKVTSKCGSLGNI

SEQ ID NO: 41 - protein, 16 aa, Homo sapiens
KCGSLGNIHHKPGGGQ

SEQ ID NO: 42 - protein, 16 aa, Homo sapiens
HHKPGGGQVEVKSEKL

SEQ ID NO: 43 - protein, 16 aa, Homo sapiens
VEVKSEKLDFKDRVQS

SEQ ID NO: 44 - protein, 16 aa, Homo sapiens
  MOD_RES (12)..(12): PHOSPHORYLATION
DFKDRVQSKIGSLDNI

SEQ ID NO: 45 - protein, 16 aa, Homo sapiens
KIGSLDNITHVPGGGN

SEQ ID NO: 46 - protein, 16 aa, Homo sapiens
THVPGGGNKKIETHKL

SEQ ID NO: 47 - protein, 16 aa, Homo sapiens
KKIETHKLTFRENAKA

SEQ ID NO: 48 - protein, 16 aa, Homo sapiens
TFRENAKAKTDHGAEI

SEQ ID NO: 49 - protein, 16 aa, Homo sapiens
KTDHGAEIVYKSPVVS

SEQ ID NO: 50 - protein, 16 aa, Homo sapiens
VYKSPVVSGDTSPRHL

SEQ ID NO: 51 - protein, 16 aa, Homo sapiens
GDTSPRHLSNVSSTGS

SEQ ID NO: 52 - protein, 16 aa, Homo sapiens
SNVSSTGSIDMVDSPQ

SEQ ID NO: 53 - protein, 16 aa, Homo sapiens
IDMVDSPQLATLADEV

SEQ ID NO: 54 - protein, 16 aa, Homo sapiens
LATLADEVSASLAKQG

SEQ ID NO: 55 - protein, 16 aa, Homo sapiens
ATLADEVSASLAKQGL

SEQ ID NO: 56 - protein, 15 aa, Homo sapiens
AFAFVTFADDQIAQS

SEQ ID NO: 57 - protein, 15 aa, Homo sapiens
FAFVTFADDQIAQSL

SEQ ID NO: 58 - protein, 15 aa, Homo sapiens
AFVTFADDQIAQSLC

SEQ ID NO: 59 - protein, 15 aa, Homo sapiens
FVTFADDQIAQSLCG

SEQ ID NO: 60 - protein, 15 aa, Homo sapiens
VTFADDQIAQSLCGE

SEQ ID NO: 61 - protein, 15 aa, Homo sapiens
TFADDQIAQSLCGED

SEQ ID NO: 62 - protein, 15 aa, Homo sapiens
FADDQIAQSLCGEDL

SEQ ID NO: 63 - protein, 15 aa, Homo sapiens
ADDQIAQSLCGEDLI

SEQ ID NO: 64 - protein, 15 aa, Homo sapiens
DDQIAQSLCGEDLII

SEQ ID NO: 65 - protein, 15 aa, Homo sapiens
DQIAQSLCGEDLIIK

SEQ ID NO: 66 - protein, 15 aa, Homo sapiens
QIAQSLCGEDLIIKG

SEQ ID NO: 67 - protein, 15 aa, Homo sapiens
IAQSLCGEDLIIKGI

SEQ ID NO: 68 - protein, 15 aa, Homo sapiens
AQSLCGEDLIIKGIS

SEQ ID NO: 69 - protein, 15 aa, Homo sapiens
QSLCGEDLIIKGISV

SEQ ID NO: 70 - protein, 15 aa, Homo sapiens
SLCGEDLIIKGISVH

SEQ ID NO: 71 - protein, 15 aa, Homo sapiens
LCGEDLIIKGISVHI

SEQ ID NO: 72 - protein, 15 aa, Homo sapiens
CGEDLIIKGISVHIS

SEQ ID NO: 73 - protein, 15 aa, Homo sapiens
GEDLIIKGISVHISN

SEQ ID NO: 74 - protein, 15 aa, Homo sapiens
EDLIIKGISVHISNA

SEQ ID NO: 75 - protein, 15 aa, Homo sapiens
DLIIKGISVHISNAE

SEQ ID NO: 76 - protein, 15 aa, Homo sapiens
LIIKGISVHISNAEP

SEQ ID NO: 77 - protein, 15 aa, Homo sapiens
IIKGISVHISNAEPK

SEQ ID NO: 78 - protein, 15 aa, Homo sapiens
IKGISVHISNAEPKH

SEQ ID NO: 79 - protein, 15 aa, Homo sapiens
KGISVHISNAEPKHN

SEQ ID NO: 80 - protein, 15 aa, Homo sapiens
GISVHISNAEPKHNS

SEQ ID NO: 81 - protein, 15 aa, Homo sapiens
ISVHISNAEPKHNSN

SEQ ID NO: 82 - protein, 15 aa, Homo sapiens
SVHISNAEPKHNSNR

SEQ ID NO: 83 - protein, 15 aa, Homo sapiens
VHISNAEPKHNSNRQ

SEQ ID NO: 84 - protein, 15 aa, Homo sapiens
HISNAEPKHNSNRQL

SEQ ID NO: 85 - protein, 15 aa, Homo sapiens
ISNAEPKHNSNRQLE

SEQ ID NO: 86 - protein, 15 aa, Homo sapiens
SNAEPKHNSNRQLER

SEQ ID NO: 87 - protein, 15 aa, Homo sapiens
NAEPKHNSNRQLERS

SEQ ID NO: 88 - protein, 15 aa, Homo sapiens
AEPKHNSNRQLERSG

SEQ ID NO: 89 - protein, 15 aa, Homo sapiens
EPKHNSNRQLERSGR

SEQ ID NO: 90 - protein, 15 aa, Homo sapiens
PKHNSNRQLERSGRF

SEQ ID NO: 91 - protein, 15 aa, Homo sapiens
KHNSNRQLERSGRFG

SEQ ID NO: 92 - protein, 15 aa, Homo sapiens
HNSNRQLERSGRFGG

SEQ ID NO: 93 - protein, 15 aa, Homo sapiens
NSNRQLERSGRFGGN

SEQ ID NO: 94 - protein, 15 aa, Homo sapiens
SNRQLERSGRFGGNP

SEQ ID NO: 95 - protein, 15 aa, Homo sapiens
NRQLERSGRFGGNPG

SEQ ID NO: 96 - protein, 15 aa, Homo sapiens
RQLERSGRFGGNPGG

SEQ ID NO: 97 - protein, 15 aa, Homo sapiens
QLERSGRFGGNPGGF

SEQ ID NO: 98 - protein, 15 aa, Homo sapiens
LERSGRFGGNPGGFG

SEQ ID NO: 99 - protein, 15 aa, Homo sapiens
ERSGRFGGNPGGFGN

SEQ ID NO: 100 - protein, 15 aa, Homo sapiens
RSGRFGGNPGGFGNQ

SEQ ID NO: 101 - protein, 15 aa, Homo sapiens
SGRFGGNPGGFGNQG

SEQ ID NO: 102 - protein, 15 aa, Homo sapiens
GRFGGNPGGFGNQGG

SEQ ID NO: 103 - protein, 15 aa, Homo sapiens
RFGGNPGGFGNQGGF

SEQ ID NO: 104 - protein, 15 aa, Homo sapiens
FGGNPGGFGNQGGFG

SEQ ID NO: 105 - protein, 15 aa, Homo sapiens
GGNPGGFGNQGGFGN

SEQ ID NO: 106 - protein, 15 aa, Homo sapiens
GNPGGFGNQGGFGNS

SEQ ID NO: 107 - protein, 15 aa, Homo sapiens
NPGGFGNQGGFGNSR

SEQ ID NO: 108 - protein, 15 aa, Homo sapiens
PGGFGNQGGFGNSRG

SEQ ID NO: 109 - protein, 15 aa, Homo sapiens
GGFGNQGGFGNSRGG

SEQ ID NO: 110 - protein, 15 aa, Homo sapiens
GFGNQGGFGNSRGGG

SEQ ID NO: 111 - protein, 15 aa, Homo sapiens
FGNQGGFGNSRGGGA

SEQ ID NO: 112 - protein, 15 aa, Homo sapiens
GNQGGFGNSRGGGAG

SEQ ID NO: 113 - protein, 15 aa, Homo sapiens
NQGGFGNSRGGGAGL

SEQ ID NO: 114 - protein, 15 aa, Homo sapiens
QGGFGNSRGGGAGLG

SEQ ID NO: 115 - protein, 15 aa, Homo sapiens
GGFGNSRGGGAGLGN

SEQ ID NO: 116 - protein, 16 aa, Homo sapiens
GGFGNSRGGGAGLGNN

SEQ ID NO: 117 - protein, 15 aa, Homo sapiens
FGNSRGGGAGLGNNQ

SEQ ID NO: 118 - protein, 15 aa, Homo sapiens
GNSRGGGAGLGNNQG

SEQ ID NO: 119 - protein, 15 aa, Homo sapiens
NSRGGGAGLGNNQGS

SEQ ID NO: 120 - protein, 15 aa, Homo sapiens
SRGGGAGLGNNQGSN

SEQ ID NO: 121 - protein, 15 aa, Homo sapiens
RGGGAGLGNNQGSNM

SEQ ID NO: 122 - protein, 15 aa, Homo sapiens
GGGAGLGNNQGSNMG

SEQ ID NO: 123 - protein, 15 aa, Homo sapiens
GGAGLGNNQGSNMGG

SEQ ID NO: 124 - protein, 15 aa, Homo sapiens
GAGLGNNQGSNMGGG

SEQ ID NO: 125 - protein, 15 aa, Homo sapiens
AGLGNNQGSNMGGGM

SEQ ID NO: 126 - protein, 15 aa, Homo sapiens
GLGNNQGSNMGGGMN

SEQ ID NO: 127 - protein, 15 aa, Homo sapiens
LGNNQGSNMGGGMNF

SEQ ID NO: 128 - protein, 15 aa, Homo sapiens
GNNQGSNMGGGMNFG

SEQ ID NO: 129 - protein, 15 aa, Homo sapiens
NNQGSNMGGGMNFGA

SEQ ID NO: 130 - protein, 15 aa, Homo sapiens
NQGSNMGGGMNFGAF

SEQ ID NO: 131 - protein, 15 aa, Homo sapiens
QGSNMGGGMNFGAFS

SEQ ID NO: 132 - protein, 15 aa, Homo sapiens
GSNMGGGMNFGAFSI

SEQ ID NO: 133 - protein, 15 aa, Homo sapiens
SNMGGGMNFGAFSIN

SEQ ID NO: 134 - protein, 15 aa, Homo sapiens
NMGGGMNFGAFSINP

SEQ ID NO: 135 - protein, 15 aa, Homo sapiens
MGGGMNFGAFSINPA

SEQ ID NO: 136 - protein, 15 aa, Homo sapiens
GGGMNFGAFSINPAM

SEQ ID NO: 137 - protein, 15 aa, Homo sapiens
GGMNFGAFSINPAMM

SEQ ID NO: 138 - protein, 15 aa, Homo sapiens
GMNFGAFSINPAMMA

SEQ ID NO: 139 - protein, 15 aa, Homo sapiens
MNFGAFSINPAMMAA

SEQ ID NO: 140 - protein, 15 aa, Homo sapiens
NFGAFSINPAMMAAA

SEQ ID NO: 141 - protein, 15 aa, Homo sapiens
FGAFSINPAMMAAAQ

SEQ ID NO: 142 - protein, 15 aa, Homo sapiens
GAFSINPAMMAAAQA

SEQ ID NO: 143 - protein, 15 aa, Homo sapiens
AFSINPAMMAAAQAA

SEQ ID NO: 144 - protein, 15 aa, Homo sapiens
FSINPAMMAAAQAAL

SEQ ID NO: 145 - protein, 15 aa, Homo sapiens
SINPAMMAAAQAALQ

SEQ ID NO: 146 - protein, 15 aa, Homo sapiens
INPAMMAAAQAALQS

SEQ ID NO: 147 - protein, 15 aa, Homo sapiens
NPAMMAAAQAALQSS

SEQ ID NO: 148 - protein, 15 aa, Homo sapiens
PAMMAAAQAALQSSW

SEQ ID NO: 149 - protein, 15 aa, Homo sapiens
AMMAAAQAALQSSWG

SEQ ID NO: 150 - protein, 15 aa, Homo sapiens
MMAAAQAALQSSWGM

SEQ ID NO: 151 - protein, 15 aa, Homo sapiens
MAAAQAALQSSWGMM

SEQ ID NO: 152 - protein, 15 aa, Homo sapiens
AAAQAALQSSWGMMG

SEQ ID NO: 153 - protein, 15 aa, Homo sapiens
AAQAALQSSWGMMGM

SEQ ID NO: 154 - protein, 15 aa, Homo sapiens
AQAALQSSWGMMGML

SEQ ID NO: 155 - protein, 15 aa, Homo sapiens
QAALQSSWGMMGMLA

SEQ ID NO: 156 - protein, 15 aa, Homo sapiens
AALQSSWGMMGMLAS

SEQ ID NO: 157 - protein, 15 aa, Homo sapiens
ALQSSWGMMGMLASQ

SEQ ID NO: 158 - protein, 15 aa, Homo sapiens
LQSSWGMMGMLASQQ

SEQ ID NO: 159 - protein, 15 aa, Homo sapiens
QSSWGMMGMLASQQN

SEQ ID NO: 160 - protein, 15 aa, Homo sapiens
SSWGMMGMLASQQNQ

SEQ ID NO: 161 - protein, 15 aa, Homo sapiens
SWGMMGMLASQQNQS

SEQ ID NO: 162 - protein, 15 aa, Homo sapiens
WGMMGMLASQQNQSG

SEQ ID NO: 163 - protein, 15 aa, Homo sapiens
GMMGMLASQQNQSGP

SEQ ID NO: 164 - protein, 15 aa, Homo sapiens
MMGMLASQQNQSGPS

SEQ ID NO: 165 - protein, 15 aa, Homo sapiens
MGMLASQQNQSGPSG

SEQ ID NO: 166 - protein, 15 aa, Homo sapiens
GMLASQQNQSGPSGN

SEQ ID NO: 167 - protein, 15 aa, Homo sapiens
MLASQQNQSGPSGNN

SEQ ID NO: 168 - protein, 15 aa, Homo sapiens
LASQQNQSGPSGNNQ

SEQ ID NO: 169 - protein, 15 aa, Homo sapiens
ASQQNQSGPSGNNQN

SEQ ID NO: 170 - protein, 15 aa, Homo sapiens
SQQNQSGPSGNNQNQ

SEQ ID NO: 171 - protein, 15 aa, Homo sapiens
QQNQSGPSGNNQNQG

SEQ ID NO: 172 - protein, 15 aa, Homo sapiens
QNQSGPSGNNQNQGN

SEQ ID NO: 173 - protein, 15 aa, Homo sapiens
NQSGPSGNNQNQGNM

SEQ ID NO: 174 - protein, 15 aa, Homo sapiens
QSGPSGNNQNQGNMQ

SEQ ID NO: 175 - protein, 15 aa, Homo sapiens
SGPSGNNQNQGNMQR

SEQ ID NO: 176 - protein, 15 aa, Homo sapiens
GPSGNNQNQGNMQRE

SEQ ID NO: 177 - protein, 15 aa, Homo sapiens
PSGNNQNQGNMQREP

SEQ ID NO: 178 - protein, 15 aa, Homo sapiens
SGNNQNQGNMQREPN

SEQ ID NO: 179 - protein, 15 aa, Homo sapiens
GNNQNQGNMQREPNQ

SEQ ID NO: 180 - protein, 15 aa, Homo sapiens
NNQNQGNMQREPNQA

SEQ ID NO: 181 - protein, 15 aa, Homo sapiens
NQNQGNMQREPNQAF

SEQ ID NO: 182 - protein, 15 aa, Homo sapiens
QNQGNMQREPNQAFG

SEQ ID NO: 183 - protein, 15 aa, Homo sapiens
NQGNMQREPNQAFGS

SEQ ID NO: 184 - protein, 15 aa, Homo sapiens
QGNMQREPNQAFGSG

SEQ ID NO: 185 - protein, 15 aa, Homo sapiens
GNMQREPNQAFGSGN

SEQ ID NO: 186 - protein, 15 aa, Homo sapiens
NMQREPNQAFGSGNN

SEQ ID NO: 187 - protein, 15 aa, Homo sapiens
MQREPNQAFGSGNNS

SEQ ID NO: 188 - protein, 15 aa, Homo sapiens
QREPNQAFGSGNNSY

SEQ ID NO: 189 - protein, 15 aa, Homo sapiens
REPNQAFGSGNNSYS

SEQ ID NO: 190 - protein, 15 aa, Homo sapiens
EPNQAFGSGNNSYSG

SEQ ID NO: 191 - protein, 15 aa, Homo sapiens
PNQAFGSGNNSYSGS

SEQ ID NO: 192 - protein, 15 aa, Homo sapiens
NQAFGSGNNSYSGSN

SEQ ID NO: 193 - protein, 15 aa, Homo sapiens
QAFGSGNNSYSGSNS

SEQ ID NO: 194 - protein, 15 aa, Homo sapiens
AFGSGNNSYSGSNSG

SEQ ID NO: 195 - protein, 15 aa, Homo sapiens
FGSGNNSYSGSNSGA

SEQ ID NO: 196 - protein, 15 aa, Homo sapiens
GSGNNSYSGSNSGAA

SEQ ID NO: 197 - protein, 15 aa, Homo sapiens
SGNNSYSGSNSGAAI

SEQ ID NO: 198 - protein, 15 aa, Homo sapiens
GNNSYSGSNSGAAIG

SEQ ID NO: 199 - protein, 15 aa, Homo sapiens
NNSYSGSNSGAAIGW

SEQ ID NO: 200 - protein, 15 aa, Homo sapiens
NSYSGSNSGAAIGWG

SEQ ID NO: 201 - protein, 15 aa, Homo sapiens
SYSGSNSGAAIGWGS

SEQ ID NO: 202 - protein, 15 aa, Homo sapiens
YSGSNSGAAIGWGSA

SEQ ID NO: 203 - protein, 15 aa, Homo sapiens
SGSNSGAAIGWGSAS

SEQ ID NO: 204 - protein, 15 aa, Homo sapiens
GSNSGAAIGWGSASN

SEQ ID NO: 205 - protein, 15 aa, Homo sapiens
SNSGAAIGWGSASNA

SEQ ID NO: 206 - protein, 15 aa, Homo sapiens
NSGAAIGWGSASNAG

SEQ ID NO: 207 - protein, 15 aa, Homo sapiens
SGAAIGWGSASNAGS

SEQ ID NO: 208 - protein, 15 aa, Homo sapiens
GAAIGWGSASNAGSG

SEQ ID NO: 209 - protein, 15 aa, Homo sapiens
AAIGWGSASNAGSGS

SEQ ID NO: 210 - protein, 15 aa, Homo sapiens
AIGWGSASNAGSGSG

SEQ ID NO: 211 - protein, 15 aa, Homo sapiens
IGWGSASNAGSGSGF

SEQ ID NO: 212 - protein, 15 aa, Homo sapiens
GWGSASNAGSGSGFN

SEQ ID NO: 213 - protein, 15 aa, Homo sapiens
WGSASNAGSGSGFNG

SEQ ID NO: 214 - protein, 15 aa, Homo sapiens
GSASNAGSGSGFNGG

SEQ ID NO: 215 - protein, 15 aa, Homo sapiens
SASNAGSGSGFNGGF

SEQ ID NO: 216 - protein, 15 aa, Homo sapiens
ASNAGSGSGFNGGFG

SEQ ID NO: 217 - protein, 15 aa, Homo sapiens
SNAGSGSGFNGGFGS

SEQ ID NO: 218 - protein, 15 aa, Homo sapiens
NAGSGSGFNGGFGSS

SEQ ID NO: 219 - protein, 15 aa, Homo sapiens
AGSGSGFNGGFGSSM

SEQ ID NO: 220 - protein, 15 aa, Homo sapiens
GSGSGFNGGFGSSMD

SEQ ID NO: 221 - protein, 15 aa, Homo sapiens
SGSGFNGGFGSSMDS

SEQ ID NO: 222 - protein, 15 aa, Homo sapiens
GSGFNGGFGSSMDSK

SEQ ID NO: 223 - protein, 15 aa, Homo sapiens
SGFNGGFGSSMDSKS

SEQ ID NO: 224 - protein, 15 aa, Homo sapiens
GFNGGFGSSMDSKSS

SEQ ID NO: 225 - protein, 15 aa, Homo sapiens
FNGGFGSSMDSKSSG

SEQ ID NO: 226 - protein, 15 aa, Homo sapiens
NGGFGSSMDSKSSGW

SEQ ID NO: 227 - protein, 15 aa, Homo sapiens
GGFGSSMDSKSSGWG

SEQ ID NO: 228 - protein, 15 aa, Homo sapiens
GFGSSMDSKSSGWGM

SEQ ID NO: 229 - protein, 15 aa, Homo sapiens
EDDGTVLLSTVTAQF

SEQ ID NO: 230 - protein, 15 aa, Homo sapiens
VLLSTVTAQFPGACG

SEQ ID NO: 231 - protein, 15 aa, Homo sapiens
MRGVRLVEGILHAPD

SEQ ID NO: 232 - protein, 15 aa, Homo sapiens
AGWGNLVYVVNYPKD

SEQ ID NO: 233 - protein, 15 aa, Homo sapiens
LVYVVNYPKDNKRKM

SEQ ID NO: 234 - protein, 15 aa, Homo sapiens
TFGEVLMVQVKKDLK

SEQ ID NO: 235 - protein, 15 aa, Homo sapiens
ETQVKVMSQRHMIDG

SEQ ID NO: 236 - protein, 15 aa, Homo sapiens
DMTEDELREFFSQYG

SEQ ID NO: 237 - protein, 15 aa, Homo sapiens
ELREFFSQYGDVMDV

SEQ ID NO: 238 - protein, 15 aa, Homo sapiens
DVMDVFIPKPFRAFA

SEQ ID NO: 239 - protein, 15 aa, Homo sapiens
FIPKPFRAFAFVTFA

SEQ ID NO: 240 - protein, 15 aa, Homo sapiens
NATRIPAKTPPAPKT

SEQ ID NO: 241 - protein, 15 aa, Homo sapiens
ATRIPAKTPPAPKTP

SEQ ID NO: 242 - protein, 15 aa, Homo sapiens
TRIPAKTPPAPKTPP

SEQ ID NO: 243 - protein, 15 aa, Homo sapiens
RIPAKTPPAPKTPPS

SEQ ID NO: 244 - protein, 15 aa, Homo sapiens
IPAKTPPAPKTPPSS

SEQ ID NO: 245 - protein, 15 aa, Homo sapiens
PAKTPPAPKTPPSSG

SEQ ID NO: 246 - protein, 15 aa, Homo sapiens
AGIGDTPSLEDEAAG

SEQ ID NO: 247 - protein, 15 aa, Homo sapiens
AKTPPAPKTPPSSGE

SEQ ID NO: 248 - protein, 15 aa, Homo sapiens
KTPPAPKTPPSSGEP

SEQ ID NO: 249 - protein, 15 aa, Homo sapiens
TPPAPKTPPSSGEPP

SEQ ID NO: 250 - protein, 15 aa, Homo sapiens
PPAPKTPPSSGEPPK

SEQ ID NO: 251 - protein, 15 aa, Homo sapiens
PAPKTPPSSGEPPKS

SEQ ID NO: 252 - protein, 15 aa, Homo sapiens
APKTPPSSGEPPKSG

SEQ ID NO: 253 - protein, 15 aa, Homo sapiens
PKTPPSSGEPPKSGD

SEQ ID NO: 254 - protein, 15 aa, Homo sapiens
KTPPSSGEPPKSGDR

SEQ ID NO: 255 - protein, 15 aa, Homo sapiens
TPPSSGEPPKSGDRS

SEQ ID NO: 256 - protein, 15 aa, Homo sapiens
SSGEPPKSGDRSGYS

SEQ ID NO: 257 - protein, 15 aa, Homo sapiens
SGEPPKSGDRSGYSS

SEQ ID NO: 258 - protein, 15 aa, Homo sapiens
GEPPKSGDRSGYSSP

SEQ ID NO: 259 - protein, 15 aa, Homo sapiens
EPPKSGDRSGYSSPG

SEQ ID NO: 260 - protein, 15 aa, Homo sapiens
PPKSGDRSGYSSPGS

SEQ ID NO: 261 - protein, 15 aa, Homo sapiens
PKSGDRSGYSSPGSP

SEQ ID NO: 262 - protein, 15 aa, Homo sapiens
KSGDRSGYSSPGSPG

SEQ ID NO: 263 - protein, 15 aa, Homo sapiens
SGDRSGYSSPGSPGT

SEQ ID NO: 264 - protein, 15 aa, Homo sapiens
GDRSGYSSPGSPGTP

SEQ ID NO: 265 - protein, 15 aa, Homo sapiens
DRSGYSSPGSPGTPG

SEQ ID NO: 266 - protein, 15 aa, Homo sapiens
RSGYSSPGSPGTPGS

SEQ ID NO: 267 - protein, 15 aa, Homo sapiens
SGYSSPGSPGTPGSR

SEQ ID NO: 268 - protein, 15 aa, Homo sapiens
GYSSPGSPGTPGSRS

SEQ ID NO: 269 - protein, 15 aa, Homo sapiens
YSSPGSPGTPGSRSR

SEQ ID NO: 270 - protein, 15 aa, Homo sapiens
SSPGSPGTPGSRSRT

SEQ ID NO: 271 - protein, 15 aa, Homo sapiens
SPGSPGTPGSRSRTP

SEQ ID NO: 272 - protein, 15 aa, Homo sapiens
PGSPGTPGSRSRTPS

SEQ ID NO: 273 - protein, 15 aa, Homo sapiens
GSPGTPGSRSRTPSL

SEQ ID NO: 274 - protein, 15 aa, Homo sapiens
SPGTPGSRSRTPSLP

SEQ ID NO: 275 - protein, 15 aa, Homo sapiens
PGTPGSRSRTPSLPT

SEQ ID NO: 276 - protein, 15 aa, Homo sapiens
GTPGSRSRTPSLPTP

SEQ ID NO: 277 - protein, 15 aa, Homo sapiens
TPGSRSRTPSLPTPP

SEQ ID NO: 278 - protein, 15 aa, Homo sapiens
PGSRSRTPSLPTPPT

SEQ ID NO: 279 - protein, 15 aa, Homo sapiens
GSRSRTPSLPTPPTR

SEQ ID NO: 280 - protein, 15 aa, Homo sapiens
SRSRTPSLPTPPTRE

SEQ ID NO: 281 - protein, 15 aa, Homo sapiens
RSRTPSLPTPPTREP

SEQ ID NO: 282 - protein, 15 aa, Homo sapiens
SRTPSLPTPPTREPK

SEQ ID NO: 283 - protein, 15 aa, Homo sapiens
RTPSLPTPPTREPKK

SEQ ID NO: 284 - protein, 15 aa, Homo sapiens
TPSLPTPPTREPKKV

SEQ ID NO: 285 - protein, 15 aa, Homo sapiens
PSLPTPPTREPKKVA

SEQ ID NO: 286 - protein, 15 aa, Homo sapiens
SLPTPPTREPKKVAV

SEQ ID NO: 287 - protein, 15 aa, Homo sapiens
TPPTREPKKVAVVRT

SEQ ID NO: 288 - protein, 15 aa, Homo sapiens
PPTREPKKVAVVRTP

SEQ ID NO: 289 - protein, 15 aa, Homo sapiens
PTREPKKVAVVRTPP

SEQ ID NO: 290 - protein, 15 aa, Homo sapiens
TREPKKVAVVRTPPK

SEQ ID NO: 291 - protein, 15 aa, Homo sapiens
REPKKVAVVRTPPKS

SEQ ID NO: 292 - protein, 15 aa, Homo sapiens
EPKKVAVVRTPPKSP

SEQ ID NO: 293 - protein, 15 aa, Homo sapiens
PKKVAVVRTPPKSPS

SEQ ID NO: 294 - protein, 15 aa, Homo sapiens
KKVAVVRTPPKSPSS

SEQ ID NO: 295 - protein, 15 aa, Homo sapiens
KVAVVRTPPKSPSSA

SEQ ID NO: 296 - protein, 15 aa, Homo sapiens
VAVVRTPPKSPSSAK

SEQ ID NO: 297 - protein, 15 aa, Homo sapiens
AVVRTPPKSPSSAKS

SEQ ID NO: 298 - protein, 15 aa, Homo sapiens
VVRTPPKSPSSAKSR

SEQ ID NO: 299 - protein, 15 aa, Homo sapiens
VRTPPKSPSSAKSRL

SEQ ID NO: 300 - protein, 15 aa, Homo sapiens
RTPPKSPSSAKSRLQ

SEQ ID NO: 301 - protein, 15 aa, Homo sapiens
TPPKSPSSAKSRLQT

SEQ ID NO: 302 - protein, 15 aa, Homo sapiens
VPMPDLKNVKSKIGS

SEQ ID NO: 303 - protein, 15 aa, Homo sapiens
PMPDLKNVKSKIGST

SEQ ID NO: 304 - protein, 15 aa, Homo sapiens
MPDLKNVKSKIGSTE

SEQ ID NO: 305 - protein, 15 aa, Homo sapiens
PDLKNVKSKIGSTEN

SEQ ID NO: 306 - protein, 15 aa, Homo sapiens
DLKNVKSKIGSTENL

SEQ ID NO: 307 - protein, 15 aa, Homo sapiens
LKNVKSKIGSTENLK

SEQ ID NO: 308 - protein, 15 aa, Homo sapiens
KNVKSKIGSTENLKH

SEQ ID NO: 309 - protein, 15 aa, Homo sapiens
NVKSKIGSTENLKHQ

SEQ ID NO: 310 - protein, 15 aa, Homo sapiens
VKSKIGSTENLKHQP

SEQ ID NO: 311 - protein, 15 aa, Homo sapiens
KSKIGSTENLKHQPG

SEQ ID NO: 312 - protein, 15 aa, Homo sapiens
SKIGSTENLKHQPGG

SEQ ID NO: 313 - protein, 15 aa, Homo sapiens
KIGSTENLKHQPGGG

SEQ ID NO: 314 - protein, 15 aa, Homo sapiens
IGSTENLKHQPGGGK

SEQ ID NO: 315 - protein, 15 aa, Homo sapiens
GSTENLKHQPGGGKV

SEQ ID NO: 316 - protein, 15 aa, Homo sapiens
STENLKHQPGGGKVQ

SEQ ID NO: 317 - protein, 15 aa, Homo sapiens
EKLDFKDRVQSKIGS

SEQ ID NO: 318 - protein, 15 aa, Homo sapiens
KLDFKDRVQSKIGSL

SEQ ID NO: 319 - protein, 15 aa, Homo sapiens
DFKDRVQSKIGSLDN

SEQ ID NO: 320 - protein, 15 aa, Homo sapiens
FKDRVQSKIGSLDNI

SEQ ID NO: 321 - protein, 15 aa, Homo sapiens
KDRVQSKIGSLDNIT

SEQ ID NO: 322 - protein, 15 aa, Homo sapiens
DRVQSKIGSLDNITH

SEQ ID NO: 323 - protein, 15 aa, Homo sapiens
RVQSKIGSLDNITHV

SEQ ID NO: 324 - protein, 15 aa, Homo sapiens
VQSKIGSLDNITHVP

SEQ ID NO: 325 - protein, 15 aa, Homo sapiens
QSKIGSLDNITHVPG

SEQ ID NO: 326 - protein, 15 aa, Homo sapiens
SKIGSLDNITHVPGG

SEQ ID NO: 327 - protein, 15 aa, Homo sapiens
KIGSLDNITHVPGGG

SEQ ID NO: 328 - protein, 15 aa, Homo sapiens
IGSLDNITHVPGGGN

SEQ ID NO: 329 - protein, 15 aa, Homo sapiens
GSLDNITHVPGGGNK

SEQ ID NO: 330 - protein, 15 aa, Homo sapiens
SLDNITHVPGGGNKK

SEQ ID NO: 331 - protein, 15 aa, Homo sapiens
AKAKTDHGAEIVYKS

SEQ ID NO: 332 - protein, 15 aa, Homo sapiens
KAKTDHGAEIVYKSP

SEQ ID NO: 333 - protein, 15 aa, Homo sapiens
AKTDHGAEIVYKSPV

SEQ ID NO: 334 - protein, 15 aa, Homo sapiens
KTDHGAEIVYKSPVV

SEQ ID NO: 335 - protein, 15 aa, Homo sapiens
TDHGAEIVYKSPVVS

SEQ ID NO: 336 - protein, 15 aa, Homo sapiens
DHGAEIVYKSPVVSG

SEQ ID NO: 337 - protein, 15 aa, Homo sapiens
HGAEIVYKSPVVSGD

SEQ ID NO: 338 - protein, 15 aa, Homo sapiens
GAEIVYKSPVVSGDT

SEQ ID NO: 339 - protein, 15 aa, Homo sapiens
AEIVYKSPVVSGDTS

SEQ ID NO: 340 - protein, 15 aa, Homo sapiens
EIVYKSPVVSGDTSP

SEQ ID NO: 341 - protein, 15 aa, Homo sapiens
IVYKSPVVSGDTSPR

SEQ ID NO: 342 - protein, 15 aa, Homo sapiens
VYKSPVVSGDTSPRH

SEQ ID NO: 343 - protein, 15 aa, Homo sapiens
YKSPVVSGDTSPRHL

SEQ ID NO: 344 - protein, 15 aa, Homo sapiens
KSPVVSGDTSPRHLS

SEQ ID NO: 345 - protein, 15 aa, Homo sapiens
SPVVSGDTSPRHLSN

SEQ ID NO: 346 - protein, 15 aa, Homo sapiens
PVVSGDTSPRHLSNV

SEQ ID NO: 347 - protein, 15 aa, Homo sapiens
VVSGDTSPRHLSNVS

SEQ ID NO: 348 - protein, 15 aa, Homo sapiens
VSGDTSPRHLSNVSS

SEQ ID NO: 349 - protein, 15 aa, Homo sapiens
SGDTSPRHLSNVSST

SEQ ID NO: 350 - protein, 15 aa, Homo sapiens
GDTSPRHLSNVSSTG

SEQ ID NO: 351 - protein, 15 aa, Homo sapiens
DTSPRHLSNVSSTGS

SEQ ID NO: 352 - protein, 15 aa, Homo sapiens
TSPRHLSNVSSTGSI

SEQ ID NO: 353 - protein, 15 aa, Homo sapiens
SPRHLSNVSSTGSID

SEQ ID NO: 354 - protein, 15 aa, Homo sapiens
PRHLSNVSSTGSIDM

SEQ ID NO: 355 - protein, 15 aa, Homo sapiens
RHLSNVSSTGSIDMV

SEQ ID NO: 356 - protein, 15 aa, Homo sapiens
HLSNVSSTGSIDMVD

SEQ ID NO: 357 - protein, 15 aa, Homo sapiens
LSNVSSTGSIDMVDS

SEQ ID NO: 358 - protein, 15 aa, Homo sapiens
SNVSSTGSIDMVDSP

SEQ ID NO: 359 - protein, 15 aa, Homo sapiens
NVSSTGSIDMVDSPQ

SEQ ID NO: 360 - protein, 15 aa, Homo sapiens
VSSTGSIDMVDSPQL

SEQ ID NO: 361 - protein, 15 aa, Homo sapiens
SSTGSIDMVDSPQLA

SEQ ID NO: 362 - protein, 15 aa, Homo sapiens
STGSIDMVDSPQLAT

SEQ ID NO: 363 - protein, 15 aa, Homo sapiens
TGSIDMVDSPQLATL

SEQ ID NO: 364 - protein, 15 aa, Homo sapiens
GSIDMVDSPQLATLA

SEQ ID NO: 365 - protein, 15 aa, Homo sapiens
SIDMVDSPQLATLAD

SEQ ID NO: 366 - protein, 15 aa, Homo sapiens
IDMVDSPQLATLADE

SEQ ID NO: 367 - protein, 15 aa, Homo sapiens
DMVDSPQLATLADEV

SEQ ID NO: 368 - protein, 15 aa, Homo sapiens
MVDSPQLATLADEVS

SEQ ID NO: 369 - protein, 15 aa, Homo sapiens
VDSPQLATLADEVSA

SEQ ID NO: 370 - protein, 15 aa, Homo sapiens
DSPQLATLADEVSAS

SEQ ID NO: 371 - protein, 15 aa, Homo sapiens
SPQLATLADEVSASL

SEQ ID NO: 372 - protein, 16 aa, Homo sapiens
KVAVVRTPPKSPSSAK

SEQ ID NO: 373 - protein, 16 aa, Homo sapiens
PAPKTPPSSGEPPKSG

SEQ ID NO: 374 - protein, 16 aa, Homo sapiens
PMPDLKNVKSKIGSTE

SEQ ID NO: 375 - protein, 16 aa, Homo sapiens
DFKDRVQSKIGSLDNI

SEQ ID NO: 376 - protein, 16 aa, Homo sapiens
DRSGYSSPGSPGTPGS

SEQ ID NO: 377 - protein, 9 aa, Homo sapiens
VFMKGLSKA

SEQ ID NO: 378 - protein, 10 aa, Homo sapiens
DVFMKGLSKA

SEQ ID NO: 379 - protein, 9 aa, Homo sapiens
GWAAAEKTK

SEQ ID NO: 380 - protein, 15 aa, Homo sapiens
VAAAEKTKQGVAEAP

SEQ ID NO: 381 - protein, 15 aa, Homo sapiens
VAAAEKTKQGVAEAA

SEQ ID NO: 382 - protein, 9 aa, Homo sapiens
AGKTKEGVL

SEQ ID NO: 383 - protein, 9 aa, Homo sapiens
PGKTKEGVL

SEQ ID NO: 384 - protein, 10 aa, Homo sapiens
AGKTKEGVLY

SEQ ID NO: 385 - protein, 10 aa, Homo sapiens
APGKTKEGVL

SEQ ID NO: 386 - protein, 10 aa, Homo sapiens
GVAEAAGKTK

SEQ ID NO: 387 - protein, 15 aa, Homo sapiens
KQGVAEAPGKTKEGV

SEQ ID NO: 388 - protein, 15 aa, Homo sapiens
PGKTKEGVLYVGSKT

SEQ ID NO: 389 - protein, 15 aa, Homo sapiens
KTKEGVLYVGSKTKK

SEQ ID NO: 390 - protein, 15 aa, Homo sapiens
KQGVAEAAGKTKEGV

SEQ ID NO: 391 - protein, 15 aa, Homo sapiens
AGKTKEGVLYVGSKT

SEQ ID NO: 392 - protein, 9 aa, Homo sapiens
VLYVGSKTK

SEQ ID NO: 393 - protein, 9 aa, Homo sapiens
LYVGSKTKK

SEQ ID NO: 394 - protein, 10 aa, Homo sapiens
YVGSKTKEGV

SEQ ID NO: 395 - protein, 9 aa, Homo sapiens
VLYVGSKTK

SEQ ID NO: 396 - protein, 10 aa, Homo sapiens
GVLYVGSKTK

SEQ ID NO: 397 - protein, 10 aa, Homo sapiens
LYVGSKTKEG

SEQ ID NO: 398 - protein, 9 aa, Homo sapiens
KTKKGWHGV

SEQ ID NO: 399 - protein, 8 aa, Homo sapiens
KTKKGWHG

SEQ ID NO: 400 - protein, 14 aa, Homo sapiens
YVGSKTKKGWHGVA

SEQ ID NO: 401 - protein, 15 aa, Homo sapiens
KTKEGVLYVGSKTKE

SEQ ID NO: 402 - protein, 8 aa, Homo sapiens
VTNVGGAW

SEQ ID NO: 403 - protein, 8 aa, Homo sapiens
GWHGVTTV

SEQ ID NO: 404 - protein, 9 aa, Homo sapiens
EEGAPQEGI

SEQ ID NO: 405 - protein, 10 aa, Homo sapiens
GSIAAATGFV

SEQ ID NO: 406 - protein, 10 aa, Homo sapiens
SIAAATGFVK

SEQ ID NO: 407 - protein, 10 aa, Homo sapiens
AGSIAAATGF

SEQ ID NO: 408 - protein, 9 aa, Homo sapiens
IAAATGFVK

SEQ ID NO: 409 - protein, 10 aa, Homo sapiens
APQEGILEDM

SEQ ID NO: 410 - protein, 10 aa, Homo sapiens
EEGAPQEGIL

SEQ ID NO: 411 - protein, 9 aa, Homo sapiens
VFMGLSKAK

SEQ ID NO: 412 - protein, 10 aa, Homo sapiens
AEAAGKTKEG

SEQ ID NO: 413 - protein, 15 aa, Homo sapiens
YVGSKTKEGVVHGVT

SEQ ID NO: 414 - protein, 9 aa, Homo sapiens
IAAATGFVK

SEQ ID NO: 415 - protein, 17 aa, Homo sapiens
DNEAYEMPSEEGYQDYH

SEQ ID NO: 416 - protein, 9 aa, Homo sapiens
PSEEGYQDY

SEQ ID NO: 417 - protein, 9 aa, Homo sapiens
YEMPSEEGY

SEQ ID NO: 418 - protein, 9 aa, Homo sapiens
MPSEEGYQD

SEQ ID NO: 419 - protein, 10 aa, Homo sapiens
AYEMPSEEGY

SEQ ID NO: 420 - protein, 10 aa, Homo sapiens
MPSEEGYQDY

SEQ ID NO: 421 - protein, 10 aa, Homo sapiens
EMPSEEGYQD

SEQ ID NO: 422 - protein, 10 aa, Homo sapiens
DNEAYEMPSE

SEQ ID NO: 423 - protein, 10 aa, Homo sapiens
YEMPSEEGYQ

SEQ ID NO: 424 - protein, 10 aa, Homo sapiens
SEEGYQDYEP

SEQ ID NO: 425 - protein, 19 aa, Homo sapiens
NTQTDRESLRNLRCYYNQS

SEQ ID NO: 426 - protein, 15 aa, Homo sapiens
GKTKEGVLYVGSKTK

SEQ ID NO: 427 - protein, 18 aa, Homo sapiens
YWDLQTRNVKAHSQTDRA

SEQ ID NO: 428 - protein, 23 aa, Homo sapiens
RNTQIFKTNTQTHRENLRIALRY

SEQ ID NO: 429 - protein, 16 aa, Homo sapiens
FLPTGGKGGSCSQAAS

SEQ ID NO: 430 - protein, 18 aa, Homo sapiens
QRMEPRAPWIEQERPAYW

SEQ ID NO: 431 - protein, 28 aa, Homo sapiens
VYSRHPAENGKSNFLNCYVSGFHPSDIE

SEQ ID NO: 432 - protein, 16 aa, Homo sapiens
PDGRLLRGYQQDAYDG

SEQ ID NO: 433 - protein, 15 aa, Homo sapiens
GHVRGVAPQIPGERE

SEQ ID NO: 434 - protein, 24 aa, Homo sapiens
HQAQVGGGPCGGAVESLPGGHVRG

SEQ ID NO: 435 - protein, 16 aa, Homo sapiens
FLPTGGKGGSCSQAAS

SEQ ID NO: 436 - protein, 21 aa, Homo sapiens
RKLEAAGVAEQLRAYLEGECV

SEQ ID NO: 437 - protein, 10 aa, Homo sapiens
DVGSDGRFLR

SEQ ID NO: 438 - protein, 19 aa, Homo sapiens
NTQTDRESLRNLRCYYNQS

SEQ ID NO: 439 - protein, 17 aa, Homo sapiens
SLPGGRVRGVAPQIPGE

SEQ ID NO: 440 - protein, 26 aa, Homo sapiens
KWEAARVAEQLRAYLEGLCVEWLRRH

SEQ ID NO: 441 - protein, 13 aa, Homo sapiens
DRETRDLTGNGKD

SEQ ID NO: 442 - protein, 21 aa, Homo sapiens
LSSWTAADTAAQITQRKLEAA

SEQ ID NO: 443 - protein, 23 aa, Homo sapiens
ILRWEPSSQPTIPIVGIIAGLVL

SEQ ID NO: 444 - protein, 15 aa, Homo sapiens
GHVRGVAPQIPGERE

SEQ ID NO: 445 - protein, 24 aa, Homo sapiens
HQAQVGGGPCGGAVESLPGGHVRG

SEQ ID NO: 446 - protein, 16 aa, Homo sapiens
FLPTGGKGGSCSQAAS

SEQ ID NO: 447 - protein, 24 aa, Homo sapiens
FLPTGGKGGSCSQAASSNSAQGSD

SEQ ID NO: 448 - protein, 9 aa, Homo sapiens
KTKQGVAEA

SEQ ID NO: 449 - protein, 9 aa, Homo sapiens
DVFMKGLSK

SEQ ID NO: 450 - protein, 9 aa, Homo sapiens
FMKGLSKAK

SEQ ID NO: 451 - protein, 10 aa, Homo sapiens
FMKGLSKAKE

SEQ ID NO: 452 - protein, 10 aa, Homo sapiens
KTKQGVAEAA

SEQ ID NO: 453 - protein, 10 aa, Homo sapiens
KAKEGVVAAA

SEQ ID NO: 454 - protein, 9 aa, Homo sapiens
KAKEGVVAA

SEQ ID NO: 455 - protein, 9 aa, Homo sapiens
KTKEGVLYV

SEQ ID NO: 456 - protein, 9 aa, Homo sapiens
APGKTKEGV

SEQ ID NO: 457 - protein, 10 aa, Homo sapiens
GVAEAPGKTK

SEQ ID NO: 458 - protein, 9 aa, Homo sapiens
KTKEGVVHG

SEQ ID NO: 459 - protein, 15 aa, Homo sapiens
YVGSKTKEGVVHGVA

SEQ ID NO: 460 - protein, 9 aa, Homo sapiens
AVVTGVTAV

SEQ ID NO: 461 - protein, 9 aa, Homo sapiens
QVTNVGGAV

SEQ ID NO: 462 - protein, 9 aa, Homo sapiens
KTKEQVTNV

SEQ ID NO: 463 - protein, 10 aa, Homo sapiens
NVGGAVVTGV

SEQ ID NO: 464 - protein, 10 aa, Homo sapiens
GAVVTGVTAV

SEQ ID NO: 465 - protein, 10 aa, Homo sapiens
GVTAVAQKTV

SEQ ID NO: 466 - protein, 10 aa, Homo sapiens
EQVTNVGGAV

SEQ ID NO: 467 - protein, 10 aa, Homo sapiens
VATVAEKTKE

SEQ ID NO: 468 - protein, 9 aa, Homo sapiens
GVVHGVATV

SEQ ID NO: 469 - protein, 15 aa, Homo sapiens
EGVVHGVTTVAEKTK

SEQ ID NO: 470 - protein, 15 aa, Homo sapiens
TTVAEKTKEQVTNVG

SEQ ID NO: 471 - protein, 15 aa, Homo sapiens
KGVVHGVATVAEKTK

SEQ ID NO: 472 - protein, 15 aa, Homo sapiens
EGVVHGVATVAEKTK

SEQ ID NO: 473 - protein, 15 aa, Homo sapiens
ATVAEKTKEQVTNVG

SEQ ID NO: 474 - protein, 9 aa, Homo sapiens
SIAAATGFV

SEQ ID NO: 475 - protein, 9 aa, Homo sapiens
AAATGFVKK

SEQ ID NO: 476 - protein, 9 aa, Homo sapiens
KTVEGAGSI

SEQ ID NO: 477 - protein, 9 aa, Homo sapiens
GSIAAATGF

SEQ ID NO: 478 - protein, 10 aa, Homo sapiens
NEEGAPQEGI

SEQ ID NO: 479 - protein, 10 aa, Homo sapiens
AATGFVKKDQ

SEQ ID NO: 480 - protein, 9 aa, Homo sapiens
FVKKDQLGK

SEQ ID NO: 481 - protein, 9 aa, Homo sapiens
PVDPDNEAY

SEQ ID NO: 482 - protein, 9 aa, Homo sapiens
MPVDPDNEA

SEQ ID NO: 483 - protein, 9 aa, Homo sapiens
DPDNEAYEM

SEQ ID NO: 484 - protein, 10 aa, Homo sapiens
EGILEDMPVD

SEQ ID NO: 485 - protein, 10 aa, Homo sapiens
MPVDPDNEAY

SEQ ID NO: 486 - protein, 10 aa, Homo sapiens
QEGILEDMPV

SEQ ID NO: 487 - protein, 15 aa, Homo sapiens
GKTKEGVLYVGSKTK

SEQ ID NO: 488 - protein, 15 aa, Homo sapiens
KTKEGVLYVGSKTKE

SEQ ID NO: 489 - protein, 15 aa, Homo sapiens
MPVDPDNEAYEMPSE

SEQ ID NO: 490 - protein, 15 aa, Homo sapiens
DNEAYEMPSEEGYQD

SEQ ID NO: 491 - protein, 15 aa, Homo sapiens
EMPSEEGYQDYEPEA

SEQ ID NO: 492 - protein, 12 aa, Homo sapiens
SEEGYQDYEPEA

SEQ ID NO: 493 - protein, 10 aa, Homo sapiens
GVLYVGSKTK

SEQ ID NO: 494 - protein, 9 aa, Homo sapiens
VLYVGSKTK

SEQ ID NO: 495 - protein, 10 aa, Homo sapiens
VLYVGSKTKK

SEQ ID NO: 496 - protein, 15 aa, Homo sapiens
MDVFMKGLSKAKEGV

SEQ ID NO: 497 - protein, 15 aa, Homo sapiens
KGLSKAKEGVVAAAE

SEQ ID NO: 498 - protein, 15 aa, Homo sapiens
AKEGVVAAAEKTKQG

SEQ ID NO: 499 - protein, 15 aa, Homo sapiens
VAAAEKTKQGVAEAA

SEQ ID NO: 500 - protein, 15 aa, Homo sapiens
KTKQGVAEAAGKTKE

SEQ ID NO: 501 - protein, 15 aa, Homo sapiens
KQGVAEAAGKTKEGV

SEQ ID NO: 502 - protein, 15 aa, Homo sapiens
VAEAAGKTKEGVLYV

SEQ ID NO: 503 - protein, 15 aa, Homo sapiens
AGKTKEGVLYVGSKT

SEQ ID NO: 504 - protein, 15 aa, Homo sapiens
GVLYVGSKTKEGVVH

SEQ ID NO: 505 - protein, 15 aa, Homo sapiens
YVGSKTKEGVVHGVA

SEQ ID NO: 506 - protein, 15 aa, Homo sapiens
GSKTKEGVVHGVATV

SEQ ID NO: 507 - protein, 15 aa, Homo sapiens
EGVVHGVATVAEKTK

SEQ ID NO: 508 - protein, 15 aa, Homo sapiens
GVATVAEKTKEQVTN

SEQ ID NO: 509 - protein, 15 aa, Homo sapiens
ATVAEKTKEQVTNVG

SEQ ID NO: 510 - protein, 15 aa, Homo sapiens
AEKTKEQVTNVGGAV

SEQ ID NO: 511 - protein, 15 aa, Homo sapiens
EQVTNVGGAVVTGVT

SEQ ID NO: 512 - protein, 15 aa, Homo sapiens
VGGAVVTGVTAVAQK

SEQ ID NO: 513 - protein, 15 aa, Homo sapiens
VTGVTAVAQKTVEGA

SEQ ID NO: 514 - protein, 15 aa, Homo sapiens
AVAQKTVEGAGSIAA

SEQ ID NO: 515 - protein, 15 aa, Homo sapiens
TVEGAGSIAAATGFV

SEQ ID NO: 516 - protein, 15 aa, Homo sapiens
GSIAAATGFVKKDQL

SEQ ID NO: 517 - protein, 15 aa, Homo sapiens
ATGFVKKDQLGKNEE

SEQ ID NO: 518 - protein, 15 aa, Homo sapiens
KKDQLGKNEEGAPQE

SEQ ID NO: 519 - protein, 15 aa, Homo sapiens
GKNEEGAPQEGILED

SEQ ID NO: 520 - protein, 15 aa, Homo sapiens
GAPQEGILEDMPVDP

SEQ ID NO: 521 - protein, 15 aa, Homo sapiens
GILEDMPVDPDNEAY

SEQ ID NO: 522 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 523 - DNA, 1108 bp, Homo sapiens
ccgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctgg

SEQ ID NO: 524 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 525 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 526 - protein, 526 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 527 - protein, 154 aa, Homo sapiens [sequence omitted]

SEQ ID NO: 528 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 529 - protein, 112 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKEGYQDYEPEA

SEQ ID NO: 530 - DNA, 3215 bp, Homo sapiens
aggagaaggagaaggaggaggactaggaggaggaggacggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 531 - DNA, 3127 bp, Homo sapiens
gccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaagagagaggcggggaggagtcggagttgtggagaagcagagggactcagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 532 - protein, 2527 aa, Homo sapiens
MASGSCQGCEEDEETLKKLIVRLNNVQEGKQIETLVQILEDLLVFTYSEHASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 533 - DNA, 9239 bp, Homo sapiens
gcgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcacgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaaaactttaaaaaaaaaaaaaaaaa

SEQ ID NO: 534 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 535 - DNA, 2259 bp, Homo sapiens
agctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggcggtgcccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccc

SEQ ID NO: 536 - protein, 266 aa, Homo sapiens
MVCLKLPGGSYMAKLTVTLMVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQSKMLSGVGGFVLGLLFLGAGLFIYFKNQKGHSGLHPTGLVS

SEQ ID NO: 537 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 538 - protein, 261 aa, Homo sapiens
MSWKKALRIPGGLRAATVTLMLAMLSTPVAEGRDSPEDFVYQFKAMCYFTNGTERVRYVTRYIYNREEYARFDSDVEVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQHGDVYTCHVEHPSLQNPITVEWRAQSESAQSKMLSGIGGFVLGLIFLGLGLIIHHRSQKGLLH

SEQ ID NO: 539 - protein, 365 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 540 - protein, 266 aa, Homo sapiens
MVCLKLPGGSCMAALTVTLMVLSSPLALAGDTQPRFLWQGKYKCHFFNGTERVQFLERLFYNQEEFVRFDSDVGEYRAVTELGRPVAESWNSQKDILEDRRGQVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSKMLSGVGGFVLGLLFLGAGLFIYFRNQKGHSGLQPTGFLS

SEQ ID NO: 541 - protein, 266 aa, Homo sapiens [sequence omitted]

SEQ ID NO: 542 - protein, 261 aa, Homo sapiens; identical to SEQ ID NO: 538

(19) United States
(12) Patent Application Publication
(10) Pub. No.: US 2020/0095296 A1
(43) Pub. Date: Mar. 26, 2020

(54) USE OF PEPTIDES AS BIOMARKERS IN THE DIAGNOSIS, CONFIRMATION AND TREATMENT OF A NEUROLOGICAL DISORDER AND TCR AND/OR HLA IMMUNOPROFILING IN NEURODEGENERATIVE DISEASE

(71) Applicants: THE TRUSTEES OF COLUMBIA UNIVERSITY IN THE CITY OF NEW YORK, New York, NY (US); LA JOLLA INSTITUTE FOR ALLERGY & IMMUNOLOGY, La Jolla, CA (US); THE RESEARCH FOUNDATION FOR MENTAL HYGIENE, INC., New York, NY (US)

(72) Inventors: David Sulzer, New York, NY (US); Alessandro Sette, La Jolla, CA (US); Cecilia Lindestam Arlehamn, La Jolla, CA (US); John Pham, La Jolla, CA (US); Bjoern Peters, La Jolla, CA (US)

(73) Assignees: THE TRUSTEES OF COLUMBIA UNIVERSITY IN THE CITY OF NEW YORK, New York, NY (US); LA JOLLA INSTITUTE FOR ALLERGY & IMMUNOLOGY, La Jolla, CA (US); THE RESEARCH FOUNDATION FOR MENTAL HYGIENE, INC., New York, NY (US)

(21) Appl. No.: 16/619,286

(22) PCT Filed: Jun. 4, 2018
(86) PCT No.: PCT/US2018/035870

§ 371 Date: Dec. 4, 2019

Related U.S. Application Data
(60) Provisional application No. 62/637,303, filed on Mar. 1, 2018.
(60) Provisional application No. 62/586,597, filed on Nov. 15, 2017.
(60) Provisional application No. 62/568,099, filed on Oct. 4, 2017.
(60) Provisional application No. 62/522,643, filed on Jun. 20, 2017.
(60) Provisional application No. 62/519,558, filed on Jun. 14, 2017.
(60) Provisional application No. 62/518,285, filed on Jun. 12, 2017.
(60) Provisional application No. 62/515,429, filed on Jun. 5, 2017.

(51) Int. Cl. C07K 14/47; G01N 33/50; C12N 5/0783; G01N 33/68; C12Q 1/6869; A61P 25/28

(52) U.S. Cl. CPC C07K 14/47; G01N 33/5047; C12N 5/0636; G01N 33/6896; A61K 38/00; A61P 25/28; G01N 2800/2835; G01N 2800/2821; G01N 2800/285; C12Q 1/6869

(57) ABSTRACT

The present invention provides methods for assessing whether a subject is at risk of developing a neurological disorder, diagnosing or confirming whether a subject is afflicted with a neurological disorder, assessing a neurological disorder is developing in a subject who has been identified as being at risk of developing the neurological disorder, assessing whether a subject afflicted with a neurological disorder is likely to benefit iron a therapy, assessing whether a subject afflicted with a neurological disorder has benefited from a therapy, treating a subject afflicted with a neurological disorder, and prophylactically treating a subject who has been identified as being at risk, of developing a neurological disorder. The present invention also provides epitopes, compounds and compositions relating to these methods.

This application is a § 371 national stage of PCT International Application No. PCT/US2018/035870, filed Jun. 4, 2018, claiming the benefit of U.S. Provisional Application Numbers 62/637,303, filed Mar. 1, 2018, 62/586,597, filed Nov. 15, 2017, 62/568,099, filed Oct. 4, 2017, 62/522,643, filed Jun. 20, 2017, 62/519,558, filed Jun. 14, 2017, 62/518,285, filed Jun. 12, 2017, and 62/515,429, filed Jun. 5, 2017, the entire contents of each of which are hereby incorporated by reference herein.

This application incorporates-by-reference nucleotide and/or amino acid sequences which are present in the file named “191204_88451-A-PCT-US_Sequence_Listing_CAS.txt”, which is 193 kilobytes in size, and which was created Dec. 4, 2019 in the IBM-PC machine format, having an operating system compatibility with MS-Windows, which is contained in the text file filed Dec. 4, 2019 as part of this application.

Throughout this application, various publications are referenced, including referenced in parenthesis. Full citations for publications referenced in parenthesis may be found listed at the end of the specification immediately preceding the claims. The disclosures of all referenced publications in their entireties are hereby incorporated by reference into this application in order to more fully describe the state of the art to which this invention pertains.

### BACKGROUND OF INVENTION

Alzheimer's Disease

Alzheimer's disease (AD) affects about 11% of people aged 65 or older and about 32% of those aged 85 or older. Merck Manual, Parkinson's Disease, last full review/revision August 2007 by David Eidelberg and Michael Pourfar, available at merckmanuals.com/home/brain,-spinal-cord,-and-nerve-disorders/delirium-and-dementia/alzheimer-disease (hereinafter “Merck Manual”).

What causes AD is unclear. According to one theory, several specific gene abnormalities may be involved (Merck Manual). One gene abnormality affects apolipoprotein E (apo E)—the protein part of certain lipoproteins, which transport cholesterol through the bloodstream (Merck Manual). There are three types of apo E: Epsilon-4, Epsilon-2, and Epsilon-3 (Merck Manual). Patients with the epsilon-4 type develop Alzheimer disease more commonly and at an earlier age than others, whereas patients with the epsilon-2 type seem to be protected against Alzheimer disease, and patients with the epsilon-3 type are neither protected nor more likely to develop the disease (Merck Manual). However, genetic testing for apo E type cannot determine whether a specific person will develop Alzheimer disease (Merck Manual).

Alzheimer disease may cause the following abnormalities to develop in brain tissue: (1) accumulation of beta-amyloid, an abnormal, insoluble protein, which accumulates because cells cannot process and remove it (beta-amyloid deposits); (2) clumps of dead nerve cells around a core of beta-amyloid (senile or neuritic plaques); (3) twisted strands of insoluble proteins in the nerve cell (neurofibrillary tangles); and/or (4) increased levels of Tau,. an abnormal protein that is a component of neurofibrillary tangles and beta-amyloid (Merck Manual). The abnormal proteins in Alzheimer disease (beta-amyloid and Tau) are misfolded and cause other proteins to misfold, and may cause the disease to progress (Merck Manual).

Improved and novel methods for diagnosing, confirming, providing biomarkers for, and treating AD are needed.

Parkinson's Disease Parkinson's disease (PD) affects about 1 of 250 people older than 40, about 1 of 100 people older than 65, and about 1 of 10 people older than 80. Merck Manual, Parkinson's Disease, last full review/revision August 2007 by David Eidelberg and Michael Pourfar, available at merckmanuals.com/home/brain_spinal_cord_and_nerve_disorders/movement_dis orders/parkinsons_disease.html (hereinafter “Merck Manual”).

What causes PD is unclear. According to one theory, Parkinson's disease may result from abnormal deposits of synuclein (a protein in the brain that helps nerve cells communicate) (Merck Manual). These deposits, called Lewy bodies, can accumulate in several regions of the brain, particularly in the substantia nigra (deep within the cerebrum) and interfere with brain function (Merck Manual). Lewy bodies often accumulate in other parts of the brain and nervous system, suggesting that they may be involved in other disorders (Merck Manual). In Lewy body dementia, Lewy bodies form throughout the outer layer of the brain (cerebral cortex). Lewy bodies may also be involved in Alzheimer's disease (Merck Manual).

Improved and novel methods for diagnosing, confirming, providing biomarkers for, and treating PD are needed.

Tauopathy

Tauopathies are a group of neurodegenerative diseases characterized by the pathological accumulation of insoluble clusters of hyperphosphorylated Tau protein in neurons and glial cells (Tacik et al., 2015). Tauopathies are divided into primary Tauopathies and secondary Tauopathies.

In primary Tauopathies, Tau inclusions are the major neuropathological abnormality. In secondary Tauopathies, Tau pathology occurs in association with another, more specific, pathology. Tauopathies include Amyotrophic Lateral Sclerosis, Alzheimer's disease, Cerebrotendinous xanthomatosis, Agyrophilic Grain disease, Corticobasal Degeneration, Myotonic Dystrophy Type 1 and 2, Familial Creutzfeldt-Jacob disease, Fatal Familial Insomnia, Frontotemproal Lovar Degeneration, Frontotemporal Dementia, Gerstmann-Straussler-Scheinker syndrome, Niemann-Pick disease, Parkinson's disease, Progressive Supranuclear Palsy, X-linked parkinsonism with spasticity, Sialic acid storage disease, Hereditary cerebral amyloid angiopathy, Kufs disease, 18q deletion syndrome, Neurodegeneration with brain iron accumulation, and Christianson syndrome (Tacik et al., 2015).

Improved and novel methods for diagnosing, confirming, providing biomarkers for, and treating Tauopathies are needed.

Amyotrophic Lateral Sclerosis

Amyotrophic Lateral Sclerosis (ALS) is the most common form of motor neuron disease. Merck Manual, Amyotrophic Lateral Sclerosis and Other Motor Neuron Diseases, last full review/revision August 2007 by David Eidelberg and Michael Pourfar, available at www.merckmanuals.com/home/brain,-spinal-cord,-and-nerve-disorders/peripheral-nerve-disorders/amyotrophic-lateral-sclerosis-and-other-motor-neuron-diseases (hereinafter “Merck Manual”).

What causes ALS is unclear. The majority of ALS cases (90 percent or more) are considered sporadic and about 5% to 7% of people who have a motor neuron disease have a hereditary type (Merck Manual). According to one theory, about 25 to 40 percent of all familial cases (and a small percentage of sporadic cases) are caused by a defect in chromosome 9 open reading frame 72, or C9ORF72. National Institute of Neurological Disorders and Stroke, Amyotrophic Lateral Sclerosis (ALS) Fact Sheet, available at www.ninds.nih.gov/Disorders/Patient-Caregiver-Education/Fact-Sheets/Amyotrophic-Lateral-Sclerosis-ALS-Fact-Sheet, last updated Oct. 18, 2004 (hereinafter “NINDS Fact Sheet”). According to another theory, another 12 to 20 percent of familial cases result from mutations in the gene that provides instructions for the production of the enzyme copper-zinc superoxide dismutase 1 (SOD1) (NINDS Fact Sheet).

Amyotrophic Lateral Sclerosis may result in degeneratation or death of both the upper motor neurons and the lower motor neurons, which stop sending messages to the muscles (NINDS Fact Sheet). Eventually, the brain loses its ability to initiate and control voluntary movements (NINDS Fact Sheet).

Improved and novel methods for diagnosing, confirming, providing biomarkers for, and treating ALS are needed.

In various neurodegenerative diseases, it has been observed that aberrant protein expression and/or aberrant protein function and/or aberrant protein macrostructure (such as protein aggregates) can be found to be associated with the disease predisposition and/or presence and/or progress.

For example, the major pathological features of Parkinson's disease (PD), a neurodegenerative movement disorder, are the death of dopaminergic neurons of the substantia nigra (a basal ganglia structure located in the midbrain that plays an important role in reward and movement), and the presence of intraneuronal protein aggregates known as Lewy bodies that are composed of α-synuclein (α-syn) [Spillantini et al., Proc. Natl Acad. Sci. USA 95, 6469-6473 (1998)].

Alzheimer's disease (AD) is characterized clinically by a progressive and gradual decline in cognitive function and neuropathologically by the presence of neuropil threads, specific neuron loss, and synapse loss in addition to the hallmark protein aggregates in the form of an accumulation of extracellular beta amyloid (Aβ) plaques and the flame-shaped neurofibrillary tangles of the microtubule binding protein tau [Cruts M, Van Broeckhoven C. (1998) Ann Med 30: 560-565; Ruis J. (2008) Rev. Infirm. 143: 14-15; Hsiao K, et al. (1996) Science 274:99-102].

Other diseases also have protein aggregates associated with the disease; in Creutzfeldt-Jakob disease (CJD) there are aggregates of prion protein [Sikorska et al., Subcell Biochem. 2012; 65: 457-96], in sporadic ALS patients there are aggregates of TDP-43 [Arai T, et al., Biochem. Biophys. Res. Commun. 2006;351:602-611], and in frontotemporal lobar degenerations (FTLD) there are aggregates of tau, TDP-43, fused in sarcoma/translocated in liposarcoma (Fus/TLS) and/or ubiquitin [Nonaka et al., Cell Rep. 2013 Jul 11; 4(1): 124-34; Neumann et al., Science. 2006;314:130-133].

Recent evidence has also suggested a role of the innate immune system in neurodegenerative diseases.

For example, recent evidence has suggested that cytokine profiles have implicated the activation of the innate immune system, suggesting a role for the acquired immune system in patients with PD [Cebrián et al., Curr. Top. Behay. Neurosci. 22, 237-270 (2015)], including T cell infiltration into the substantia nigra [Brochard, V. et al. J. Clin. Invest. 119, 182-192 (2009)]. Experimental, genetic and epidemiological data also indicate a crucial role for activation of the innate immune system as a disease-promoting factor in AD, where the sustained formation and deposition of Aβ aggregates causes chronic activation of the immune system and disturbance of microglial clearance functions [Heneka et al., Nature Immunology 16, 229-236 (2015)].

Practical systems, processes and methods for diagnosing, confirming, and/or providing biomarkers for neurodegenerative diseases are still needed.

### SUMMARY OF THE INVENTION

This Summary is provided to introduce a selection of concepts in a simplified form that are further described below in the Detailed Description. This Summary is not intended to identify key aspects or essential aspects of the claimed subject matter.

In the present disclosure, the invention proposed can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term “processor” refers to one or more devices, circuits, and/or processing cores configured to process data, such as computer program instructions.

The present inventors propose a working model where T-cell recognition of peptides derived from proteins associated with neurodegenerative diseases may be a potential element in the neurodegenerative disease predisposition or presence thereof, and/or responsiveness to therapeutic treatment of the disease. Such proteins may be, for example, proteins that have an aberrant protein expression and/or aberrant protein function and/or aberrant protein macrostructure (such as protein aggregates). The present disclosure relates to processes, methods and systems, which make use of this working model. Accordingly, it is proposed that protein antigens can act as autoantigens in neurodegenerative diseases such that such antigens can be the source of biomarkers and diagnostics.

The present invention provides methods for assessing whether a subject is at risk of developing, or for diagnosing or confirming whether a subject is afflicted with an α-synucleinopathy, a Tauopathy, Parkinson's disease (PD), amyotrophic lateral sclerosis (ALS), Lewy Body dementia

(LBD), or Alzheimer's disease (AD) comprising

a)
i) obtaining leukocytes from the subject;
ii) contacting the leukocytes with an epitope peptide;
iii) determining whether the leukocytes have increased activation after contact with the epitope peptide; and
iv) identifying the subject as at risk of developing, or as afflicted with the α-synucleinopathy, PD, ALS, LBD or AD if in step
iii) the leukocytes are determined to have increased activation after contact with the epitope peptide, and identifying the subject as not at risk of developing, or as not afflicted with the α-synucleinopathy, PD, ALS, LBD or AD if in step iii) the leukocytes are determined to not have increased activation after contact with the epitope peptide, or

b)
i) obtaining leukocytes from the subject;
ii) separating the leukocytes into 2 or more pools of leukocytes and Contacting each pool with an epitope peptide, wherein each pool is contacted with a different epitope;
iii) determining whether each pool has increased activation after contact with the epitope peptide; and
iv) identifying the subject as at risk of developing, or as afflicted with the α-synucleinopathy, Tauopathy, PD, ALS, LBD, or AD if and only if in step iii) 1 or more pools is determined to have increased activation after contact with the epitope peptide.

The present invention also provides a method for assessing whether an α-synucleinopathy, a Tauopathy, Parkinson's disease (PD), amyotrophic lateral sclerosis (ALS), Lewy Body dementia (LBD), or Alzheimer's disease (AD) has progressed or is developing in a subject afflicted with or who has been identified as being at risk of developing the α-synucleinopathy, PD, ALS, LBD or AD comprising

a) performing each of the following steps i) to iii):
i) obtaining leukocytes from the subject;
ii) contacting the leukocytes with an epitope peptide that was previously identified to increase activation of the leukocytes; and
iii) determining the level of activation of the leukocytes after contact with the epitope peptide at a first and a second point in time, and then
iv) concluding that the α-synucleinopathy, PD, ALS, LBD or AD has progressed or is developing in the subject if the leukocytes are determined to be more activated in step iii) performed at the second point in time compared to the level of activation in step iii) performed at the first point in time, or

b) performing each of the following steps i) to iii):
i) obtaining leukocytes from the subject;
ii) separating the leukocytes into two or more pools of leukocytes and contacting each pool with an epitope peptide, wherein each pool is contacted with a different epitope;
iii) determining whether each pool has increased activation after contact with the epitope peptide at a first and a second point in time, and then

concluding that the α-synucleinopathy, PD, ALS, LBD or AD has progressed or is developing in the subject if more pools of leukocytes are determined to be activated in step iii) performed at the second point in time compared to the number of pools that are determined to be activated in step iii) performed at the first point in time.

The present invention also provides methods for assessing whether a subject afflicted with an α-synucleinopathy, a Tauopathy, Parkinson's disease (PD), amyotrophic lateral sclerosis (ALS), Lewy Body dementia (LBD), or Alzheimer's disease (AD) is likely to benefit from a therapy, wherein the therapy is directed to leukocytes that are activated by an epitope peptide, the method comprising

a)
i) obtaining leukocytes from the subject;
ii) contacting the leukocytes with the epitope peptide;
iii) determining whether the leukocytes have increased activation after contact with the epitope peptide; and
iv) identifying the subject as likely to benefit from the therapy if in step iii) the leukocytes are determined to have increased activation after contact with the epitope peptide, and identifying the subject as unlikely to benefit from the therapy if in step iii) the leukocytes are determined to not have increased activation after contact with the epitope peptide, or

b)
i) obtaining leukocytes from the subject;
ii) contacting the leukocytes with the epitope peptide;
iii) determining whether the leukocytes have increased activation after contact with the epitope peptide; and
iv) identifying the subject as having benefited from the therapy if in step iii) if the leukocytes are determined to have increased activation after contact with the epitope peptide, and identifying the subject as not having benefitted from the therapy if in step iii) the leukocytes are determined to not have increased activation after contact with the epitope peptide.

The present invention also provides methods for assessing whether a subject afflicted with a disease or condition involving an inflammatory response or related to inflammation, or a neurodegenerative disease or disorder is likely to benefit or has benefitted from a therapy, wherein the therapy comprises administration of an effective amount of a T cell receptor for a particular antigen:MHC complex, the method comprising:

a)
(i) obtaining leukocytes from the subject;
(ii) contacting the leukocytes with the antigen bound to an MHC molecule;
(iii) determining whether the leukocytes have increased activation after contact with the antigen bound to an MHC molecule; and
(iv) identifying the subject as likely to benefit from the therapy if in step (iii) the leukocytes are determined to have increased activation after contact with the antigen bound to an MHC molecule, and identifying the subject as unlikely to benefit from the therapy if in step (iii) the leukocytes are determined to not have increased activation after contact with the antigen bound to an MHC molecule; or

b)
(i) obtaining leukocytes from the subject;
(ii) contacting the leukocytes with the antigen bound to an MHC molecule;
(iii) determining whether the leukocytes have increased activation after contact with the antigen bound to an MHC molecule; and
(iv) identifying the subject as having benefited from the therapy if in step (iii) the leukocytes are determined to have increased activation after contact with the antigen bound to an MHC molecule, and identifying the subject as not having benefitted from the therapy if in step (iii) the leukocytes are determined to not have increased activation after contact with the antigen bound to an MHC molecule.

The present invention also provides methods for treating a subject afflicted with an α-synucleinopathy, a Tauopathy, Parkinson's disease (PD), amyotrophic lateral sclerosis (ALS), Lewy Body dementia (LBD), or Alzheimer's disease (AD)comprising
a) administering to the subject a compound that is approved for use in treating subjects afflicted with the α-synucleinopathy, PD, ALS, LBD or AD, wherein the subject has been diagnosed or confirmed to be afflicted with α-synucleinopathy, PD, ALS, LBD or AD;
b) diagnosing or confirming the Subject to be afflicted with the α-synucleinopathy, PD, ALS, LBD or AD according to the method, and administering to the subject a compound that is approved for use in treating subjects afflicted with α-synucleinopathy, PD, ALS, LBD or AD;
c) administering to the subject a therapy that is directed to leukocytes that are activated by an epitope peptide, wherein leukocytes of the subject have been determined to have increased activation after contact with the epitope peptide;
d) administering an immunosuppressant therapy to the subject, wherein the subject has been identified as being likely to benefit therefrom by the methods; or
e) administering an immunosuppressant therapy to the subject, wherein the subject has been identified as being likely to benefit from a therapy directed to leukocytes that are activated by an epitope peptide according to the methods.

The present invention also provides methods for assessing whether leukocytes of a subject afflicted with an α-synucleinopathy, a Tauopathy, Parkinson's disease (PD), amyotrophic lateral sclerosis (ALS), Lewy Body dementia (LBD), or Alzheimer's disease (AD) are activated by an epitope peptide, comprising
i) obtaining leukocytes from the subject;
ii) contacting the leukocytes with the epitope peptide;
iii) determining whether the leukocytes have increased activation after contact with the epitope peptide; and
iv) identifying the leukocytes of the subject as activated by the epitope peptide if in step iii) the leukocytes are determined to have increased activation after contact with the epitope peptide, and identifying the leukocytes of the subject as not activated by the epitope peptide if in step iii) the leukocytes are determined to not have increased activation after contact with the epitope peptide, wherein
   a. the epitope peptides is represented by an amino acid sequence selected from the group of Tau derived sequences represented by SEQ ID NO: 1-55 or 240-376,
   b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or.
   c. wherein the epitope peptide is represented by the amino acid sequence selected from the group of TDP43 derived sequences represented by SEQ ID NO: 56-239.

The present invention, also provides methods for assessing whether a test compound comprises an epitope peptide to which leukocytes of a subject suffering from a neurological disorder are responsive comprising
i) obtaining leukocytes from the subject;
ii) contacting the leukocytes with the test compound;
iii) determining whether the leukocytes has increased activation after contact with the test compound; and
iv) identifying the test compound as comprising an epitope peptide to which the leukocytes are responsive if in step iii) the leukocytes are determined to have increased activation after contact with the test compound, and identifying the test compound as not comprising an epitope to which the leukocytes are responsive if in step iii) the leukocytes are determined to not have increased activation after contact with the test compound, wherein
a. the epitope peptides is represented by an amino acid sequence selected from the group of Tau derived sequences represented by SEQ ID NO: 1-55 or 240-376,
b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or.
c. wherein the epitope peptide is represented by the amino acid sequence selected from the group of TDP43 derived sequences represented by SEQ ID NO: 56-239.

The present invention also provides for compounds for treating an α-synucleinopathy, a Tauopathy, Parkinson's disease (PD), amyotrophic lateral sclerosis (ALS), Lewy Body dementia (LBD), or Alzheimer's disease (AD), comprising i) a major histocompatibility complex (MHC) Tetramer having four MHC molecules, wherein each MHC molecule is associated with an epitope peptide, and ii) a toxin, wherein
a. the epitope peptides is represented by an amino acid sequence selected from the group of Tau derived sequences represented by SEQ ID NO: 1-55 or 240-376,
b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or.
c. wherein the epitope peptide is represented by the amino acid sequence selected from the group of TDP43 derived sequences represented by SEQ ID NO: 56-239.

The present invention also provides processes for assessing whether a subject is at risk of developing an α-synucleinopathy, a Tauopathy, Parkinson's disease (PD), amyotrophic lateral sclerosis (ALS), Lewy Body dementia (LBD), or Alzheimer's disease (AD), which involves an array of testing, the improvement comprising including in the array of testing the steps of:
a)
   i) obtaining leukocytes from the subject;
   ii) contacting the leukocytes with an epitope peptide;
   iii) determining whether the leukocytes have increased activation after contact with the epitope peptide; and
   iv) identifying the subject as at risk of developing α-synucleinopathy, PD, ALS, LBD or AD if in step iii) the leukocytes are determined to have increased activation after contact with the epitope peptide, and identifying the subject as not at risk of developing the α-synucleinopathy, PD, ALS, LBD or AD if in step iii) the leukocytes are determined to not have increased activation after contact with the epitope peptide, wherein
   a. the epitope peptides is represented by an amino acid sequence selected from the group of Tau derived sequences represented by SEQ ID NO: 1-55 or 240-376,
   b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or.
   c. wherein the epitope peptide is represented by the amino acid sequence selected from the group of TDP43 derived sequences represented by SEQ ID NO: 56-239
   or
b)
   i) obtaining leukocytes from the subject;
   ii) separating the leukocytes into 2 or more pools of leukocytes and contacting each pool with an epitope peptide, wherein each pool is contacted with a different epitope;
   iii) determining whether each pool has increased activation after contact with the epitope peptide; and
   iv) identifying the subject as at risk of developing the α-synucleinopathy, PD, ALS, LBD or AD if in step iii) 1 or more pools is determined to have increased activation after contact with the epitope peptide, wherein
   a. the epitope peptides is represented by an amino acid sequence selected from the group of Tau derived sequences represented by SEQ ID NO: 1-55 or 240-376,
   b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or
   c. wherein the epitope peptide is represented by the amino acid sequence selected from the group of TDP43 derived sequences represented by SEQ ID NO: 56-239.

The present invention also provides processes for diagnosing or confirming whether a subject is afflicted with an α-synucleinopathy, a Tauopathy, Parkinson's disease (PD), amyotrophic lateral sclerosis (ALS), Lewy Body dementia (LBD), or Alzheimer's disease (AD), which involves an array of testing, the improvement comprising including in the array of testing the steps of:
a)
   i) obtaining leukocytes from the subject;
   ii) separating the leukocytes into 1 or more pools of leukocytes and contacting each pool with an epitope peptide, wherein each pool is contacted with a different epitope peptide;
   iii) determining whether each pool has increased activation after contact with the epitope peptide; and
   iv) identifying the subject as afflicted with the α-synucleinopathy, PD, ALS, LBD or AD if and only if in step iii) 1 or more pools is determined to have increased activation after contact with an epitope peptide, or
b)
   i) obtaining leukocytes from the subject;
   ii) separating the leukocytes into 1 or more pools of leukocytes and contacting each pool with an epitope peptide, wherein each pool is contacted with a different epitope peptide;
   iii) determining whether each pool has increased activation after contact with the epitope peptide; and
   iv) identifying the subject as afflicted with the α-synucleinopathy, PD, ALS, LBD or AD if and only if in step iii) 1 or more pools is determined to have increased activation after contact with an epitope peptide, wherein
      a. the epitope peptides is represented by an amino acid sequence selected from the group of Tau derived sequences represented by SEQ ID NO: 1-55 or 240-376,
      b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or.
      c. wherein the epitope peptide is represented by the amino acid sequence selected from the group of TDP43 derived sequences represented by SEQ ID NO: 56-239.

The present invention also provides for pharmaceutical compositions for treating an α-synucleinopathy, a Tauopathy, Parkinson's disease (PD), amyotrophic lateral sclerosis (ALS), Lewy Body dementia (LBD), or Alzheimer's disease (AD), comprising
i) a protein comprising an amino acid sequence selected from the group of
   a. the epitope peptides is represented by an amino acid sequence selected from the group of Tau derived sequences represented by SEQ ID NO: 1-55 or 240-376,
   b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or.
   c. wherein the epitope peptide is represented by the amino acid sequence selected from the group of TDP43 derived sequences represented by SEQ ID NO: 56-239, and
ii) a pharmaceutically acceptable carrier.

The present invention also provides a method comprising:
a. providing a biological sample from a subject;
b. processing the biological sample to determine presence of a T cell receptor (TCR) specific to a peptide, wherein the peptide is a fragment from a protein associated with said neurodegenerative disease.

The present invention also provides a method comprising:
a) providing a biological sample from a subject;
b) processing the biological sample to determine presence of a human leukocyte antigen (HLA) capable of presenting a peptide, wherein the peptide is a fragment from a protein associated with said neurodegenerative disease; and
c) processing the biological sample to determine presence of a T cell receptor (TCR) specific to said peptide.

As embodied and broadly described herein, the present disclosure relates to a method comprising: providing a biological sample from a subject; processing the biological sample to determine presence of a T cell receptor (TCR) specific to a peptide, wherein the peptide is a fragment from a protein having an aberrant protein expression and/or aberrant protein function and/or aberrant protein macrostructure in a patient having a neurodegenerative disease.

As embodied and broadly described herein, the present disclosure relates to a method comprising: providing a biological sample from a subject; processing the biological sample to determine presence of a human leukocyte antigen (HLA) capable of presenting a peptide, wherein the peptide is a fragment from a protein having an aberrant protein expression and/or aberrant protein function and/or aberrant protein macrostructure in a patient having a neurodegenerative disease; and processing the biological sample to determine presence of a T cell receptor (TCR) specific to said peptide.

In one embodiment, the peptide is a fragment from a protein that forms aggregates in a patient having the neurodegenerative disease.

As embodied and broadly described herein, the present disclosure relates to a system for processing biological data, comprising: one or more processors; and one or more memories coupled to the one or more processors. The one or more memories are configured to provide the one or more processors with instructions which when executed cause the one or more processors to receive first and second biological data elements for an individual from a biological data source, wherein the first biological data element comprises data pertaining to the individual's human leukocyte antigen (HLA) typing and the second biological data element comprises data pertaining to the individual's T cell receptor (TCR) repertoire. Further, the one or more memories are configured to provide the one or more processors with instructions which when executed cause the one or more processors to merge the first and second biological data elements from the biological data source to obtain a set of merged biological data associated with the individual, including to identify data in the first and second biological data elements that indicates a reciprocity, the identified data corresponding to a reciprocal presence of an HLA typing value in the first biological data element and of a TCR repertoire value in the second biological data element; compare the identified data with at least one of an element of HLA typing values and TCR repertoire values stored on the one or more memories, said values stored on the one or more memories being associated with reference individuals; and determine a likelihood or predisposition score based on at least the identified data and on the comparison. Further, the one or more memories are configured to provide the one or more processors with instructions which, when executed, cause the one or more processors to display the likelihood or predisposition score in a graphical user interface (GUI).

In one non-limiting embodiment, the neurodegenerative diseases may include at least one of alpha-synucleinopathy, Parkinson's disease (PD), Lewy Body dementia (LBD), and Alzheimer's disease (AD).

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

FIG. 1: Tau-specific responses for Parkinson's Disease donors as compared to healthy control donors for each of the HC Young (donors under 35 years old) and HC Age-Matched (donors above 50 years old) cohorts.

FIG. 2A: Analysis of response magnitude per donor for each of the Parkinson's Disease, HC Young (HC<35) and HC Age-Matched (HC>50) cohorts. Response magnitude for IFN-γ (IFNg), IL-5 and the sum of both cytokines is shown.

FIG. 2B: Analysis of response magnitude for each individual peptide for each of the Parkinson's Disease, HC Young (HC<35) and HC Age-Matched (HC>50) cohorts. The responses observed in each donor against each individual peptide are plotted for IFN-γ (IFNg), IL-5 and the sum of both cytokines.

FIG. 3A: Overall response as plotted against age. Overall responses are not correlated with age in either controls or PD patients.

FIG. 3B: Total reactivity as a function of time since onset of symptoms.

FIG. 3C: Intercellular Cytokine Staining analysis of cytokine response for each of the Parkinson's Disease, HC Young (HC<35) and HC Age-Matched (HC>50) cohorts.

FIG. 3D: ELISPOT analysis of cytokine response for each of the Parkinson's Disease, HC Young (HC<35) and HC Age-Matched (HC>50) cohorts.

FIG. 4A: Breadth of response per donor. The number of epitopes responded to per donor is plotted for each of the Parkinson's Disease, HC Young (HC<35) and HC Age-Matched (HC>50) cohorts.

FIG. 4B: Magnitude of response per epitope per donor. The magnitude of response per epitope per donor is plotted for each of the Parkinson's Disease, HC Young (HC<35) and HC Age-Matched (HC>50) cohorts.

FIG. 5: Tau-specific response as compared to α-syn specific response. Tau-specific cytonkine response was plotted in comparison to α-syn specific response.

FIG. 6A: α-Syn autoimmune responses are directed against two regions. Sequence of α-syn. Antigenic regions are highlighted with dashed lines with amino acids Y39 and S129 in bold.

FIG. 6B: α-Syn autoimmune responses are directed against two regions. Magnitude of IFNγ responses expressed as (SFC/10⁶ PBMC) per peptide/participant combination. Left panels; response to all overlapping native α-syn 15 mer peptides in PD (n=733) and Control (n=372). Right panels indicate responses against specific 15 mers. Grey shading indicates antigenic region containing Y39. As many participants showed no response, many points are at the limit of resolution (100 SFC).

FIG. 6C: α-Syn autoimmune responses are directed against two regions. Magnitude of IL-5 responses expressed as (SFC/10⁶ PBMC) per peptide/participant combination. Left panels; response to all overlapping native α-syn 15 mer peptides in PD (n=733) and Control (n=372). Right panels indicate responses against specific 15 mers. Grey shading indicates antigenic region containing Y39. As many participants showed no response, many points are at the limit of resolution (100 SFC).

FIG. 6D: α-Syn autoimmune responses are directed against two regions. Magnitude of total (IFNγ & IL-5) response expressed as (SFC/10⁶ PBMC) per peptide/participant combination. Left panels; response to all overlapping native α-syn 15 mer peptides in PD (n=733) and Control (n=372). Right panels indicate responses against specific 15 mers. Grey shading indicates antigenic region containing Y39. As many participants showed no response, many points are at the limit of resolution (100 SFC).

FIG. 6E: α-Syn autoimmune responses are directed against two regions. Magnitude of IFNγ responses. Left panels; responses to all native and phosphorylated S129 α-syn 15 mer peptides in PD (n=150) and Control (n=72). Right panels; responses against specific S129 peptides. Closed circles, PD (n=19, indicated by *, all other n=25); open circles, Control (n=12 participants). Two-tailed Mann Whitney, ns, not significant. As many participants showed no response, many points are at the limit of resolution (100 SFC).

FIG. 6F: α-Syn autoimmune responses are directed against two regions. Magnitude of IL-5 responses. Left panels; responses to all native and phosphorylated S129 α-syn 15 mer peptides in PD (n=150) and Control (n=72). Right panels; responses against specific S129 peptides. Closed circles, PD (n=19, indicated by *, all other n=25); open circles, Control (n=12 participants). Two-tailed Mann Whitney, ns, not significant. As many participants showed no response, many points are at the limit of resolution (100 SFC).

FIG. 6G: α-Syn autoimmune responses are directed against two regions. Magnitude of total (IFNγ & IL-5) response. Left panels; responses to all native and phosphorylated S129 α-syn 15 mer peptides in PD (n=150) and Control (n=72). Right panels; responses against specific S129 peptides. Closed circles, PD (n=19, indicated by *, all other n=25); open circles, Control (n=12 participants). Two-tailed Mann Whitney, ns, not significant. As many participants showed no response, many points are at the limit of resolution (100 SFC).

FIG. 7A: T cell reactivity against α-syn peptides (wild type and posttranslationally modified). Magnitude of responses, expressed as the total magnitude (SFC/10⁶ PBMC) of IFNγ response per peptide/participant combination. Responses against any α-syn 15 mer peptide spanning S129 and Y39, “any peptide”, PD (n=209), Control (n=132), and responses against individual α-syn 15 mer peptides spanning S129 and Y39. Each dot represents a peptide/participant combination. Closed circles, PD (n=19); open circles, Control (n=12). Two-tailed Mann Whitney, *, p<0.05, **, p<0.01, ***, p<0.001.

FIG. 7B: T cell reactivity against α-syn peptides (wild type and posttranslationally modified). Magnitude of responses, expressed as the total magnitude (SFC/10⁶ PBMC) of IL-5 response per peptide/participant combination. Responses against any α-syn 15 mer peptide spanning S129 and Y39, “any peptide”, PD (n=209), Control (n=132), and responses against individual α-syn 15 mer peptides spanning S129 and Y39. Each dot represents a peptide/participant combination. Closed circles, PD (n=19); open circles, Control (n=12). Two-tailed Mann Whitney, *, p<0.05, **, p<0.01, ***, p<0.001.

FIG. 7C: T cell reactivity against α-syn peptides (wild type and posttranslationally modified). Magnitude of responses, expressed as the total magnitude (SEC/10⁶ PBMC) of total (IFNγ and IL-5 combined) response per peptide/participant combination. Responses against any α-syn 15 mer peptide spanning S129 and Y39, “any peptide”, PD (n=209), Control (n=132), and responses against individual α-syn 15 mer peptides spanning S129 and Y39. Each dot represents a peptide/participant combination. Closed circles, PD (n=19); open circles, Control (n=12). Two-tailed Mann Whitney, *, p<0.05, **, p<0.01, ***, p<0.001.

FIG. 8A: Reactivity to native and modified α-syn peptides in PD patients. Magnitude of IFNγ responses against native and modified α-syn 15 mer S129 and Y39 region peptides as (SEC/10⁶ PBMC). Each point represents a peptide/participant combination. Closed circles, PD (n=403 peptide/participant combinations “any peptide”, KTKEGVLYVGSKTKE n=63 participants (\hat{ }), modified peptides marked with * are tested in 19 participants, unmodified peptides are tested in n=41); open circles, control (n=228 any peptide, \hat{ } n=36, *n=12 and unmodified peptides n=24).

FIG. 8B: Reactivity to native and modified α-syn peptides in PD patients. Magnitude of IL-5 responses against native and modified α-syn 15 mer S129 and Y39 region peptides as (SFC/10⁶ PBMC). Each point represents a peptide/participant combination. Closed circles, PD (n=403 peptide/participant combinations “any peptide”, KTKEGVLYVGSKTKE n=63 participants (\hat{ }), mddified peptides marked with * are tested in 19 participants, unmodified peptides are tested in n=41); open circles, control (n=228 any peptide, \hat{ } n=36, *n=12 and unmodified peptides n=24).

FIG. 8C: Reactivity to native and modified α-syn peptides in PD patients. Magnitude of total (IFNγ & IL-5 combined) responses against native and modified α-syn 15 mer S129 and Y39 region peptides as (SFC/10⁶ PBMC). Each point represents a peptide/participant combination. Closed circles, PD (n=403 peptide/participant combinations “any peptide”, KTKEGVLYVGSKTKE n=63 participants (\hat{ }), modified peptides marked with * are tested in 19 participants, unmodified peptides are tested in n=41); open circles, control (n=228 any peptide, \hat{ } n=36, *n=12 and unmodified peptides n=24).

FIG. 8D: Reactivity to native and modified α-syn peptides in PD patients. Combined IL-5 and IFNγ responses against individual native and modified α-syn peptides by PD. Black points, IFNγ responses; red points, IL-5 responses. Two-tailed Mann Whitney, *, p<0.05, **, p<0.01, ***, p<0.001. As many participants showed no response, many points are at the limit of resolution (100 SFC).

FIG. 9A: Characterization of α-syn specific responses in PD. Gating strategy. T cells were gated based on CD3 expression. Boolean gating was used to define cytokine-producing cells expressing CD4 and/or CD8.

FIG. 9B: Characterization of α-syn specific responses in PD. Percent total cytokine detected from CD3+ T cells in response to α-syn peptides. Each point represents one participant (n=9); median±interquartile range is indicated. Dotted line indicates 0.05% cut-off for specific cytokine production by CD3+ T cells.

FIG. 9C: Characterization of α-syn specific responses in PD. Percentage of total cytokines produced for IFNγ, IL-4, IL-10, and IL-17. Each point represents one participant that exceeded the cut-off (n=6), median±interquartile range is indicated.

FIG. 9D: Characterization of α-syn specific responses in PD. Percentage of total cytokines produced by CD4+, CD8+, CD4−CD8−, or CD4+CD8+ T cells. Each point represents one participant (n=6), median±interquartile range is indicated.

FIG. 10: Specific T cell reactivity against native or fibrilized α-syn. Magnitude of responses, expressed as the average spots per 10⁶ PBMC, of response per protein/PD participant or peptide/PD participant combination (n=12 PD participants, each represented by a different symbol). The lines connect discrete values from each individual participant and are present to provide a means to compare responses within and between individuals. The difference between response to unstimulated compared to peptides, the native α-syn and PFF groups is significant by the Wilcoxon one-tailed test (values are shown in the figure). No significant difference (Wilcoxon two-tailed test) in response to PFF and native protein was apparent in this relatively small sample.

FIG. 11A: HLA-DR surface expression across DRB1*15:01+ or DRB1*15:01− PD and HC participants. Gating strategy for FACS analysis. After eliminating non-lymphocytes and doublet cells by forward- and side-scatter, cells were gated based on HLA-DR expression.

FIG. 11B: HLA-DR surface expression across DRB1 15:01+ or DRB1*15:01− PD and HC participants. HLA-DR and CD3 expression of participant cells (black; HLA-DR antibody, red; isotype control) of PD patients that carry (n=5) DRB1*15:01 allele.

FIG. 11C: HLA-DR surface expression across DRB1*15:01+ or DRB1*15:01− PD and HC participants. HLA-DR and CD3 expression of participant cells (black; HLA-DR antibody, red; isotype control) of PD patients that do not carry (n=5) DRB1*15:01 allele.

FIG. 11D: HLA-DR surface expression across DRB1*15:01+ or DRB1*15:01− PD and HC participants. HLA-DR and CD3 expression of participant cells (black; HLA-DR antibody, red; isotype control) of HC that carry (n=3) DRB1*15:01 allele

FIG. 11E: HLA-DR surface expression across DRB1*15:01+ or DRB1*15:01− PD and HC participants. HLA-DR and CD3 expression of participant cells (black; HLA-DR antibody, red; isotype control) of HC that do not carry (n=5) DRB1*15:01 allele.

FIG. 11F: HLA-DR surface expression across DRB1*15:01+ or DRB1*15:01− PD and HC participants. 721.221 cells are used as controls that do not express HLA class II.

FIG. 11G: HLA-DR surface expression across DRB1*15:01+ or DRB1*15:01− PD and HC participants. RM3 cells are used as controls that do express HLA class II.

FIG. 11H: HLA-DR surface expression across DRB1*15:01+ or DRB1*15:01− PD and HC participants. Mean fluorescent intensities (MFI)±standard deviations of HLADR expression for each participant cohort.

FIG. 11I: HLA-DR surface expression across DRB1*15:01+ or DRB1*15:01− PD and HC participants. Percentage of living cells that express HLA-DR, mean±SD.

FIG. 12A: HLA class I surface expression across DRB1*15:01+ or DRB1*15:01− PD and HC participants. Gating strategy for FACS analysis. After eliminating non-lymphocytes and doublet cells by forward- and side-scatter, cells were gated based on HLA-ABC expression.

FIG. 12B: HLA class I surface expression across DRB1*15:01+ or DRB1*15:01− PD and HC participants. HLA-ABC and CD3 expression of participant cells (black: HLA-ABC antibody, red: isotype control) of PD patients that carry (n=5) DRB1*15:01 allele.

FIG. 12C: HLA class I surface expression across DRB1*15:01+ or DRB1*15:01− PD and HC participants. HLA-ABC and CD3 expression of participant cells (black: HLA-ABC antibody, red: isotype control) of PD patients that do not carry (n=5) DRB1*15:01 allele.

FIG. 12D: HLA class I surface expression across DRB1*15:01+ or DRB1*15:01− PD and HC participants. HLA-ABC and CD3 expression of participant cells (black: HLA-ABC antibody, red: isotype control) of HC that carry (n=3) DRB1*15:01 allele.

FIG. 12E: HLA class I surface expression across DRB1*15:01+ or DRB1*15:01− PD and HC participants. HLA-ABC and CD3 expression of participant cells (black: HLA-ABC antibody, red: isotype control) of HC that do not carry (n=5) DRB1*15:01 allele.

FIG. 12F: HLA class I surface expression across DRB1*15:01+ or DRB1*15:01− PD and HC participants. 721.221 cells are used as controls that do not express HLA class I.

FIG. 12G: HLA class I surface expression across DRB1*15:01+ or DRB1*15:01− PD and HC participants. RM3 cells are used as controls that do express HLA class I.

FIG. 12H: HLA class I surface expression across DRB1*15:01+ or DRB1*15:01− PD and HC participants. Mean fluorescent intensities (MFI)±standard deviations of HLAABC expression for each participant cohort.

FIG. 13A: HLA association of Y39 epitope and identification of A*11:01 restricted 9-10 aa length Y39 epitopes. Overlapping but largely independent associations between DRB1*15:01, DQB1*03:04 and A*11:01 for PD (13 participants) responding to the Y39 epitope.

FIG. 13B: HLA association of Y39 epitope and identification of A*11:01 restricted 9-10aa length Y39 epitopes. Magnitude of responses by PD (n=19), as (SFC/10⁶ PBMC) of response per peptide/participant combination to α-syn 9-10 mer peptides spanning the protein. In some cases, response to overlapping peptides are combined, with additional residues of the longer peptide in parentheses. top panel, IFNγ; middle, IL-5; bottom, total (IFNγ & IL-5 combined) response. As many participants showed no T cell response, many points are at the limit of resolution (100 SFC).

FIG. 13C: HLA association of Y39 epitope and identification of A*11:01 restricted 9-10aa length Y39 epitopes. Magnitude of Total (IFNγ & IL-5 combined) responses by control participants (n=12), as (SFC/10⁶ PBMC) of response per peptide/participant combination to α-syn 9-10 mer peptides spanning the protein. In some cases, response to overlapping peptides are combined, with additional residues of the longer peptide in parentheses. As many participants showed no T cell response, many points are at the limit of resolution (100 SFC).

FIG. 14A: Magnitude of IFNγ responses expressed as (SFC/10⁶ PBMC) per peptide/participant combination. Response to selected TDP43 15 mer peptides by ALS patients and healthy controls.

FIG. 14B: Magnitude of IL-5 responses expressed as (SFC/10⁶ PBMC) per peptide/participant combination. Response to selected TDP43 15 mer peptides by ALS patients and healthy controls.

FIG. 14C: Magnitude of IL-10 responses expressed as (SFC/10⁶ PBMC) per peptide/participant combination. Response to selected TDP43 15 mer peptides by ALS patients and healthy controls.

FIG. 14D: Magnitude of any cytokine responses expressed as (SFC/10⁶ PBMC) per peptide/participant combination. Response to selected TDP43 15 mer peptides by ALS patients and healthy controls.

FIG. 15: High-level functional block diagram of a system for assessing a neurodegenerative disease patient in accordance with a specific example of implementation of the present invention;

FIG. 16: Functional block diagram of an apparatus for generating neurodegenerative disease patient information suitable for use in the system depicted in FIG. 15 in accordance with a first specific example of implementation of the present invention.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION OF THE INVENTION

Abbreviations

α-syn, alpha synuclein; AD, Alzheimer's disease; AIM, Activation Induced Marker; ALS, amyotrophic lateral sclerosis; β2 m, beta 2 microglobulin; BF, brightfield; BrdU, 5-bromo-2-deoxyuridine; BSA, bovine seroalbumin; ConA, concananycin A; CMA, chaperone-mediated autophagy; CNS, central nervous system; CTLs, cytotoxic T cells CTRL, control; DA, dopamine/dopaminergic; DCs, dendritic cells; ELISA, enzyme-linked immunosorbent assay; ELISPOT, enzyme-linked immunospot assay; GSEA, Gene Set Enrichment Analysis; HC, healthy control(s); hES, human stem cells; HLA, human leukocyte antigen; IEDB, Immune Epitope Database; IFN-γ, interferon gamma; IL-1p, interleukin 1-beta; IL-6, interleukin 6; IPA, Integrated Pathway Analysis; KO, knocked out; LC, locus coeruleus; LGN, lateral geniculate nucleus; LPS, lipopolysaccharide; MAP-2, microtubule associated protein-2; MHC, major histocompatibility complex; MHC-I, major histocompatibility complex class I; MHC-II, major histocompatibility complex class II; MN, spinal motor neurons; Mut-α-syn, mutated (A53T) alpha synuclein; NE, norepinephrine/norepinephrinergic; Nit-α-syn, nitrated alpha synuclein; NM, neuromelanin; OVA, ovalbumin; PBMC, peripheral blood mononuclear cells; PD, Parkinson's disease; PET, positron emission tomography; PSP, progressive supranuclear palsy; PTM, post-translational modification; RATE, Restrictor Analysis Tool for Epitopes; SEM, standard error of the mean; SFC, spot-forming cells; SN, substantia nigra; TCR, T cell receptor; TH, tyrosine hydroxylase; UPDRS, Unified Parkinson's Disease Rating Scale; Veh, vehicle; VM, ventral midbrain; VTA, ventral tegmental area; WGCNA, Gene Co-expression Network Analysis.

A detailed description of one or more embodiments of the invention is provided below along with accompanying figures that illustrate the principles of the invention. The invention is described in connection with such embodiments, but the invention is not limited to any embodiment. The scope of the invention is limited only by the claims.

Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

The present invention provides methods for assessing whether a subject is at risk of developing, or for diagnosing or confirming whether a subject is afflicted with an α-synucleinopathy, a Tauopathy, Parkinson's disease (PD), amyotrophic lateral sclerosis (ALS), Lewy Body dementia (LBD), or Alzheimer's disease (AD) comprising

a)
i) obtaining leukocytes from the subject;
ii) contacting the leukocytes with an epitope peptide;
iii) determining whether the leukocytes have increased activation after contact with the epitope peptide; and
iv) identifying the subject as at risk of developing, or as afflicted with the α-synucleinopathy, PD, ALS, LBD or AD if in step
iii) the leukocytes are determined to have increased activation after contact with the epitope peptide, and identifying the subject as not at risk of developing, or as not afflicted with the α-synucleinopathy, PD, ALS, LBD or AD if in step iii) the leukocytes are determined to not have increased activation after contact with the epitope peptide, or

b)
i) obtaining leukocytes from the subject;
ii) separating the leukocytes into 2 or more pools of leukocytes and contacting each pool with an epitope peptide, wherein each pool is contacted with a different epitope;
iii) determining whether each pool has increased activation after contact with the epitope peptide; and
iv) identifying the subject as at risk of developing, or as afflicted with the α-synucleinopathy, Tauopathy, PD, ALS, LBD, or AD if and only if in step iii) 1 or more pools is determined to have increased activation after contact with the epitope peptide.

The present invention also provides a method for assessing whether an α-synucleinopathy, a Tauopathy, Parkinson's disease (PD), amyotrophic lateral sclerosis (ALS), Lewy Body dementia (LBD), or Alzheimer's disease (AD) has progressed or is developing in a subject afflicted with or who has been identified as being at risk of developing the α-synucleinopathy, PD, ALS, LBD or AD comprising

a) performing each of the following steps i) to iii):
i) obtaining leukocytes from the subject;
ii) contacting the leukocytes with an epitope peptide that was previously identified to increase activation of the leukocytes; and
iii) determining the level of activation of the leukocytes after contact with the epitope peptide at a first and a second point in time, and then
iv) concluding that the α-synucleinopathy, PD, ALS, LBD or AD has progressed or is developing in the subject if the leukocytes are determined to be more activated in step iii) performed at the second point in time compared to the level of activation in step iii) performed at the first point in time, or

b) performing each of the following steps i) to iii):
i) obtaining leukocytes from the subject;
ii) separating the leukocytes into two or more pools of leukocytes and contacting each pool with an epitope peptide, wherein'each pool is contacted with a different epitope;
iii) determining whether each pool has increased activation after contact with the epitope peptide at a first and a second point in time, and then

concluding that the α-synucleinopathy, PD, ALS, LBD or AD has progressed or is developing in the subject if more pools of leukocytes are determined to be activated in step iii) performed at the second point in time compared to the number of pools that are determined to be activated in step iii) performed at the first point in time.

The present invention also provides methods for assessing whether a subject afflicted with an α-synucleinopathy, a Tauopathy, Parkinson's disease (PD), amyotrophic lateral sclerosis (ALS), Lewy Body dementia (LBD), or Alzheimer's disease (AD) is likely to benefit from a therapy, wherein the therapy is directed to leukocytes that are activated by an epitope peptide, the method comprising

a)
i) obtaining leukocytes from the subject;
ii) contacting the leukocytes with the epitope peptide;
iii) determining whether the leukocytes have increased activation after contact with the epitope peptide; and
iv) identifying the subject as likely to benefit from the therapy if in step iii) the leukocytes are determined to have increased activation after contact with the epitope peptide, and identifying the subject as unlikely to benefit from the therapy if in step iii) the leukocytes are determined to not have increased activation after contact with the epitope peptide, or

b)
i) obtaining leukocytes from the subject;
ii) contacting the leukocytes with the epitope peptide;
iii) determining whether the leukocytes have increased activation after contact with the epitope peptide; and
iv) identifying the subject as having benefited from the therapy if in step iii) if the leukocytes are determined to have increased activation after contact with the epitope peptide, and identifying the subject as not having benefitted from the therapy if in step iii) the leukocytes are determined to not have increased activation after contact with the epitope peptide.

The present invention also provides methods for assessing whether a subject afflicted with a disease or condition involving an inflammatory response or related to inflammation, or a neurodegenerative disease or disorder is likely to benefit or has benefitted from a therapy, wherein the therapy comprises administration of an effective amount of a T cell receptor for a particular antigen:MHC complex, the method comprising:

a)
(i) obtaining leukocytes from the subject;
(ii) contacting the leukocytes with the antigen bound to an MHC molecule;
(iii) determining whether the leukocytes have increased activation after contact with the antigen bound to an MHC molecule; and
(iv) identifying the subject as likely to benefit from the therapy if in step (iii) the leukocytes are determined to have increased activation after contact with the antigen bound to an MHC molecule, and identifying the subject as unlikely to benefit from the therapy if in step (iii) the leukocytes are determined to not have increased activation after contact with the antigen bound to an MHC molecule; or

b)
(i) obtaining leukocytes from the subject;
(ii) contacting the leukocytes with the antigen bound to an MHC molecule;
(iii) determining whether the leukocytes have increased activation after contact with the antigen bound to an MHC molecule; and
(iv) identifying the subject as having benefited from the therapy if in step (iii) the leukocytes are determined to have increased activation after contact with the antigen bound to an MHC molecule, and identifying the subject as not having benefitted from the therapy if in step (iii) the leukocytes are determined to not have increased activation after contact with the antigen bound to an MHC molecule.

In some embodiments, in step ii) the leukocytes are separated into 2, 3, 4, 5, 6, 7, 8, 9, 10, 11-50 or more pools, and in step iv) the subject is identified as at risk of developing or as afflicted with the α-synucleinopathy, Tauopathy, PD, ALS, LBD or AD if and only if in step iii) 1, 2, 3, 4, 5, 6, 7, 8, 9, 10, 11-50 or more pools is determined to have increased activation after contact with the epitope peptide.

In some embodiments, the subject the subject
a) is at least about 35, 40, 45, 50, 55, 60, 65, 70, 75 or 80 years of age;
b) is less than about 35, 40, 45, 50, 55, 60, 65, 70, 75 or 80 years of age;
c) has a symptom that has preceded the onset of the α-synucleinopathy, Tauopathy, PD, ALS, LBD or AD in subjects who have developed α-synucleinopathy, Tauopathy, PD, ALS, LBD or AD;
d) has a symptom that has preceded the onset of the α-synucleinopathy, Tauopathy, PD, ALS, LBD or AD in subjects who have developed the α-synucleinopathy, Tauopathy, PD, ALS, LBD or AD, wherein the symptom has preceded the onset of the α-synucleinopathy, Tauopathy, PD, ALS, LBD or AD in the subjects by at least about 5, 10, 15, 20, 25, 30 or 5-30 years;
e) is afflicted with cognitive decline, constipation or orthostatic hypotension
f) is afflicted with cognitive decline, and the cognitive decline is reduced spatial reasoning ability and/or reduced memory ability.
g) is afflicted with fasciculations or muscle twitches in the arm leg, shoulder, or tongue, muscle cramps, spasticity or tight and stiff muscles, muscle weakness affecting an arm, a leg, neck or diaphragm, slurred and nasal speech, and/or difficulty chewing or swallowing; or
h) is afflicted with cognitive decline, and the cognitive decline is reduced language or decision-making.

In some embodiments; the subject is the subject to
a) be monitored more frequently for the α-synucleinopathy, Tauopathy, PD, ALS, LBD or AD; or
b) receive additional diagnostic testing for the α-synucleinopathy, Tauopathy, PD, ALS, LBD or AD,

if the subject is identified as at risk of developing the α-synucleinopathy, Tauopathy, PD, ALS, LBD or AD.

In some embodiments the presence of at least one human leukocyte antigen (HLA) allele, one T cell receptor (TCR) allele, or one MAPT allele is determined in the subject.

In some embodiments, the subject is identified as at risk of developing the α-synucleinopathy, PD, ALS, LBD or AD or identified as afflicted with the α-synucleinopathy, PD, ALS, LBD or AD if
a) the leukocytes are determined to have increased activation after contact with the epitope peptide, or 1 or more pools is determined to have increased activation after contact with the epitope peptide, and
b) the subject has at least one HLA allele.

In some embodiments, the subject has the HLA allele DRB5*01:01, DRB1*15:01, DQB1*03:04, A*11:01, DRB1*09:01, DRB1*15, DRB1*04, DQB1*06, DRB1*01:01, DRB1*04:04, DRB1*07:01, DRB1*11:04, DRB3*02:02, DQA1*05:01, DQB1*03:01, DQB1*03:02, DQB1*03:03, DQB1*04:02, DRB1*15:01/DQB1*06:02 or DRB1*04:02/DQB1*03:02. In a further embodiment, the subject has the HLA alleles DRB5*01:01, DRB1*15:01, DQB1*03:04, and A*11:01.

In some embodiments, the subject has the HLA allele DRB1*11:04 and the amino acid sequence is SEQ ID NO: 19, the subject has the HLA allele DQB1*03:03 and the amino acid sequence is SEQ ID NO: 31, the subject has the HLA allele DQA1*05:01 and the amino acid sequence is SEQ ID NO: 32, the subject has the HLA allele DRB1*01:01 and the amino acid sequence is SEQ ID NO: 40, the subject has the HLA allele DRB1*04:04 and the amino acid sequence is SEQ ID NO: 49, the subject has the HLA allele DQB1*04:02 and the amino acid sequence is SEQ ID NO: 52, or the subject has the HLA allele DRB3*02:02 and the amino acid sequence is SEQ ID NO:

29.

In some embodiments the method assesses whether AD, ALS or PD is developing in a subject who has been identified as being at risk of developing AD, ALS or PD, or assesses whether a subject afflicted with AD, ALS or PD is likely to benefit from a therapy.

The present invention also provides methods for treating a subject afflicted with an α-synucleinopathy, a Tauopathy, Parkinson's disease (PD), amyotrophic lateral sclerosis (ALS), Lewy Body dementia (LBD), or Alzheimer's disease (AD)comprising
a) administering to the subject a compound that is approved for use in treating subjects afflicted with the α-synucleinopathy, PD, ALS, LBD or AD, wherein the subject has been diagnosed or confirmed to be afflicted with α-synucleinopathy, PD, ALS, LBD or AD according to the methods;
b) diagnosing or confirming the subject to be afflicted with the α-synucleinopathy, PD, ALS, LBD or AD according to the methods, and administering to the subject a compound that is approved for use in treating subjects afflicted with α-synucleinopathy, PD, ALS, LBD or AD;
c) administering to the subject a therapy that is directed to leukocytes that are activated by an epitope peptide, wherein leukocytes of the subject have been determined to have increased activation after contact with the epitope peptide;
d) administering an immunosuppressant therapy to the subject, wherein the subject has been identified as being likely to benefit therefrom by the methods; or
e) administering an immunosuppressant therapy to the subject, wherein the subject has been identified as being likely to benefit from a therapy directed to leukocytes that are activated by an epitope peptide according to the methods.

In some embodiments, the therapy is tolerization therapy, and the tolerization therapy is specific for leukocytes that are activated by the epitope, preferably wherein administering the tolerization therapy comprises administering to the subject the epitope peptide in an amount that is effective to reduce activation of leukocytes in the subject by the epitope peptide.

In some embodiments, the therapy comprises selectively killing the leukocytes that are activated by the epitope peptide in the subject, preferably wherein selectively killing the leukocytes that are activated by the epitope peptide in the subject comprises administering to the subject an effective amount of a compound comprising a major histocompatibility complex (MHC) Tetramer and a toxin to the subject, wherein the MHC Tetramer comprises the epitope peptide.

In some embodiments, the immunosuppressant therapy comprises tolerization therapy, selectively killing the leukocytes that are activated by an epitope peptide in the subject, or administering an effective amount of an immunosuppressive compound to the subject, preferably wherein the immunosuppressive compound is a calcineurin inhibitor, a compound that blocks a chemokine receptor that is expressed by a leukocyte, a glucocorticoid, a mTOR inhibitor, an anti-metabolic compound, a phosphodiesterase-5 inhibitor, an antibody, or a leukocyte function antigen-3 (LFA-3)/Fc fusion protein.

The present invention also provides methods for assessing whether leukocytes of a subject afflicted with an α-synucleinopathy, a Tauopathy, Parkinson's disease (PD), amyotrophic lateral sclerosis (ALS), Lewy Body dementia (LBD), or Alzheimer's disease (AD) are activated by an epitope peptide, comprising
i) obtaining leukocytes from the subject;
ii) contacting the leukocytes with the epitope peptide;
iii) determining whether the leukocytes have increased activation after contact with the epitope peptide; and
iv) identifying the leukocytes of the subject as activated by the epitope peptide if in step iii) the leukocytes are determined to have increased activation after contact with the epitope peptide, and identifying the leukocytes of the subject as not activated by the epitope peptide if in step iii) the leukocytes are determined to not have increased activation after contact with the epitope peptide, wherein
   a. the epitope peptides is represented by an amino acid sequence selected from the group of Tau derived sequences represented by SEQ ID NO: 1-55 or 240-376,
   b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or
   c. wherein the epitope peptide is represented by the amino acid sequence selected from the group of TDP43 derived sequences represented by SEQ ID NO: 56-239.

In an embodiment, the assessment is made as to whether the leukocytes of a subject afflicted with AD, ALS or PD are activated by the epitope peptide.

In further embodiments, the epitope peptide:
a) is or comprises part of a compound that is produced by neurons in subjects afflicted with the α-synucleinopathy, PD, ALS, LBD or AD;
b) comprises consecutive amino acids that are identical to a stretch of consecutive amino acids in a protein that is produced by the neurons;
c) comprises consecutive amino acids that are identical to a stretch of consecutive amino acids in a Tau mutant;
d) comprises about 16, at least 15, 5-50, 8-11, or 8-14 amino acids;
e) is phosphorylated, acetylated, nitrated, or dopamine modified;
f) comprises a phosphorylated serine or a phosphorylated tyrosine;
g) comprises a phosphorylated serine or a phosphorylated tyrosine, wherein the phosphorylated serine or phosphorylated tyrosine is within a stretch of consecutive amino acids that is identical to a stretch of consecutive amino acids comprising the serine at position 199, 202, 214, 262, 356, or 422 of Tau or the tyrosine at position 181, 205, 212, 231, or 262 of Tau.
h) is or comprises part of a compound that is produced by neurons in subjects afflicted with the α-synucleinopathy, PD, ALS, LBD or AD, wherein the neurons are in the ventral midbrain, the substantia nigra, the locus coeruleus, or the ventral tegmental area;
i) is or comprises part of a compound that is produced by neurons in subjects afflicted with the α-synucleinopathy, PD, ALS, LBD or AD, wherein the neurons are catecholamine neurons;
j) comprises consecutive amino acids that are identical to a stretch of consecutive amino acids in an α-syn mutant;
k) comprises consecutive amino acids that are identical to a stretch of consecutive amino acids in an α-syn mutant, wherein the α-syn mutant is an α-syn A53T or A30P mutant;
l) comprises a phosphorylated serine or a phosphorylated tyrosine, wherein the phosphorylated serine or phosphorylated tyrosine is within a stretch of consecutive amino acids that is identical to a stretch of consecutive amino acids comprising the serine at position 129 of α-syn or the tyrosine at position 39 of α-syn;
m) is or comprises part of a compound that is produced by neurons in subjects afflicted with the ALS, wherein the neurons are in the motor area;
n) is or comprises part of a compound that is produced by neurons in subjects afflicted with ALS, wherein the neurons are motor neurons;
o) comprises consecutive amino acids that are identical to a stretch of consecutive amino acids in TDP43, FUS, or SOD-1;
p) comprises consecutive amino acids that are identical to a stretch of consecutive amino acids in TDP43 mutant, FUS mutant, or SOD-1 mutant;
q) comprises a deamidated asparagine, an oxidized threonine, or a phosphorylated tyrosine.

In further embodiments, the epitope peptide comprises consecutive amino acids in the sequence set forth as MRGVRLVEGILHAPD (SEQ ID NO: 231), LVYVVNYPKDNKRKM (SEQ ID NO: 233), DMTEDELREFFSQYG (SEQ ID NO: 236), ELREFFSQYGDVMDV (SEQ ID NO: 237), EDLIIKGISVHISNA (SEQ ID NO: 74), EDDGTVLLSTVTAQF (SEQ ID NO: 229), AGWGNLVYVVNYPKD (SEQ ID NO: 232), DVMDVFIPKPFRAFA (SEQ ID NO: 238), or FIPKPFRAFAFVTFA (SEQ ID NO: 239).

In some embodiments, in step iii) the leukocytes are determined to have increased activation after contact with the epitope peptide,
a) if the leukocytes express or release more of at least one cytokine compared to corresponding leukocytes not contacted with the epitope peptide;
b) if the leukocytes release at least one cytokine;
c) if the leukocytes release at least one cytokine, wherein in step iii) the leukocytes are determined to have released the at least one cytokine if there are over 20 spot-forming cells (SFC) per million cells as measured by an ELISpot assay comprising the colorimetric detection of the at least one cytokine.

In some embodiments, the leukocytes are T cells. In a further embodiment, the cytokine is interferon-gamma (IFN-□) or IL-5. In a further embodiment, the cytokine is TNFα, IL-4, IL-17, IL-10, or IL-21. In a further embodiment, the cytokine is two or more cytokines, wherein the two or more cytokines are at least IFN-γ and IL-5. In a further embodiment, the leukocytes are T cells which are CD4+ T cells, CD8+ T cells, and/or CD4+CD8+ T cells. In a further embodiment, the leukocytes are IL-4-producing CD4+ T cells, IFN-γ-producing CD4+ T cells, or IFN-γ-producing CD8+ T cells.

In some embodiments, the at least one cytokine is at least interferon-gamma (IFN-□), IL-4 or IL-5, wherein the at least one cytokine that is expressed or released from the leukocytes is assayed through a process comprising an enzyme-linked immunosorbent assay (ELISA), enzyme-lined immunospot (ELISPOT), intracellular cytokine staining (ICS), or quantitative RT-PCR.

In some embodiments, the leukocytes are CD4+ T cells.

In some embodiments, determining whether the leukocytes have increased activation comprises
a) contacting the leukocytes with compound comprising a major histocompatibility complex (MHC) Tetramer having four MHC molecules, wherein each MHC molecule is associated with an epitope peptide; and
b) identifying leukocytes that become bound to the compound as activated.

The present invention also provides methods for assessing whether a test compound comprises an epitope peptide to which leukocytes of a subject suffering from a neurological disorder are responsive comprising
i) obtaining leukocytes from the subject;
ii) contacting the leukocytes with the test compound;
iii) determining whether the leukocytes has increased activation after contact with the test compound; and
iv) identifying the test compound as comprising an epitope peptide to which the leukocytes are responsive if in step iii) the leukocytes are determined to have increased activation after contact with the test compound, and identifying the test compound as not comprising an epitope to which the leukocytes are responsive if in step iii) the leukocytes are determined to not have increased activation after contact with the test compound, wherein
   a. the epitope peptides is represented by an amino acid sequence selected from the group of Tau derived sequences represented by SEQ ID NO: 1-55 or 240-376,
   b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or.
   c. wherein the epitope peptide is represented by the amino acid sequence selected from the group of TDP43 derived sequences represented by SEQ ID NO: 56-239.

In an embodiment the test compound is or comprises part of a compound that is produced by neurons in subjects afflicted with an α-synucleinopathy, a Tauopathy, Parkinson's disease (PD), amyotrophic lateral sclerosis (ALS), Lewy Body dementia (LBD), or Alzheimer's disease (AD).

In a specific embodiment the amino acid sequence is selected from the group of sequences consisting of SEQ ID NO: 136-165. In a further embodiment, the amino acid sequence is selected from the group of sequences consisting of SEQ ID NO: 136-138, SEQ ID NO: 140-143, SEQ ID NO: 145-146, SEQ ID NO: 148-152, SEQ ID NO: 154, and SEQ ID NO: 158-159.

The present invention also provides for a kit comprising an epitope peptide.

The present invention also provides for compounds for treating an α-synucleinopathy, a Tauopathy, Parkinson's disease (PD), amyotrophic lateral sclerosis (ALS), Lewy Body dementia (LBD), or Alzheimer's disease (AD), comprising i) a major histocompatibility complex (MHC) Tetramer having four MHC molecules, wherein each MHC molecule is associated with an epitope peptide, and ii) a toxin, wherein
a. the epitope peptides is represented by an amino acid sequence selected from the group of Tau derived sequences represented by SEQ ID NO: 1-55 or 240-376,
b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or.
c. wherein the epitope peptide is represented by the amino acid sequence selected from the group of TDP43 derived sequences represented by SEQ ID NO: 56-239.

The present invention also provides processes for assessing whether a subject is at risk of developing an α-synucleinopathy, a Tauopathy, Parkinson's disease (PD), amyotrophic lateral sclerosis (ALS), Lewy Body dementia (LBD), or Alzheimer's disease (AD), which involves an array of testing, the improvement compriing including in the array of testing the steps of:
a)
   i) obtaining leukocytes from the subject;
   ii) contacting the leukocytes with an epitope peptide;
   iii) determining whether the leukocytes have increased activation after contact with the epitope peptide; and
   iv) identifying the subject as at risk of developing α-synucleinopathy, PD, ALS, LBD or AD if in step iii) the leukocytes are determined to have increased activation after contact with the epitope peptide, and identifying the subject as not at risk of developing the α-synucleinopathy, PD, ALS, LBD or AD if in step iii) the leukocytes are determined to not have increased activation after contact with the epitope peptide, wherein
   a. the epitope peptides is represented by an amino acid sequence selected from the group of Tau derived sequences represented by SEQ ID NO: 1-55 or 240-376,
   b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or.
   c. wherein the epitope peptide is represented by the amino acid sequence selected from the group of TDP43 derived sequences represented by SEQ ID NO: 56-239
   or
b)
   i) obtaining leukocytes from the subject;
   ii) separating the leukocytes into 2 or more pools of leukocytes and contacting each pool with an epitope peptide, wherein each pool is contacted with a different epitope;
   iii) determining whether each pool has increased activation after contact with the epitope peptide; and
   iv) identifying the subject as at risk of developing the α-synucleinopathy, PD, ALS, LBD or AD if in step iii) 1 or more pools is determined to have increased activation after contact with the epitope peptide, wherein
   a. the epitope peptides is represented by an amino acid sequence selected from the group of Tau derived sequences represented by SEQ ID NO: 1-55 or 240-376,
   b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or
   c. wherein the epitope peptide is represented by the amino acid sequence selected from the group of TDP43 derived sequences represented by SEQ ID NO: 56-239.

The present invention also provides processes for diagnosing or confirming whether a subject is afflicted with an α-synucleinopathy, a Tauopathy, Parkinson's disease (PD), amyotrophic lateral sclerosis (ALS), Lewy Body dementia (LBD), or Alzheimer's disease (AD), which involves an array of testing, the improvement comprising including in the array of testing the steps of:
a)
   i) obtaining leukocytes from the subject;
   ii) separating the leukocytes into 1 or more pools of leukocytes and contacting each pool with an epitope peptide, wherein each pool is contacted with a different epitope peptide;
   iii) determining whether each pool has increased activation after contact with the epitope peptide; and
   iv) identifying the subject as afflicted with the α-synucleinopathy, PD, ALS, LBD or AD if and only if in step iii) 1 or more pools is determined to have increased activation after contact with an epitope peptide, or
b)
   i) obtaining leukocytes from the subject;
   ii) separating the leukocytes into 1 or more pools of leukocytes and contacting each pool with an epitope peptide, wherein each pool is contacted with a different epitope peptide;
   iii) determining whether each pool has increased activation after contact with the epitope peptide; and
   iv) identifying the subject as afflicted with the α-synucleinopathy, PD, ALS, LBD or AD if and only if in step iii) 1 or more pools is determined to have increased activation after contact with an epitope peptide, wherein
a. the epitope peptides is represented by an amino acid sequence selected from the group of Tau derived sequences represented by SEQ ID NO: 1-55 or 240-376,
b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or.
c. wherein the epitope peptide is represented by the amino acid sequence selected from the group of TDP43 derived sequences represented by SEQ ID NO: 56-239.

In an embodiment, the leukocytes have increased activation after contact with native alpha-synuclein protein or fibrilized alpha-synuclein protein.

The present invention also provides for pharmaceutical compositions for treating an α-synucleinopathy, a Tauopathy, Parkinson's disease (PD), amyotrophic lateral sclerosis (ALS), Lewy Body dementia (LBD), or Alzheimer's disease (AD), comprising
i) a protein comprising an amino acid sequence selected from the group of
   a. the epitope peptides is represented by an amino acid sequence selected from the group of Tau derived sequences represented by SEQ ID NO: 1-55 or 240-376,
   b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or.
   c. wherein the epitope peptide is represented by the amino acid sequence selected from the group of TDP43 derived sequences represented by SEQ ID NO: 56-239, and
ii) a pharmaceutically acceptable carrier.

In an embodiment, the the amino acid sequence is selected from the group of sequences SEQ ID NO: 74, 139, 144 and 230-239, or wherein the amino acid sequence is selected from the group of sequences SEQ ID NO: 231, 233, 236, and 237, or wherein the amino acid sequence is selected from the group of sequences SEQ ID NO: 74, 229, 231, 232, 239, or 239.

The present invention also provides a method comprising:
a. providing a biological sample from a subject;
b. processing the biological sample to determine presence of a T cell receptor (TCR) specific to a peptide, wherein the peptide is a fragment from a protein associated with said neurodegenerative disease.

In some embodiments, the processing step includes contacting T cells from said sample with said peptide, and detecting activation of a T cell having said TCR. In a further embodiment, the processing step includes performing gene sequencing on at least a cellular fraction of said biological sample to amplify a gene encoding the TCR specific to said peptide, and detecting presence of said gene encoding said TCR, preferably wherein said at least a cellular fraction of said biological sample includes peripheral blood mononuclear cells (PBMC), preferably leukocytes.

In some embodiments, the peptide associated with a neurodegenerative disease is tau, alpha-synuclein, or transactive response DNA binding protein 43 kDa (TDP-43). In a further embodiment, the peptide is selected from any one of tables 1 to 4.

The present invention also provides a method comprising:
a) providing a biological sample from a subject;
b) processing the biological sample to determine presence of a human leukocyte antigen (HLA) capable of presenting a peptide, wherein the peptide is a fragment from a protein associated with said neurodegenerative disease; and
c) processing the biological sample to determine presence of a T cell receptor (TCR) specific to said peptide.

In some embodiments, the peptide is a fragment from a protein that forms aggregates in a patient having the neurodegenerative disease.

In some embodiments, the method of step c) for processing the biological sample includes contacting T cells present in said sample with said peptide, and detecting activation of a T cell having said TCR.

In some embodiments, the method of step b) for processing the biological sample includes performing gene sequencing on at least a cellular fraction of said biological sample to amplify a gene encoding the HLA capable of presenting said peptide, and detecting presence of said gene encoding said HLA and c) includes performing gene sequencing on at least a cellular fraction of said biological sample to amplify a gene encoding the TCR specific to said peptide, and detecting presence of said gene encoding said TCR. In some embodiments, at least a cellular fraction of said biological sample includes peripheral blood mononuclear cells (PBMC), preferably leukocytes. In some embodiments, the protein that forms aggregates in a patient having a neurodegenerative disease is tau, alpha-synuclein, or transactive response DNA binding protein 43 kDa (TDP-43).

In some embodiments, the protein is tau, preferably wherein the peptide derived from the tau protein includes a phosphorylated serine and/or tyrosine. In some embodiments, the protein is TDP-43, preferably wherein the peptide derived from the TDP-43 protein includes a phosphorylated serine and/or tyrosine. In further embodiments, the peptide is selected from any one of tables 1 to 4, preferably wherein the peptide is selected from any one of GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495).

In some embodiments, the HLA is DRB5*01:01, DRB1*15:01, DQB1*03:04, A*11:01, DRB1*07:01, DRB1*09:01, or DQBl*03:01.

In some embodiments, the method comprises detecting a peptide:MHC complex comprising any one of the peptides and any one of the HLAs listed in Table 5.

a) In some embodiments, the presence of the TCR and the HLA is indicative that the subject is predisposed to, at risk of, or has a neurodegenerative disease, preferably wherein the presence of the TCR is indicative that the subject is predisposed to, at risk of or has a neurodegenerative disease, preferably wherein the neurodegenerative disease or disorder is alpha-synucleinopathy, a Tauopathy, Parkinson's disease (PD), Lewy Body dementia (LBD), or Alzheimer's disease (AD).

Amyotrophic Lateral Sclerosis

ALS is often diagnosed by a neurologist who can evaluate symptoms and their severity. National Institute of Neurological Disorders and Stroke, Amyotrophic Lateral Sclerosis (ALS) Fact Sheet, available at www.ninds.nih.gov/Disorders/Patient-Caregiver-Education/Fact-Sheets/Amyotrophic-Lateral-Sclerosis-ALS-Fact-Sheet, last updated Oct. 18, 2004 (hereinafter ‘NINDS Fact Sheet“). According to the National Institute of Neurological Disorders and Stroke, there is currently no test that can clearly identify the disease (NINDS Fact Sheet). The presence of upper and lower motor neuron symptoms strongly suggests the presence of the disease (NINDS Fact Sheet). Additionally, muscle and imaging tests, laboratory tests, and tests for other diseases and disorders can help doctors decide if a patient has true ALS or some other disorder that resembles it (NINDS Fact Sheet).

The present invention provides methods for identifying. subjects afflicted with ALS that would previously have remained undetected. Aspects of the present invention enable the detection of ALS in presymptomatic stages. Additionally, the present invention provides methods for identifying those who might eventually develop ALS. ALS has an increased prevalence with age. See, for example, the NINDS Fact Sheet, the entire content of each of which is hereby incorporated herein by reference. Without wishing to be bound by any scientific theory, the peripheral immune response of ALS may begin in a subject decades before ALS may be diagnosed by a neurologist, and subjects having the peripheral immune response may be identified for earlier therapy using methods of the invention. The present invention provides methods for diagnosing causes of these symptoms, and be used to identify subjects who may benefit from prophylactic treatment for ALS.

Aspects of the present invention provide an epitope useful as a test/biomarker for ALS, which could include identifying patients in preclinical stages or in danger of ALS, and to measure disease progression and/or response. As described herein, aspects of the invention provide means to detect these T cells in patient blood. Similar approaches for identifying tuberculosis patients will be known to those skilled in the art. For use of a similar test in TB diagnosis, see en.wikipedia.org/wiki/QuantiFERON, the entire contents of which are hereby incorporated by reference.

Additionally, aspects of the invention define which precise T cells and antigens individual patients have, and provide individualized therapy that spare other important immune functions. The test/biomarker test for ALS has already been conducted and is effective, as described in the Examples disclosed herein.

Without wishing to be bound by any scientific theory, the T cells identified in embodiments of the invention may be a step in the disease. The present invention provides means to treat ALS, as blocking these T cells arrest the disease progression. An example would be tolerization:

particular epitopes the T cells recognize are determined, and patients are exposed to the epitope in a form that alters the immune system to recognize it as self, and halt or reduce making killer T cells. In addition to TDP43, there are additional mutant proteins that are detected in ALS, including FUS and SOD-1. These markers are similarly useful for diagnosing, confirming, providing biomarkers for, and treating ALS.

Alzheimer's Disease

AD is often diagnosed by a neurologist who can evaluate symptoms and their severity. National Institute on Aging, Alzheimer's disease, available at www.nia.nih.gov/alzheimers (hereinafter “NIA”). According to the National Institute on Aging, there is currently no test that can clearly identify the disease (NIA). Tests of memory, problem solving, attention, counting, and language can help doctors decide if a patient having memory problems has “possible Alzheimer's disease” (dementia may be due to another cause), “probable Alzheimer's disease” (no other cause for dementia be found), or some other problem (NIA). Additionally, standard medical tests, such as blood and urine tests, and brain scans, such as computed tomography (CT), magnetic resonance imaging (MRI), or positron emission tomography (PET), may be used to identify other possible causes for symptoms (NIA). These tests may be repeated to give doctors information about how the person's memory and other cognitive functions are changing over time (NIA). However, the diagnosis of Alzheimer's disease can be confirmed when a sample of brain tissue is removed (after death, during an autopsy) and examined under a microscope (Merck Manual). The characteristic loss of nerve cells, neurofibrillary tangles, and senile plaques containing beta-amyloid can be seen throughout the brain, particularly in the area of the temporal lobe that is involved in forming new memories (Merck Manuel).

The present invention provides methods for identifying subjects afflicted with AD that would previously have remained undetected. Aspects of the present invention enable the detection of AD in presymptomatic stages. Additionally, the present invention provides methods for identifying those who might eventually develop AD. AD has an increased prevalence with age. See, for example, the NIA; and Mayeaux and Stern, (2012) Epidemiology of Alzheimer Disease, Cold Spring Harb Perspect Med. 2 (8): a006239, the entire content of each of which is hereby incorporated herein by reference. Without wishing to be bound by any scientific theory, the peripheral immune response of AD may begin in a subject decades before AD may be diagnosed by a neurologist, and subjects having the peripheral immune response may be identified for earlier therapy using methods of the invention. The present invention provides methods for diagnosing causes of these symptoms, and be used to identify subjects who may benefit from prophylactic treatment for AD.

Aspects of the present invention provide an epitope useful as a test/biomarker for AD, which could include identifying patients in preclinical stages or in danger of AD, and to measure disease progression and/or response. As described herein, aspects of the invention provide means to detect these T cells in patient blood. Similar approaches for identifying tuberculosis patients will be known to those skilled in the art. For use of a similar test in TB diagnosis, see en.wikipedia.org/wiki/QuantiFERON, the entire contents of which are hereby incorporated by reference.

Additionally, aspects of the invention define which precise T cells and antigens individual patients have, and provide individualized therapy that spare other important immune functions. The test/biomarker test for AD has already been conducted and is effective, as described in the Examples disclosed herein.

Without wishing to be bound by any scientific theory, the T cells identified in embodiments of the invention may be a step in the disease. The present invention provides means to treat AD, as blocking these T cells arrest the disease progression. An example would be tolerization: particular epitopes the T cells recognize are determined, and patients are exposed to the epitope in a form that alters the immune system to recognize it as self, and halt or reduce making killer T cells. In addition to Tau, there are additional mutant proteins that cause AD, including β-amyloid. These markers are similarly useful for diagnosing, confirming, providing biomarkers for, and treating AD.

Parkinson' Disease

PD is often diagnosed by a neurologist who can evaluate symptoms and their severity. National Institute of Neurological Disorders and Stroke, Parkinson's Disease Backgrounder, available at www.ninds.nih.gov/disorders/parkinsons_disease/parkinsons_disease_back grounder.htm, last updated Oct. 18, 2004 (hereinafter “NINDS Backgrounder”. According to the National Institute of Neurological disorders and Stroke, there is currently no test that can clearly identify the disease. (NINDS Backgrounder). Sometimes people with suspected PD are given anti-Parkinson's drugs to see if they respond (NINDS Backgrounder). Other tests, such as brain scans, can help doctors decide if a patient has true PD or some other disorder that resembles it (NINDS Backgrounder). Microscopic brain structures called Lewy bodies, which can be seen only during an autopsy, are regarded as a hallmark of classical PD (NINDS Backgrounder). Autopsies have uncovered Lewy bodies in a surprising number of older persons without diagnosed PD—8% of people over 50, almost 13% of people over 70, and almost 16% of those over 80, according to one study (NINDS Backgrounder). As a result, some experts believe PD is something of an “iceberg; phenomenon,” lurking undetected in as many as 20 people for each known Parkinson's patient (NINDS Backgrounder). Without wishing to be bound by any scientific theory, a few researchers contend that almost everyone would develop Parkinson's eventually if they lived long enough (NINDS Backgrounder).

The present invention provides methods for identifying subjects afflicted with PD that would previously have remained undetected. Aspects of the present invention enable the detection of PD in presymptomatic stages. Additionally, the present invention provides methods for identifying those who might eventually develop PD. PD has an increased prevalence with age. See, for example, the NINDS Backgrounder; and Van Den Eeden et al., (2003) Incidence of Parkinson's Disease: Variation by Age, Gender, and Race/Ethnicity, Am. J. Epidemiol. 157 (11): 1015-1022, the entire content of each of which is hereby incorporated herein by reference. PD ranks among the most common late-life neurodegenerative diseases, affecting approximately 1.5% to 2.0% of people aged 60 years and older (Patrick Sweeney, Parkinson's Disease, Cleveland Clinic, available at clevelandclinicmeded.com/medicalpubs/diseasemanagement/neurology/parkinsons-disease/). Without wishing to be bound by any scientific theory, the peripheral immune response of PD may begin in a subject decades before PD may be diagnosed by a neurologist, and subjects having the peripheral immune response may be identified for earlier therapy using methods of the invention. There are a number of peripheral symptoms associated with PD including orthostatic hypotension and constipation. The present invention provides methods for diagnosing causes of these symptoms, and be used to identify subjects who may benefit from prophylactic treatment for PD.

Aspects of the present invention provide an epitope peptide useful as a test/biomarker for PD, which could include identifying patients in preclinical stages or in danger of PD, and to measure disease progression and/or response. As described herein, aspects of the invention provide means to detect these T cells in patient blood. Similar approaches for identifying tuberculosis patients will be known to those skilled in the art. For use of a similar test in TB diagnosis, see en.wikipedia.org/wiki/QuantiFERON, the entire contents of which are hereby incorporated by reference.

Additionally, aspects of the invention define which precise T cells and antigens individual patients have, and provide individualized therapy that spare other important immune functions. The test/biomarker test for PD has already been conducted and is effective, as described in the Examples disclosed herein.

Without wishing to be bound by any scientific theory, the T cells identified in embodiments of the invention may kill the neurons in PD, and thus be a step in the disease. The present invention provides means to treat PD, as blocking these T cells arrest the disease progression. An example would be tolerization: particular epitopes the T cells recognize are determined, and patients are exposed to the epitope in a form that alters the immune system to recognize it as self, and halt or reduce making killer T cells. In addition to α-synuclein, there are additional mutant proteins that cause PD, including LRRK2 and glucocerebrosidase. These markers are similarly useful for diagnosing, confirming, providing biomarkers for, and treating PD.

Each embodiment disclosed herein is contemplated as being applicable to each of the other disclosed embodiments. Thus, all combinations of the various elements described herein are within the scope of the invention.

It is understood that where a parameter range is provided, all integers within that range, and tenths thereof, are also provided by the invention.

For example, “0.2-5 mg/kg” is a disclosure of 0.2 mg/kg, 0.3 mg/kg, 0.4 mg/kg, 0.5 mg/kg, 0.6 mg/kg etc. up to 5.0 mg/kg.

Proteins

In the present disclosure, methods, systems and procedures are presented from which the person of skill can reasonably predict that various proteins that are associated with a neurodegenerative disease, for example where the protein forms aggregates associated with the disease, may contain epitopes that are recognized as autoantigens by T cells and such proteins or peptides derived therefrom may thus be useful in such methods, systems and procedures.

Examples of proteins that may illustrate the concept underlying the present disclosure are discussed below. For example, these proteins may include peptides that may be useful in the herein described processes, systems and methods that make use of assessing the presence of TCR specific to a given peptide to determine neurodegenerative disease predisposition or presence thereof, and/or to determine responsiveness to therapeutic treatment of the disease.

In one non-limiting embodiment, the TAR DNA-binding protein 43 (TDP-43, transactive response DNA binding protein 43 kDa) may represent an example of a protein that may contain epitopes that are recognized as autoantigens by T cells at least based on one or more of the following observations:
a. hyper-phosphorylated, ubiquitinated and cleaved form of TDP-43 —known as pathologic TDP43 —is the major disease protein in ubiquitin-positive, tau-, and alpha-synuclein-negative frontotemporal dementia (FTLD-TDP) and in Amyotrophic lateral sclerosis (ALS). [Neumann et al., (2006). Science, 314 (5796): 130-3].
b.Elevated levels of the TDP-43 protein have been identified in individuals diagnosed with chronic traumatic encephalopathy, a condition that often mimics ALS and that has been associated with athletes who have experienced multiple concussions and other types of head injury.
c. Abnormalities of TDP-43 occur in an important subset of Alzheimer's disease patients, correlating with clinical and neuropathologic features indexes. [Tremblay et al., (2011), J Neuropathol Exp Neurol. 70 (9): 788-98].

In one non-limiting embodiment, the tau protein may represent another example of a protein that may contain epitopes that are recognized as autoantigens by T cells at least based on one or more of the following observations:
a. Tau is the protein product of the microtubule-associated protein tau (MAPT) gene. This protein, in highly phosphorylated aggregates, has long been associated with Alzheimer's disease (AD), progressive supranuclear palsy (PSP) and other dementias.
b. MAPT has been identified as a risk factor for Parkinson's disease (PD) by genome-wide association study (GWAS) [Sharma et al., Neurology, 2012, Aug 14; 79(7):659-67], but not AD itself.
c. Phospho-tau immunolabel can also be high in PD and particularly brain of LBD (Parkinson's disease dementia and dementia with Lewy bodies), while phospho-tau is higher in AD, and there is often significant overlap in patient brain pathology between the disorders [Arnold et al., J Comp Neurol, 2013, Dec 15; 521(18): 4339-55].
d. Tau and alpha-syn have many parallel features including association with PD by GWAS, phosphorylation under disease conditions, presence of both proteins [Hampel et al., Nat Rev Drug Discov., 2010 Jul; 9(7): 560-74; Foulds et al., Sci Rep., 2013; 3: 2540; Zetterberg et al., Alzheimers Res Ther., 2013, Mar 28; 5(2): 9], and autoantibodies in blood [Bartos et al., J Neuroimmunol., 2012, Nov 15; 252(1-2): 100-5; Koehler et al., PLoS One, 2013, May 31;8(5):e64649], and similar degradation by chaperone-mediated autophagy (CMA) that is disturbed by mutation [Wang et al., Hum Mol Genet., 2009, Nov 1; 18(21): 4153-70]. They may even form joint oligomers in some patients. [Sengupta et al., Biol Psychiatry, 2015, Nov 15; 78(10): 672-83].
e. Phosphorylated candidate epitopes are important for tau, which has about 40 potential phosphorylated sites (Sharma et al., 2012; Yin et al., 2013), of which 20 were identified in AD patients (Duka et al., 2013); 10 phosphorylated sites were identified in PD striata (S202, 235, 262, 356, 396/404, 409, 413, 422 and T205, 212); and seven sites in LBD (S214, 238, 396/404, 422 and T212, 217).
f. In PD patients, there are 3 clusters of phospho-tau (202, 205, 212; 356, 396, 404; and 409, 413, 422).

Amino acid and nucleotide sequences of tau are accessible in public databases by the NCBI Gene ID: 4137.

In one non-limiting embodiment, the alpha-synuclein protein may represent another example of a protein that may contain epitopes that are recognized as autoantigens by T cells at least based on one or more of the following observations:
a. T cells restricted by DRB5*01:01, DRB1*15:01, DQB1*03:04, A*11:01, DRB1*07:01, DRB1*09:01, or DQB1*03:01 have been shown to recognize specifically certain alpha-synuclein peptides in PD patients. [Sulzer et al., Nature, In press, doi:10.1038].

The reader will readily understand that the present disclosure is not limited in terms of practical implementation to the use of any particular protein from the above non-exhaustive list of proteins.

Peptides

In some embodiments, the epitope described herein comprises consecutive amino acids that are identical to a stretch of consecutive amino acids in the herein described protein. In other words, it is in the form of a peptide.

In some embodiments, the peptide is phosphorylated or nitrated. In some embodiments, the epitope comprises a phosphorylated serine and/or a phosphorylated tyrosine. In some embodiments, the epitope comprises a phosphorylated serine.

In some embodiments, the peptide comprises consecutive amino acids that are identical to a stretch of consecutive amino acids of the tau protein, where at least one phosphorylated serine and/or phosphorylated tyrosine is within the stretch of consecutive amino acids. In some embodiments, the stretch of consecutive amino acids of the tau protein comprises the serine at position 195, 198, 202, 214, 235, 237, 238, 262, 356, 396/404, 400, 409, 413, or 422, or the tyrosine at position 181, 184, 205, 212, 217, or 231.

In some embodiments, the peptide comprises a non-amino acid polymer that is produced by the neurons. In some embodiments, the peptide is neuromelanin or a portion thereof. Motor symptoms of PD are caused by cell death in the substantia nigra, which may be partly due to oxidative stress. This oxidation may be relieved by neuromelanin.

In some embodiments, the epitope comprises consecutive amino acids in the peptide sequences derived from tau, as set forth in Table 1.

TABLE 1
peptide sequences derived from tau

| SEQ ID NO | Peptide | Start | End |
|---|---|---|---|
| 1 | MAEPRQEFEVMEDHAG | 1 | 16 |
| 2 | EVMEDHAGTYGLGDRK | 9 | 24 |
| 3 | TYGLGDRKDQGGYTMH | 17 | 32 |
| 4 | DQGGYTMHQDQEGDTD | 25 | 40 |
| 5 | QDQEGDTDAGLKESPL | 33 | 48 |
| 6 | AGLKESPLQTPTEDGS | 41 | 56 |
| 7 | QTPTEDGSEEPGSETS | 49 | 64 |
| 8 | EEPGSETSDAKSTPTA | 57 | 72 |
| 9 | DAKSTPTAEDVTAPLV | 65 | 80 |
| 10 | EDVTAPLVDEGAPGKQ | 73 | 88 |
| 11 | DEGAPGKQAAAQPHTE | 81 | 96 |
| 12 | AAAQPHTEIPEGTTAE | 89 | 104 |
| 13 | IPEGTTAEEAGIGDTP | 97 | 112 |
| 14 | EAGIGDTPSLEDEAAG | 105 | 120 |
| 15 | SLEDEAAGHVTQARMV | 113 | 128 |
| 16 | HVTQARMVSKSKDGTG | 121 | 136 |
| 17 | SKSKDGTGSDDKKAKG | 129 | 144 |
| 18 | SDDKKAKGADGKTKIA | 137 | 152 |
| 19 | ADGKTKIATPRGAAPP | 145 | 160 |
| 20 | TPRGAAPPGQKGQANA | 153 | 168 |
| 21 | GQKGQANATRIPAKTP | 161 | 176 |
| 22 | TRIPAKTPPAPKTPPS | 169 | 184 |
| 23 | PAPKTPPSSGEPPKSG | 177 | 192 |
| 24 | SGEPPKSGDRSGYSSP | 185 | 200 |
| 25 | DRSGYSSPGSPGTPGS | 193 | 208 |
| 26 | GSPGTPGSRSRTPSLP | 201 | 216 |
| 27 | RSRTPSLPTPPTREPK | 209 | 224 |
| 28 | TPPTREPKKVAVVRTP | 217 | 232 |
| 29 | KVAVVRTPPKSPSSAK | 225 | 240 |
| 30 | PKSPSSAKSRLQTAPV | 233 | 248 |
| 31 | SRLQTAPVPMPDLKNV | 241 | 256 |
| 32 | PMPDLKNVKSKIGSTE | 249 | 264 |
| 33 | KSKIGSTENLKHQPGG | 257 | 272 |
| 34 | NLKHQPGGGKVQIINK | 265 | 280 |
| 35 | GKVQIINKKLDLSNVQ | 273 | 288 |
| 36 | KLDLSNVQSKCGSKDN | 281 | 296 |
| 37 | SKCGSKDNIKHVPGGG | 289 | 304 |
| 38 | IKHVPGGGSVQIVYKP | 297 | 312 |
| 39 | SVQIVYKPVDLSKVTS | 305 | 320 |
| 40 | VDLSKVTSKCGSLGNI | 313 | 328 |
| 41 | KCGSLGNIHHKPGGGQ | 321 | 336 |
| 42 | HHKPGGGQVEVKSEKL | 329 | 344 |
| 43 | VEVKSEKLDFKDRVQS | 337 | 352 |
| 44 | DFKDRVQSKIGSLDNI | 345 | 360 |
| 45 | KIGSLDNITHVPGGGN | 353 | 368 |
| 46 | THVPGGGNKKIETHKL | 361 | 376 |
| 47 | KKIETHKLTFRENAKA | 369 | 384 |
| 48 | TFRENAKAKTDHGAEI | 377 | 392 |
| 49 | KTDHGAEIVYKSPVVS | 385 | 400 |
| 50 | VYKSPVVSGDTSPRHL | 393 | 408 |
| 51 | GDTSPRHLSNVSSTGS | 401 | 416 |
| 52 | SNVSSTGSIDMVDSPQ | 409 | 424 |
| 53 | IDMVDSPQLATLADEV | 417 | 432 |
| 54 | LATLADEVSASLAKQG | 425 | 440 |
| 55 | ATLADEVSASLAKQGL | 426 | 441 |

In some embodiments, the epitope comprises consecutive amino acids in the peptide sequences derived from TDP-43, as set forth in Table 2.

TABLE 2
peptide sequences derived from TDP-43

| SEQ ID NO | Peptide |
|---|---|
| 56 | AFAFVTFADDQIAQS |
| 57 | FAFVTFADDQIAQSL |
| 58 | AFVTFADDQIAQSLC |
| 59 | FVTFADDQIAQSLCG |
| 60 | VTFADDQIAQSLCGE |
| 61 | TFADDQIAQSLCGED |
| 62 | FADDQIAQSLCGEDL |
| 63 | ADDQIAQSLCGEDLI |
| 64 | DDQIAQSLCGEDLII |
| 65 | DQIAQSLCGEDLIIK |
| 66 | QIAQSLCGEDLIIKG |
| 67 | IAQSLCGEDLIIKGI |
| 68 | AQSLCGEDLIIKGIS |
| 69 | QSLCGEDLIIKGISV |
| 70 | SLCGEDLIIKGISVH |
| 71 | LCGEDLIIKGISVHI |
| 72 | CGEDLIIKGISVHIS |
| 73 | GEDLIIKGISVHISN |
| 74 | EDLIIKGISVHISNA |
| 75 | DLIIKGISVHISNAE |
| 76 | LIIKGISVHISNAEP |
| 77 | IIKGISVHISNAEPK |
| 78 | IKGISVHISNAEPKH |
| 79 | KGISVHISNAEPKHN |
| 80 | GISVHISNAEPKHNS |
| 81 | ISVHISNAEPKHNSN |
| 82 | SVHISNAEPKHNSNR |
| 83 | VHISNAEPKHNSNRQ |
| 84 | HISNAEPKHNSNRQL |
| 85 | ISNAEPKHNSNRQLE |
| 86 | SNAEPKHNSNRQLER |
| 87 | NAEPKHNSNRQLERS |
| 88 | AEPKHNSNRQLERSG |
| 89 | EPKHNSNRQLERSGR |
| 90 | PKHNSNRQLERSGRF |
| 91 | KHNSNRQLERSGRFG |
| 92 | HNSNRQLERSGRFGG |
| 93 | NSNRQLERSGRFGGN |
| 94 | SNRQLERSGRFGGNP |
| 95 | NRQLERSGRFGGNPG |
| 96 | RQLERSGRFGGNPGG |
| 97 | QLERSGRFGGNPGGF |
| 98 | LERSGRFGGNPGGFG |
| 99 | ERSGRFGGNPGGFGN |
| 100 | RSGRFGGNPGGFGNQ |
| 101 | SGRFGGNPGGFGNQG |
| 102 | GRFGGNPGGFGNQGG |
| 103 | RFGGNPGGFGNQGGF |
| 104 | FGGNPGGFGNQGGFG |
| 105 | GGNPGGFGNQGGFGN |
| 106 | GNPGGFGNQGGFGNS |
| 107 | NPGGFGNQGGFGNSR |
| 108 | PGGFGNQGGFGNSRG |
| 109 | GGFGNQGGFGNSRGG |
| 110 | GFGNQGGFGNSRGGG |
| 111 | FGNQGGFGNSRGGGA |
| 112 | GNQGGFGNSRGGGAG |
| 113 | NQGGFGNSRGGGAGL |
| 114 | QGGFGNSRGGGAGLG |
| 115 | GGFGNSRGGGAGLGN |
| 116 | GGFGNSRGGGAGLGNN |
| 117 | FGNSRGGGAGLGNNQ |
| 118 | GNSRGGGAGLGNNQG |
| 119 | NSRGGGAGLGNNQGS |
| 120 | SRGGGAGLGNNQGSN |
| 121 | RGGGAGLGNNQGSNM |
| 122 | GGGAGLGNNQGSNMG |
| 123 | GGAGLGNNQGSNMGG |
| 124 | GAGLGNNQGSNMGGG |
| 125 | AGLGNNQGSNMGGGM |
| 126 | GLGNNQGSNMGGGMN |
| 127 | LGNNQGSNMGGGMNF |
| 128 | GNNQGSNMGGGMNFG |
| 129 | NNQGSNMGGGMNFGA |
| 130 | NQGSNMGGGMNFGAF |
| 131 | QGSNMGGGMNFGAFS |
| 132 | GSNMGGGMNFGAFSI |
| 133 | SNMGGGMNFGAFSIN |
| 134 | NMGGGMNFGAFSINP |
| 135 | MGGGMNFGAFSINPA |
| 136 | MGGGMNFGAFSINPA |
| 137 | GGMNFGAFSINPAMM |
| 138 | GMNFGAFSINPAMMA |
| 139 | MNFGAFSINPAMMAA |
| 140 | NFGAFSINPAMMAAA |
| 141 | FGAFSINPAMMAAAQ |
| 142 | GAFSINPAMMAAAQA |
| 143 | AFSINPAMMAAAQAA |
| 144 | FSINPAMMAAAQAAL |
| 145 | SINPAMMAAAQAALQ |
| 146 | INPAMMAAAQAALQS |
| 147 | NPAMMAAAQAALQSS |
| 148 | PAMMAAAQAALQSSW |
| 149 | AMMAAAQAALQSSWG |
| 150 | MMAAAQAALQSSWGM |
| 151 | MAAAQAALQSSWGMM |
| 152 | AAAQAALQSSWGMMG |
| 153 | AAQAALQSSWGMMGM |
| 154 | AQAALQSSWGMMGML |
| 155 | QAALQSSWGMMGMLA |
| 156 | AALQSSWGMMGMLAS |
| 157 | ALQSSWGMMGMLASQ |
| 158 | LQSSWGMMGMLASQQ |
| 159 | QSSWGMMGMLASQQN |
| 160 | SSWGMMGMLASQQNQ |
| 161 | SWGMMGMLASQQNQS |
| 162 | WGMMGMLASQQNQSG |
| 163 | GMMGMLASQQNQSGP |
| 164 | MMGMLASQQNQSGPS |
| 165 | MGMLASQQNQSGPSG |
| 166 | GMLASQQNQSGPSGN |
| 167 | MLASQQNQSGPSGNN |
| 168 | LASQQNQSGPSGNNQ |
| 169 | ASQQNQSGPSGNNQN |
| 170 | SQQNQSGPSGNNQNQ |
| 171 | QQNQSGPSGNNQNQG |
| 172 | QNQSGPSGNNQNQGN |
| 173 | NQSGPSGNNQNQGNM |
| 174 | QSGPSGNNQNQGNMQ |
| 175 | SGPSGNNQNQGNMQR |
| 176 | GPSGNNQNQGNMQRE |
| 177 | PSGNNQNQGNMQREP |
| 178 | SGNNQNQGNMQREPN |
| 179 | GNNQNQGNMQREPNQ |
| 180 | NNQNQGNMQREPNQA |
| 181 | NQNQGNMQREPNQAF |
| 182 | QNQGNMQREPNQAFG |
| 183 | NQGNMQREPNQAFGS |
| 184 | QGNMQREPNQAFGSG |
| 185 | GNMQREPNQAFGSGN |
| 186 | NMQREPNQAFGSGNN |
| 187 | MQREPNQAFGSGNNS |
| 188 | QREPNQAFGSGNNSY |
| 189 | REPNQAFGSGNNSYS |
| 190 | EPNQAFGSGNNSYSG |
| 191 | PNQAFGSGNNSYSGS |
| 192 | NQAFGSGNNSYSGSN |
| 193 | QAFGSGNNSYSGSNS |
| 194 | AFGSGNNSYSGSNSG |
| 195 | FGSGNNSYSGSNSGA |
| 196 | GSGNNSYSGSNSGAA |
| 197 | SGNNSYSGSNSGAAI |
| 198 | GNNSYSGSNSGAAIG |
| 199 | NNSYSGSNSGAAIGW |
| 200 | NSYSGSNSGAAIGWG |
| 201 | SYSGSNSGAAIGWGS |
| 202 | YSGSNSGAAIGWGSA |
| 203 | SGSNSGAAIGWGSAS |
| 204 | GSNSGAAIGWGSASN |
| 205 | SNSGAAIGWGSASNA |
| 206 | NSGAAIGWGSASNAG |
| 207 | SGAAIGWGSASNAGS |
| 208 | GAAIGWGSASNAGSG |
| 209 | AAIGWGSASNAGSGS |
| 210 | AIGWGSASNAGSGSG |
| 211 | IGWGSASNAGSGSGF |
| 212 | GWGSASNAGSGSGFN |
| 213 | WGSASNAGSGSGFNG |
| 214 | GSASNAGSGSGFNGG |
| 215 | SASNAGSGSGFNGGF |
| 216 | ASNAGSGSGFNGGFG |
| 217 | SNAGSGSGFNGGFGS |
| 218 | NAGSGSGFNGGFGSS |
| 219 | AGSGSGFNGGFGSSM |
| 220 | GSGSGFNGGFGSSMD |
| 221 | SGSGFNGGFGSSMDS |
| 222 | GSGFNGGFGSSMDSK |
| 223 | SGFNGGFGSSMDSKS |
| 224 | GFNGGFGSSMDSKSS |
| 225 | FNGGFGSSMDSKSSG |
| 226 | NGGFGSSMDSKSSGW |
| 227 | GGFGSSMDSKSSGWG |
| 228 | GFGSSMDSKSSGWGM |
| 229 | EDDGTVLLSTVTAQF |
| 230 | VLLSTVTAQFPGACG |
| 231 | MRGVRLVEGILHAPD |
| 232 | AGWGNINYVVNYPKD |
| 233 | LVYVVNYPKDNKRKM |
| 234 | TFGEVLMVQVKKDLK |
| 235 | ETQVKVMSQRHMIDG |
| 236 | DMTEDELREFFSQYG |
| 237 | ELREFFSQYGDVMDV |
| 238 | DVMDVFIPKPFRAFA |
| 239 | FIPKPFRAFAFVTFA |

In some embodiments, the epitope comprises consecutive amino acids in the peptide sequences derived from tau, as set forth in Table 3.

TABLE 3
peptide sequences derived from tau

| SEQ ID NO | Peptide |
|---|---|
| 240 | NATRIPAKTPPAPKT |
| 241 | ATRIPAKTPPAPKTP |
| 242 | TRIPAKTPPAPKTPP |
| 243 | RIPAKTPPAPKTPPS |
| 244 | IPAKTPPAPKTPPSS |
| 245 | PAKTPPAPKTPPSSG |
| 246 | AGIGDTPSLEDEAAG |
| 247 | AKTPPAPKTPPSSGE |
| 248 | KTPPAPKTPPSSGEP |
| 249 | TPPAPKTPPSSGEPP |
| 250 | PPAPKTPPSSGEPPK |
| 251 | PAPKTPPSSGEPPKS |
| 252 | APKTPPSSGEPPKSG |
| 253 | PKTPPSSGEPPKSGD |
| 254 | KTPPSSGEPPKSGDR |
| 255 | TPPSSGEPPKSGDRS |
| 256 | SSGEPPKSGDRSGYS |
| 257 | SGEPPKSGDRSGYSS |
| 258 | GEPPKSGDRSGYSSP |
| 259 | EPPKSGDRSGYSSPG |
| 260 | PPKSGDRSGYSSPGS |
| 261 | PKSGDRSGYSSPGSP |
| 262 | KSGDRSGYSSPGSPG |
| 263 | SGDRSGYSSPGSPGT |
| 264 | GDRSGYSSPGSPGTP |
| 265 | DRSGYSSPGSPGTPG |
| 266 | RSGYSSPGSPGTPGS |
| 267 | SGYSSPGSPGTPGSR |
| 268 | GYSSPGSPGTPGSRS |
| 269 | YSSPGSPGTPGSRSR |
| 270 | SSPGSPGTPGSRSRT |
| 271 | SPGSPGTPGSRSRTP |
| 272 | PGSPGTPGSRSRTPS |
| 273 | GSPGTPGSRSRTPSL |
| 274 | SPGTPGSRSRTPSLP |
| 275 | PGTPGSRSRTPSLPT |
| 276 | GTPGSRSRTPSLPTP |
| 277 | TPGSRSRTPSLPTPP |
| 278 | PGSRSRTPSLPTPPT |
| 279 | GSRSRTPSLPTPPTR |
| 280 | SRSRTPSLPTPPTRE |
| 281 | RSRTPSLPTPPTREP |
| 282 | SRTPSLPTPPTREPK |
| 283 | RTPSLPTPPTREPKK |
| 284 | TPSLPTPPTREPKKV |
| 285 | PSLPTPPTREPKKVA |
| 286 | SLPTPPTREPKKVAV |
| 287 | TPPTREPKKVAVVRT |
| 288 | PPTREPKKVAVVRTP |
| 289 | PTREPKKVAVVRTPP |
| 290 | TREPKKVAVVRTPPK |
| 291 | REPKKVAVVRTPPKS |
| 292 | EPKKVAVVRTPPKSP |
| 293 | PKKVAVVRTPPKSPS |
| 294 | KKVAVVRTPPKSPSS |
| 295 | KVAVVRTPPKSPSSA |
| 296 | VAVVRTPPKSPSSAK |
| 297 | AVVRTPPKSPSSAKS |
| 298 | VVRTPPKSPSSAKSR |
| 299 | VRTPPKSPSSAKSRL |
| 300 | RTPPKSPSSAKSRLQ |
| 301 | TPPKSPSSAKSRLQT |
| 302 | VPMPDLKNVKSKIGS |
| 303 | PMPDLKNVKSKIGST |
| 304 | MPDLKNVKSKIGSTE |
| 305 | PDLKNVKSKIGSTEN |
| 306 | DLKNVKSKIGSTENL |
| 307 | LKNVKSKIGSTENLK |
| 308 | KNVKSKIGSTENLKH |
| 309 | NVKSKIGSTENLKHQ |
| 310 | VKSKIGSTENLKHQP |
| 311 | KSKIGSTENLRHQPG |
| 312 | SKIGSTENLKHQPGG |
| 313 | KIGSTENLKHQPGGG |
| 314 | IGSTENLKHQPGGGK |
| 315 | GSTENLKHQPGGGKV |
| 316 | STENLKHQPGGGKVQ |
| 317 | EKLDFKDRVQSKIGS |
| 318 | KLDFKDRVQSKIGSL |
| 319 | DFKDRVQSKIGSLDN |
| 320 | FKDRVQSKIGSLDNI |
| 321 | KDRVQSKIGSLDNIT |
| 322 | DRVQSKIGSLDNITH |
| 323 | RVQSKIGSLDNITHV |
| 324 | VQSKIGSLDNITHVP |
| 325 | QSKIGSLDNITHVPG |
| 326 | SKIGSLDNITHVPGG |
| 327 | KIGSLDNITHVPGGG |
| 328 | IGSLDNITHVPGGGN |
| 329 | GSLDNITHVPGGGNK |
| 330 | SLDNITHVPGGGNKK |
| 331 | AKAKTDHGAEIVYKS |
| 332 | KAKTDHGAEIVYKSP |
| 333 | AKTDHGAEIVYKSPV |
| 334 | KTDHGAEIVYKSPVV |
| 335 | TDHGAEIVYKSPVVS |
| 336 | DHGAEIVYKSPVVSG |
| 337 | HGAEIVYKSPVVSGD |
| 338 | GAEIVYKSPVVSGDT |
| 339 | AEIVYKSPVVSGDTS |
| 340 | EIVYKSPVVSGDTSP |
| 341 | IVYKSPVVSGDTSPR |
| 342 | VYKSPVVSGDTSPRH |
| 343 | YKSPVVSGDTSPRHL |
| 344 | KSPVVSGDTSPRHLS |
| 345 | SPVVSGDTSPRHLSN |
| 346 | PVVSGDTSPRHLSNV |
| 347 | VVSGDTSPRELSNVS |
| 348 | VSGDTSPRHLSNVSS |
| 349 | SGDTSPRHLSNVSST |
| 350 | GDTSPRHLSNVSSTG |
| 351 | DTSPRHLSNVSSTGS |
| 352 | TSPRHLSNVSSTGST |
| 353 | SPRHLSNVSSTGSID |
| 354 | PRHLSNVSSTGSIDM |
| 355 | RHLSNVSSTGSIDMV |
| 356 | HLSNVSSTGSIDMVD |
| 357 | LSNVSSTGSIDMVDS |
| 358 | SNVSSTGSIDMVDSP |
| 359 | NVSSTGSIDMVDSPQ |
| 360 | VSSTGSIDMVDSPQL |
| 361 | SSTGSIDMVDSPQLA |
| 362 | STGSIDMVDSPQLAT |
| 363 | TGSIDMVDSPQLATL |
| 364 | GSIDMVDSPQLATLA |
| 365 | SIDMVDSPQLATLAD |
| 366 | IDMVDSPQLATLADE |
| 367 | DMVDSPQLATLADEV |
| 368 | MVDSPQLATLADEVS |
| 369 | VDSPQLATLADEVSA |
| 370 | DSPQLATLADEVSAS |
| 371 | SPQLATLADEVSASL |
| 372 | KVAVVRTPPKSPSSAK |
| 373 | PAPKTPPSSGEPPKSG |
| 374 | PMPDLKNVKSKIGSTE |
| 375 | DFKDRVQSKIGSLDNI |
| 376 | DRSGYSSPGSPGTPGS |

In some embodiments, the epitope comprises consecutive amino acids in the peptide sequences derived from alpha-synuclein, as set forth in Table 4.

TABLE 4
peptide sequences derived from alpha-synuclein

| Seq ID | |
|---|---|
| NO | Peptide |
| 377 | VFMKGLSKA |
| 378 | DVFMKGLSKA |
| 379 | GWAAAEKTK |
| 380 | VAAAEKTKQGVAEAP |
| 381 | VAAAEKTKQGVAEAA |
| 382 | AGKTKEGVL |
| 383 | PGKTKEGVL |
| 384 | AGKTKEGVLY |
| 385 | APGKTKEGVL |
| 386 | GVAEAAGKTK |
| 387 | KQGVAEAPGKTKEGV |
| 388 | PGKTKEGVLYVGSKT |
| 389 | KTKEGVLYVGSKTKK |
| 390 | KQGVAEAAGKTKEGV |
| 391 | AGKTKEGVINVGSKT |
| 392 | VLYVGSKTK |
| 393 | LYVGSKTKK |
| 394 | YVGSKTKEGV |
| 395 | VLYVGSKTK |
| 396 | GVLYVGSKTK |
| 397 | LYVGSKTKEG |
| 398 | KTKKGWHGV |
| 399 | KTKKGWHG |
| 400 | YVGSKTKKGWHGVA |
| 401 | KTKEGVLYVGSKTKE |
| 402 | VTNVGGAW |
| 403 | GWHGVTTV |
| 404 | EEGAPQEGI |
| 405 | GSIAAATGFV |
| 406 | SIAAATGFVK |
| 407 | AGSIAAATGF |
| 408 | IAAATGFVK |
| 409 | APQEGILEDM |
| 410 | EEGAPQEGIL |
| 411 | VFMGLSKAK |
| 412 | AEAAGKTKEG |
| 413 | YVGSKTKEGVVHGVT |
| 414 | IAAATGFVK |
| 415 | DNEAYEMPSEEGYQDY |
| 416 | PSEEGYQDY |
| 417 | YEMPSEEGY |
| 418 | MPSEEGYQD |
| 419 | AYEMPSEEGY |
| 420 | MPSEEGYQDY |
| 421 | EMPSEEGYQD |
| 422 | DNEAYEMPSE |
| 423 | YEMPSEEGYQ |
| 424 | SEEGYQDYEP |
| 425 | NTQTDRESLRNLRCYYNQS |
| 426 | GKTKEGVLYVGSKTK |
| 427 | YWDLQTRNVKAHSQTDRA |
| 428 | RNTQIFKTNTQTHRENLRIALRY |
| 429 | FLPTGGKGGSCSQAAS |
| 430 | QRMEPRAPWIEQERPAYW |
| 431 | VYSRITPAENGKSNFLNCYVSGFHPSDIE |
| 432 | PDGRLLRGYQQDAYDG |
| 433 | GHVRGVAPQIPGERE |
| 434 | HQAQVGGGPCGGAVESLPGGHVRG |
| 435 | FLPTGGKGGSCSQAAS |
| 436 | RKLEAAGVAEQLRAYLEGECV |
| 437 | DVGSDGRFLR |
| 438 | NTQTDRESLRNLRCYYNQS |
| 439 | SLPGGRVRGVAPQIPGE |
| 440 | KWEAARVAEQLRAYLEGLCVEWLRRH |
| 441 | DRETRDLTGNGKD |
| 442 | LSSWTAADTAAQITQRKLEAA |
| 443 | ILRWEPSSQPTIPIVGIIAGLVL |
| 444 | GHVRGVAPQIPGERE |
| 445 | HQAQVGGGPCGGAVESLPGGHVRG |
| 446 | FLPTGGKGGSCSQAAS |
| 447 | FLPTGGKGGSCSQAASSNSAQGSD |
| 448 | KTKQGVAEA |
| 449 | DVFMKGLSK |
| 450 | FMKGLSKAK |
| 451 | FMKGLSKAKE |
| 452 | KTKQGVAEAA |
| 453 | KAKEGVVAAA |
| 454 | KAKEGVVAA |
| 455 | KTKEGVLYV |
| 456 | APGKTKEGV |
| 457 | GVAEAPGKTK |
| 458 | KTKEGVVHG |
| 459 | YVGSKTKEGVVHGVA |
| 460 | AVVTGVTAV |
| 461 | QVTNVGGAV |
| 462 | KTKEQVTNV |
| 463 | NVGGAVVTGV |
| 464 | GAVVTGVTAV |
| 465 | GVTAVAQKTV |
| 466 | EQVTNVGGAV |
| 467 | VATVAEKTKE |
| 468 | GVVHGVATV |
| 469 | EGVVHGVTTVAEKTK |
| 470 | TTVAEKTKEQVTNVG |
| 471 | KGVVHGVATVAEKTK |
| 472 | EGVVHGVATVAEKTK |
| 473 | ATVAEKTKEQVTNVG |
| 474 | SIAAATGFV |
| 475 | AAATGFVKK |
| 476 | KTVEGAGSI |
| 477 | GSIAAATGF |
| 478 | NEEGAPQEGI |
| 479 | AATGFVKKDQ |
| 480 | FVKKDQLGK |
| 481 | PVDPDNEAY |
| 482 | MPVDPDNEA |
| 483 | DPDNEAYEM |
| 484 | EGILEDMPVD |
| 485 | MPVDPDNEAY |
| 486 | QEGILEDMPV |
| 487 | GKTKEGVLYVGSKTK |
| 488 | KTKEGVLYVGSKTKE |
| 489 | MPVDPDNEAYEMPSE |
| 490 | DNEAYEMPSEEGYQD |
| 491 | EMPSEEGYQDYEPEA |
| 492 | SEEGYQDYEPEA |
| 493 | GVLYVGSKTK |
| 494 | VLYVGSKTK |
| 495 | VLYVGSKTKK |
| 496 | MDVFMKGLSKAKEGV |
| 497 | KGLSKAKEGVVAAAE |
| 498 | AKEGVVAAAEKTKQG |
| 499 | VAAAEKTKQGVAEAA |
| 500 | KTKQGVAEAAGKTKE |
| 501 | KQGVAEAAGKTKEGV |
| 502 | VAEAAGKTKEGVLYV |
| 503 | AGKTKEGVLYVGSKT |
| 504 | GVLYVGSKTKEGVVH |
| 505 | YVGSKTKEGVVHGVA |
| 506 | GSKTKEGVVHGVATV |
| 507 | EGVVHGVATVAEKTK |
| 508 | GVATVAEKTKEQVTN |
| 509 | ATVAEKTKEQVTNVG |
| 510 | AEKTKEQVTNVGGAV |
| 511 | EQVTNVGGAVVTGVT |
| 512 | VGGAVVTGVTAVAQK |
| 513 | VTGVTAVAQKTVEGA |
| 514 | AVAQKTVEGAGSIAA |
| 515 | TVEGAGSIAAATGFV |
| 516 | GSIAAATGFVKKDQL |
| 517 | ATGFVKKDQLGKNEE |
| 518 | KKDQLGKNEEGAPQE |
| 519 | GKNEEGAPQEGILED |
| 520 | GAPQEGILEDMPVDP |
| 521 | GILEDMPVDPDNEAY |

Biomarker Processing

In a practical implementation, a method is described whereby a biological sample is processed to determine the presence of a TCR specific to an epitope contained in a protein associated with a neurodegenerative disease, as previously discussed. For example, in one embodiment, the protein may form ‘aggregates in a subject, where the aggregates are associated with the neurodegenerative disease. The person of skill will recognize that there are various practical approaches to making such determination.

In a first variant, the person of skill can determine the presence of the TCR specific to such epitope by detecting an increase activation of leukocytes contained in the sample, after contacting the leukocytes with the epitope or test compound. For example, the epitope or compound may include at least one peptide derived from the protein associated with the neurodegenerative disease. In another example, the epitope or compound may include at least one peptide derived from the protein that forms aggregates in the subject, where the aggregates are associated with the neurodegenerative disease. In another example, the epitope may include at least one of the peptides listed in any one of Tables 1 to 4.

In some embodiments, the peptide may be linked or associated to a carrier, for example, a major histocompatibility complex (MHC) molecule or an inert carrier, such as streptavidin or avidin beads. General methods for linking a peptide to such carrier are readily available to the person of skill and will, thus, not be further discussed here.

General methods for assaying whether a leukocyte has increased activation are known to those of skill in the art, and may include techniques such as ELISpot assay Western Blot Analysis and ELISA for detecting/assessing cytokine release of activated leukocytes; cell counting and fluorescence-activated cell sorting (FACS) for assaying increased proliferation and differentiation of activated leukocytes; PCR, RT-PCR, Northern Blot Analysis, and microarray analysis for assaying differential gene expression of activated leukocytes; and the like. For sake of conciseness, and since such techniques are readily available to the person of skill, these techniques are not further discussed here.

In a second variant, the person of skill can determine the presence of the TCR specific to such epitope by detecting the presence of the particular TCR using a gene detection approach. For example, the person of skill may make use of techniques such as PCR, RT-PCR, Northern Blot Analysis, and the like. Such techniques are readily available to the person of skill. Once a particular TCR specific to a particular peptide is known, the person of skill can design and/or use particular primers or probe for detecting the presence of the TCR in a particular biological sample, a cell fraction thereof, or a cell culture derived therefrom. Again, for sake of conciseness, and since such techniques are readily available to the person of skill, these techniques are not further discussed here.

In other embodiments of the present invention, there is provided a method comprising: providing a biological sample from a subject; processing the biological sample to determine presence of a human leukocyte antigen (HLA) capable of presenting a peptide, wherein the peptide is a fragment from a protein having an aberrant protein expression and/or aberrant protein function and/or aberrant protein macrostructure in a patient having a neurodegenerative disease and the HLA allele is associated with a neurodegenerative disease. Identification of the HLAs expressed in a sample or by a patient, i.e. “HLA typing” can be done using methods known in the art, including gene detection approaches. In certain embodiments, the present method determines detecting the presence of one or more of the following HLAs: DRB5*01:01, DRB1*15:01, DQB1*03:04, A*11:01, DRB1*07:01, DRB1*09:01, or DQB1*03:01.

In a practical implementation, a method is described whereby the presence of the TCR specific to such epitope or the HLA capable of presenting such peptide is indicative of the patient being predisposed, at risk of or having a neurodegenerative disease or being a potential candidate for treatment of neurodegenerative disease. Non-limiting examples of therapies that are directed to leukocytes that are activated by an epitope include administration of a compound that selectively kills leukocytes that are capable of becoming activated when they are contacted with the epitope, and tolerization therapy. For example, tolerization therapy may be implemented by exposing the patient to the particular epitope in a form that alters the immune system to recognize it as self, and halt or reduce making killer T cells.

Such practical implementation may be based, without being limited to any particular theory, on the following scientific rational. The specificity of T cells towards their target antigens is determined by their heterodimeric, hyper-variable T cell receptor (TCR) molecules, which recognize antigenic peptides that are presented by MHC molecules. In immune defense situations, the MHC molecules are of “self”-origin, whereas the antigenic peptides are “non-self”, i.e. they are derived from viral or microbial peptides. Typically, class-I MHC molecules present peptides of intracellular (viral) origin to CD8+ T cells, whereas class-II MHC molecules present phagocytosed (microbial) peptides to CD4+ T cells. In addition, “self” MHC molecules also present “self” peptides, but these are normally ignored because of T cell tolerance. It is assumed that in autoimmune diseases the tolerance is broken and recognition of “self” peptides results in chronic inflammation, disturbed organ function or tissue destruction.

In further embodiments, the present methods comprising detecting the presence of both (i) a human leukocyte antigen (HLA) capable of presenting a peptide, wherein the peptide is a fragment from a protein having an aberrant protein expression and/or aberrant protein function and/or aberrant protein macrostructure in a patient having a neurodegenerative disease and the HLA allele is associated with a neurodegenerative disease and (ii) a TCR specific to an epitope contained in the peptide associated with a neurodegenerative disease. In certain embodiments, the methods comprises (i) providing a biological sample from a subject; processing the biological sample to determine presence of a human leukocyte antigen (HLA) capable of presenting a peptide, wherein the peptide is a fragment from a protein having an aberrant protein expression and/or aberrant protein function and/or aberrant protein macrostructure in a patient having a neurodegenerative disease; (ii) if the HLA is detected in the biological sample, contacting cells in the sample with the peptide to expand the leukocytes and determine the presence of a TCR specific to the epitope. In certain embodiments, the detection in the patient sample of a HLA allele associated with neurodegenerative disease in combination with detection of a TCR specific to an epitope contained in a protein associated with a neurodegenerative disease indicative of the patient being predisposed, at risk of or having a neurodegenerative disease or being a potential candidate for treatment of neurodegenerative disease.

In specific embodiments of the present method, the method comprises determining in the biological patient sample the presence of an HLA allele listed in Table 5 and the presence of a TCR that binds an epitope listed in Table 5, wherein the presence of such an HLA allele and such a TCR is indicative of the patient being predisposed, at risk of or having a neurodegenerative disease or being a potential candidate for treatment of neurodegenerative disease.

TABLE 5

| HLA Allele | KTKEGVLYVGSKTKE | Phospho-Y | MPVDPDNEAYEMPSE | Phospho-S |
|---|---|---|---|---|
| DPB1*02:01 | — | 620 | — | — |
| DPB1*03:01 | — | 480 | — | — |
| DPB1*04:01 | — | 937 | — | — |
| DPB1*04:02 | 2285 | 1602 | — | — |
| DPB1*05:01 | 9635 | 777 | — | — |
| DPB1*14:01 | 4108 | nd | — | — |
| DQB1*02:01 | — | 4429 | 1716 | 7401 |
| DQB1*03:01 | 225 | nd | — | — |
| DQB1*03:02 | — | 441 | 1419 | 6991 |
| DQB1*04:02 | — | — | 508 | 719 |
| DQB1*05:01 | — | 2019 | 179 | 258 |
| DQB1*06:02 | 9642 | 2539 | — | — |
| DRB1*01:01 | 7124 | nd | — | — |
| DRB1*03:01 | 5089 | 541 | 6962 | — |
| DRB1*04:01 | 5140 | nd | 397 | 817 |
| DRB1*04:05 | 2450 | 547 | 9136 | 3600 |
| DRB1*07:01 | 177 | nd | — | 6384 |
| DRB1*09:01 | 83 | 128 | — | 6629 |
| DRB1*11:01 | — | nd | 1568 | — |
| DRB1*12:01 | — | 2517 | — | — |
| DRB1*13:02 | — | 190 | — | — |
| DRB1*15:01 | 3 | 49 | 5539 | 8977 |
| DRB3*01:01 | 7181 | 1211 | 5671 | 411 |
| DRB3*02:02 | 9245 | 879 | — | — |
| DRB4*01:01 | — | 198 | — | — |
| DRB5*01:01 | 8 | 26 | — | — |

| HLA Allele | DNEAYEMPSEEGYQD | Phospho-S | EMPSEEGYQDYEPEA | Phospho-S |
|---|---|---|---|---|
| DPB1*02:01 | — | — | — | — |
| DPB1*03:01 | — | — | — | — |
| DPB1*04:01 | — | — | — | — |
| DPB1*04:02 | — | — | — | — |
| DPB1*05:01 | — | — | — | — |
| DPB1*14:01 | — | — | — | — |
| DQB1*02:01 | 1639 | 3306 | 2424 | 4164 |
| DQB1*03:01 | — | — | — | — |
| DQB1*03:02 | 349 | 1158 | 5801 | — |
| DQB1*04:02 | 51 | 568 | 661 | 7869 |
| DQB1*05:01 | 7 | 21 | 99 | 96 |
| DQB1*06:02 | — | — | — | — |
| DRB1*01:01 | — | — | — | — |
| DRB1*03:01 | — | — | — | — |
| DRB1*04:01 | 76 | 9129 | 7502 | 4955 |
| DRB1*04:05 | 1941 | — | — | — |
| DRB1*07:01 | — | — | — | — |
| DRB1*09:01 | 4603 | — | — | — |
| DRB1*11:01 | — | — | — | — |
| DRB1*12:01 | — | — | — | — |
| DRB1*13:02 | — | — | — | — |
| DRB1*15:01 | — | — | — | 1983 |
| DRB3*01:01 | 787 | 9184 | — | — |
| DRB3*02:02 | — | — | — | — |
| DRB4*01:01 | — | — | — | — |
| DRB5*01:01 | — | — | — | — |

—: no binding detected,; nd: not done.

Also provided is a method for assessing whether a subject afflicted with a disease or condition involving an inflammatory response or related to inflammation is likely to benefit or has benefitted from a therapy, wherein the therapy comprises administration of a T cell receptor for a particular antigen:MHC complex (e.g. as provided on a cell through adoptive T cell therapy), the method comprising, consisting, or alternatively consisting essentially of:(a) (i) obtaining leukocytes from the subject; (ii) contacting the leukocytes with the antigen bound to an MHC molecule; (iii) determining whether the leukocytes have increased activation after contact with the a antigen bound to an MHC molecule; and(iv) identifying the subject as likely to benefit from the therapy if in step (iii) the leukocytes are determined to have increased activation after contact with the antigen bound to an MHC molecule, and identifying the subject as unlikely to benefit from the therapy if in step (iii) the leukocytes are determined to not have increased activation after contact with the antigen bound to an MHC molecule; or (b) (i) obtaining leukocytes from the subject; (ii) contacting the leukocytes with the antigen bound to an MHC molecule; (iii) determining whether the leukocytes have increased activation after contact with the antigen bound to an MHC molecule; and (iv) identifying the subject as having benefited from the therapy if in step (iii) the leukocytes are determined to have increased activation after contact with the antigen bound to an MHC molecule, and identifying the subject as not having benefitted from the therapy if in step (iii) the leukocytes are determined to not have increased activation after contact with the antigen bound to an MHC molecule.

In some embodiments, provided herein is a method for assessing whether a subject afflicted with a neurodegenerative disease or disorder is likely to benefit or has benefitted from a therapy, wherein the therapy comprises administration of an effective amount of a T cell receptor for a particular antigen:MHC complex (e.g. as provided on a cell through adoptive T cell therapy), the method comprising, consisting, or alternatively consisting essentially of:(a) (i) obtaining leukocytes from the subject; (ii) contacting the leukocytes with the antigen bound to an MHC molecule; (iii) determining whether the leukocytes have increased activation after contact with the antigen bound to an MHC molecule; and (iv) identifying the subject as likely to benefit from the therapy if in step (iii) the leukocytes are determined to have increased activation after contact with the antigen bound to an MHC molecule, and identifying the subject as unlikely to benefit from the therapy if in step (iii) the leukocytes are determined to not have increased activation after contact with the antigen bound to an MHC molecule; or (b) (i) obtaining leukocytes from the subject; (ii) contacting the leukocytes with the antigen bound to an MHC molecule; (iii) determining whether the leukocytes have increased activation after contact with the antigen bound to an MHC molecule; and (iv) identifying the subject as having benefited from the therapy if in step (iii) the leukocytes are determined to have increased activation after contact with the antigen bound to an MHC molecule, and identifying the subject as not having benefitted from the therapy if in step (iii) the leukocytes are determined to not have increased activation after contact with the antigen bound to an MHC molecule. In certain embodiments, the neurodegenerative disease or disorder is α-synucleinopathy, Parkinson's disease, Lewy Body dementia, or Alzheimer's disease.

The following examples are illustrative of procedures which can be used in various instances in carrying the disclosure into effect.

In one non-limiting embodiment, the herein described methods, processes, and systems can be used alone or in combination with one another, for example as diagnostics.

Systems

The herein described methods, systems and procedures may be useful in practical applications for assisting a medical expert in assessing whether a subject is predisposed to a neurodegenerative disease or that the subject is afflicted with the disease. The person of skill in view of the teachings of the present text will readily understand how to design and perform a system for making such assessments.

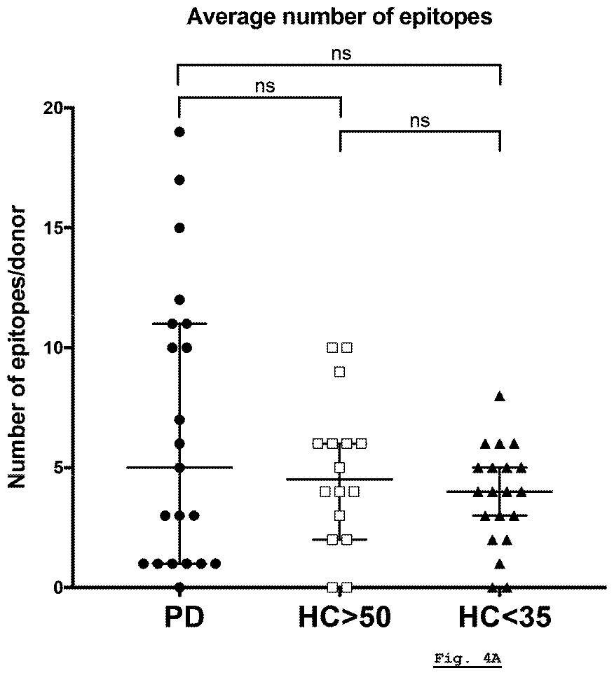

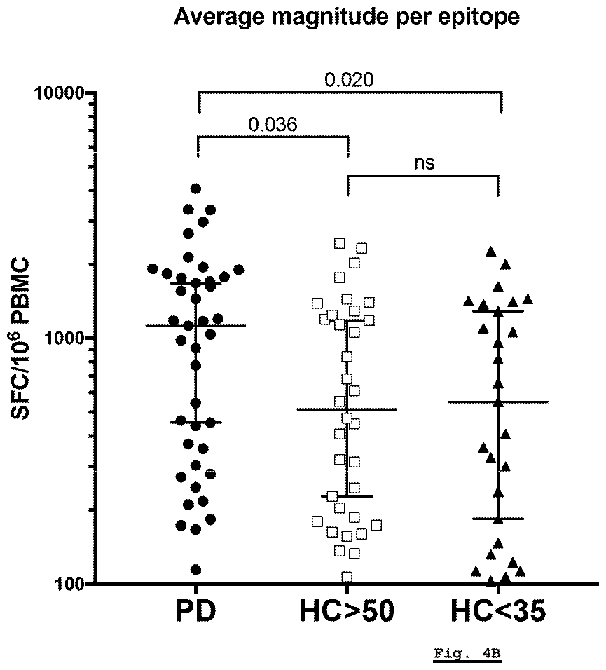

With reference to FIG. 6, there is shown a configuration of a system 100 for assessing a neurodegenerative disease patient. The system 100 comprises a user interface 102, an apparatus 101 including a processing unit 104, and an output unit 106.

The user interface 102 includes any one or a combination of a keyboard, a pointing device, a touch sensitive surface, a speech recognition unit or any other suitable device allowing information to be entered by a user. Alternatively, the user interface 102 may be in the form of a data input device such as, but not limited to, a disk drive, CD-ROM, a port connected to a data stream and flash memory. The user interface 102 enables a user to provide a set of information data elements associated to a certain neurodegenerative disease patient.

The set of biological information data elements may include information pertaining to the presence of a human leukocyte antigen (HLA) capable of presenting a peptide, wherein the peptide is a fragment from a protein that forms aggregates in a patient having a neurodegenerative disease. Additionally or alternatively, the set of information data elements may include information pertaining to the presence of a T cell receptor (TCR) specific to such peptide.

Optionally, the set of information data elements may also include information derived from cognitive assessment test results associated with suspected AD; measurement of motor manifestations, assessment of ability to perform daily functional activities, and symptomatic response to medication with suspected PD; measurement or assessment of loss of function or gradual, slowly progressive, painless weakness in one or more regions of the body, without changes in the ability to feel, with suspected ALS; patient age; and the like.

Other suitable information data elements may also be provided through user interface 102 in alternative implementations.

The apparatus 101 is configured to receive the set of information data elements. The apparatus 101 processes the set of information data elements to generate information associated with the neurodegenerative disease patient. In the specific embodiment shown in FIG. 15, apparatus 101 includes a processing unit 104, an input 110 and an output 114. Input 110 is operative for receiving signals from the user interface 102 indicative of a set of information data elements associated to the patient. As shown in FIG. 15, the processing unit 104 is in communication with input 110 for receiving the signal or signals indicative of a set of information data elements associated to the patient. As will be described in more detail below, on the basis of the signal or signals received at input 110, the processing unit 104 is operative to generate information associated with the neurodegenerative disease patient. The information conveys the likelihood of the predisposition or presence of the neurodegenerative disease.

The apparatus 101 releases at output 114 a signal for causing output unit 106 to convey the information to a user. The output unit 106 may be in the form of any suitable device for conveying information to the physician or other health care professional. In a specific example of implementation, the output unit 106 can include a display screen, or in an alternative example of implementation, the output unit 106 can include a printing device for displaying the data in printed form.

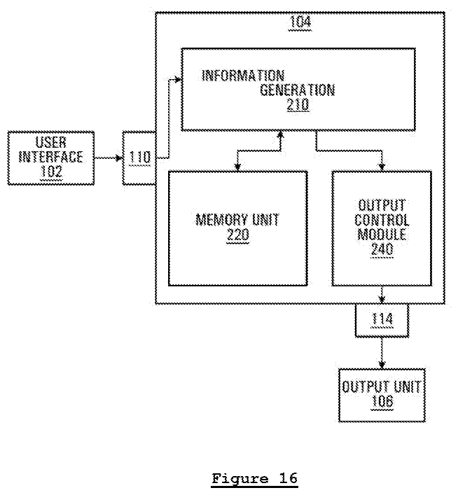

As shown in FIG. 16, the processing unit 104, in accordance with a first specific embodiment, includes a neurodegenerative disease generation module 210, a memory unit 220 and an output control module 240.

Memory unit 220 stores a plurality of instructions and is configured to provide these instructions to the processing unit 104. When executed, these instructions cause the processing unit 104 to:
(i) receive first and second biological data elements for an individual from a biological data source, wherein the first biological data element comprises data pertaining to the individual's human leukocyte antigen (HLA) typing and the second biological data element comprises data pertaining to the individual's T cell receptor (TCR) repertoire;
(ii) merge the first and second biological data elements from the biological data source to obtain a set of merged biological data associated with the individual, including to:
   1) identify data in the first and second biological data elements that indicates a reciprocity, the identified data corresponding to a reciprocal presence of an HLA typing value in the first biological data element and of a TCR repertoire value in the second biological data element;
   2) compare the identified data with at least one of an element of HLA typing values and TCR repertoire values stored on the one or more memories, said values stored on the one or more memories being associated with reference individuals; and
   3) determine a likelihood or predisposition score based on at least the identified data and on the comparison; and
(iii) display the likelihood or predisposition score in a graphical user interface (GUI).

Those skilled in the art should appreciate that in some non-limiting embodiments, all or part of the functionality previously described herein with respect to the components of the system 100 for assessing a neurodegenerative disease patient to perform operations for providing the TCR and/or HLA immune-profiling functionality to a user as described throughout this specification, may be implemented as pre-programmed hardware or firmware elements (e.g., application specific integrated circuits (ASICs), electrically erasable programmable read-only memories (EEPROMs), etc.), or other related components.

In other non-limiting embodiments, all or part of the functionality previously described herein with respect to the system 100 for assessing a neurodegenerative disease patient to perform operations for providing TCR and/or HLA immune-profiling functionality to a user as described throughout this specification, may be implemented as software consisting of a series of program instructions for execution by one or more computing units. The series of program instructions can be tangibly stored on one or more tangible computer readable storage media (e.g., removable diskette, CD-ROM, ROM, PROM, EPROM or fixed disk), or the instructions can be tangibly stored remotely but transmittable to the one or more computing unit via a modem or other interface device (e.g., a communications adapter) connected to a network over a transmission medium. The transmission medium may be either a tangible medium (e.g., optical or analog communications lines) or a medium implemented using wireless techniques (e.g., microwave, infrared or other transmission schemes).

Those skilled in the art should further appreciate that the program instructions may be written in a number of programming languages for use with many computer architectures or operating systems.

Terms

Unless otherwise defined, all technical and scientific terms used herein have the same meaning as commonly understood by a person of ordinary skill in the art to which this invention belongs. As used herein, and unless stated otherwise or required otherwise by context, each of the following terms shall have the definition set forth below.

As used herein, “about” in the context of a numerical value or range means±10% of the numerical value or range recited or claimed, unless the context requires a more limited range.

As used herein, a “subject afflicted with” a condition, e.g. PD, LBD, ALS or AD, means a subject who was been affirmatively diagnosed to have the condition.

Embodiments of the present invention relate to determining whether leukocytes have increase activation after contact with an epitope or test compound. It will be understood that the “increased activation” of the leukocytes is in response to contact with the epitope or the test compound. General methods for assaying whether a leukocyte has increased activation will be known to those of ordinary skill in the art. Additionally, assays for determining increased activation that are described for particular epitopes or test compounds in the Examples herein may be applied to other epitopes and test compounds of the invention.

In some embodiments, the leukocytes are determined to have increased activation after contact with the epitope if the leukocytes release a cytokine. In some embodiments, the leukocytes are determined to have increased activation after contact with an epitope or test compound if the leukocytes release a cytokine that is not released by corresponding leukocytes not contacted with the epitope or test compound. In some embodiments, a cytokine is determined to be released if there is a minimum of about 5, 10, 15, 20, 25, 30, 35, 40, 45, 50, 100, or about 10-50 spot-forming cells (SFC) per million cells are measured using an ELISpot assay comprising the colorimetric detection of the cytokine. In some embodiments, the leukocytes are determined to have released the at least one cytokine if there are over 20 spot-forming cells (SFC) per million cells as measured by an ELISpot assay comprising the colorimetric detection of the at least one cytokine. A person having ordinary skill in the art will readily be able to perform the ELISpot assay in embodiments of the invention using the disclosures herein. ELISpot assays are described in Czerkinsky et al. (1988) Reverse ELISpot Assay for Clonal Analysis of Cytokine Production. I. Enumeration of gamma-Interferon-Secreting Cells. J. Immunol. Methods, 110:29; Sedgwick and Holt, (1983) A Solid-Phase Immunoenzymatic technique for the Enumeration of Specific Antibody-Secreting Cells. J. Immunol. Methods, 57: 301; and Czerkinsky et al. (1983) A Solid-Phase Enzyme-Linked Immunospot (ELISPOT) Assay for Enumeration of Specific Antibody Secreting Cells. J. Immunol. Methods, 65:109, the entire content of each of which is incorporated herein by reference.

In some embodiments, the leukocytes are determined to have increased activation after contact with an epitope or test compound if the leukocytes release more of a cytokine than corresponding leukocytes not contacted with the epitope or test compound. In some embodiments, the leukocytes are determined to have increased activation after contact with the epitope or test compound if the leukocytes release about 1, 2, 3, 4, 5, 10, 15, 20, 25, 30, 35, 40, 45, 50, 55, 60, 65, 70, 80, 90, 100, 150, 200, 300, 400, 500, 750, 1,000, or 1-5,000% more of a cytokine than corresponding leukocytes not contacted with the epitope or test compound. Additional thresholds can be defined based on comparison with reactivity of, for example, non-PD donors (ie, healthy controls). Methods for assaying increased cytokine release include but are not limited to the ELISpot assay, Western Blot Analysis and ELISA, which will be well understood to those in the art.

In some embodiments, the leukocytes are determined to have increased activation after contact with the epitope or test compound if the leukocytes proliferate, and corresponding leukocytes not contacted with the epitope or test compound do not proliferate. In some embodiments, the leukocytes are determined to have increased activation after contact with the epitope or test compound if the leukocytes proliferate more than corresponding leukocytes not contacted with the epitope or test compound. In some embodiments, the leukocytes are determined to have increased activation after contact with the epitope or test compound if the leukocytes proliferate about 1, 2, 3, 4, 5, 10, 15, 20, 25, 30, 35, 40, 45, 50, 55, 60, 65, 70, 80, 90, 100, 150, or 200% more than corresponding leukocytes not contacted with the epitope or test compound. In some embodiments the leukocytes are determined to have increased activation after contact with the epitope or test compound if the leukocytes become differentiated after contact with the epitope or test compound, and corresponding leukocytes not contacted with the epitope or test compound do not become differentiated. In some embodiments the leukocytes are determined to have increased activation after contact with the epitope or test compound if the leukocytes are more differentiated than corresponding leukocytes not contacted with the epitope or test compound. Methods for assaying increased proliferation and differentiation are well known in the art, and include cell counting and fluorescence-activated cell sorting (PACS).

In some embodiments, the leukocytes are determined to have increased activation after contact with the epitope or test compound if the leukocytes express a gene at a higher or lower level than corresponding leukocytes not contacted with the epitope or test compound. In some embodiments, the expression is about 1, 2, 3, 4, 5, 10, 15, 20, 25, 30, 35, 40, 45, 50, 55, 60, 65, 70, 80, 90, 100, 150, 200, 300 400, 500, 750, 1,000, or 1-5,000% higher or lower than in the corresponding leukocytes. In some embodiments, the gene encodes a cytokine. Examples of genes which are differentially expressed in activated T cells are described in Teague et al. (1999) Activation changes the spectrum but not the diversity of genes expressed by T cells. PNAS Vol. 96, No. 22, 12691-12696, the entire content of which is hereby incorporated herein by reference. Methods for assaying gene expression are well known in the art, and include PCR, RT-PCR, Northern Blot Analysis, and microarray analysis.

The release, proliferation, differentiation or change in expression may be measured at, for example, about 0.5, 1, 2, 3, 4, 5, 6, 10, 12, 18, 24, 30, 36, 42, 48, or 72 hours after the leukocytes are contacted with the epitope or test compound.

As used herein, the term “T-cell receptor”, or “TCR”, is a molecule found on the surface of T cells, or T lymphocytes, which is responsible for recognizing fragments of antigen as peptides bound to major histocompatibility complex (MHC) molecules. When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors.

As used herein, the term “biological sample” includes a biological sample that contains immune cells. Such immune cells are generally isolated from peripheral blood, secondary lymphoid tissue and effector sites of activated immune cell populations (e.g. lung, gut, or intestine). The herein described sample can be obtained by any known technique, for example by drawing, by non-invasive techniques, or from sample collections or banks, etc. The sample may be processed so as to isolate a cellular fraction thereof. For example, in the case of blood, the cellular fraction can be fractionated from whole blood by centrifugation, using for instance gentle centrifugation at about 300-800× g for about five to about ten minutes, or fractionated by other standard methods.

As used herein, the term “peptide” describes a group of molecules consisting of up to 50 amino acids. Peptides may further form dimers, trimers and higher oligomers, i.e. consisting of more than one molecule which may be identical or non-identical. The corresponding higher order structures are, consequently, termed homo- or heterodimers, homo- or heterotrimers etc. The term “peptide” (wherein “polypeptide” is interchangeably used with “protein”) also refers to naturally modified peptides wherein the modification is effected e.g. by glycosylation, acetylation, phosphorylation and the like. Such modifications are well-known in the art. Preferably, the peptides have a minimum length of at least 4 amino acids, such as for example at least 5, at least 6, at least 7, at least 8, at least 9 or at least 10 amino acids. Also preferred is that the peptides have a length of at the most 50 amino acids, such as for example at most 45, such as at most 40, at most 35, at most 30, at most 25, at most 20 amino acids. Any of the intermediate numbers not explicitly mentioned are also envisaged herein. More preferably, peptides represented by MHC class I molecules have a length of between 4 and 20 amino acids. Thus, said peptides may be 4, 5, 6, 7, 8, 9, 10, 11, 12, 13, 14, 15, 16, 17, 18, 19 or 20 amino acids in length. Also preferred is that peptides represented by MHC class II molecules have a length of between 4 and 50 amino acids. The class II peptides may in principle be infinitely long, because they may reach out from the MHC binding groove at both sides. The epitope itself is normally 8 to 10 amino acids long. Thus, said peptides may be 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length.

As used herein, a stretch of “consecutive amino acids” means a plurality of amino acids arranged in a chain, each of which is joined to a preceding amino acid by a peptide bond, excepting that the first amino acid in the chain may optionally not be joined to a preceding amino acid. The amino acids of the chain may be naturally or non-naturally occurring, or may comprise a mixture thereof. The amino acids, unless otherwise indicated, may be genetically encoded, naturally-occurring but not genetically encoded, or non-naturally occurring, and any selection thereof. In some embodiments, the epitope peptide may be post-translationally modified and/or shortened and/or elongated on both sides

As used herein a therapy that is “directed to leukocytes that are activated by an epitope” is a therapy that selectively reduces or prevents the activation of the leukocytes by the epitope. It will be understood that selectively reducing or preventing the activation of leukocytes by an epitope includes killing the leukocytes or reducing the viability or proliferation of leukocytes that are capable of becoming activated when they are contacted with the epitope. Non-limiting examples of therapies that are directed to leukocytes that are activated by an epitope include administration of a compound that selectively kills leukocytes that are capable of becoming activated when they are contacted with the epitope, and tolerization therapy.

As used herein, “tolerization therapy” or “antigen-specific tolerization” comprises exposing a subject with an epitope in a way that alters the subject's immune system to have reduced activation by the epitope. Tolerization therapy results in a decrease in the activation of a leukocyte of the subject, such as a T cell, by the epitope. Tolerization therapy is discussed in Coppieters et al. (2013) Clinical Immunology, 149, 345-355; Billetta et al. (2012) Clin Immunol, 145(2):94-101; and Lutterotti and Martin (2014) Expert Opinion on Investigational Drugs, Vol. 23, No. 1, pages 9-20, the entire content of each of which is hereby incorporated herein by reference.

Aspects of the present invention relate to a compound comprising a major histocompatibility complex (MHC) Tetramer and a toxin. MHC Tetramers are complexes of four Major Histocompatibility Complex (MHC) molecules which are each associated with a specific molecule. The specific molecule may be an epitope of the invention. In some embodiments, the four MHC molecules are associated with each other via a tetramerization agent. In some embodiments, the MHC Tetramer comprises four MHC monomer fusion proteins, wherein each MHC monomer fusion protein comprises a MHC molecule and biotin. In some embodiments, the tetramerization agent is streptavidin or avidin. In the compound, a MHC Tetramer may be coupled to a toxin by, e.g., a covalent bond, a linker, a streptavidin-biotin interaction or a streptavidin-avidin interaction. In some embodiments, the toxin is covalently bound to the streptavidin or the avidin. There are two types of Tetramers, Class I and Class II. In some embodiments, the MHC molecules of a Class I Tetramer are mutated to minimize binding of the MHC molecule to CD8+ cell surfaces. These Class I Tetramers show diminished CD8− mediated binding to the general CD8+ lymphocyte population, but retain MHC peptide-specific binding to TCR thus facilitating targeting of specific T cells that are activated by the epitope. MHC Tetramers are described in U.S. Patent Application Publication No. 2004/0137642 A1, published July 15, 2004; Hess et al., (2007) Selective deletion of antigen-specific CD8+ T cells by MHC class I tetramers coupled to the type I ribosome-inactivating protein saporin. Blood, 106:3300-3307; and at www.beckmancoulter.com/wsrportal/wsr/research/-and-discovery/products-and-services/flow-cytometry/class-I-itag-nhc-tetramers/index.htm, the entire content of each of which is hereby incorporated by reference.

It will be understood that by “treating” a subject there are multiple possible outcomes. For instance, treating a subject may comprise substantially reducing, slowing, stopping, preventing or reversing the progression of a disease, particularly a neurological disorder such as PD, LBD, or AD. Additionally, treating a subject may comprise substantially reducing, slowing, stopping, preventing or reversing a symptom of a disease. In the most favorable case, reduction is equivalent to prevention.

As used herein, a “symptom” associated with PD, LBD, ALS or AD includes any clinical or laboratory manifestation associated with PD, LBD, ALS or AD and is not limited to what the subject can feel or observe. Common symptoms of PD include but are not limited to tremors, muscle stiffness, difficulty maintaining balance, difficulty maintaining posture, bradykinesia, akinesia, rigid limbs, a shuffling gait, and a stooped posture. Other symptoms of PD include but are not limited to depression, personality changes, dementia, sleep disturbances, speech impairments, and sexual difficulties. Common symptoms of ALS include but are not limited to fasciculations (muscle twitches) in the arm, leg, shoulder, or tongue, muscle cramps, tight and stiff muscles (spasticity), muscle weakness affecting an arm, a leg, neck or diaphragm, slurred and nasal speech, difficulty chewing or swallowing, muscle atrophy. Other symptoms of ALS include but are not limited to depression, personality changes, dementia, sleep disturbances, speech impairments, and sexual difficulties.

The term “MHC” is used interchangeably with HLA herein. The genetic loci involved in the rejection of foreign organs are known as the major histocompatibility complex (MHC), and highly polymorphic cell surface molecules are encoded by the MHC. The human MHC is called the HLA (Human Leukocyte Antigen) system. When the specific HLA gene encoding the MHC recognized by the T cell receptor under investigation is not known, then a preceding experiment may be performed to screen the complete set of all MHC (HLA) molecules expressed from all alleles of the subject, e.g. of a patient, using methods well established in the art, such as for example as discussed in Robinson J, et al. (2003) Nucleic Acids Research, 31:311-314; Bettinotti et al. (2003) J. Immunol. Meth. 279:143-148; Marsh et al (2010) Tissue Antigens 75:291-455.

The term “genetic data” as used herein refers to information derived from a laboratory assay whereby a biological sample is processed in order to determine genetic data contained therein. For example, such genetic data may include data obtained from sequencing. Methods for sequencing comprise, without being limiting, approaches of sequence analysis by direct sequencing, fluorescent SSCP in an automated DNA sequencer and pyro-sequencing. These methods are well known in the art, see e.g. Adams et al. (Ed.), “Automated DNA Sequencing and Analysis”, Academic Press, 1994; Alphey, “DNA Sequencing: From Experimental Methods to Bioinformatics”, Springer Verlag Publishing, 1997; Ramon et al., J. Transl. Med. 1 (2003).sub.9; Meng et al., J. Clin. Endocrinol. Metab. 90 (2005) 3419-3422.

As used herein, the terms “individual,” “subject,” and “patient,” generally refer to a human subject, unless indicated otherwise.

As used herein, the term “treating” a subject includes multiple possible outcomes. For instance, treating a subject may comprise substantially reducing, slowing, stopping, preventing or reversing the progression of a disease, particularly a neurological disorder such as PO, LBO, or AD. Additionally, treating a subject may comprise substantially reducing, slowing, stopping, preventing or reversing a symptom of a disease. In the most favorable case, reduction is equivalent to prevention.

The terms “determining,” “measuring,” “evaluating,” “assessing,” and “assaying,” as used herein, generally refer to any form of measurement, and include determining if an element is present or not in a biological sample. These terms include both quantitative and/or qualitative determinations, which require sample processing and transformation steps of the biological sample. Assessing may be relative or absolute. The phrase “assessing the presence of” can include determining the amount of something present, as well as determining whether it is present or absent.

The term “stringent assay conditions” generally refers to conditions that are compatible to produce binding pairs of nucleic acids, e.g., probes and target TCR gene, of sufficient complementarity to provide for the desired level of specificity in the assay while being generally incompatible to the formation of binding pairs between binding members of insufficient complementarity to provide for the desired specificity. The term “stringent assay conditions” generally refers to the combination of hybridization and wash conditions.

A “label” or a “detectable moiety” in reference to a nucleic acid, generally refers to a composition that, when linked with a nucleic acid, renders the nucleic acid detectable, for example, by spectroscopic, photochemical, biochemical, immunochemical, or chemical means. Exemplary labels include but are not limited to radioactive isotopes, magnetic beads, metallic beads, colloidal particles, fluorescent dyes, enzymes, biotin, digoxigenin, haptens, and the like. A “labeled nucleic acid or oligonucleotide probe” is generally one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe can be detected by detecting the presence of the label bound to the nucleic acid or probe.

In one non-limiting embodiment, the herein described detection agent comprises a nucleic acid primer (or probe) having a sequence of 6-50, or 10-30, or 15-30, or 20-30 contiguous nucleotides of the target TCR, including any length between the stated ranges. Such primer may be present, if desired, on a microarray.

Primers (or probes) are usually single-stranded for maximum efficiency in amplification/hybridization, but may alternatively be double-stranded. If double-stranded, the primers (or probes) are usually first treated to separate the strands before use; this denaturation step is typically done by heat, but may alternatively be carried .out using alkali, followed by neutralization.

By way of a non-limiting example, the primers (or probes) for detecting a circulating microRNA may be labeled, using labeling techniques that are known to one skilled in the art, to facilitate detection, including but not limited to radioisotope labels or fluorescent labels. The primers (or probes) can hybridize to nucleic acid molecules that are either or both strands of the double stranded nucleic acid molecule portion of the microRNA.

A “label”’ or a “detectable moiety” in reference to a detecting agent, in particular in the case of primers (or probes), generally refers to a compound that, when linked with at least one detecting agent, renders it detectable, for example, by spectroscopic, photochemical, biochemical, immunochemical, or chemical means. An example of a “label” or a “detectable” moiety” includes but is not limited to radioactive isotopes, magnetic beads, metallic beads, colloidal particles, fluorescent dyes, enzymes, biotin, digoxigenin, haptens, and the like. In this context, “labeled” primers (or probe) includes primers (or probe) that are bound, either covalently, through a linker or a chemical bond, or noncovalently, through ionic bonds, van der Waals forces, electrostatic attractions, hydrophobic interactions, or hydrogen bonds, to a label such that the presence of the primers (or probe) can be detected by detecting the presence of the label bound to the primers (or probe).

A detectable label may be included in an amplification reaction. Suitable labels include fluorochromes, e.g., fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,W-tetramethyl-6-carboxyrhodamine (TAMRA), radioactive labels, e.g. 32P, 35S, 3H; etc. The label may be a two stage system, where the amplified DNA is conjugated to biotin, haptens, etc. having a high affinity binding partner, e.g., avidin, specific antibodies, etc., where the binding partner is conjugated to a detectable label. The label may be conjugated to one or both of the primers. Alternatively, the pool of nucleotides used in the amplification may be labeled, so as to incorporate the label into the amplification product. All of these and other labels are well known in the art and one can select corresponding suitable means for detecting such labels without departing from the present invention.

Hybridization primers (or probes) may be coupled to labels for detection. As with amplification primers, several methods and compositions for derivitizing oligonucleotides with reactive functionalities that permit the addition of a label are known in the art. For example, several approaches are available for biotinylating probes so that radioactive, fluorescent, chemiluminescent, enzymatic, or electron dense labels can be attached via avidin. See, e.g., Broken et al., Nucl. Acids Res. (1978) 5:363-384 which discloses the use of ferritin-avidin-biotin labels; and Chollet et al. Nucl. Acids Res. (1985) 13:1529-1541 which discloses biotinylation of the 5′ termini of oligonucleotides via an aminoalkylphosphoramide linker arm. Several methods are also available for synthesizing amino-derivatized oligonucleotides which are readily labeled by fluorescent or other types of compounds derivatized by amino-reactive groups, such as isothiocyanate, N-hydroxysuccinimide, or the like, see, e.g., Connolly (1987) Nucl. Acids Res. 15:3131-3139, Gibson et al. (1987) Nucl. Acids Res. 15:6455-6467 and U.S. Pat. No. 4,605,735 to Miyoshi et al. Methods are also available for synthesizing sulfhydryl-derivatized oligonucleotides which can be reacted with thiol-specific labels, see, e.g., U.S. Pat. No. 4,757,141, Connolly et al. (1985) Nuc. Acids Res. 13:4485-4502 and Spoat et al. (1987) Nucl. Acids Res. 15:4837-4848. A comprehensive review of methodologies for labeling DNA fragments is provided in Matthews et al., Anal. Biochem. (1988) 169:1-25.

For example, probes may be fluorescently labeled by linking a fluorescent molecule to the non-ligating terminus of the probe. Guidance for selecting appropriate fluorescent labels can be found in Smith et al., Meth. Enzymol. (1987) 155:260-301; Karger et al., Nucl. Acids Res. (1991) 19:4955-4962; Haugland (1989) Handbook of Fluorescent Probes and Research Chemicals (Molecular Probes, Inc., Eugene, Oreg.). In one embodiment, fluorescent labels include fluorescein and derivatives thereof, such as disclosed in U.S. Pat. No. 4,318,846 and Lee et al., Cytometry (1989) 10:151-164, and 6-FAM, JOE, TAMRA, ROX, HEX-1, HEX-2, ZOE, TET-1 or NAN-2, and the like.

Additionally, probes can be labeled with an acridinium ester (AE). Current technologies allow the AE label to be placed at any location within the probe. See, e.g., Nelson et al. (1995) “Detection of Acridinium Esters by Chemiluminescence” in Nonisotopic Probing, Blotting and Sequencing, Kricka L. J. (ed) Academic Press, San Diego, Calif.; Nelson et al. (1994) “Application of the Hybridization Protection Assay (HPA) to PCR” in The Polymerase Chain Reaction, Mullis et al. (eds.) Birkhauser, Boston, Mass.; Weeks et al., Clin. Chem. (1983) 29:1474-1479; Berry et al., Clin. Chem. (1988) 34:2087-2090. An AE molecule can be directly attached to the probe using non-nucleotide-based linker arm chemistry that allows placement of the label at any location within the probe. See, e.g., U.S. Pat. Nos. 5,585,481 and 5,185,439.

Hybridization (e.g., formation of a nucleic acid duplex) refers to the ability of a strand of nucleic acid to join with a complementary strand via base pairing. Hybridization occurs when complementary nucleic acid sequences in the two nucleic acid strands contact one another under appropriate conditions.

Nucleic acid hybridization is affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringency conditions depend on the length and base composition of the nucleic acid, which can be determined by techniques well known in the art. Generally, stringency can be altered or controlled by, for example, manipulating temperature and salt concentration during hybridization and washing. For example, a combination of high temperature and low salt concentration increases stringency. Such conditions are known to those skilled in the art and can be found in, for example, Strauss, W. M. “Hybridization With Radioactive Probes,” in Current Protocols in Molecular Biology 6.3.1-6.3.6, (John Wiley & Sons, N.Y. 2000). Both aqueous and non-aqueous conditions as described in the art can be used.

An example of stringent hybridization conditions is hybridization in 0.1×SSC (15 mM sodium chloride/1.5 mM sodium citrate) at 50 degree C. or higher. Another example of stringent hybridization conditions is hybridization overnight at 42 degree C. in 50% formamide, 1×SSC (150 mM NaCl, 15 mM sodium citrate), 50 mM sodium phosphate (pH 7.6), 5× Denhardt's solution, 10% (w/v) dextran sulfate, and 20 pg/ml denatured, sheared salmon sperm DNA, followed by washing in 0.1× SSC at about 65 degree C. Highly stringent conditions can include, for example, aqueous hybridization (e.g., free of formamide) in 6× SSC (where 20× SSC contains 3.0 M NaCl and 0.3 M sodium citrate), 1% (w/v) sodium dodecyl sulfate (SDS) at 65 degree C. for about 8 hours (or more), followed by one or more washes in 0.2× SSC, 0.1% SDS at 65 degree C.

Moderately stringent hybridization conditions permit a nucleic acid to bind a complementary nucleic acid that has at least about 60%, at least about 75%, at least about 85%, or greater than about 90% identity to the complementary nucleic acid. Stringency of hybridization is generally reduced by decreasing hybridization and washing temperatures, adding formamide to the hybridization buffer, or increasing salt concentration of the washing buffer, either individually or in combination. Moderately stringent conditions can include, for example, aqueous hybridization (e.g., free of formamide) in 6× SSC, 1% (w/v) SDS at 65 degree C. for about 8 hours (or more), followed by one or more washes in 2× SSC, 0.1% SDS at room temperature. Another exemplary hybridization under moderate stringency comprises hybridization in 6× SSC, 5× Denhardt's reagent, 0.5% (w/v) SDS, and optionally 100 pg/ml sonicated salmon or herring sperm DNA, at about 42 degree C., followed by washing in 2× SSC, 0.1% (w/v) SDS at 65 degree C. Other permutations and possibilities will be readily apparent to those of ordinary skill in the art, and are considered as equivalents within the scope of the instant invention.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-CAGT-3′,” is complementary to the sequence “5′-ACTG-3′.” Complementarity can be “partial” or “total.” “Partial” complementarity is where one or more nucleic acid bases are not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

In one non-limiting embodiment, substantially complementary nucleic acids have at least 50%, at least 60%, at least 70%, at least 80%, at least 90%, at least 95%, at least 98%, or at least 99% identical nucleotides.

As used herein, the term “about” for example with respect to a value relating to a particular parameter (e.g. concentration, such as “about 100 mM”) relates to the variation, deviation or error (e.g. determined via statistical analysis) associated with a device or method used to measure the parameter. For example, in the case where the value of a parameter is based on a device or method which is capable of measuring the parameter with an error of .+−.10%, “about” would encompass the range from less than 10% of the value to more than 10% of the value.

As used herein, “effective” when referring to an amount of a compound administered to a subject for the treatment of a neurological disease refers to the quantity of the compound that is sufficient to yield a desired therapeutic response without undue adverse side effects (such as toxicity, irritation, or allergic response) commensurate with a reasonable benefit/risk ratio when used in the manner of this invention.

Amino acid sequence of Tau is accessible in public databases by the accession number P10636-1 and is set forth herein as SEQ ID NO: 522. Nucleotide sequences for tau is accessible in public databases by the accession number J03778.1, which is set forth herein as SEQ ID NO: 523. Amino acid and nucleotide sequences of Tau are also accessible in public databases by the NCBI Gene ID: 4137. The name of the Tau gene is microtubule-associated protein Tau (MAPT). The amino acid sequence of amyloid β is accessible in public databases by the accession number P_05067-1 and is set forth herein as SEQ ID NO: 524.

The amino acid sequence of TDP43 is accessible in public databases by the accession number Q13148 and is set forth herein as SEQ ID NO: 525. The amino acid sequence of RNA-binding protein FUS is accessible in public databases by the accession number NP 004951.1 and is set forth herein as SEQ ID NO: 526. The amino acid sequence of superoxide dismutase is accessible in public databases by the accession number NP 000445.1 and is set forth herein as SEQ ID NO: 527.

Amino acid sequences of α-syn are accessible in public databases by the accession numbers NP_000336 and NP_009292, which are set forth herein as SEQ ID NOs: 528 and 529 respectively. Nucleotide sequences for α-syn is accessible in public databases by the accession numbers NM_000345 and NM_007308, which are set forth herein as SEQ ID NOs: 530 and 531 respectively. The amino acid sequence of leucine-rich repeat kinase 2 (LRRK2) is accessible in public databases by the accession number NP_940980 and is set forth herein as SEQ ID NO: 532. A nucleotide sequence for LRRK2 is accessible in public databases by the accession number NM_198578, which is set forth herein as SEQ ID NO: 533. The amino acid sequence of glucocerebrosidase is accessible in public databases by the accession number BAA02545 and is set forth herein as SEQ ID NO: 534. A nucleotide sequence for glucocerebrosidase is accessible in public databases by the accession number D13286, which is set forth herein as SEQ ID NO: 535. Amino acid and nucleotide sequences of tau are accessible in public databases by the NCBI Gene ID: 4137. The name of the tau gene is microtubule-associated protein tau (MAPT).

Aspects of the present invention relate to the HLA alleles. Additional information HLA alleles is available in Yokoyama et al. (2016) Association Between Genetic Traits for Immune-Mediated Diseases and Alzheimer Disease. JAMA Neurol, 73 (6):691-697, the entire content of which is incorporated herein by reference. Aspects of the present invention relate to the specific HLA alleles DRB5*01:01, DRB1*15:01, DQB1*03:04, A*11:01, DRB1*07:01, DRB1*09:01, or DQB1*03:01. The amino acid sequence for the DRB5*01:01 protein sequence is set forth herein as SEQ ID NO: 536 and the amino acid sequence for the DRB1*15:01 protein sequence is set forth herein as SEQ ID NO: 537. The amino acid sequence for the DQB1*03:04 protein sequence is set forth herein as SEQ ID NO: 538. The amino acid sequence for the A*11:01 protein sequence is set forth herein as SEQ ID NO: 539. The amino acid sequence for the DRB1*07:01 protein sequence is set forth herein as SEQ ID NO: 540. The amino acid sequence for the DRB1*09:01 protein sequence is set forth herein as SEQ ID NO: 541. The amino acid sequence for the DQB1*03:01 protein sequence is set forth herein as SEQ ID NO: 542. Additional information about these and other HLA alleles is available in Wissemann et al. (2013) Association of Parkinson disease with structural and regulatory variants in the HLA region. Am J Hum Genet, 93:984-993, PMC3824116, the entire content of which is incorporated herein by reference. Additional information, including sequence information, relating to these alleles and other alleles disclosed herein is available at www.ebi.ac.uk/ipd/imgt/hla/allele.html. It will be understood that persons skilled in the art are able to identify and obtain sequences and genomic locations for the HLA alleles disclosed herein using knowledge in the art.

Non-limiting examples of compounds which may be used in the treatment of AD in embodiments of the invention include holinesterase inhibitors (e.g., donepezil, rivastigmine, galantamine, and tacrine), N-methyl-d-aspartate receptor antagonist (e.g., ,memantine), high-dose vitamin E (1000 IU po once/day or bid), selegiline, NSAIDs, Ginkgo biloba extracts, and statins.

Non-limiting examples of compounds which may be used in the treatment of PD in embodiments of the invention include growth factors (e.g., GDNF), cell transplantation, deep brain stimulation, anti-inflammatory drugs.

Non-limiting examples of compounds which may be used in the treatment of PD in embodiments of the invention include dopamine precursors (e.g., levodopa and carbidopa), dopamine agonists (e.g., bromocriptine, pramipexole, ropinirole, apomorphine, and rotigotine), MAO-B inhibitors (e.g., rasagiline, selegiline, and pargyline), COMT inhibitors (e.g., entacapone and tolcapone), anticholinergic compounds (e.g., trihexyphenidyl, benztropine, amitiriptyline and diphenhydramine) antiviral compounds (e.g., amantadine), beta-blockers (e.g., propranolol), calcium channel blocker (e.g. isradipine and dihydropyridine), and antioxidants.

Non-limiting examples of compounds which may be used in the treatment of ALS in embodiments of the invention include riluzole (Rilutek) and edaravone (Radicava), baclofen, quinine or phenytoin, anticholinergic drug (eg, glycopyrrolate, amitriptyline, benztropine, trihexyphenidyl, transdermal hyoscine, atropine, amitriptyline, fluvoxamine, or a combination of dextromethorphan.

Non-limiting examples of compounds which may be used in the treatment of AD, PD or ALS in embodiments of the invention also include immunosuppressive compounds. In some embodiments, an immunosuppressive compound targets an autoimmune component in AD, PD or ALS, for example T cell activation or function. Non-limiting examples of approaches for suppressing the immune system, or a component thereof, in embodiments of the subject invention include:
1. Blocking receptors of chemokines such as CCR5 present on cytotoxic Tcells. This can be achieved by using antagonist drugs such as maraviroc. It will be understood that CCR5 is one of the HIV-1 receptors and such drugs have been in use for years to treat HIV patients.
2. Administering a glucocorticoid such as prednisone or prednisolone, which are effective immunosuppressive agents. They inhibit the activation of cytotoxic T cells. Additionally, they cross the blood brain barrier and are used to treat multiple sclerosis (MS).
3. Administering a calcineurin inhibitor such as cyclosporine or tacrolimus, which are potent immunosuppressive agents. They inhibit calcineurin, which blocks phosphatase activity, and thus T cell activation. They are used to inhibit transplant rejection.
4. Administering an inhibitor of mTOR such as rapamaycin, which blocks cell cycle at G1>S phase. Rapamaycin inhibits T cell activation and proliferation. It is used to treat transplant rejection.
5. Administering an anti-metabolic drug including azathioprine, micophenolate or mofetil to block killer T cells.
6. Administeration of antibodies for LFA-3Igl fusion protein, which interferes with T cell activation. This has been used in psoriasis.
7. Administering a phosphodiesterase-5 inhibitor such as sildenafil or paclitaxel, which have been used in melanoma to lead to cell-mediated T cell immunosupression.

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

As used herein, the term “about” for example with respect to a value relating to a particular parameter (e.g. concentration, such as “about 100 mM”) relates to the variation, deviation or error (e.g. determined via statistical analysis) associated with a device or method used to measure the parameter. For example, in the case where the value of a parameter is based on a device or method which is capable of measuring the parameter with an error of .+−.10%, “about” would encompass the range from less than 10% of the value to more than 10% of the value.

It must be noted that, as used herein and in the appended claims, the singular forms “a,” “an,” and “the” include plural referents and plural referents include singular forms unless the context clearly dictates otherwise. Thus, for example, reference to “a subject polypeptide” includes a plurality of such polypeptides, reference to the agent” includes reference to one or more agents and equivalents thereof known to those skilled in the art, reference to “nucleic acid molecules” includes reference to one or more nucleic acid molecules, and reference to “antibodies” includes reference to one or more antibodies and so forth.

With respect to ranges of values, it is contemplated that these encompass the upper and lower limits and each intervening value between the upper and lower limits of the range to at least a tenth of the upper and lower limit's unit, unless the context clearly indicates otherwise. Further, the invention encompasses any other stated intervening values.

The foregoing is considered as .illustrative only of the principles of the invention. Further, since numerous modifications and changes will readily occur to those skilled in the art, it is not desired to limit the invention to the exact examples and embodiments shown and described, and accordingly, all suitable modifications and equivalents may be resorted to, falling within the scope of the claims.

All publications and other references mentioned herein are incorporated by reference in their entirety, as if each individual publication or reference were specifically and individually indicated to be incorporated by reference. Publications and references cited herein are not admitted to be prior art.

This invention will be better understood by reference to the Experimental Details which follow, but those skilled in the art will readily appreciate that the specific experiments detailed are only illustrative of the invention as defined in the claims which follow thereafter.

### EXPERIMENTAL DETAILS

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Example 1

### Phosphorylated Tau is Recognized as an Autoantigen

Identification of specific Tau antigens that act as autoantigens. This can be used as the source of biomarkers, diagnostics and therapeutics via tolerization and related approaches.

Tau, the protein product of the MAPT gene, in highly phosphorylated aggregates has long been associated with Alzheimer's disease (AD), progressive supranuclear palsy (PSP) and other dementias; in addition, MAPT has been identified as a risk factor for Parkinson's disease (PD) by GWAS (Sharma et al., 2012), but not AD itself. Phospho-Tau immunolabel can also be high in PD and particularly LBD brain, while phospho-Tau is higher in AD, and there is often significant overlap in patient brain pathology between the disorders (Arnold et al., 2013). Tau and α-syn have many parallel features including association with PD by GWAS, phosphorylation under disease conditions, presence of both proteins (Hampel et al., 2010; Foulds et al., 2013; Zetterberg et al., 2013), and autoantibodies in blood (Bartos et al., 2012; Koehler et al., 2013), and similar degradation by CMA that is disturbed by mutation (Wang et al., 2009). They may even form joint oligomers in some patients (Sengupta et al., 2015).

Phosphorylated Tau is recognized as an autoantigen by T cells in the blood in PD. Without wishing to be bound by any scientific theory, an autoimmune response in Tauopathies such as Alzheimer's and other dementias can be the basis of new means for diagnosis, biomarkers, and clinical therapies.

Phosphorylated candidate epitopes are important for Tau, which has ˜40 potential phosphorylated sites (Sharma et al., 2012; Yin et al., 2013), of which 20 were identified in AD patients (Duka et al., 2013); 10 phosphorylated sites were identified in PD striata (S202, 235, 262, 356, 396/404, 409, 413, 422 and T205, 212); and seven sites in LBD (S214, 238, 396/404, 422 and T212, 217). Interestingly, in PD there are 3 clusters of phospho-Tau (202,205,212; 356, 396, 404; 409, 413, 422). To conduct this assay, 15 mer epitopes were analyzed that contain the following phosphorylated residues of Tau, each of which are reported in dementias: T181, 5199, 5202, T205, T212, 5214, T231, 5262, 5356, 5422. These peptides were incubated in PBMCs obtained from 3 PD patients and one age-matched control and recorded T cell activation following the Sette lab's published protocols. Each individual including the control was found to have T cell responses to at least some set of these epitopes. The precise antigens are determined in assays as the pooled peptides are segregated. The precise T cell types and HLA alleles involved are also identified. Without wishing to be bound by any scientific theory, the observation that T cell response is quite common may underlie the very high population that exhibits AD and related dementias.

### Example 2

### Cytokine Release in Controls and PD Patients

Blood from age matched controls and PD patients were obtained and mononuclear cells were isolated by gradient centrifugation.

Release of the cytokine interferon-gamma (IFNg), which measures activation of CD4+ and/or CD8+ T cells, and the interleukin, IL-5, which measures activation of CD4+ T cells was measured by ELISPOT assay. Briefly, the isolated cells were plated in wells that have colorimetric detection of IFNg and IL-5, and were stimulated with pools of 95 epitopes of α-synuclein that the Sette lab determined would potentially be displayed by MHC-I or MHC-II antigen-presenting proteins in humans. After 22 hours of stimulation at 37 C, the cells were removed and release of cytokines was measured by colorimetric detection of spot-forming cells (SFC). Confirmed release of cytokine is determined by the presence of a minimum of 20 SFC per million cells.

Cytokine release was detected in only 15 of 160 stimulations to α-synuclein epitopes across controls (n=12), while 43 of 248 stimulations in PD patients had antigenic responses, yielding a probability of p=0.029 that PD patients are a different population (two-tailed Fisher exact test). Thus, the data shows that PD patients are more likely to have T cells in blood that recognize and are activated by α-synuclein than unaffected individuals.

### Example 3

### Tolerization Therapy Specific for Epitopes of Tau are Useful in Treating Subjects Afflicted with AD

Epitopes to which T cells are responsive in subjects afflicted with AD are identified by

i) obtaining T cells from each subject;

ii) contacting the T cells with a test compound;

iii) determining whether the T cells have increased activation after contact with the test compound; and

iv) identifying the test compound as an epitope to which the T cells are responsive if in step iii) the T cells are determined to have increased activation after contact with the test compound, and identifying the test compound as not an epitope to which the T cells are responsive if in step iii) the T cells are determined to not have increased activation after contact with the test compound. This method is repeated sequentially or in parallel for thousands of test compounds, each having an amino acid sequence identical to a stretch of consecutive amino acids in the Tau protein. Epitopes for Tau are identified in individual subjects.

The subjects afflicted with PD are then separated into one of two groups: 1) a test group that receives tolerization therapy, or 2) a control group that does not receive tolerization therapy.

Within the test group, an effective amount of a Tau epitope is administered orally, nasally, or subcutaneously to each subject (i.e., tolerization therapy specific for, the epitope). Within the control group, a polypeptide having a random sequence is administered to each subject.

Compared to the control group, subjects in the test group have a statistically significant reduction in symptoms of AD. Additionally, a statistically significant proportion of the subjects have little or no progression of AD.

Less or no activation of T cells by the epitope is observed in subjects who receive and respond to tolerization therapy, but not in subjects who do not receive or who do not respond to tolerization therapy.

### Example 4

### Autoimmune Features of Neurodegenerative Disorders

Without wishing to be bound by any scientific theory, at least some AD is in part an autoimmune disorder.

Without wishing to be bound by any scientific theory, the T cells recognize Tau.

### Example 5

### Parkinson's Disease is Associated with HLA Class II Restricted CD4 T Cell Responses Targeting the Tau Antigen

The Tau protein is known to accumulate with age and in a number of different disease conditions. Whether the aggregated proteins are recognized by T cell responses is currently unknown, and it is also unknown whether differential recognition occurs between healthy people and patients affected by several different neurodegenerative diseases, such as Parkinson's (PD), Alzheimer's, Dementia, ALS, Schizophrenia and others. This experiment investigated whether peptides derived from the Tau protein are preferentially recognized in PD patients.

A) Accrual of PD Patients and Control Donor Cohorts

Features, diagnosis, recruitment, age, gender are described in Table 6 below.

TABLE 6
Demographics of Study Participants

| | Parkinson's | | Healthy controls | | Healthy controls | |
|---|---|---|---|---|---|---|
| | cases | | (>50 years old) | | (<35 years old) | |
| | (n = 22) | | (n = 21) | | (n = 22) | |
| Mean age in | 65.5 | (6.2) | 62.3 | (6.1) | 25.0 | (4.5) |
| years, (SD) | | | | | | |
| Male, % (n) | 72.7 | (16) | 33.3 | (7.0) | 63.6 | (14) |
| Subjects with | 9.1 | (2) | 14.3 | (3) | Unknown | |
| family history of | | | | | | |
| Parkinson's | | | | | | |
| disease in first- | | | | | | |
| degree relative, % | | | | | | |
| (n) | | | | | | |
| Mean Parkinson's | 56.6 | (10.4) | N/A | | N/A | |
| age-at-onset, (SD) | | | | | | |
| Caucasian, % (n) | 100 | (22) | 85.7 | (18) | 77.3 | (17) |

B) Tau-Specific T cells are Detected in Both PD and Control Donors, Throughout the Tau Antigen Sequence

It was investigated whether T cell responses against Tau were detectable in the PD and healthy control (HC) donors. To this end, a panel of overlapping peptides spanning through the entire sequence of the Tau protein was synthesized. Since it is known that the Tau protein is post-translationally modified (mainly by phosphorylation), in several instances phosphorylated peptides were also synthesized. In all, 55 non-modified 16-mers overlapping by 8 and 14 modified 16-mers were synthesized.

C) Results

PD: n=16, peptides tested 14-16 times; HC above 50 years old denominated as age-matched: n=11 (9-11 times) and HC below 35 years old (HC Young): n=13 (10-13 times).

These peptides were tested utilizing the following assay strategy. Briefly, PBMC from each donor were stimulated in vitro for 14 days as described (Hinz, D., 2015) with pools of 10 to 16 peptides each. After 14 days, the T cell cultures were assayed with IFNg/IL-5 dual ELISPOT assays (ref). Positive pools were deconvoluted at day 17 to identify the specific individual epitopes recognized.

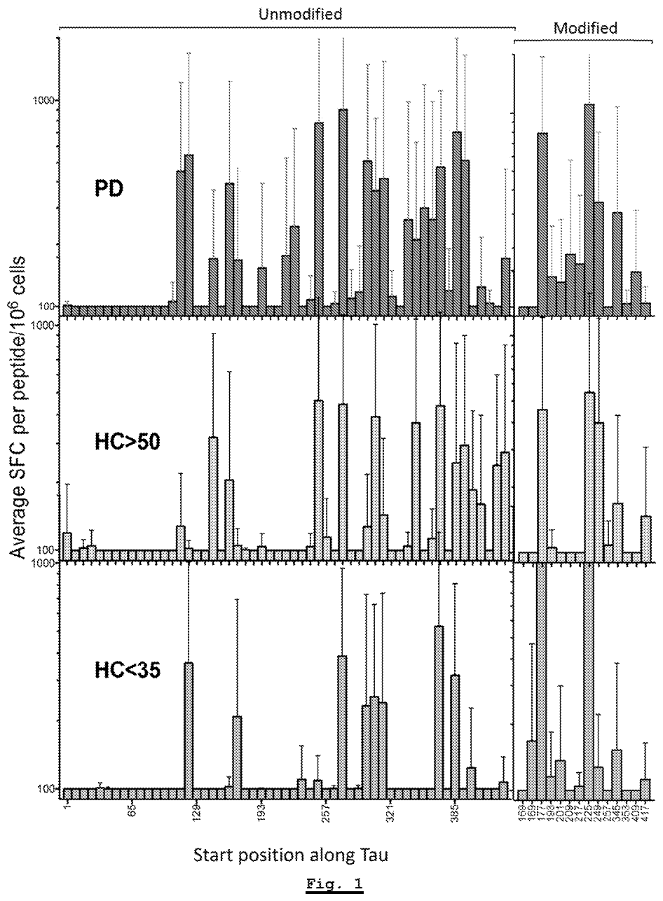

The results shown in FIG. 1 for PD, HC Young and HC Age-matched donors show that Tau specific T cells were detected in both PD and control donors, throughout the Tau antigen sequence, both in the case of unmodified peptides, as well as the modified versions, also shown in separate panels.

D) Analysis of Overall Response Reveals Higher Responses in PD Versus Control Donors

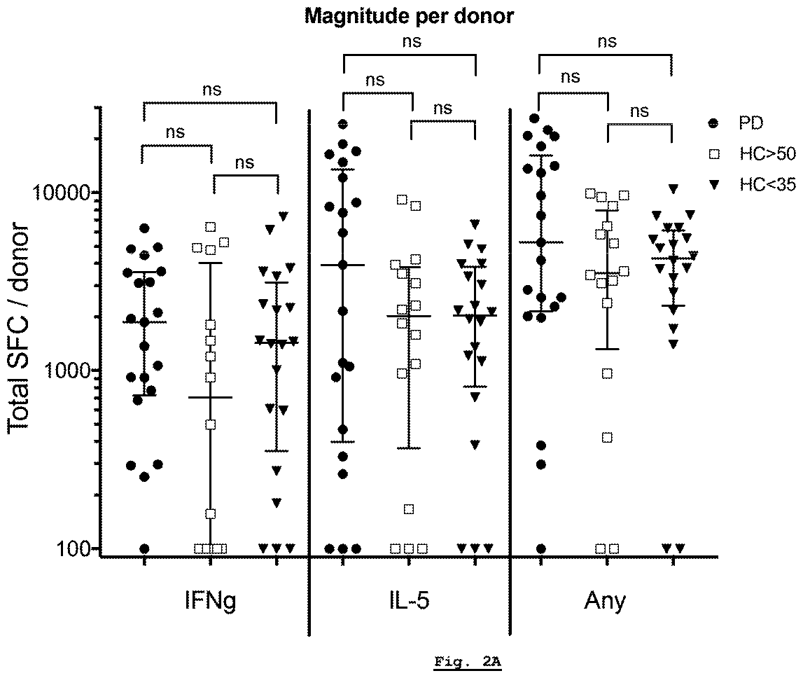

Further analysis showed that while the reactivity of healthy young and age-matched controls was similar, the reactivity of PD donors was higher than the age-matched controls FIG. 2A and 2B. More specifically, in FIG. 2A responses detected for each donor are shown, reporting separately IFNg, IL5 or the sum of both cytokines. There was a trend for responses in the PD cohort being higher than for either the young or age-matched controls. A significant difference was actually detected in terms of IFNg responses between the PD and age-matched controls.

In FIG. 2B, the responses observed in each donor against each individual peptide are plotted. By this analysis, no significant difference was seen between the young and age-matched controls. However, significant differences were noted between PD and either group of controls, when the IFNg, IL5 or both cytokines combined was considered.

E) Phosphorylated Sequences are More Recognized Than Unmodified Ones

Next, the magnitude and frequency of responses to unmodified and phosphorylated peptides were compared. The results are shown in the following Table 7 for the seven cases where pairs of phosphorylated and non-phosphorylated peptides were tested and responses were detected in at least four donors against one of the peptide pairs. Strikingly, in seven out of seven cases phosphorylated sequences were recognized more frequently than unmodified ones, in higher frequencies and/or magnitude.

TABLE 7
Phosphorylated Sequences are More Recognized Than Unmodified
Ones

| | | | | | PD | | HC > 50 | | HC < 35 | |
|---|---|---|---|---|---|---|---|---|---|---|
| | | | Total | Total | Total | Total | Total | Total | Total | Total |
| Position | Sequencee | Modification | Responders | SFC | Responders | SFC | Responders | SFC | Responders | SFC |
| 177 | PAPKTPPSSG | | 1 | 107 | 0 | 0 | 1 | 107 | 0 | 0 |
| | EPPKSG | | | | | | | | | |
| | PAPKXPPSSG | X = pT | 26 | 42624 | 9 | 14672 | 5 | 6207 | 12 | 21745 |
| | EPPKSG | | | | | | | | | |
| 193 | DRSGYSSPGS | | 2 | 1332 | 1 | 1175 | 1 | 157 | 0 | 0 |
| | PGTPGS | | | | | | | | | |
| | DRSGYSSPGZ | X = pT, | 9 | 5499 | 5 | 1360 | 1 | 187 | 3 | 3952 |
| | PGXPGS | Z = pS | | | | | | | | |
| 217 | TPPTREPKKV | | 1 | 1707 | 1 | 1707 | 0 | 0 | 0 | 0 |
| | AVVRTP | | | | | | | | | |
| | TPPTREPKKV | X = pT | 7 | 2210 | 4 | 1813 | 0 | 0 | 3 | 397 |
| | AVVRXP | | | | | | | | | |
| 225 | KVAVVRTPPK | | 5 | 4556 | 5 | 4556 | 0 | 0 | 0 | 0 |
| | SPSSAK | | | | | | | | | |
| | KVAVVRXPPK | X = pT | 23 | 46324 | 12 | 22410 | 3 | 6987 | 8 | 16927 |
| | SPSSAK | | | | | | | | | |
| 249 | PMPDLKNVKS | | 10 | 19815 | 5 | 13363 | 3 | 6084 | 2 | 368 |
| | KIGSTE | | | | | | | | | |
| | PMPDLKNVKS | Z = pS | 11 | 11063 | 5 | 5175 | 4 | 5168 | 2 | 720 |
| | KIGZTE | | | | | | | | | |
| 345 | DFKDRVQSKI | | 5 | 6727 | 2 | 2397 | 3 | 4330 | 0 | 0 |
| | GSLDNI | | | | | | | | | |
| | DFKDRVQSKI | Z = pS | 7 | 6807 | 4 | 4490 | 2 | 1220 | 1 | 1097 |
| | GZLDNI | | | | | | | | | |
| 409 | SNVSSTGSID | | 3 | 1767 | 2 | 710 | 1 | 1057 | 0 | 0 |
| | MVDSPQ | | | | | | | | | |
| | SNVSSTGSID | Z = pS | 4 | 1484 | 4 | 1484 | 0 | 0 | 0 | 0 |
| | MVDZPQ | | | | | | | | | |
| 417 | IDMVDSPQLA | | 1 | 173 | 1 | 173 | 0 | 0 | 0 | 0 |
| | TLADEV | | | | | | | | | |
| | IDMVDZPQLA | Z = pS | 5 | 1703 | 1 | 197 | 2 | 943 | 2 | 563 |
| | TLADEV | | | | | | | | | |

F) Overall Responses are not Correlated with Age in Either Controls or PD Patients

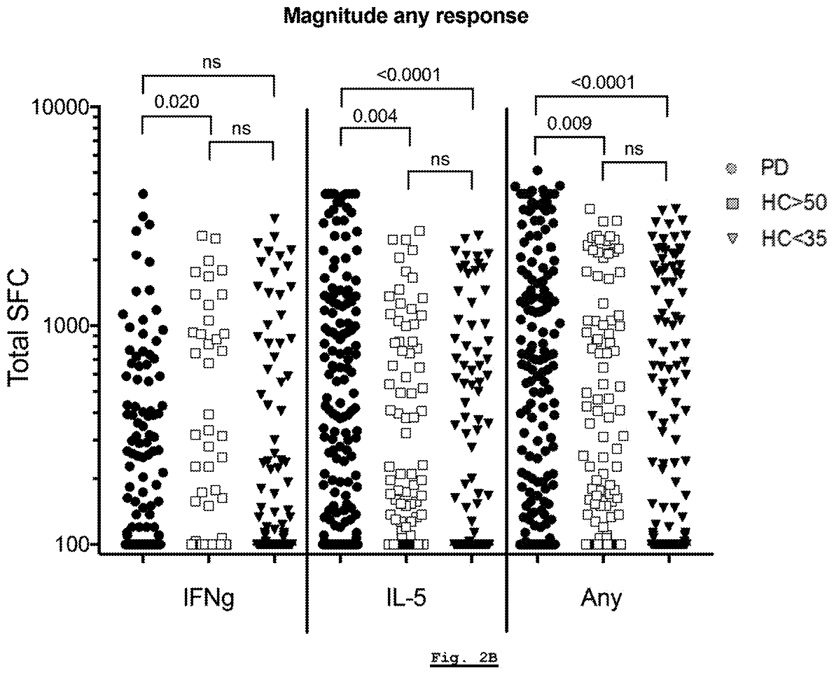

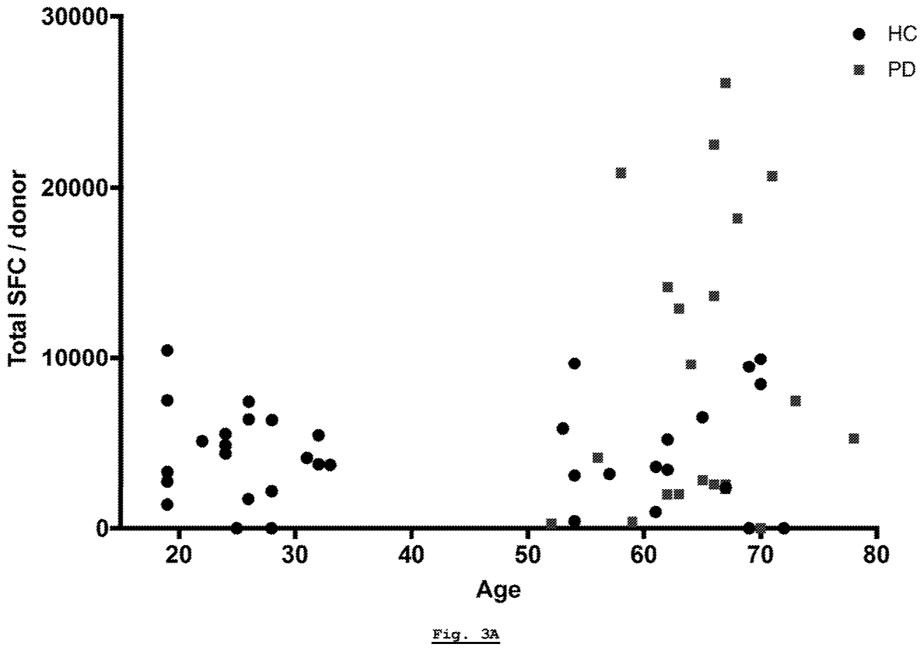

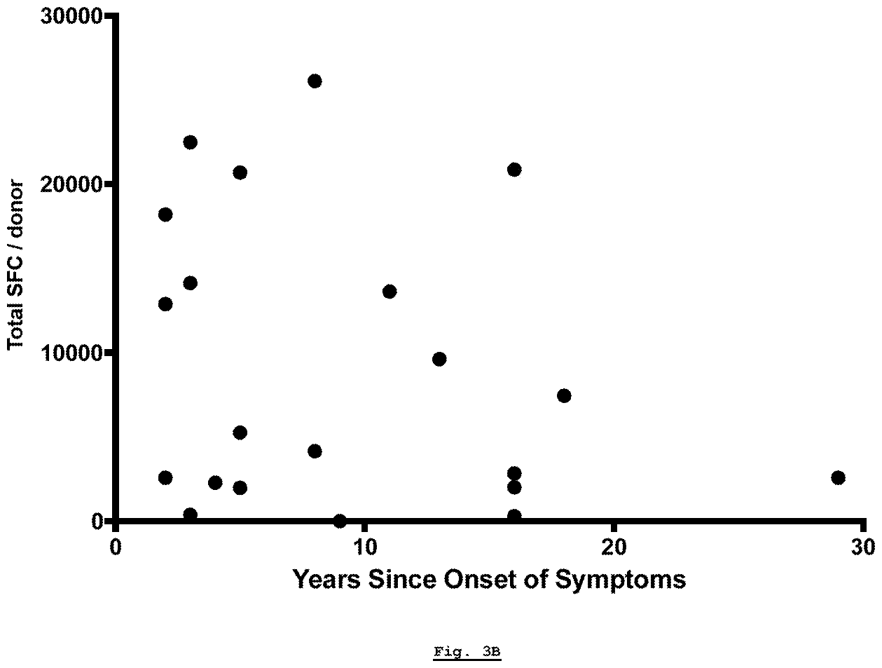

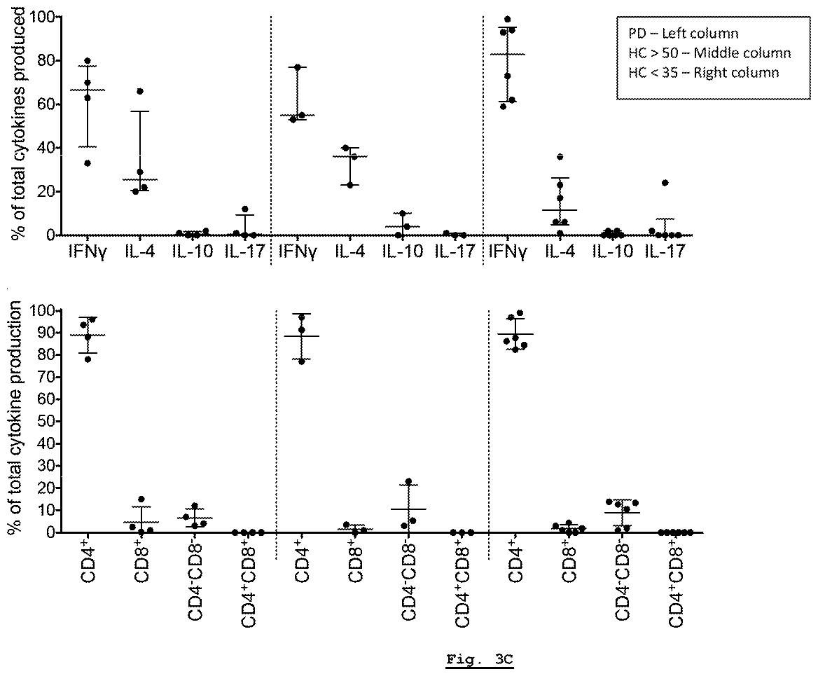

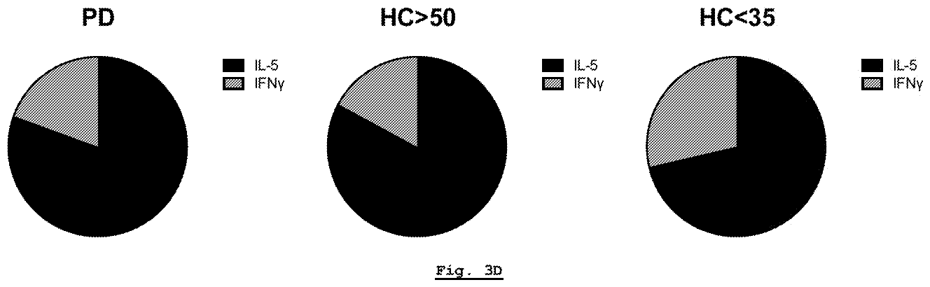

The potential relationship between age and Tau reactivity by plotting reactivity for each donor as a function of age was addressed. In FIG. 3A, the PD donors are shown in red, while the controls (both age-matched and young) are shown with black symbols. While there is a mild trend toward correlation with age, the trend is not significant. We also show a plot, for the PD donors of total reactivity as a function of time since symptoms onset (FIG. 3B). Here again no significant correlation is detected.

G) Intercellular Cytokine Staining Indicates that Responses are Polarized Towards CD4, IFNg and IL-4

To further characterize responses, we also analyzed responses using

Intercellular Cytokine Staining (ICS) to ascertain whether the three different cohorts differed in the patterns of cytokine produced in response to antigenic stimulation. When using ICS, in all three cohorts IFNγ and IL-4 were the predominant cytokines detected (FIG. 3C). Notably, no IL-10 was produced. Further, the Tau epitope response was produced almost entirely by CD4⁺ cells.

H) ELISPOT Indicates that Responses are Polarized Towards IL-5

To further characterize responses, we also analyzed responses using Enzyme-Linked ImmunoSport (ELISPOT) to ascertain whether the three different ,cohorts differed in the patterns of cytokine produced in response to antigenic stimulation. When using ELISPOT, in all three cohorts IL-5 was the predominant cytokine detected (FIG. 3D).

I) Differences Between the Donor Cohorts are to be Ascribed to Breadth, and not per Epitope Magnitude

In the next series of analyses, the basis for the higher responses detected in the PD cohort was investigated in more detail. Specifically, it was addressed separately whether the reason for the higher response was to be ascribed to a larger number of epitopes being recognized (breadth), a higher magnitude of positive responses (magnitude) or both.

It was found that both breadth (FIG. 4A) and magnitude of response/epitope (FIG. 4B) were increased in the PD donors with respect to controls. These results suggest that both factors contribute to the difference in response observed.

J) Tau-Specific Responses are Stronger than those Against α-syn

Next, the cytokine response as determined by ELISPOT assay to Tau epitopes was compared to the response to α-syn. It was found that the Tau-specific response was significantly stronger than the α-syn specific response (FIG. 5).

K) Definition of Dominant Epitopes, and PD Specific Ones

The most dominantly recognized epitopes were defined as those accounting for 90% of the total response; those epitopes are listed in the following Table 8. The most dominant epitopes were recognized in all three cohorts in most cases. In a few cases, the epitopes were selectively recognized in PD donors. The following Table 9 lists the selectivity of the dominantly recognized epitopes.

TABLE 8
Dominant Epitopes Across All Cohorts

| | Start | | Total | | | |
|---|---|---|---|---|---|---|
| | position | | SFC | Average | % of | Frequency |
| | along | | per | SFC per | total | of |
| Sequence | Tau | Modification | epitope | responder | response | response |
| KVAVVRXPPKSPS | 225 | X = pT | 46324 | 12014 | 13.2 | 43.4 |
| SAK | | | | | | |
| PAPKXPPSSGEPP | 177 | X = pT | 42624 | 1639 | 12.2 | 49.1 |
| KSG | | | | | | |
| GKVQIINKKLDLS | 273 | | 30211 | 1511 | 8.6 | 37.7 |
| NVQ | | | | | | |
| KTDHGAEIVYKSP | 385 | | 20142 | 1679 | 5.7 | 22.6 |
| VVS | | | | | | |
| PMPDLKNVKSKIG | 249 | | 19815 | 1982 | 5.7 | 18.9 |
| STE | | | | | | |
| KKIETHKLTFREN | 369 | | 18790 | 1174 | 5.4 | 30.2 |
| AKA | | | | | | |
| HVTQARMVSKSKD | 121 | | 14677 | 1468 | 4.2 | 18.9 |
| GTG | | | | | | |
| SVQIVYKPVDLSK | 305 | | 13888 | 731 | 4.0 | 35.8 |
| VTS | | | | | | |
| VYKSPVVSGDTSP | 393 | | 13408 | 1915 | 3.8 | 13.2 |
| RHL | | | | | | |
| IKHVPGGGSVQIV | 297 | | 11875 | 1319 | 3.4 | 17.0 |
| YKP | | | | | | |
| PMPDLKNVKSKIG | 249 | Z = pS | 11063 | 1006 | 3.2 | 20.8 |
| ZTE | | | | | | |
| VDLSKVTSKCGSL | 313 | | 9861 | 1409 | 2.8 | 13.2 |
| GNI | | | | | | |
| GQKGQANATRIPA | 161 | | 8164 | 1361 | 2.3 | 11.3 |
| KTP | | | | | | |
| SLEDEAAGHVTQA | 113 | | 7698 | 1283 | 2.2 | 11.3 |
| RMV | | | | | | |
| DFKDRVQSKIGZL | 345 | Z = pS | 6807 | 972 | 1.9 | 13.2 |
| DNI | | | | | | |
| DFKDRVQSKIGSL | 345 | | 6727 | 1345 | 1.9 | 9.4 |
| DNI | | | | | | |
| DRSGYSSPGZPGX | 193 | X = pTZ = pS | 5499 | 611 | 1.6 | 17.0 |
| PGS | | | | | | |
| ADGKTKIATPRGA | 145 | | 5210 | 868 | 1.5 | 11.3 |
| APP | | | | | | |
| ATLADEVSASLAK | 426 | | 4997 | 999 | 1.4 | 9.4 |
| QGL | | | | | | |
| KVAVVRTPPKSPS | 225 | | 4556 | 911 | 1.3 | 9.4 |
| SAK | | | | | | |
| KIGSLDNITHVPG | 353 | | 4070 | 4070 | 1.2 | 1.9 |
| GGN | | | | | | |
| TRIPAKTPPAPKT | 169 | | 3897 | 1299 | 1.1 | 5.7 |
| PPS | | | | | | |
| GDTSPRHLSNVSS | 401 | | 3798 | 633 | 1.1 | 11.3 |
| TGS | | | | | | |
| LATLADEVSASLA | 425 | | 3793 | 948 | 1.1 | 7.5 |
| KQG | | | | | | |

TABLE 9
Selectivity in Recognition of Dominant Epitopes

| | | | | | | | PD/HC > | PD/HC > |
|---|---|---|---|---|---|---|---|---|
| | Start | | | | | | 50 | 50 |
| | position | | PD | % of | HC > 50 | % of | Ratio | Ratio |
| | along | | Total | Total | Total | Total | of | of % |
| Sequence | Tau | Modification | SFC | Responders | SFC | Responders | Response | Responders |
| KVAVVRTPPK | 225 | | 4556 | 100.0 | 0 | 0.0 | inf | inf |
| SPSSAK | | | | | | | | |
| KIGSLDNITH | 353 | | 4070 | 100.0 | 0 | 0.0 | inf | inf |
| VPGGGN | | | | | | | | |
| HVTQARMVSK | 121 | | 8916 | 50.0 | 133 | 10.0 | 66.9 | 5.0 |
| SKDGTG | | | | | | | | |
| IKHVPGGGSV | 297 | | 8565 | 44.4 | 737 | 33.3 | 11.6 | 1.3 |
| QIVYKP | | | | | | | | |
| SLEDEAAGHV | 113 | | 7058 | 66.7 | 640 | 33.3 | 11 | 2.0 |
| TQARMV | | | | | | | | |
| TRIPAKTPPA | 169 | | 145 | 33.3 | 180 | 33.3 | 8.1 | 1.0 |
| PKTPPS | | | | | | | | |
| VDLSKVTSKC | 313 | | 671 | 57.1 | 897 | 28.6 | 7.5 | 2.0 |
| GSLGNI | | | | | | | | |
| DRSGYSSPGZ | 193 | X = pT, | 1360 | 55.6 | 187 | 11.1 | 7.3 | 5.0 |
| PGXPGS | | Z = pS | | | | | | |
| KTDHGAEIVY | 385 | | 13336 | 58.3 | 2437 | 8.3 | 5.5 | 7.0 |
| KSPVVS | | | | | | | | |
| DFKDRVQSKI | 345 | Z = pS | 4490 | 57.1 | 1220 | 28.6 | 3.7 | 2.0 |
| GZLDNI | | | | | | | | |
| GQKGQANATR | 161 | | 6247 | 66.7 | 1770 | 16.7 | 3.5 | 4.0 |
| IPAKTP | | | | | | | | |
| GKVQIINKKL | 273 | | 18290 | 50.0 | 5560 | 20.0 | 3.3 | 2.5 |
| DLSNVQ | | | | | | | | |
| KVAVVRXPPK | 225 | X = pT | 22410 | 52.2 | 6987 | 13.0 | 3.2 | 4.0 |
| SPSSAK | | | | | | | | |
| VYKSPVVSGD | 393 | | 8951 | 42.9 | 3397 | 42.9 | 2.6 | 1.0 |
| TSPRHL | | | | | | | | |
| PAPKXPPSSG | 177 | X = pT | 14672 | 34.6 | 6207 | 19.2 | 2.4 | 1.8 |
| EPPKSG | | | | | | | | |
| PMPDLKNVKS | 249 | | 13363 | 50.0 | 6084 | 30.0 | 2.2 | 1.7 |
| KIGSTE | | | | | | | | |
| KKIETHKLTF | 369 | | 8190 | 43.8 | 5898 | 31.3 | 1.4 | 1.4 |
| RENAKA | | | | | | | | |
| SVQIVYKPVD | 305 | | 6203 | 36.8 | 5460 | 42.1 | 1.1 | 0.9 |
| LSKVTS | | | | | | | | |
| PMPDLKNVKS | 249 | Z = pS | 5175 | 45.5 | 5168 | 36.4 | 1 | 1.3 |
| KIGZTE | | | | | | | | |
| ATLADEVSAS | 426 | | 2193 | 60.0 | 2803 | 40.0 | 0.8 | 1.5 |
| LAKQGL | | | | | | | | |
| DFKDRVQSKI | 345 | | 2397 | 40.0 | 4330 | 60.0 | 0.6 | 0.7 |
| GSLDNI | | | | | | | | |
| GDTSPRHLSN | 401 | | 997 | 16.7 | 1657 | 50.0 | 0.6 | 0.3 |
| VSSTGS | | | | | | | | |
| ADGKTKIATP | 145 | | 1633 | 50.0 | 3577 | 50.0 | 0.5 | 1.0 |
| RGAAPP | | | | | | | | |
| LATLADEVSA | 425 | | 1263 | 25.0 | 2530 | 75.0 | 0.5 | 0.3 |
| SLAKQG | | | | | | | | |

A genetic inference approach to attribute potential HLA restriction to specific epitopes was used (Paul et al., 2017). This approach was used to identify potential restriction of Tau epitopes. This analysis allowed assigning restriction for many of the most prominently recognized epitopes. The results are shown in the following Table 10.

TABLE 10
Inferred HLA Restrictions

| | | | | | | Relative | Odds | |
|---|---|---|---|---|---|---|---|---|
| Peptide Sequence | Allele | A+R+ | A−R+ | A+R− | A−R− | Frequency | Ratio | P-value |
| ADGKTKIATPRGAAPP | DRB1*11:04 | 3 | 3 | 1 | 46 | 6.6 | 46.0 | 0.003 |
| SRLQTAPVPMPDLKNV | DQB1*03:03 | 2 | 0 | 3 | 49 | 10.8 | inf | 0.007 |
| PMPDLKNVKSKIGSTE | DQA1*05:01 | 8 | 2 | 11 | 31 | 2.2 | 11.3 | 0.003 |
| VDLSKVTSKCGSLGNI | DRB1*01:01 | 4 | 3 | 4 | 44 | 3.9 | 14.7 | 0.006 |
| KTDHGAEIVYKSPVVS | DRB1*04:04 | 3 | 9 | 0 | 44 | 4.7 | inf | 0.008 |
| SNVSSTGSIDMVDSPQ | DQB1*04:02 | 2 | 1 | 0 | 52 | 18.3 | inf | 0.002 |
| KVAVVRXPPKSPSSAK | DRB3*02:02 | 6 | 7 | 8 | 24 | 1.6 | 6.9 | 0.002 |

### Example 6

### Further Experiments With Tau Antigens

Experiments are conducted with an additional 95 non-modified 16-mer peptides based on Tau beyond the 55 16-mers used in Example 5, in accordance with the experimental methods used in Example 5. Further, additional modified Tau epitopes are tested, including a 16-mer Tau epitope phosphorylated at residue T18, four Tau epitopes nitrated at residues T18, T29, T197 or T394 and two Tau epitopes acetylated at residues K280 or K174. Finally, full-length wild-type Tau will be tested, as well as pre-formed fibrils.

### Example 7

### Determination of Human Leukocyte Antigen Restriction of Identified Epitopes

It is hypothesized that recognition of disease associated Tau-derived epitopes in PD patients is dependent on cofactors such as expression of specific HLA molecules that can bind the Tau-derived peptides, the presence of T cells expressing specific TCRs, and/or the presence of particular Tau haplotypes (H1 vs. H2)

HLA binding predictions are performed, along with assaying for binding with purified HLA molecules. Restriction is confirmed with HLA transfected cell lines in selected examples.

Cellular assays and validated genetic inference methods are used to determine the HLA restriction for each epitope. Proliferation of the specific T cells responding to selected epitope/HLA combination is induced by restimulation with specific epitopes, the T cell receptors that recognize those specific HLA-epitope combinations are determined.

Cellular assays and validated genetic inference methods are used to determine the HLA restriction for each epitope. First, it is determined whether responses are MHC class I or class II restricted. To accomplish this goal, epitope responsive T cells are studied for their CD4/CD8 phenotype, since a CD4 phenotype is indicative of class II presentation, while a CD8 is indicative of class I presentation. It is expected that most responses will be class II restricted CD4 T cells. If, however, CD8 class I restricted responses are detected, algorithmic protocols developed for peptides for class I are used (Paul et al., 2013; Kim et al., 2014). In this approach, each possible amino acid sequence that can be derived from the entirety of a particular protein, including pathogenic alleles, is predicted for the specific MHC alleles (Paul et al., 2013; Kim et al., 2014) expressed by a particular patient or HC. This approach has been shown to successfully predict MHC-peptide binding of>90% of the human population (Weiskopf et al., 2013). Each peptide with significant binding potential is synthesized and tested for recognition by the specific donor.

The HLA restriction of Tau epitopes is next confirmed. First, potential HLA restrictions are identified using the genetic inference Restrictor Analysis Tool for Epitope (RATE) approach, which matches likely HLA restriction for class I and class II alleles (Paul et al., 2015). Table 10 above demonstrates Tau alleles for which Tau epitope restriction has been identified. Such inferences are verified by determining which of the potential restricting alleles bind the epitopes in in vitro assays with purified HLA allelic variants (Sidney et al., 2013), and directly map restricting alleles with cell lines transfected with single HLA molecules (McKinney et al., 2013).

Methods

Donors are HLA typed by an American Society for Histocompatibility and Immunogenetics (ASHI)-accredited laboratory at the Institute for Immunology & Infectious Diseases (IIID), Murdoch University (Western Australia). HLA typing for class I (HLA A; B; C) and class II (DQA1; DQB1; DRB1 3,4,5; DPB1) is performed using locus-specific PCR amplification on genomic DNA. Primers used for amplification employ patient-specific barcoded primers. Amplified products are quantified and pooled by subject and up to 48 subjects are pooled. An unindexed (454 eight lane runs) or indexed (8 indexed MiSeq runs) library is quantified using Kappa universal QPCR library quantification kits. Sequencing is performed using either a Roche 454 FLX+ sequencer with titanium chemistry or an Illumina MiSeq using 2×300 paired end chemistry. Reads are quality-filtered and passed through a proprietary allele-calling algorithm and analysis pipeline using the latest IMGT HLA allele database as a reference. The algorithm was developed at IIID and relies on periodically updated versions of the freely available international immunogenetics information system and an ASHI-accredited HLA allele caller software pipeline, IIID HLA Analysis Suite.

Potential HLA-epitope restrictions are inferred using the RATE software program (Paul et al., 2015). Briefly, a computational method that infers HLA restriction of epitopes from T cell response data in HLA typed subjects is used. RATE infers HLA restrictions by considering the presence or absence of a response to a given epitope as the biological outcome, and calculating the relative frequency of the subjects responding to a given epitope and expressing a given allele as compared to the general test population and associated statistical significance. Binding predictions are performed using the consensus prediction method publicly available through the Immune Epitope Database (IEDB) Analysis Resource (Kim et al., 2012).

Next, classical competition assays are performed to quantitatively measure peptide-binding affinities for HLA class I and II MHC molecules, based on inhibition of binding of high affinity radiolabeled peptides to purified MHC molecules. In brief, 0.1-1 nM of radiolabeled peptide is co-incubated at room temperature or 37° C. with purified MHC in the presence of a cocktail of protease inhibitors (and, for Class I, exogenous human β2- microglobulin). Following a two to four day incubation, MHC-bound radioactivity (c.p.m.) is determined by capturing MHC-peptide complexes on plates coated with either HLA DR (L243), DQ (HB180), DP (B7/21) or Class I (W6/32) specific monoclonal antibodies. Bound c.p.m. is measured and the concentration of peptide yielding 50% inhibition of binding of the radiolabeled peptide is calculated. Under the conditions used, where [label]<[MHC] and IC50≥[MHC], measured IC50 values are reasonable approximations of true Kd. Each competitor peptide is tested at six different concentrations covering a 100,000-fold range, and in three or more independent experiments. As a positive control, the unlabeled version of the radiolabeled probe is also tested in each experiment. A threshold of 1,000 nM binding affinity is associated with immunogenicity of HLA class II T cell epitopes, and most epitopes bind in the 1-100 nm range, with affinities in the 1-10 nM considered to be of high affinity.

Results

The HLA haplotype associated with the recognition of specific Tau epitopes is identified. Whether the HLA haplotype is a risk factor for PD and/or is associated with PD disease symptoms is determined.

### Example 8

### “Megapool” Experiments

These experiments focus on the generation of a “megapool” of PD peptides, and are tested in either re-stimulation mode, or also directly ex vivo, utilizing intracellular cytokine staining (ICS) or AIM assays (Dan et al., 2016). In these experiments FACS staining for various markers is incorporated. This establishes whether the responses are mediated as excepted by CD4 responses, and which cytokines in addition to IL5 and IFNg are released by the Tau-specific T cells, thus more precisely establishing their functionality. IL10 is tested, to assess whether T cells with potential regulatory activity can be detected.

Further, epitope, epitope pool or megapool stimulations are used to confirm the results, in an independent cohort, of the main findings of higher responses in PD versus control donors, CD4 phenotype, and patterns of cytokines secretion. In addition, whole Tau stimulation is used to demonstrate recognition of natural antigen.

### Example 9

### Characterize the T Cell Receptor Repertoire Associated with Recognition of Tau Epitopes

Recognition of disease associated autoimmune epitopes in PD is dependent on expression of specific HLA molecules that bind the autoimmune peptides, and presence of T cells expressing specific T cell receptors (TCRs). During development in the thymus, each T cell generates a unique TCR stochastically by recombining segments of V, D and J genes. This experiment investigates if the increased frequency of T cells responding to specific autoimmune epitopes in PD vs. HC participants is associated with the presence of shared, ‘public’ TCRs in PD patients recognizing these epitopes.

PD patients and HC subjects that show T cell reactivity to Tau are selected. To identify TCRs recognizing Tau-derived epitopes, peripheral blood mononuclear cells (PBMC) are stimulated with peptide epitopes and culture for 14 days to expand epitope-specific cells. The T cell repertoire in the expanded culture is compared with the repertoire of input cells to determine which TCRs are specifically expanded. Peptides unrelated to PD, e.g., from common bacteria, are used as negative control stimuli.

Whether shared TCRs are present in the Tau-epitope specific T cells in the population is determined, and importantly, if these shared TCRs are present in PD but not HC samples is determined. This analysis of TCR sharing is performed both for specific TCR sequences as well as for motifs in the TCR CDR3 regions that are shared between related clonotypes with shared specificities.

Methods

During their maturation, T cells undergo a stochastic recombination process in which a unique receptor sequence is formed by recombination of genes to encode a mature TCR consisting of a TCR alpha and beta chain. The antigen-specificity of a T cell is determined by its receptor, but the phenotype of the mounted response can change over time as a T cell undergoes differentiation. The formation of memory cells and their proliferation gives rise to ‘clonotypes’ of cells that can be tracked back to a common origin. The CDR3 region of the TCR-beta chain is the most polymorphic, and is in direct contact with the epitope. Thus, sequencing this region alone is sufficient to generate a useful marker of epitope specific T cells.

A protocol to determine the antigen-specificity of T cell receptors is used, similar to other published approaches (Klinger et al., 2015), but specifically targeted to reproducibly detect relatively rare antigen-specific CD4+ T cells. Briefly, PBMC are stimulated with individual epitopes or epitope pools, driving the proliferation of epitope-specific T cells. The TCR repertoire is sequenced from the DNA of cells extracted post-expansion, and cross-compared to the TCR repertoire in PBMC ex vivo, to identify TCRs from T cells that have undergone expansion, and to the TCR repertoire in cells stimulated with different epitopes (to remove potentially unspecific T cells). To reliably detect antigen-specific cells, it is important to run culture repeats, and cross compare proliferation with different antigens, as some T cells will proliferate unspecifically in a bystander fashion. When comparing the identified TCRs based on this proliferation approach to those obtained by isolating epitope-specific T cells directly ex vivo using tetramer sorting, it is shown that the vast majority of the identified cells are epitope specific (>95% specificity). This approach is utilized to identify if there are common TCRs that are found across multiple PD donors, which would suggest that presence of specific TCRs could serve as a diagnostic. If no identical TCR sequences are found, the publicly available Glyph package (Glanville et al., 2017) is used to search for conserved motifs in the epitope specific TCRs.

Results

The TCR haplotype associated with the recognition of specific Tau epitopes is identified. Whether the TCR haplotype is a risk factor for PD and/or is associated with PD disease symptoms is determined.

### Example 10

### Determination of Whether MAPT Haplotypes are Associated with Responses to Tau-derived Epitopes

Two major haplotypes (H1 vs. H2) of MAPT have been discovered and associated with different prevalence of neurodegenerative diseases including PD. In this experiment, both PD and HC patients are genotyped to determine their MAPT haplotype. Using these data, it is determined if there is evidence that the recognition of particular epitopes in the Tau-protein is associated with particular MAPT haplotypes. This could result from different MAPT having different levels of expression for the various Tau-splice isoforms and their PTMs.

Methods

To determine whether recognition of particular epitopes derived from Tau protein is associated with MAPT haplotype, eight intragenic polymorphic markers in exons 1, 7 and 9 and introns 2, 3 and 13 are used to infer MAPT haplotypes (Ghidoni et al., 2006). Polymorphisms of exons 1 (g.75859 g>a), 7 (g.104964 g>a; P176P), 9 (g.109929, A227A; g.110013 t>c, N255N; g.110058 g>a, P270P) and of introns 2 (g.85372 c>t), 3 (g.87889 a>g) and 13 (g.137615 t>c) are analyzed by PCR amplification followed by sequencing.

Results

The MAPT haplotype associated with the recognition of specific Tau epitopes is identified. Whether the MAPT haplotype is a risk factor for PD and/or is associated with PD disease symptoms is determined.

### Example 11

### Investigation of the Immunophenotypes of Tau-Epitope Responsive T cells in PD

In this experiment, T cells that are responsive to Tau epitopes are functionally characterized. To do so, epitope-specific T cells are analyzed by flow cytometry to establish their memory phenotype using CD45RA/CCR7 staining (naive, CD45RA+CCR7+; central memory, CD45RA−CCR7+; effector memory, CD45RA−CCR7-; effector memory re-expressing CD45RA, CD45RA+CCR7−). The functional characterization utilizes ICS assays to determine the specific pattern of cytokine secretion including IFNγ, TNFα, IL-4, IL-17, IL-10, and IL-21. Additional staining for specific surface markers, such as CCR6, CCR4, CXCR3, CXCR5, PD-1, CD4OL, and CD69 determines their differentiation state and general phenotype. To increase sensitivity, the recently described Activation Induced Marker (AIM) assay (Dan et al., 2016), which was developed to allow capture of rare T cell subsets and specificities, is utilized.

As a parallel approach to characterize specific T cells, HLA multimers are designed, which use specific allele and epitope combinations to measure the presence of reactive T cells (Cecconi et al., 2010). For determined and dominant HLA allele-epitope combinations, multimers are produced, which stain and phenotype reactive cells. This technique could be used as a new means to identify preclinical PD, and by extension may by adapted for other neurodegenerative disorders that show autoimmune features.

Ex vivo analysis is used in cytokine capture assays for IL-5 and other cytokines with the AIM assay and multimer staining to characterize responses without in vitro manipulation, and to provide isolation of specific T cells for identification of micro mRNA-Seq. RNA-Seq (RNA sequencing) uses procedures that allow the study of rare antigen-specific T cells (Arlehamn et al., 2014). First, mRNA profiles of PD and HC are compared at the level of bulk memory subsets. This determines a baseline for comparison with isolated antigen-specific T cells and addresses whether a discriminatory signature is present in bulk subsets. mRNA signatures are then compared in bulk subsets with epitope-specific T cells. Distinct mRNA signatures are associated with epitope-specific responses from patient cohorts mRNA profiles from epitopespecific T cells are compared for donors to identify genes that are consistently up- or down-regulated, and associated molecular programs and functions are pinpointed by standard gene network analysis.

Methods

RNA-Seq is performed using the LJI Sequencing and Bioinformatics Core. Following collection of epitope-specific T cells during FACS sorting, RNA isolation and libraries are prepared for sequencing by a HiSeq2500 (Illumina Platform) sequencer. The sequencing data is passed to the Bioinformatics Core at LJI for analysis in their automated next generation sequencing (NGS) pipeline. Bioinformatics analysis defines genes significantly upregulated in the two subsets. Expression levels of housekeeping genes such as β2 microglobulin (B2M) are compared to ensure consistency and highly reproducible data normalization between samples.

Genes differentially regulated are identified at a P_{adj}<0.05. Pathway analysis of genes significantly upregulated in the various subsets and the associated gene modules is then performed. For this purpose, the web tool Gene Set Enrichment Analysis (GSEA) is used to determine which pathways are significantly represented (Mootha et al., 2003; Subramanian et al., 2005). GSEA is a computational method that determines whether an a priori defined set of genes shows statistically significant differences, in this case between differentially regulated gene set and the gene set represented by the full genome annotated genes. In addition, the ‘Integrated Pathway Analysis’ (IPA) software is used to determine in more detail the directionality of the overrepresented functions and the common upstream regulators for the given set of genes (Kramer et al., 2014). A complementary approach follows a modular analysis that identifies clusters of genes that share a similar expression profiles. In particular the Gene Co-expression Network Analysis (WGCNA) algorithm (Langfelder and Horvath, 2008) is used.

Results

The analysis of Tau epitopes by cytokine, AIM assays and multimer technology determines the number and phenotypes of responsive T cells in PD and yields insight into their biological roles in neurodegenerative pathogenesis. The ability to isolate epitope specific T cells also allows for the identification of their mRNA signatures.

These experiments also provide a means to quantify specific autoimmune reactive T cells in individual patients; the numbers of specific T cells are correlated with UPDRS scores to determine if this assay might provide a progression biomarker. A battery of multimers provides general screening to identify “prodromal” individuals and could lead to individualized therapies.

### Example 12

### T-Cells of Parkinson's Disease Patients Recognize α-synuclein Peptides.

Abnormal processing of self-proteins can produce epitopes presented by major histocompatibility complex (MHC) proteins to be recognized by specific T cells that escaped tolerance during thymic selection (Marrack and Kappler, 2012). Such actions by the acquired immune system are implicated in autoimmune disorders including Type-1 diabetes (T1D). While not considered to possess autoimmune features, neurodegenerative diseases are characterized by altered protein processing. The major pathological features of Parkinson's disease (PD), the most common neurodegenerative movement disorder, are the death of substantia nigra (SN) dopaminergic neurons, and the presence of intraneuronal aggregates known as Lewy bodies composed of α-synuclein (α-syn) (Spillantini et al., 1998). Activated microglia have been reported in PD SN for nearly a century (Foix and Nicolesco, 1925) and cytokine profiles implicate activation of the innate immune system (Cebrian et al., 2015). More recent evidence suggests a role for the acquired immune system (Cebrian et al., 2015), including T cell infiltration to PD SN (Brochard et al., 2009). Genome wide association studies associate PD with an immune haplotype (Wissemann et al., 2013) present in ˜15% of the general population including the MHC class II gene alleles DRB5*01 and DRB1*15:01 (Greenbaum et al., 2011), and a polymorphism in a non-coding region that may increase MHC class II expression (Hamza et al., 2010; Kannarkat et al., 2015). Antigen presentation by MHC class I expression in SN dopamine neurons was reported in adult human brain of PD patients and age matched controls., It was further demonstrated that SN dopamine neurons express MHC class I upon activation by cytokines released from microglia activated by α-syn or neuromelanin, and that CD8+ T cells kill neurons that present the appropriate combination of MHC class I and peptide (Cebrain et al., 2014). Native (Mor et al., 2003; Theodore et al., 2008) and modified (nitrated) synuclein-derived peptides (Benner et al., 2008) elicit T cell responses in rats and mice, and Standaert and coworkers recently demonstrated that SN neuronal death in a α-syn overexpression model is absent in MHC II null mice (Harms et al., 2013).

To address if PD is associated with T cell recognition of epitopes derived from α-syn presented by specific MHC alleles, 67 PD participants and 36 age-matched non-PD healthy controls (HC) were recruited. Participants were 46-83 years of age (PD, median 66, range 46-83; HC, median 64, range 52-83) and 66% were male (PD 75%; HC 50%) (Tables 11a, 11b, and 12). While ˜15% of HC carried DRB1*15:01/DRB5*01:01 alleles, ˜⅓ of PD carried these alleles (difference between PD and HC, p=0.036 and 0.022 for DRB1*15:01/DRB5*01:01), indicating association of HLA DR allelic variants with PD in our cohort (Table 13).

TABLE 11a
Demographics of study participants

| | Parkinson's cases | | Controls | | |
|---|---|---|---|---|---|
| | (n = 67) | | (n = 36) | | p-value |
| Mean age in years, | 65.2 | (8.6) | 64.2 | (7.4) | 0.533 |
| (SD) | | | | | |
| Male, % (n) | 73.1 | (49) | 50.0 | (18) | 0.029 |
| Recruitment site, % | 86.6 | (58) | 63.9 | (23) | 0.011 |
| Columbia University | | | | | |
| (n) | | | | | |
| Subjects with family | 19.4 | (13) | 8.3 | (3) | 0.165 |
| history of Parkinson's | | | | | |
| disease in first- | | | | | |
| degree relative, % (n) | | | | | |
| Mean Parkinson's age- | 58.2 | (10.1) | N/A | | N/A |
| at-onset, (SD) | | | | | |
| Caucasian, % (n) | 89.6 | (60) | 86.1 | (31) | 0.599 |

TABLE 11b
Demographics of study participants with Unified
Parkinson's disease rating scale (UPDRS) scores

| | Parkinson's cases | | Controls | | |
|---|---|---|---|---|---|
| | (n = 58) | | (n = 23) | | p-value |
| Mean age in years, | 65.5 | (8.2) | 64.9 | (8.3) | 0.754 |
| (SD) | | | | | |
| Male, % (n) | 75.9 | (44) | 47.8 | (11) | 0.5539 |
| Recruitment site, % | 100 | (58) | 100 | (23) | |
| Columbia University | | | | | |
| (n) | | | | | |
| Subjects with family | 0.21 | (12) | 0.043 | (1) | 0.3714 |
| history of Parkinson's | | | | | |
| disease in first- | | | | | |
| degree relative, % (n) | | | | | |
| Mean Parkinson's age- | 58 | (9.44) | N/A | | N/A |
| at-onset, (SD) | | | | | |
| Mean UPDRS part III, | 16.87 | (9.49) | 1.48 | (2.06) | <0.0001 |

| | | |
|---|---|---|
| (SD) | (range 5-46) | (range 0-6) |

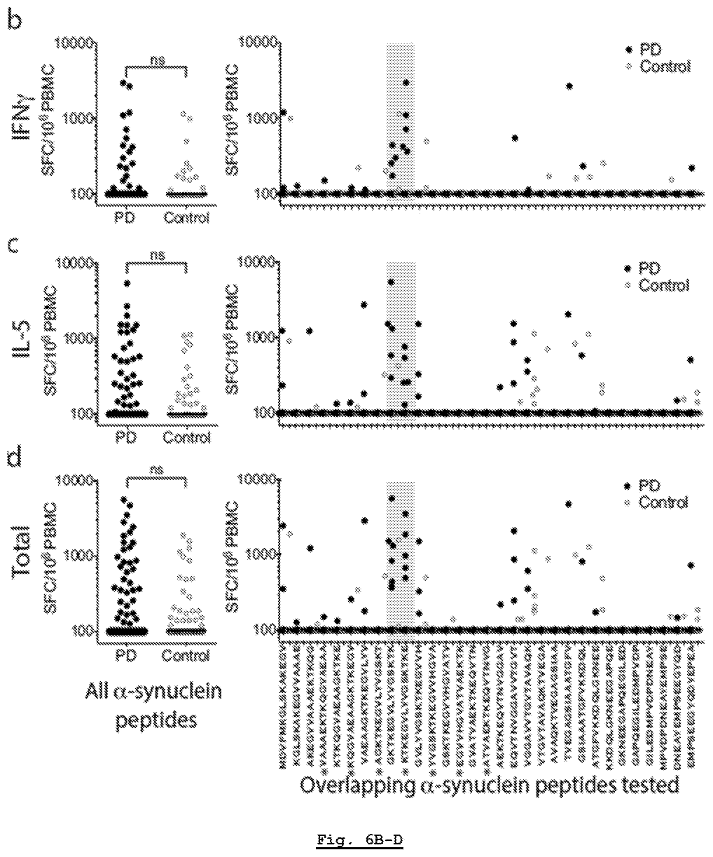

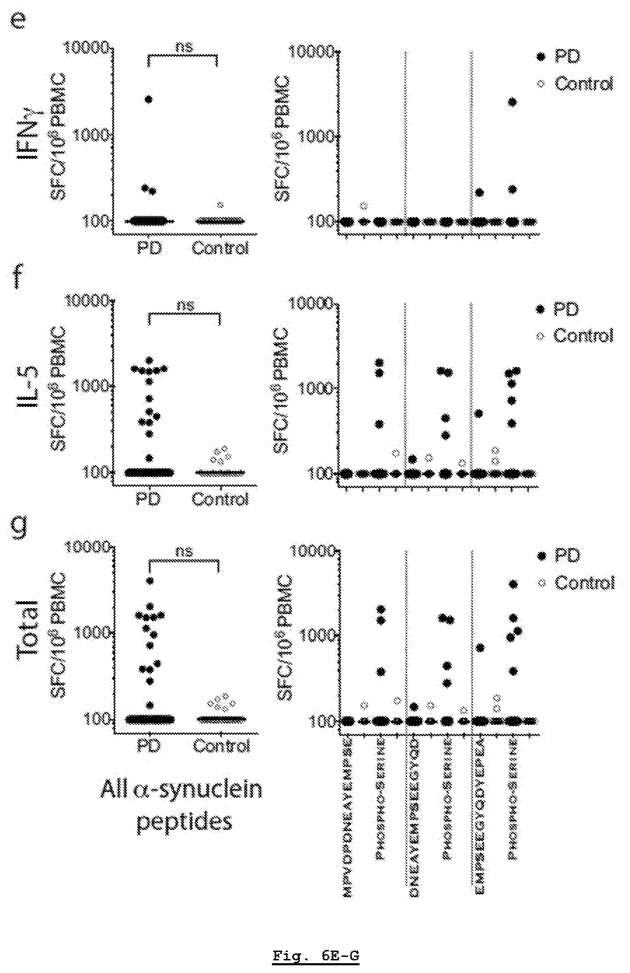

TABLE 12
Demographics of additional study participants
in Supplemental FIG. 8a.

| | Parkinson's cases | |
|---|---|---|
| | (n = 8) | |
| Mean age in years, | 65.5 | (5.4) |
| (SD) | | |
| Male, % (n) | 62.5% | (5) |
| Recruitment site, % | 100% | (8) |
| Columbia University | | |
| (n) | | |
| Subjects with family | 37.5% | (3) |
| history of Parkinson's | | |
| disease in first- | | |
| degree relative, % (n) | | |
| Mean Parkinson's age- | 55.4 | (5.2) |
| at-onset, (SD) | | |
| Mean UPDRS part III, | 17.5 | (7) |
| (SD) | | |

| | |
|---|---|
| Median UPDRS part III | 19 |
| Range UPDRS part III | Min: 7; Max: 28 |

**Note:
total n = 12; demographics collected only for n = 8.

TABLE 13
HLA association of subjects.

| | DRB1*15:01 | | DRB5*01:01 | |
|---|---|---|---|---|
| HLA | individuals | individuals | individuals | individuals |
| Allele | with allele | without allele | with allele | without allele |
| PD | 23 | 44 | 24 | 43 |
| HC | 5 | 31 | 5 | 31 |

| | | |
|---|---|---|
| Fisher's | 0.036 | 0.022 |
| exact two- | | |
| tailed p- | | |
| value | | |

To determine whether α-syn derived peptides were recognized by T cells, responses were assayed to pools that each contained ˜twenty 9-10aa peptides predicted to bind common HLA class I types 15, and 15aa peptides spanning the protein that could elicit HLA class II responses. PBMCs from PD and HC were stimulated for 14 days, and IFNγ and IL-5 responses were measured by dual color ELISPOT, enabling quantification of responsive cells. Positive pools were deconvoluted to identify the peptides eliciting cytokine responses. IFNγ was used as a representative cytokine to detect CD8+/HLA class I and CD4+ Th1/Class II T cells, and IL-5 as a representative cytokine secreted by CD4+ Th2/Class II T cells. Each pool was tested in an initial cohort in 19-25 randomly selected PD and 12 HC. The majority of PBMC responses to the 15aa peptides produced IL-5 (68% of total), indicating a prominent CD4+ Th2 phenotype, and the remainder of the responses were to IFNγ (32%). No cells producing both IL-5 and IFNγ were detected.

Two antigenic regions were identified in α-syn, the first near the N terminus, composed of aa31GKTKEGVLYVGSKTK aa45 and aa32KTKEGVLYVGSKTKE aa46 (referred to as the Y39 region) (FIG. 1a), which elicited an apparent Class II restricted IL-5 and IFNγ response (FIG. 1b-d). Residue aa32 is a plasmin cleavage site 16 and chymotrypsin cleavage sites are at aa31/32 and aa45/46 17.

The second antigenic region was near the C terminus (aa116-140) (referred to as the S129 region) (FIG. 6a), and required phosphorylation of amino acid residue 5129. The three phosphorylated aaS129 epitopes (aall6MPVDPDNEAYEMPSEaa130, aa121DNEAYEMPSEEGYQDaa135, aa126EMPSEEGYQDYEPEAaa140) produced markedly higher IL-5 responses in PD than HC (p=0.02, Fisher's exact test, 300 SFC threshold) (FIG. 6e-g). Phosphorylated aaS129 residues are present at high levels in PD Lewy bodies (Fukiwara et al., 2002), and PD Lewy bodies contain α-syn fragments with cleavage sites at approximately aa115, 119, 133, and 135 (Anderson et al., 2006), and include the fragment aa129SEEGYQDYEPEAaa140, which is contained within one of the aaS129 epitopes. Caspase-1 (Wang et al., 2016) and neurosyn (Kasai et al., 2008) can cleave α-syn at aa121, chymotrypsin and cathepsin can cleave at aa116, aa125/126, and aa135/136 (Hossain et al., 2001), proteasome may cleave at aa119/120 (Li et al., 2005), and calpain can cleave at aa122, with resulting fragments identified in PD brain (Dufty et al., 2007).

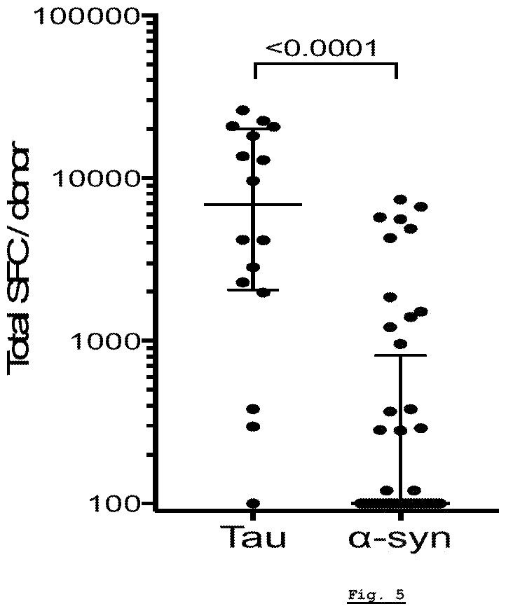

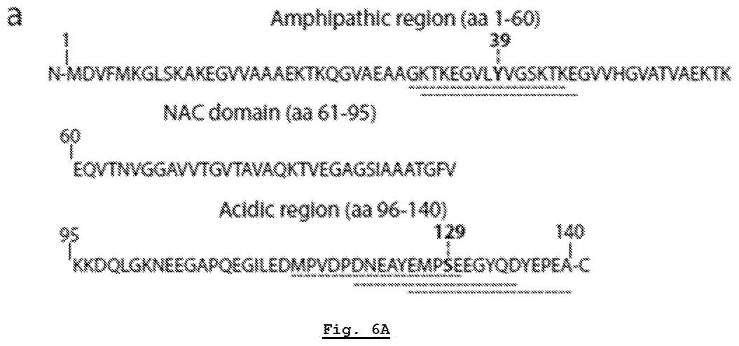

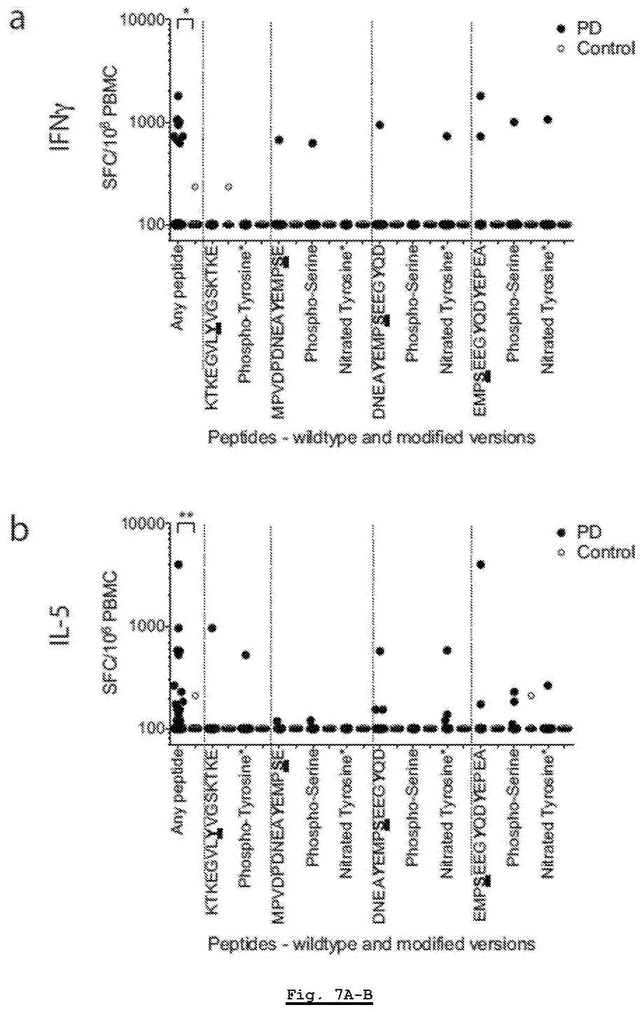

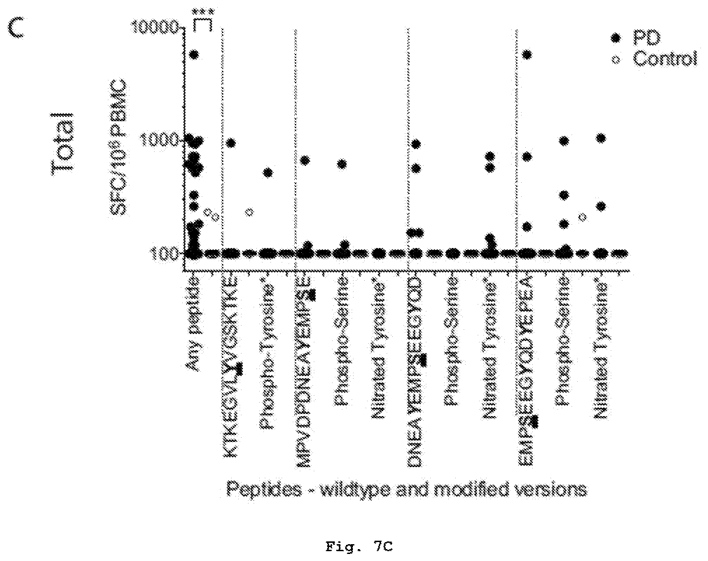

The immune responses to aa39 and aa129 region epitopes, including a second cohort of 19 PD and 12 HC assayed for response to additional phosphorylated and nitrated modifications (FIG. 7), were different between PD and HC for secretion of both IFNγ (two-tailed Mann-Whitney test, p<0.05) and IL-5 (two-tailed Mann-Whitney test, p<0.001), and combined responses (two-tailed Mann-Whitney test, p<0.001) (FIG. 8a-c). While residue aa39 is highly phosphorylated in PD patients 24, Y39 phosphorylation was not required for antigenic response. The response was primarily polarized towards IL-5 in PD (71% IL-5 and 29% IFNγ; FIG. 8d). This polarization was PD specific, and the relatively rare HC responses were not similarly polarized (46% IL-5 and 54% IFNγ). To identify specific sets of T cells that respond to α-syn epitopes, we measured response to a pool of the 11 α-syn antigenic peptides by 9 PD participants (FIG. 9). Approximately 0.2% of CD3+ T cells responded to the α-syn peptides. Of the responsive T cells, ˜50% produced IL-4 and 50% produced IFNγ, with no detectable IL-10 or IL-17 production. In most cases, responses were mediated by CD4+ T cells, but response by one PD was mostly mediated by IFNγ-producing CD8+ T cells. Thus, T cell response to α-syn antigenic peptides was largely mediated by IL-4 or IFNγ-producing CD4+ T cells, with potential contributions from CD8+/IFNγ producing T cells.

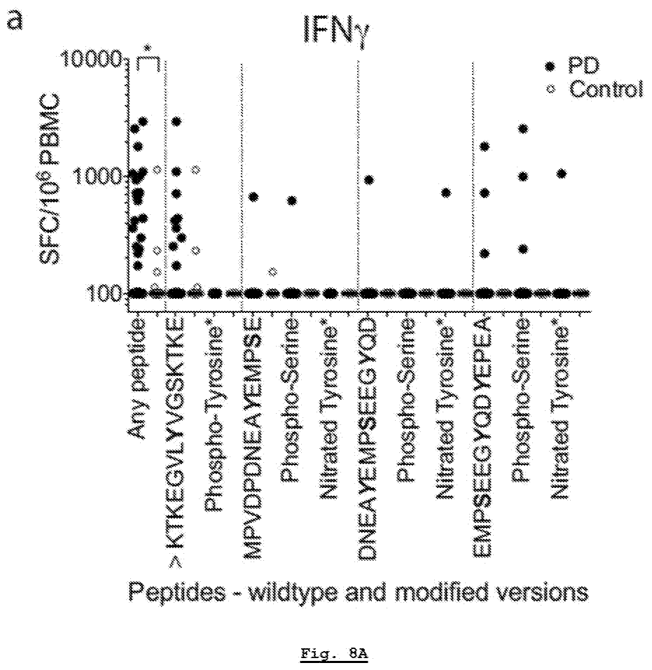

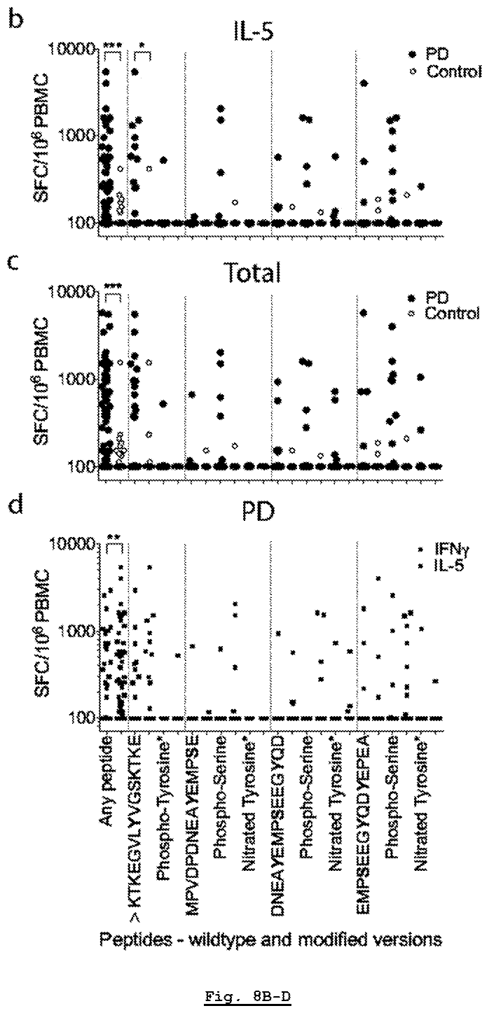

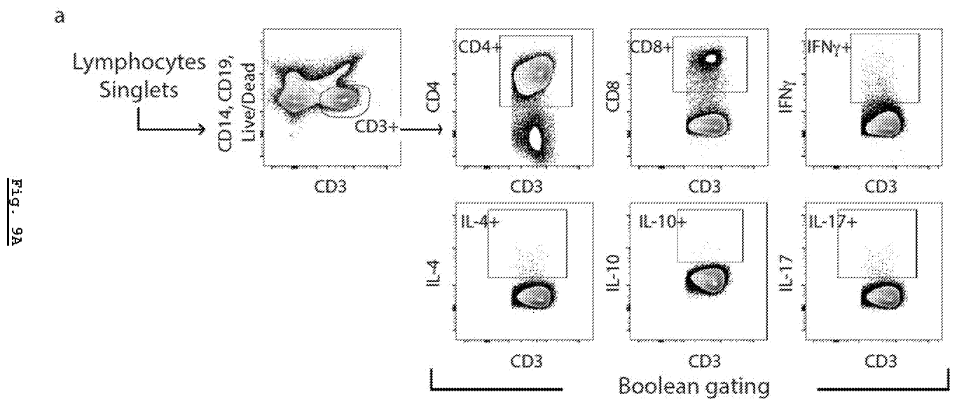

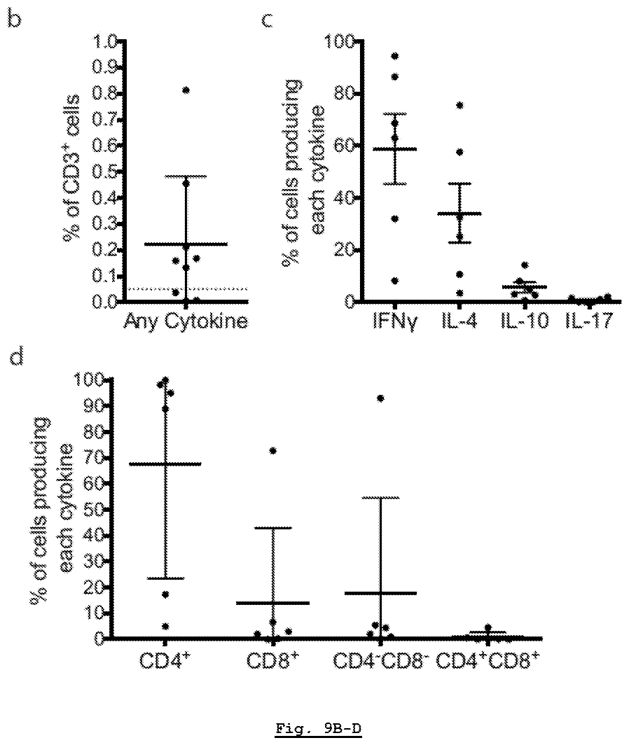

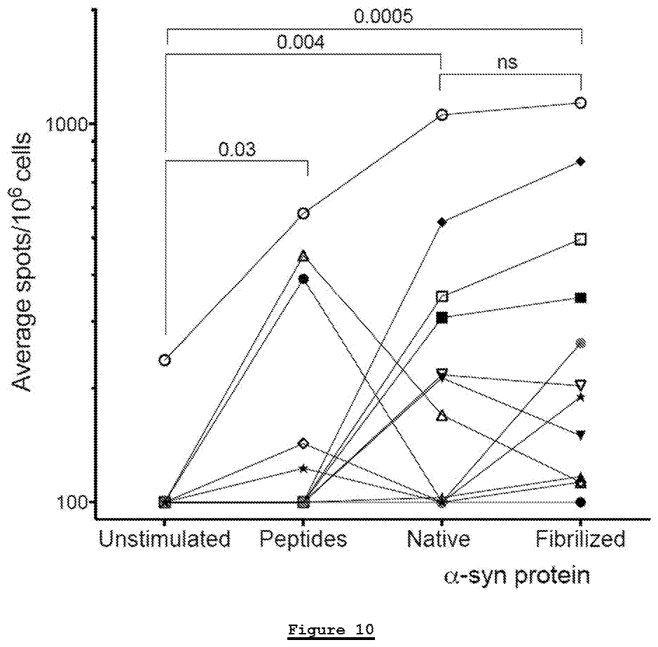

To test if the α-syn epitopes arise from processing of native and/or fibrilized α-syn, PBMCs were stimulated with α-syn epitopes for 14 days. The cultures were then assayed with α-syn peptides, 25 μg/ml fibrilized (PFF) α-syn, 25 μg/ml native α-syn, or media alone. FIG. 10 shows that T cells lines specific for the α-syn epitopes were activated by antigen presenting cells pulsed with native or PFF protein in 7/12 and 11/12 cases. There was significantly higher response to native α-syn (p=0.004) and PFF α-syn (p=0.0005) than media alone. Thus, T cells can respond to α-syn epitopes arising from natural processing of extracellular native α-syn, which is present in blood, and the fibrilized α-syn associated with PD.

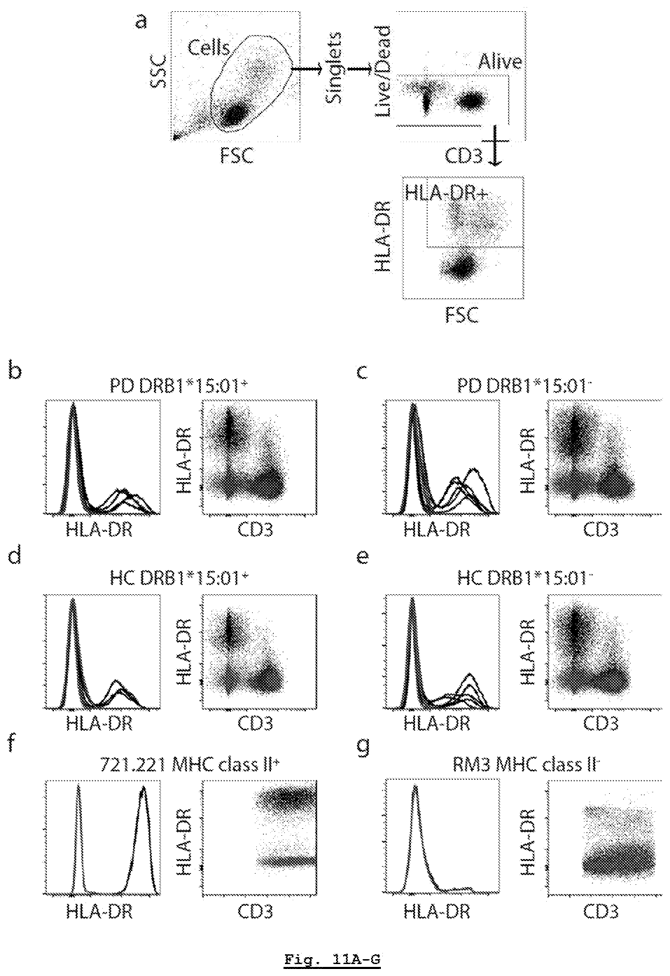

We then identified the HLA alleles that present α-syn peptides by in vitro binding to a panel of HLAs representing the common alleles expressed in worldwide populations 1. A threshold of 1,000 nM binding affinity is associated with immunogenicity of HLA class II T cell epitopes, and most epitopes bind in the 1-100 nm range, with affinities in the 1-10 nM considered to be of high affinity. Of 26 common HLA class II alleles tested, five bound to aa32KTKEGVLYVGSKTKEaa46 (Table 5). The HLA class II variants DRB1*15:01 and DRB5*01:01 bound the epitope with high affinity (2.8 nM and 8.1 nM, respectively), while DRB1*07:01, B1*09:01 and DQB1*03:01 bound in the 80-250 nM range. The aa32KTKEGVLYVGSKTKEaa46 epitope phosphorylated at Y39 also bound DRB1*15:01 and DRB5*01:01 with high affinity. Comparison of PD with and without DRB1*15:01 alleles found no difference in levels of HLA class I or class II protein expression (FIGS. 11 & 12). Thus, epitopes in the Y39 region of α-syn strongly bind two HLA class II β chain alleles associated with PD.

In contrast, the C terminus peptides spanning 5129 and its post-translational forms bound HLA class II alleles weakly, with the exception of aa121DNEAYEMPSEEGYQDaa135, which in both native and phosphorylated S129 forms strongly bound DQB1*05:01. The aall6MPVDPDNEAYEMPSEaa130 epitope bound several alleles with lower affinity, and the aa126EMPSEEGYQDYEPEAaa140 epitope bound DQB1*04:02 and DQB1*05:01 with low affinity. Thus, antigenic peptides in the C terminus 5129 antigenic region demonstrated relatively little clear restriction, suggesting that they are recognized promiscuously.

DRB1*15:01 and DRB5*01:01 alleles are in linkage disequilibrium, and participants expressing one allele likely express both. Of PD participants, 8/13 responders to the aa32KTKEGVLYVGSKTKEaa46 epitope expressed both DRB1*15:01 and DRB5*01:01, while only 12/45 (DRB1*15:01) and 13/43 (DRB5*01:01) non-responders expressed the alleles, indicating association between the alleles and antigenic response (odd ratios of 4.4 and 3.7, p values of 0.04 and 0.05, respectively) (Table 14).

TABLE 14
HLA association of Y39 responses

| | | Individuals with allele | | Individuals lacking allele | | | | |
|---|---|---|---|---|---|---|---|---|
| | | | Neg. | | Neg. | | | |
| | | Pos. epitope | epitope | Pos. epitope | epitope | Rel. | Odds | |
| | HLA Allele | response | response | response | response | freq. | ratio | p-value |
| PD | DRB1*15:01 | 8 | 12 | 5 | 33 | 1.8 | 4.4 | 0.04 |
| | DQB1*03:04 | 2 | 0 | 11 | 45 | 4.5 | inf. | 0.05 |
| | DRB5*01:01 | 8 | 13 | 5 | 30 | 1.6 | 3.7 | 0.05 |
| | DRB3*02:02 | 1 | 19 | 12 | 24 | 0.2 | 0.1 | 0.021 |
| | A*11:01 | 8 | 9 | 5 | 36 | 2.1 | 6.4 | 0.012 |
| HC | DRB1*15:01/DQB1*03:04/ | 13 | 18 | 0 | 27 | 1.9 | inf. | 0.00007 |
| | DRB5*01:01/A*11:01 | | | | | | | |
| | DRB1*15:01/DQB1*03:04/ | 3 | 5 | 0 | 26 | 4.3 | inf. | 0.009 |
| | DRB5*01:01/A*11:01 | | | | | | | |

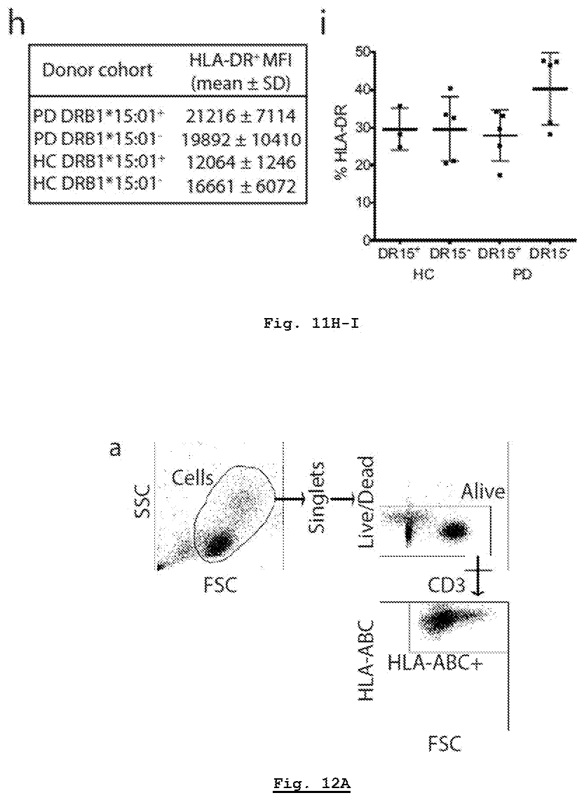

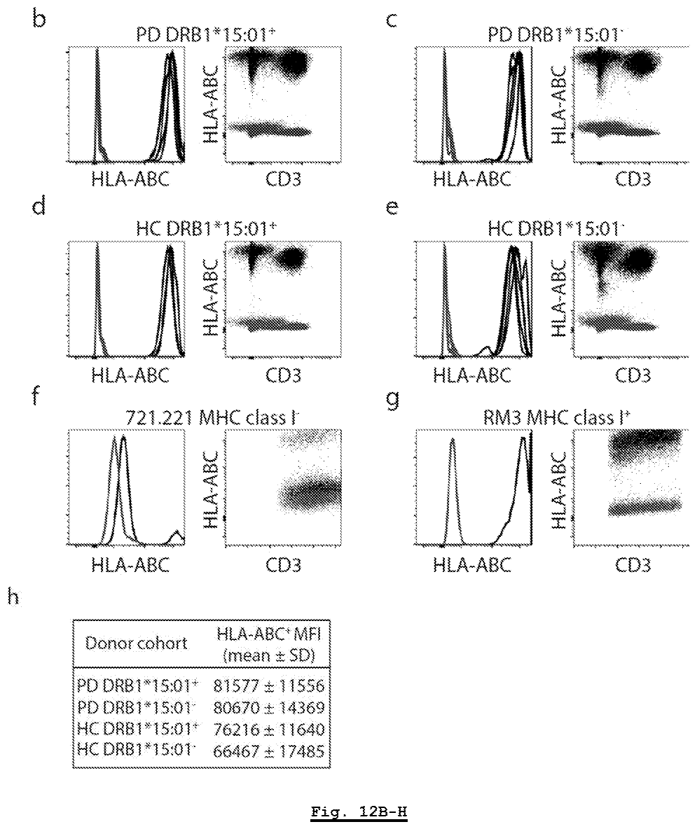

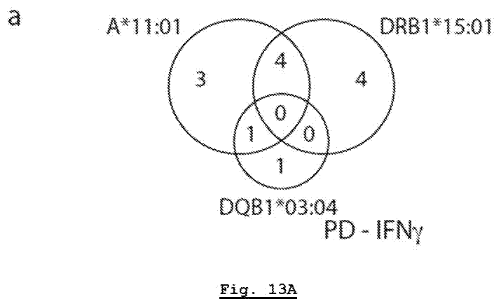

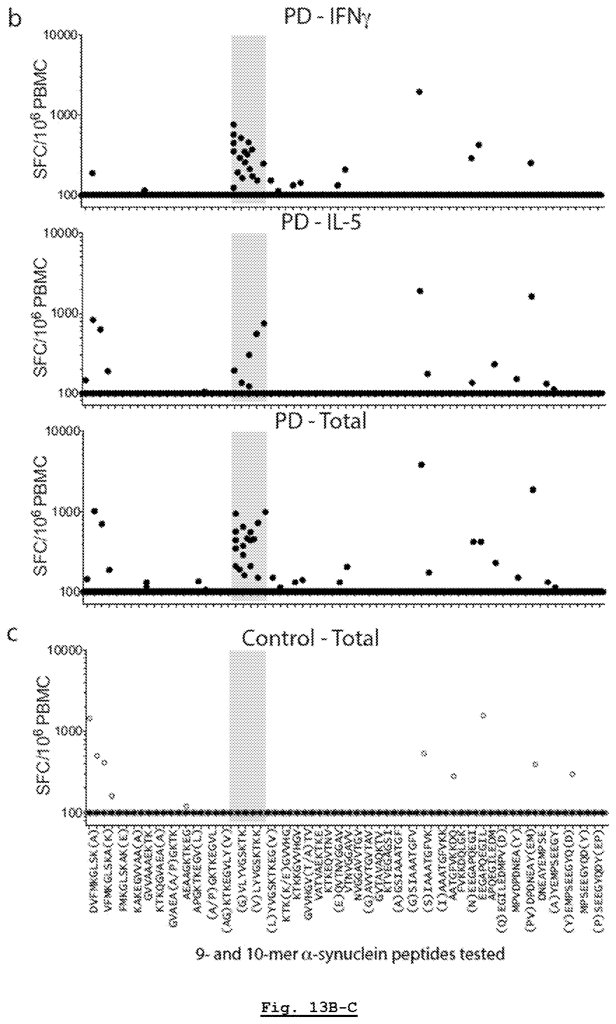

This analysis detected additional associations, with 2/13 responders expressing DQB1*03:04 (p=0.05) compared to 0/45 non-responders, as well as the HLA class I allele A*11:01, with 8/13 responders expressing A*11:01 compared to 9/45 non-responders (p=0.012). While A*11:01 is in relatively mild linkage disequilibrium with DRB1*15:01 and DRB1*01:01, the associations were largely independent (FIG. 13a). In general, PD participants showed a trend towards higher expression of HLA molecules, particularly HLA class II. This is consistent with an inflammatory component of PD, and higher HLA class II expression and induction in PBMCs of PD vs. HC 3. Little or no difference in HLA class II expression was found between participants expressing DRB1*15:01 vs. other DRB1 alleles (FIG. 11). A similar but still less pronounced trend was noted for HLA class I (FIG. 12). This suggests that the association between DRB1*15:01 and PD is not based on differential expression of the protein. We detected negative association between recognition of aa32KTKEGVLYVGSKTKEaa46 and the DRB3*02:02 allele, suggesting this allele might be protective. The four alleles DRB1*15:01, DRB5*01:01, DQB1*03:04 and A*11:01 accounted for every single individual responding to the aa39 epitope (p=0.00007 for PD, Table 14). This association was far more significant in PD than HC (p=0.009). The combined association for the four alleles for PD vs. HC was significant (p=0.008 two-tailed Fisher's exact test compared to individual DRB1*15:01, p=0.05, and DRB5*01:01, p=0.03), with ˜ half of the PD (31 with alleles and 27 without) carrying one of the four alleles, whereas only ˜20% of the HC (8 with alleles and 26 without) expressed one of the four (Table 14).

Following detection of association of response to the Y39 region with the MHC class I allele HLA A*11:01, PD responses to shorter α-syn derived peptide candidates were evaluated for class I presentation. It was found that 5/19 PD responded to these short peptides while 0/12 HC responded (FIG. 13B & 13C) (two-tailed Chi square=3.765, 1df, p=0.0523). Reactivity occurred mostly on peptides contained within the Y39 region, involving three peptides (aa36GVLYVGSKTKaa45, aa37VLYVGSKTKaa45, aa37VLYVGSKTKKaa46) predicted as potential A*11:01 binders 15. Each peptide was tested for binding to purified HLA A*11:01 molecules in vitro, and found that the 9 mer aa37VLYVGSKTKaa45, which is nested within the two 10 mers, bound with good affinity (IC50=161 nM), while the other two bound poorly, indicating that the 9 mer is responsible for T cell recognition. Reactivity to short peptides was mostly mediated by IFNγ producing cells and most pronounced for the A11 binding peptides. Thus, immune responses to α-syn associated with PD have both MHC class I and II restricted components.

Discussion

Genetic studies associate Parkinson's disease with alleles of the major histocompatibility complex (Greenbaum et al., 2011; Hamza et al., 2010; Kannarkat et al., 2015). A defined set of peptides derived from α-synuclein, a protein aggregated in Parkinson's disease4, was found to act as antigenic epitopes displayed by these alleles and drive helper and cytotoxic T cell responses in Parkinson's disease patients. Without wishing to be bound by any scientific theory, these responses may explain the association of Parkinson's disease with alleles of the acquired immune system.

Alleles of over twenty genes are associated with familial PD (Hernandez et al., 2016), many of which encode proteins implicated in lysosomal degradation pathways including mitochondrial turnover. For example, mutations in α-syn or dopamine-modified α-syn (Martinex-Vincente et al., 2008; Cuervo et al., 2004), and LRRK2 (Orenstein et al.; 2013) interfere with protein degradation by chaperone-mediated autophagy, a process that becomes less efficient with age. Extracellular oligomeric α-syn may be acquired by brain cells during PD pathogenesis (Luk et al., 2012). These reports suggest that altered degradation of proteins including α-syn could produce antigenic epitopes that trigger immune reactions during aging and PD.

The results herein indicate that peptides derived from two regions of α-syn produce immune response in PD patients; their roles in additional synucleinopathies are untested. Epitopes derived from the Y39 region (˜aa31/32 to 45/46) are specifically displayed by two MHC class II beta chain alleles, DRB5*01:01 and DRB1*15:01, associated with PD, as well as an additional MHC class II allele and an MHC class I allele not previously associated with PD. This response is enacted mostly by IL-5 secreting CD4+ T cells, as well as IFN□ CD8+ cytotoxic T cells. α-Syn is not to our knowledge endogenously expressed by cells that express MHC class II, but is in CSF (Atik et al., 2016), from where it can be acquired by MHC class II expressing cells. This situation is analogous to the experimental autoimmune encephalitis model of multiple sclerosis, as myelin proteins used to produce autoimmunity are not endogenous to MHC class II expressing cells, but are accumulated and processed for MHC class II display by antigen presenting cells and microglia. The Y39 antigenic region is strikingly close to the α-syn mutations that cause PD (A30P, E46K, H50Q, G51D, A53T) (Hernandez et al., 2016). The second antigenic region encompasses S129 and requires S129 phosphorylation, a form present in Lewy bodies (Fujiwara et al., 2002): antigenic epitopes from that region are not strongly restricted and can drive immune responses in PD patients who do not express HLA alleles that recognize the Y39 region.

Approximately 40% of the PD participants in our cohort exhibited immune responses to α-syn epitopes, and these responses may reflect variations in disease progression or environmental factors. The fraction of patients who display such responses in classic autoimmune disorders such as T1D, rheumatoid arthritis and multiple sclerosis is often ˜20-50% (Petrick de Marquesini et al., 2010). Without wishing to be bound by any scientific theory, as with T1D, which features epitopes derived from both preproinsulin and additional proteins, it may be that PD-related epitopes are derived from α-syn and additional proteins. In classic autoimmune disorders, MHC class II response may precede MHC class I (Marrack et al., 2012), and it is noted that exposing microglia to α-syn triggers MHC class I expression by dopamine neurons (Cebrain et al., 2014). Without wishing to be bound by any scientific theory, the PD-associated proteins parkin and PINK1 may regulate antigenic presentation of mitochondrial peptides (Matheoud et al., 2016), and it is possible that an autoimmune presentation of antigenic epitopes unites lysosomal and mitochondrial mechanisms of PD pathogenesis.

### Example 13

### Materials and Methods for Study of T-Cell Reactivity to Epitopes in Parkinson's Disease Patients

A) Study Subjects

All participants provided written informed consent for participation in the study. Ethical approval was obtained from the LJI and Columbia University institutional review boards. 67 participants with PD and 36 age-matched healthy controls (HCs) were recruited from the greater San Diego (PD, n=9; HC, n=13) and New York City (PD, n=58; HC, n=23) areas. The New York cohort was recruited from the Center for Parkinson's Disease at Columbia University Medical Center through the Spot study 34. PD was defined based on the United Kingdom Parkinson's Disease Brain Bank criteria, without excluding cases with a family history of PD 35. Demographics and disease characteristics were collected including age, age of onset, sex, medications, comorbidities and motor disease severity as measured by the Unified Parkinson's Disease Rating Scale (UPDRS) motor score (UPDRS-III). Also, family history of PD in first-degree relatives was collected. The data are reported in Tables 11a & 11b. In the San Diego cohort, demographic data was collected and PD was self-reported.

Samples used for additional assays in FIG. 13 and FIG. 10 were collected from consecutive individuals based on the schedule of their appointment: the demographics and PD characteristics of these participants are displayed in Tables 12 and 13. HCs were recruited through a convenience sample of consecutive non-blood related individuals, and were mostly spouses of PD participants. At Columbia University, PD and HC were recruited only if there was no history of immune modulatory medications (e.g., steroids) or overt autoimmune disorder (e.g., lupus). No significant difference was detected in response rates as a function of sex or geographical location. Three participants with PD had a history of Crohn's disease and one patient had a history of Hashimoto's thyroiditis. Two of the three participants with Crohn's disease showed antigenic response to α-syn and the participant with Hashimoto's thyroiditis did not. Experimental blinding was accomplished by labeling the blood samples in a coded fashion without information on age/gender or PD status. The cohort was predominantly Caucasian (88.3%) and no firm conclusions between Crohn's (Harms et al., 2013) disease and PD could be drawn because of the limited number of Crohn's disease patients studied.

B) Peptides

Peptides were synthesized as crude material on a small (1 mg) scale by A and A (San Diego, Calif.). Peptides were 40 15 mers overlapping by 10-14 residues and 70 9- or 10 mers predicted to bind common HLA-class I alleles. Briefly, each possible 9- and 10 mer from α-syn were scored for their capacity to bind a panel of 27 common HLA class I A and B molecules (Paul et al., 2013). For each allele 4 peptides were synthesized (two 9 mers and two 10 mers, n=61 after removing redundant sequences that were selected for 2 or more alleles). In addition, any peptide that scored at the 2 percentile level or better for predicted binding, but were not within the 4 selected per allele were synthesized (n=9). Posttranslationally modified peptides (n=7) were synthesized as purified material (>95% by reversed phase HPLC) by A and A (San Diego). Peptides were combined into pools of 14 peptides (range 11-16).

An alternative mode of stimulation would be to use whole α-syn, but it was opted for synthetic peptides due to their well-characterized and uniform chemical species, in contrast to α-syn preparations that contain varying amounts of different post-translational modifications, and as it is unclear which form(s) are processed by APCs during PD. In addition to a lower cost, synthetic peptides better provide mapping of specific epitopes and measurement of HLA binding.

C) PBMC Isolation and Culture

Venous blood was collected in heparin-containing blood bags or tubes. Peripheral blood mononuclear cells (PBMC) were purified from whole blood by density-gradient centrifugation, according to the manufacturer's instructions. Cells were cryopreserved in liquid nitrogen suspended in FBS containing 10% (vol/vol) DMSO. Culturing of PBMCs for in vitro expansion was performed by incubating in RPMI (Omega Scientific) supplemented with 5% human AB serum (Gemini Bioscience), 15 GlutaMAX (Gibco), and penicillin/streptomycin (Omega Scientific) at 2×106 per mL in the presence of individual peptide pools at 5 μg/ml. Every 3 days, 10 U/ml IL-2 in media were added to the cultures.

D) ELISPOT Assays

After 14 days of culture with individual peptide pools (5 μg/ml), the response to pools and individual peptides (5 μg/ml) was measured by IFNγ and IL-5 dual ELISPOT 37. ELISPOT antibodies, mouse anti-human IFNγ (clone 1-D1K), mouse anti-human IL-5 (clone TRFK5), mouse anti-human IFNγ-HRP (clone 7-B6-1), mouse anti-human IL-5 biotinylated (clone 5A10) were all from Mabtech. To be considered positive, a response had to match three criteria: 1) elicit at least 100 spot-forming cells (SFC) per 106 PBMC, 2) p≤0.05 by Student' s t-test or by a Poisson distribution test, 3) stimulation index ≥2.

For the experiments with fibrilized or native α-syn, PBMCs were stimulated with epitopes derived from α-syn for 14 days. These cultures were then stimulated with α-syn peptides, 25 μg/ml fibrilized α-syn or 25 μg/ml native α-syn.

E) HLA Typing, Restriction, Binding Predictions and Assays

Participants were HLA typed at the La Jolla Institute or by an ASHI-accredited laboratory at Murdoch University (Western Australia). Typing at LJI was performed by next generation sequencing 38. Specifically, amplicons were generated from the appropriate class II locus for exons 2 through 4 by PCR amplification. From these amplicons, sequencing libraries were generated (Illumina Nextera XT) and sequenced with MiSeq Reagent Kit v3 as per manufacturer instructions (Illumina, San Diego, Calif.).

Sequence reads were matched to HLA alleles and participant genotyping assigned. HLA typing in Australia for Class I (HLA A; B; C) and Class II (DQAl; DQB1, DRB1 3,4,5; DPB1) was performed using locus-specific PCR amplification on genomic DNA. Primers used for amplification employed patientspecific barcoded primers. Amplified products were quantitated and pooled by subject and up to 48 subjects were pooled. An unindexed (454 8-lane runs) or indexed (8 indexed MiSeq runs) library was then quantitated using Kappa universal QPCR library quantification kits. Sequencing was performed using either a Roche 454 FLX+sequencer with titanium chemistry or an Illumina MiSeq using 2×300 paired-end chemistry. Reads were quality-filtered and passed through a proprietary allele calling algorithm and analysis pipeline using the latest IMGT HLA allele database as a reference.

The algorithm was developed by co-authors EP and SM and relies on periodically updated versions of the freely available international immunogenetics information system (http://www.imgt.org) and an ASHI-accredited HLA allele caller software pipeline, IIID HLA Analysis Suite (www.iiid.com.au/laboratory-testing/).

Potential HLA-epitope restrictions were inferred using the RATE program (Paul et al., 2015). HLA A*11:01 binding predictions were performed using the consensus prediction method publicly available through the IEDB Analysis Resource (available at www.iedb.org) (Vita et al., 2015).

Classical competition assays to quantitatively measure peptide binding affinities for HLA class I and II MHC molecules, based on inhibition of binding of high affinity radiolabeled peptides to purified MHC molecules, were performed as detailed elsewhere 40. Briefly, 0.1-1 nM of radiolabeled peptide was co-incubated at room temperature or 37° C. with purified MHC in the presence of a cocktail of protease inhibitors (and, for class I, exogenous human β2-microglobulin). Following a two to four day incubation, MHC bound radioactivity (cpm) was determined by capturing MHC/peptide complexes on Lumitrac 600 plates (Greiner Bio-one, Frickenhausen, Germany) coated with either HLA DR (L243), DQ (HB180), DP (B7/21) or class I (W6/32) specific monoclonal antibodies. Bound cpm was measured using the TopCount microscintillation counter (Packard Instrument Co., Meriden, CT). The concentration of peptide yielding 50% inhibition of binding of the radiolabeled peptide was calculated. Under the conditions utilized, where [label]<[MHC] and IC50≥[MHC], measured IC50 values are reasonable approximations of true Kd (Cheng and Prusoff, 1973; Gulukota et al., 1997). Each competitor peptide was tested at six different concentrations covering a 100,000-fold range, and in three or more independent experiments. As a positive control, the unlabeled version of the radiolabeled probe was also tested in each experiment.

A threshold of 1,000 nM binding affinity is associated with immunogenicity of HLA class II T cell epitopes, and most epitopes bind in the 1-100 nm range, with affinities in the 1-10 nM considered to be of high affinity *Sidney et al., 2010).

F) Intracellular Cytokine Staining

After 14 days of culture PBMC were stimulated in the presence of 5 μg/ml α-syn peptide pool for 2 h in complete RPMI medium at 37° C. with 5% CO2. After 2 h, 2.5 μg/ml each of BFA and monensin was added for an additional 4 h at 37° C. Unstimulated PBMCs were used to assess nonspecific/background cytokine production and PHA stimulation at 5 μg/ml was used as a positive control. After a total of 6 h, cells were harvested and stained for cell surface antigens CD4 (anti-CD4-APCEf780, RPA-T4, eBioscience), CD3 (anti-CD3-AF700, UCHT1, BD Pharmingen), CD8 (anti-CD8-BV650, RPA-T8, BioLegend), CD14 (anti-CD14-V500, M5E2, BD Pharmingen), CD19 (anti-CD19-V500, HIB19, BD Pharmingen), and fixable viability dye eFluor 506 (eBioscience). After washing, cells were fixed using 4% paraformaldehyde and permeabilized using saponin buffer. Cells were stained for IFNγ (anti-IFNγ-APC, 4S.B3, eBioscience), IL-17 (anti-IL-17-PECy7, eBio64DEC17, eBioscience), IL-4 (anti-IL-4-PE/Dazzle594, MP4-25D2, BioLegend), and IL-10 (anti-IL-10-AF488, JES3-9D7, eBioscience) in saponin buffer containing 10% FBS. Samples were acquired on a BD LSR II flow cytometer.

Frequencies of CD3+ T cells responding to α-syn peptide pool were quantified by determining the total number of gated CD3+ and cytokine+ cells and background values subtracted (as determined from the medium alone control) using FlowJo X Software (FlowJo, Ashland, Oreg.). Combinations of cytokine producing cells were determined using Boolean gating.

G) HLA-DR and -ABC Expression

PBMCs from DRB1*15:01+ or DRB1*15:01− PD (n=5 for both) and HC (n=3 DRB1*15:01+ and n=5 DRB1*15:01−) were assessed for HLA-DR and HLA-ABC (as a control) expression. 721.221 and RM3 cells (both sourced from ATCC, mycoplasma free) were used as controls for HLA-DR and HLAABC expression. 721.221 cells lack HLA-ABC and express HLA-DR, whereas RM3 cells lack HLA-DR and express HLA-ABC. All cells were stained for cell surface antigens CD14 (anti-CD14-APC, 61D3, Tonbo biosciences), CD3 (anti-CD3-AF700, UCHT1, BD Pharmingen), HLA-ABC (anti-HLA-ABC-AF488, W6/32; pan HLA class I, BioLegend), HLA-DR (anti-HLA-DR-PE, L243; pan HLA-DR, eBioscience), and fixable viability dye eFluor 506 (eBioscience) or isotype controls for HLA-ABC (AF488 Mouse IgG2a, κ, catalogue number 400233, BioLegend) or HLA-DR (PE Mouse IgG2a, κ, catalogue number 12-4724, eBioscience). After washing, cells were fixed using 4% paraformaldehyde. Samples were acquired on a BD LSR II flow cytometer. The fraction of living cells expressing HLA-ABC or HLA-DR was determined using FlowJo X Software.

H) α-Syn Purification and α-syn PFF Preparation

Recombinant α-syn monomer was purified as previously described 44. α-Syn pre-formed fibrils (PFF) were prepared by agitating α-syn monomer in a transparent glass vial with a magnetic stirrer (350 rpm at 37° C.). After 5-7 days of agitation, the clear α-syn monomer solution became turbid, indicative that α-syn fibrils were generated. The α-syn fibrils were then sonicated for 30 seconds at 10% amplitude to generate α-syn PFF (Branson Digital Sonifier, Danbury, Conn., USA). α-Syn monomer and PFF were aliquoted and kept at −80° C.

I) Statistics and Reproducibility

A power analysis was not conducted a priori as there was no means to estimate effect size. Future validation studies will test whether the Y39 antigenic region is recognized significantly higher in donors with PD compared to HC. The recognition frequency of this peptide was 17% in PD and 3% in HC, which achieves 61% power to detect a response difference between response rates of 14 percentage points. To achieve 80% power in a repeat study to detect a similar effect size, a total of 62 PD and 62 HC should be included. Additionally validation studies will test whether the overall recognition of the peptides is significantly higher in donors with PD compared to HC. Based on our combined cohort data the recognition frequency of a pool of peptides was 37% in PD and 8% in HC. To obtain 80% .power in a validation study a cohort size of 43 in both PD and HC will be required to detect the same effect.

The Fisher's exact (two-tailed) test was used to evaluate the contingency between carriers and non-carriers of the DRB1*15:01 and DRB5*01:01 alleles in the PD and HC donors (Supplemental Table 13), between the responses to phosphorylated aaS129 epitopes of PD and HC donors (FIG. 1e-g), and between DRB1*01/DRB5*01:01/DQB1*03:04/A*11:01 carriers and non-carriers in PD and HC donors (Table 15). A non-parametric test was used because the data is not normally distributed. Fisher's exact test that provides exact p values for the analysis of contingency tables and is available in most professional statistical analysis packages.

The Mann Whitney test (two-tailed) was used to assess whether the number of SFCs of HC donors would be less or greater than those of PD donors (FIG. 1b-g, FIG. 2, FIG. 3a-c). The Mann Whitney test (two-tailed) was used to determine if the number of IFNγ SFC was different than IL-5 SFCs of PD donors (FIG. 3d). A non-parametric test was used because the data is not normally distributed. T-tests were used to analyze parametric differences in demographics between PD and HC donors (Table 11a, 11b, 12).

The Wilcoxon test was used to analyze differences in population means of the repeated measurements of number of SFCs induced by media and different isoforms of α-syn (FIG. 9). A non-parametric test was used because the data is not normally distributed. It was hypothesized that responses to proteins and peptides would be higher than media alone, therefore a one-tailed test was used for those comparisons. Comparison between PFF and native α-syn was two-tailed.

A power analysis could not be run for this prior to the experiments as there were no means to estimate effect size. Future validation studies will test whether the Y39 antigenic region is recognized significantly higher in donors with PD compared to HC. The recognition frequency of this peptide was 17% in PD and 3% in HC, which achieves 61% power to detect a response difference between the response rates of 14 percentage points. To achieve 80% power in a repeat study to detect a similar effect size, a total of 62 PD and 62 HC should be included. Additionally, validation studies will test whether the overall recognition of the 11 peptides is significantly higher in donors with PD compared to HC. Based on our combined cohort data the recognition frequency of a pool of peptides was 37% in PD and 8% in HC. To obtain 80% power in a validation study a cohort size of 43 in both PD and HC will be required to detect the same effect. The present invention is useful to diagnose, confirm, provide a biomarker for, and treat PD.

Aspects of the present invention relate to the surprising discovery that epitope peptides that activate leukocytes are expressed on the surface of neurons in subjects afflicted with PD. Surprisingly, these epitopes are useful in diagnostic and treatment methods for PD. The present invention provides improved and novel methods for diagnosing, confirming, providing biomarkers for, and treating PD are needed. Additionally, specific treatments tailored for individual patients are provided herein.

### Example 14

### TAR DNA Binding Protein 43 (TDP43) is Recognized as an Autoantigen

Identification of specific TAR DNA binding protein 43 (TDP43) antigens that act as autoantigens. This can be used as the source of biomarkers, diagnostics and therapeutics via tolerization and related approaches.

Amyotrophic lateral sclerosis (ALS) patients undergo an extraordinarily rapid death of neurons, prominently including motor neurons, leading to death at a mean duration of three years following diagnosis. While aggregates in the surviving neurons clearly point to a disturbance in normal protein handling during the disease process, despite much research and multiple theories, the field has not identified the means by which these neurons die. In this proposal we explore a novel hypothesis, that these neurons may be killed by autoimmune T cells that recognize particular epitopes from misprocessed disease-linked proteins.

The work herein characterizes autoimmune epitopes in ALS patients.

ALS is presently associated with mutations in over twenty genes and many more are likely to be involved. Mutations in the gene for transactivation response DNA-binding protein 43 (TDP43) are rare, but there appears to be a convergence during ALS pathogenesis, as 97% of patients diagnosed with ALS feature TDP43 intraneuronal cytosolic aggregates (Neumann et al., 2006; Arai et al., 2006; Ling et al., 2013). TDP43 is nominally a nuclear ribonucleoprotein implicated in RNA handling, and so the presence of cytosolic aggregates strongly indicates abnormal protein handling and degradation of the protein associated with the disease (Blokhuis et al., 2013). The TDP43 protein within the aggregates features phosphorylated, deamidated, and cleaved residues in the glycine-rich C terminal region (Kametani et al., 2016). Without wishing to be bound by any scientific theory, methods of the present invention focus on TDP43 due to its near ubiquity in ALS aggregates, its protein modifications associated with disease, and its likely role as a substrate for chaperone-mediated autophagy (CMA). Without wishing to be bound by any scientific theory, there are multiple additional proteins also found in ALS aggregates that may be important as discussed herein.

Summary

Specific autoimmune damage in ALS stems from two overall lines of recent findings:

A) Finding 1

Although neurodegenerative disorders of aging are not considered to be autoimmune disorders, recent findings in press at Nature from the collaboration between the Columbia and LJI teams (Sulzer et al., 2017) show that there are helper T cell and cytotoxic CD8+ T cell (CTL) autoimmune responses in Parkinson's disease (PD), and that this is due to specific epitopes, in this case derived from α-synuclein (α-syn) presented by specific MHC-I and II alleles. These epitopes include a phosphorylated residue of α-syn, 5129, which is the classic component of Lewy bodies, which are PD aggregates with multiple analogies to ALS aggregates. This PD autoimmune response may in turn stem from the decrease in CMA activity that occurs with aging (Cuervo et al., 2005), and the Sulzer group in collaboration with Ana Maria Cuervo (Einstein University) introduced CMA as the means by which α-syn is degraded and by which pathogenic α-syn blocks normal protein degradation (Cuervo et al., 2004; Martinez-Vincente et al., 2008), including for other PD-linked proteins (Orenstein et al., 2013).

Without wishing to be bound by any scientific theory, it is noted that TDP43 possesses a CMA consensus sequence, 134QVKKD138, that is extremely close to the α-syn CMA sequence, 95VKKDQ99, and that TDP43 is likely to be a CMA substrate as well. CMA has already been shown to be blocked by pathogenic mechanisms such as amino acid modification. Thus, TDP43 is a strong candidate as an autoantigen, in a manner similar to α-syn. Without wishing to be bound by any scientific theory, this response could engender both biomarkers and new treatments for ALS patients. It is noted that in a recent publication, the protein annexin A-11 was reported as a relatively rare cause of ALS, and this protein is already implicated in the classical autoimmune disorders, systemic lupus erythematosus and sarcoidosis (Smith et al., 2017).

B) Finding 2

Adult CNS neurons were long thought to not present antigen, but a recent study demonstrates in human pathological specimens that midbrain dopamine and norepinephrine neurons that die in PD express MHC-I (Cebrian et al., 2014). In rodent models, the MHC-I expression in the neurons is driven by cytokines, particularly interferon-y released from activated microglia, and the appropriate combination of T cells and antigens kill these neurons. In the specific case of motor neurons, multiple earlier studies demonstrate MHC-I presentation by mature and aged motor neurons in rodents 13-15, and a new study confirms MHC-I presentation by motor neurons in SOD1 ALS mouse models (Nardo et al., 2016). In ALS human pathology, multiple publications report activation of microglia and monocytes (Butovsky et al., 2012; Zhao et al., 2017), a feature also observed in ALS mouse models (Chiu et al., 2009; Chiu et al., 2013). In human ALS, there appear to be variable levels of T cell infiltration: Appel and collaborators first suggested autoimmune features of ALS, although with normal numbers of T cells in most patients 21, while early reports found a 90-fold increase in cytotoxic CD8+ T cells (CTLs) in ALS spinal cord over age-matched control patients, and 27-fold increase in helper T cells (McGreer et al., 1993; Kawamata et al., 1992). To our knowledge, there have been no reports of MHC-I or II on neurons or astrocytes in human ALS specimens. It is noted that studies of the periphery often assume that MHC-I provides a neuroprotective role, but that in ALS pathology (Chiu et al., 2008), as with dopamine neurons and other cells that express MHCI, recognition by CTLs may lead to cell death.

Without wishing to be bound by any scientific theory, it is hypothesized that aberrant degradation and processing of TDP43, and likely additional proteins, lead to the production of specific TDP43-derived autoimmune epitopes displayed by MHC-II that activates specific helper T cells and by MHC-I that activate specific CTLs. Future work would determine the role of autoimmune function in neuronal death, if autoimmune response provides ALS biomarkers, and how blockade of these steps could halt ALS pathogenesis.

Aim of Experiments

C) Aim 1

All possible 15-mer peptides of TDP43 overlapping by 10 amino acid residues covering the entire protein sequence will be produced, corresponding to approximately 40 peptides. Additionally, a smaller number of peptides will be manufactured that feature deamidated asparagine and oxidized methionine residues reported in ALS aggregates (Kametani et al., 2016). While initial focus is on TDP43 as discussed above, a smaller number (˜5 each) of peptide candidates will be determined in FUS and SOD-1, which are less ubiquitous but relatively common additional proteins observed in ALS aggregates (Blokhuis et al., 2013), in contrast to annexin-A11, which is only observed in particular familial samples (Smith et al., 2017). All of these peptides (approximately 60) will be arranged in 6 pools of approximately 10-15 peptides each. Clinical analysis will be performed and 30 cc fresh blood samples obtained from 40 sporadic ALS and 40 age-matched controls (HC) that will be sent for analysis of specific T cell reactivity. For this purpose, PBMC will be separated by the use of standard magnetic bead protocols (Miltenyi Biotec), in subsets corresponding to CD4+ T cells including effector and regulatory subtypes, CD8+ T cells, and remaining CD3-cells (containing B cells, DC and macrophages to be used as APC). CD4+ T cells and CD8+ T cells will be separately stimulated in the presence of APC with the different peptide pools. After in vitro restimulation, T cell recognition will be assessed by triple FLUOROSPOT analysis for detection of IL-5 (indicating helper T cells mainly of the Th2 subset), IL-17 (for Th17 cells), IFN-γ (for CTLs/helper Th1 cells) and IL-10 (for regulatory T regs) to pools of epitopes under conditions in which diagnosis is blinded. Responsive samples will be deconvoluted and the specific epitopes responsible identified. T cell lines that undergo activation in response to specific epitopes will be analyzed by flow cytometry assays to confirm their CD4/CD8 identity as CTLs or specific helper subtypes, and additional phenotypic characterization will be performed in terms of patterns of cytokines secreted. All data will be examined blind, will not be traceable to donors, and will be considered exempt (under Title 32, Federal Regulations, Part 219, Section 101(b) (32 CFR 219.101[b]).

D) Aim 2

The epitopes identified above derived from TDP43, FUS and SOD-1 will be characterized. Aim 2a). The correlation will be analyzed between T cell recognition and location of the immunogenic sequences. Without wishing to be bound by any scientific theory, it is expected that the epitopes will concentrate in particular regions near the carboxy terminus, based on reports of TDP43 fragments found in ALS aggregates (aa252-263, 276-293, 409-414) (Neumann et al., 2006), and recently reported TDP43 amino acid modifications in ALS patients (Kametani et al., 2016), including 18 phosphorylated disease-linked serine residues near the carboxyl terminus (from aa 242 to 409). Aim 2b) The HLA molecules that act as restriction elements will be determined. For this purpose, HLA typing will be determined by next-generation sequencing methods, and the association between responses to individual alleles and particular HLA molecules determined by genetic inference (Paul et al., 2015). The putative restrictions will be independently confirmed by performing quantitative HLA-peptide binding measurements with the HLA molecules expressed in the responding donors. Aim 2c) These confirmed restriction data will be utilized to produce tetrameric and dextrameric staining reagents, isolate specific responding T cells, and determine the patterns of TCR gene expression (TCR sequencing), of interest for the development of ALS biomarkers.

E) Aim 3

The ALS and HC samples will be genotyped for HLA alleles and for known causes of familial ALS (including and not limited to specific alleles in the TDP43 gene, TARDBP, HNRNPA1, PFN1, C9ORF72, UBQLN2, OPTN, VCP, ANXA11, and FUS). T cell receptors responsible for recognition of specific epitope/HLA combinations will be sequenced. After analysis, the results will be unblinded and analyzed for the relationship between specific autoimmune responses and disease status, duration, age, sex, and HLA genotype, as well as known ALS-linked genes.

Discussion

F) Innovation

There have been no analyses to address if ALS has autoimmune responses to epitopes from specific disease-linked proteins, nor characterization of the HLA alleles, T cells, or T cell receptors involved in such responses. Indeed, this approach has only become possible with the introduction of new technical approaches to identify specific epitope-allele interactions by the Sette group and collaborators. More broadly, the only publication describing such autoimmune response in neurodegenerative diseases of aging is the recent collaborative paper in PD patients (Sulzer et al., 2017), which relied on work on protein degradation, and the identification of candidate HLAs and epitopes. It is noted that multiple sclerosis is a classic autoimmune degenerative disorder of the nervous system, but that the principal cellular targets of the immune system are oligodendrocytes.)

The autoimmune responses to misfolded and/or aggregated signature proteins in ALS will provide clear consequences for the development of biomarkers and therapy.

G) Impact

Together, this experimental design provides a clear means to address if ALS has autoimmune responses to epitopes of specific disease-linked proteins. Once specific epitope-HLA restriction patterns are discerned, ALS patients can be characterized for their specific autoimmune responses using “multimer” technology, which provides a rapid and effective means to assay the number of specific reactive T cells. This would define specific subtypes of ALS, and contribute a valuable and independent means to genotyping. It is noted that many sporadic ALS patients have no known associated disease allele, and in analogy to our findings from responses to o syn-derived epitopes in PD, the autoimmune responses may show convergence from multiple causes. This may be particularly true for TDP43 epitopes, which would be present in the vast majority of aggregates of nearly all ALS patients.

The identification of specific autoimmune T cells may provide a predictive biomarker, and may provide a biomarker for the efficacy of treatments for particular individuals. It is acknowledged that the treatment for autoimmune disorders has been very challenging, and multiple immunosuppressive drugs (glucocorticoids, cyclophosphamide, azathioprine, cyclosporine and others) have been ineffective in ALS patients. It is further noted there are however recent developments that provide very effective immunomodulatory treatments for many relapsing-remitting multiple sclerosis patients that could be examined for ALS treatment (e.g., terifflunomide, dimethyl fumarate, natalizumab). Perhaps central to the development of effective treatment is that the immunosuppressive drugs that to our knowledge have been examined in ALS are not specific for T cell epitope combinations, and there are multiple new approaches under development to specifically modulate epitopespecific reactions. Additional approaches may include means to decrease MHC presentation by microglia, motor neurons, astrocytes, and epithelial cells. A “personalized” approach based on detection of the antigen-HLA conformation and T cell receptors of that patient may be required due to the diversity of HLAs, epitopes and T cell receptors.

This study would also contribute to the basic knowledge of how neurons die in ALS, a central issue that remains unclear and promises additional therapeutic development. Given the evidence above for recognition of MHC-II epitopes, and the expression by MHC-I by motor neurons, the results from this proposal may provide the means for follow-up studies to specifically test T cell-mediated responses in animal ALS models, as well as iPSC-derived neurons from particular ALS patients with defined HLA alleles and restricted epitope responses.

### Example 15

### Cytokine Release in Controls and ALS Patients

Blood from age matched controls and ALS patients were obtained and mononuclear cells were isolated by gradient centrifugation.

Release of the cytokines gamma-interferon, which measures activation of CD4+ and/or CD8+ T cells, the interleukin, IL-5, which measures activation of CD4+ T cells, and the interleukin IL-10 were measured by ELISpot assay. Briefly, the isolated cells were plated in wells that have colorimetric detection of gamma-interferon, IL-5, and IL-10, and were stimulated with pools of epitopes of TDP40 that the Sette lab determined would potentially be displayed by MHC-I or MHC-II antigen-presenting proteins in humans.

After two weeks of stimulation, the cells were harvested and release of cytokines was measured by colorimetric detection of spot-forming cells (SFC). Confirmed release of cytokine is determined by the presence of a minimum of 20 SFC per million cells.

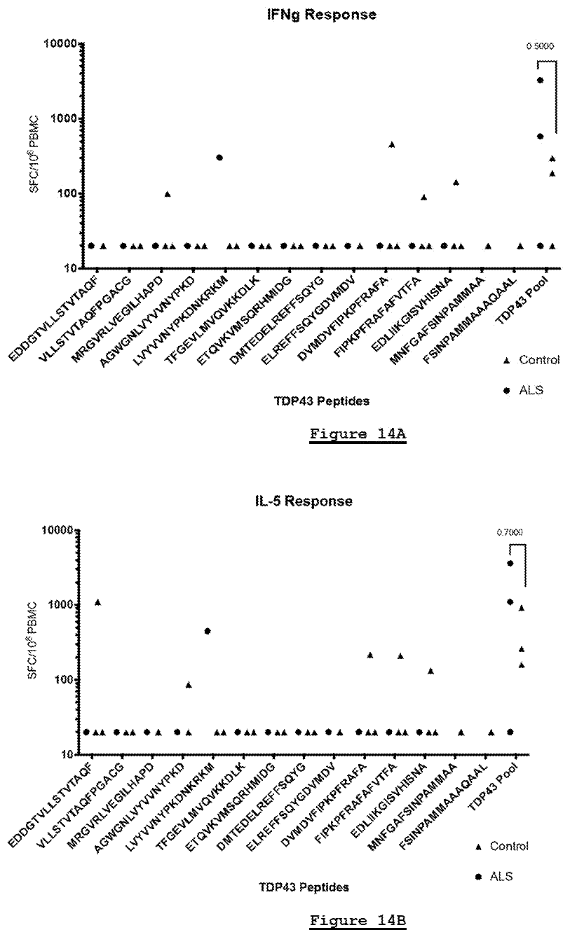

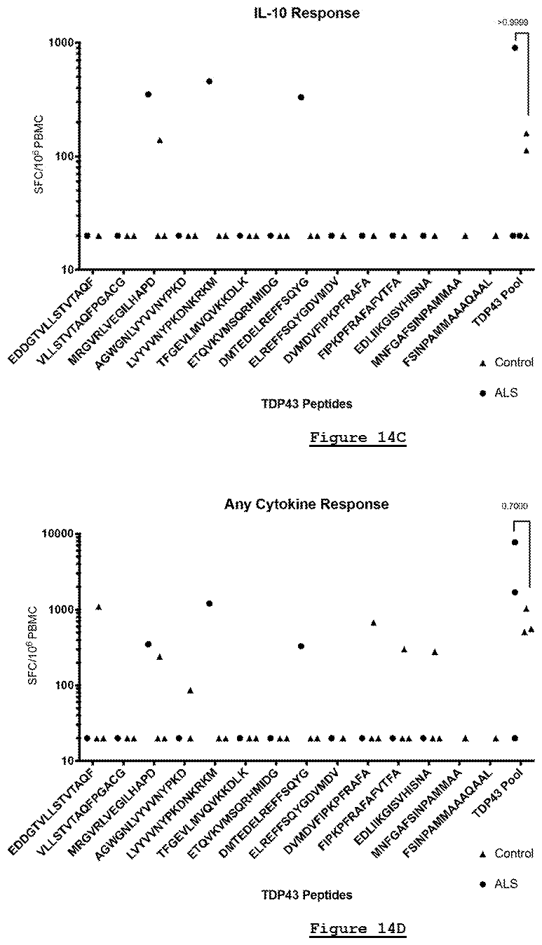

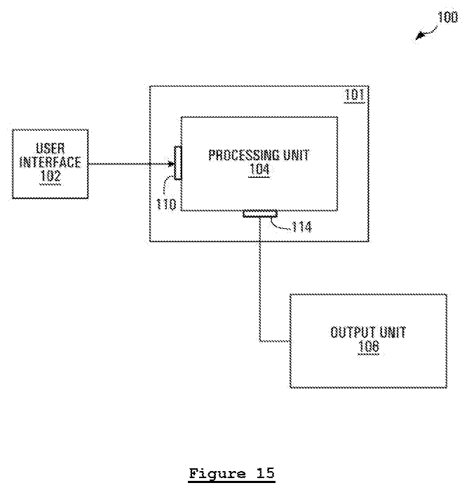

Preliminary results indicated that ALS patients have a high reactivity to TDP43 peptides than the control groups (FIG. 14A-14D).

ALS patients are more likely to have T cells in blood that recognize and are activated by TDP43, FUS or SOD-1 than unaffected individuals.

### Example 16

### Tolerization Therapy Specific for Epitopes of TDP43, FUS or SOD-1 are Useful in Treating Subjects Afflicted with ALS

Epitopes to which T cells are responsive in subjects afflicted with ALS are identified by

i) obtaining T cells from each subject;

ii) contacting the T cells with a test compound;

iii) determining whether the T cells have increased activation after contact with the test compound; and

iv) identifying the test compound as an epitope to which the T cells are responsive if in step iii) the T cells are determined to have increased activation after contact with the test compound, and identifying the test compound as not an epitope to which the T cells are responsive if in step iii) the T cells are determined to not have increased activation after contact with the test compound. This method is repeated sequentially or in parallel for thousands of test compounds, each having an amino acid sequence identical to a stretch of consecutive amino acids in the TDP43 protein. Epitopes for TDP43 are identified in individual subjects.

Epitopes to which T cells are responsive in subjects afflicted with ALS are identified by

i) obtaining T cells from each subject;

ii) contacting the T cells with a test compound;

iii) determining whether the T cells have increased activation after contact with the test compound; and

iv) identifying the test compound as an epitope to which the T cells are responsive if in step iii) the T cells are determined to have increased activation after contact with the test compound, and identifying the test, compound as not an epitope to which the T cells are responsive if in step iii) the T cells are determined to not have increased activation after contact with the test compound. This method is repeated sequentially or in parallel for thousands of test compounds, each having an amino acid sequence identical to a stretch of consecutive amino acids in the FUS protein. Epitopes for FUS are identified in individual subjects.

Epitopes to which T cells are responsive in subjects afflicted with ALS are identified by

i) obtaining T cells from each subject;

ii) contacting the T cells with a test compound;

iii) determining whether the T cells have increased activation after contact with the test compound; and

iv) identifying the test compound as an epitope to which the T cells are responsive if in step iii) the T cells are determined to have increased activation after contact with the test compound, and identifying the test compound as not an epitope to which the T cells are responsive if in step iii) the T cells are determined to not have increased activation after contact with the test compound. This method is repeated sequentially or in parallel for thousands of test compounds, each having an amino acid sequence identical to a stretch of consecutive amino acids in the SOD-1 protein. Epitopes for SOD-1 are identified in individual subjects.

The subjects afflicted with ALS are then separated into one of two groups: 1) a test group that receives tolerization therapy, or 2) a control group that does not receive tolerization therapy.

Within the test group, an effective amount of an epitope to which T cells are responsive in subjects afflicted with ALS is administered orally, nasally, or subcutaneously to each subject (i.e., tolerization therapy specific for the epitope). Within the control group, a polypeptide having a random sequence is administered to each subject.

Compared to the control group, subjects in the test group have a statistically significant reduction in symptoms of ALS. Additionally, a statistically significant proportion of the subjects have little or no progression of ALS.

Less or no activation of T cells by the epitope is observed in subjects who receive and respond to tolerization therapy, but not in subjects who do not receive or who do not respond to tolerization therapy.

### Example 17

### Autoimmune Features of Neurodegenerative Disorders

Without wishing to be bound by any scientific theory, at least some ALS is in part an autoimmune disorder.

Without wishing to be bound by any scientific theory, the T cells recognize TDP43, PUS, or SOD-1.

Aspects of the present invention relate to the surprising discovery that epitopes that activate leukocytes are expressed on the surface of neurons in subjects afflicted with ALS. Surprisingly, these epitopes are useful in diagnostic and treatment methods for ALS. The present invention provides improved and novel methods for diagnosing, confirming, providing biomarkers for, and treating ALS are needed. Additionally, specific treatments tailored for individual patients are provided herein.

### Example 18

### T Cells from Patients with Parkinson's Disease Recognize α-synuclein Peptides

This example describes a specific epitope screen to identify disease-relevant antigens for Parkinson's disease. It should be understood that these methods can be broadly applied to other neurodegenerative diseases and disorders that involve an inflammatory response and/or inflammation, nonlimiting examples of such are disclosed herein.

Genetic studies have shown the association of Parkinson's disease with alleles of the major histocompatibility complex (Greenbaum, J. et al. Functional classification of class II human leukocyte antigen (HLA) molecules reveals seven different supertypes and a surprising degree of repertoire sharing across supertypes. Immunogenetics 63, 325-335 (2011); Hamza, T. H. et al. Common genetic variation in the HLA region is associated with late-onset sporadic Parkinson's disease. Nat. Genet. 42, 781-785 (2010); Kannarkat, G. T. et al. Common genetic variant association with altered HLA Expression, synergy with pyrethroid exposure, and risk for Parkinson's Disease: an observational and case-control study. NPJ Parkinson's Dis. 1, 15002 (2015)).

Described herein is a defined set of peptides that are derived from α-synuclein, a protein aggregated in Parkinson's disease (Spillantini, M. G., Crowther, R. A., Jakes, R., Hasegawa, M. & Goedert, M. α-synuclein in filamentous inclusions of Lewy bodies from Parkinson's disease and dementia with lewy bodies. Proc. Natl Acad. Sci. USA 95, 6469-6473 (1998)) act as antigenic epitopes displayed by these alleles and drive helper and cytotoxic T cell responses in patients with Parkinson's disease. Without being bound by theory, these responses may explain the association of Parkinson's disease with specific major histocompatibility complex (MHC) alleles.

Abnormal processing of self-proteins can produce epitopes, which are presented MHC proteins to be recognized by specific T cells that have escaped tolerance during thymic selection (Marrack, P. & Kappler, J. W. Do MHCII-presented neoantigens drive type 1 diabetes and other autoimmune diseases? Cold Spring Harb. Perspect. Med. 2, a007765 (2012)). Such actions by the acquired immune system have been implicated in autoimmune disorders, including type-1 diabetes. While not considered to possess autoimmune features, neurodegenerative diseases are characterized by altered protein processing. The major pathological features of Parkinson's disease, the most common neurodegenerative movement disorder, are the death of dopaminergic neurons of the substantia nigra, and the presence of intraneuronal aggregates known as Lewy bodies that are composed of α-synuclein (α-syn) (Spillantini, M. G., Crowther, R. A., Jakes, R., Hasegawa, M. & Goedert, M. α-synuclein in filamentous inclusions of Lewy bodies from Parkinson's disease and dementia with lewy bodies. Proc. Natl Acad. Sci. USA 95, 6469-6473 (1998)). Activated microglia have been reported in the substantia nigra of patients with Parkinson's disease for nearly a century and cytokine profiles have implicated the activation of the innate immune system (Cebrián, C., Loike, J. D. & Sulzer, D. Neuroinflammation in Parkinson's disease animal models: a cell stress response or a step in neurodegeneration? Curr. Top. Behay. Neurosci. 22, 237-270 (2015)).

More recent evidence has suggested a role for the acquired immune system (Cebrián, C., Loike, J. D. & Sulzer, D. Neuroinflammation in Parkinson's disease animal models: a cell stress response or a step in neurodegeneration? Curr. Top. Behay. Neurosci. 22, 237-270 (2015)). including T cell infiltration into the substantia nigra of patients with Parkinson's disease (Brochard, V. et al. Infiltration of CD4+ lymphocytes into the brain contributes to neurodegeneration in a mouse model of Parkinson disease. J. Clin. Invest. 119, 182-192 (2009)).

Genome-wide association studies have shown the association of Parkinson's disease with an immune haplotype (Wissemann, W. T. et al. Association of Parkinson disease with structural and regulatory variants in the HLA region. Am. J. Hum. Genet. 93, 984-993 (2013)) that is present in approximately 15% of the general population including the MHC class II gene alleles DRB501 and DRB1*15:01and a polymorphism in a non-coding region that may increase MHC class II expression. (Hamza, T. H. et al. Common genetic variation in the HLA region is associated with late-onset sporadic Parkinson's disease. Nat. Genet. 42, 781-785 (2010); Kannarkat, G. T. et al. Common genetic variant association with altered HLA Expression, synergy with pyrethroid exposure, and risk for Parkinson's Disease: an observational and case-control study. NPJ Parkinson's Dis. 1, 15002 (2015)). Antigen presentation by MHC class I expression in dopamine neurons of the substantia nigra in adult human brains of patients with Parkinson's disease and age-matched controls is reported. It has been further demonstrated that dopamine neurons of the substantia nigra express MHC class I upon activation by cytokines that are released from microglia, which are activated by α-syn or neuromelanin, and that CD8⁺ T cells kill neurons that present the appropriate combination of MHC class I and peptide. (Cebrián, C. et al. MHC-I expression renders catecholaminergic neurons susceptible to T-cell-mediated degeneration. Nat. Commun. 5, 3633 (2014)). Native and modified (nitrated) synuclein-derived peptides (Mor, F., Quintana, F., Mimran, A. & Cohen, I. R. Autoimmune encephalomyelitis and uveitis induced by T cell immunity to self α-synuclein. J. Immunol. 170, 628-634 (2003); Theodore, S., Cao, S., McLean, P. J. & Standaert, D. G. Targeted overexpression of human α-synuclein triggers microglial activation and an adaptive immune response in a mouse model of Parkinson disease. J. Neuropathol. Exp. Neurol. 67, 1149-1158 (2008); Benner, E. J. et al. Nitrated α-synuclein immunity accelerates degeneration of nigral dopaminergic neurons. PLoS ONE 3, e1376 (2008)) elicit T cell responses in rats and mice, and it was previously demonstrated that neuronal death in the substantia nigra in an α-syn overexpression model is absent in MHC II null mice (Harms, A. S. et al. MHCII is required for α-synuclein -induced activation of microglia, CD4 T cell proliferation, and dopaminergic neurodegeneration. J. Neurosci. 33, 9592-9600 (2013)).

To address whether Parkinson's disease is associated with T cell recognition of epitopes that are derived from α-syn presented by specific MHC alleles, 67 participants with Parkinson's disease and 36 age-matched non-Parkinson's disease healthy controls were recruited. Participants were 46-83 years of age (Parkinson's disease, median 66, range 46-83; healthy controls, median 64, range 52-83) and 66% were male (Parkinson's disease, 75%; healthy controls, 50%) (Tables 11A, 11B, 12). Whereas approximately 15% of healthy controls carried DRB1*15: DRB5*01:01 alleles, around one-third of patients with Parkinson's disease carried these alleles (difference between patients with Parkinson's disease and healthy controls, P=0.036 and 0.022 for DRB1*15:01 and DRB5*01:01, respectively), indicating association of HLA DR allelic variants with Parkinson's disease in our cohort (Table 13).

To determine whether α-syn-derived peptides were recognized by T cells, responses to pools that each contained approximately twenty peptides of 9-10 amino acids (a.a.) predicted to bind common HLA class I types, (Vita, R. et al. The immune epitope database (IEDB) 3.0. Nucleic Acids Res. 43, D405-D412 (2015)) and peptides of 15 amino acids spanning the protein that could elicit HLA class II responses were assayed. Peripheral blood mononuclear cells from patients with Parkinson's disease and healthy controls were stimulated for 14 days. Interferon-γ (IFNγ) and interleukin-5 (IL-5) responses were measured by dual-colour enzyme-linked immunospot (ELISPOT) assay, enabling quantification of responsive cells. Positive pools were deconvoluted to identify the peptides eliciting cytokine responses. IFNy was used as a representative cytokine to detect CD8⁺ HLA class I and CD4⁺ T helper 1 (Tₕ1) class II T cells, and IL-5 as a representative cytokine secreted by CD4⁺ Tₕ2 class II T cells. Each pool was tested in an initial cohort of 19-25 randomly selected patients with Parkinson's disease and 12 healthy controls. The majority of PBMC responses to the peptides of 15 amino acids produced IL-5 (68% of total responses), indicating a prominent CD4⁺ Tⱼ2 phenotype, and the remainder of the responses were to IFNγ (32%). No cells producing both IL-5 and IFNγ were detected.

Two antigenic regions in α-syn were identified, the first near the N terminus, composed of a.a.31GKEGVLYVGSKTKa.a.45 and a.a.32KEGVLYVGSKTKEa.a.46 (referred to as the Y39 region) (FIG. 6A), which elicited an apparent class II restricted IL-5 and IFNy response (FIGS. 6B-FIG. 6D). 32 is a plasmin-cleavage site (Kim, K. S. et al. Proteolytic cleavage of extracellular α-synuclein by plasmin: implications for Parkinson disease. J. Biol. Chem. 287, 24862-24872 (2012)) and chymotrypsin-cleavage digestion sites are at 32 and 45 (ref. 17). (Hossain, S. et al. Limited proteolysis of NACP α-synuclein. J. Alzheimers Dis. 3, 577-584 (2001)).

The second antigenic region was near the C terminus (a.a. 116-140) (referred to as the S129 region) (FIG. 6A) and required phosphorylation of amino acid residue S129. The three phosphorylated S129 epitopes (a.a.116MPVDPDNEAYEMPSEa.a.130, a.a.121DNEAYEMPSEEGYQDa.a.135, a.a.126EMPSEEGYQDYEPEAa.a.140) produced markedly higher IL-5 responses in patients with Parkinson's disease than in healthy controls (P=0.02, Fisher's exact test, threshold of at least 300 spot-forming cells (SFC) (FIG. 6E-FIG. 6G). Phosphorylated S129 residues are present at high levels in Lewy bodies of patients with Parkinson's disease, (Fujiwara, H. et al. α-synuclein is phosphorylated in synucleinopathy lesions. Nat. Cell Biol. 4, 160-164 (2002)) and Lewy bodies of patients with Parkinson's disease contain α-syn fragments with cleavage sites approximately at amino acids 115, 119, 133 and 135 (Anderson, J. P. et al. Phosphorylation of Ser-129 is the dominant pathological modification of α-synuclein in familial and sporadic Lewy body disease. J. Biol. Chem. 281, 29739-29752 (2006)) and include the fragment a.a.129SEEGYQDYEPEAa.a.140, which is contained within one of the S129 epitopes. Caspase-1 (Anderson, J. P. et al. Phosphorylation of Ser-129 is the dominant pathological modification of α-synuclein in familial and sporadic Lewy body disease. J. Biol. Chem. 281, 29739-29752 (2006)) and neurosyn (Kasai, T. et al. Cleavage of normal and pathological forms of α-synuclein by neurosin in vitro. Neurosci. Lett. 436, 52-56 (2008)) can cleave α-syn at a.a.121, chymotrypsin and cathepsin D digestion sites are at a.a.116, a.a.125 and a.a.136 (Hossain, S. et al. Limited proteolysis of NACP α-synuclein. J. Alzheimers Dis. 3, 577-584 (2001)) proteasome may cleave between a.a.119 and a.a.120 (Li, W. et al. Aggregation promoting C-terminal truncation of α-synuclein is a normal cellular process and is enhanced by the familial Parkinson's disease-linked mutations. Proc. Natl Acad. Sci. USA 102, 2162-2167 (2005)) and calpain can cleave at a.a.122, with resulting fragments that have been identified in brains of patients with Parkinson's disease (Dufty, B. M. et al. Calpain-cleavage of α-synuclein: connecting proteolytic processing to disease-linked aggregation. Am. J. Pathol. 170, 1725-1738 (2007)).

The immune responses to a.a.39 and a.a.129 region epitopes, which included analysis of a second cohort of 19 patients with Parkinson's disease and 12 healthy controls that were assayed for response to additional phosphorylated and nitrated modifications (FIG. 7) were different between patients with Parkinson's disease and healthy controls for secretion of IFNγ (two-tailed Mann-Whitney U-test, P<0.05), IL-5 (two-tailed Mann-Whitney U-test, P<0.001) and both combined responses (two-tailed Mann-Whitney U-test, P<0.001) (FIG. 8A-8C). While residue is highly phosphorylated in patients with Parkinson's disease, (Brahmachari, S. et al. Activation of tyrosine kinase c-Ab1 contributes to α-synuclein-induced neurodegeneration. J. Clin. Invest. 126, 2970-2988 (2016)), Y39 phosphorylation was not required for antigenic response. The response was primarily polarized towards IL-5 in patients with Parkinson's disease (71% IL-5 and 29% IFNγ; FIG. 8D). This polarization was specific to patients with Parkinson's disease, and the relatively rare responses in healthy controls were not similarly polarized (46% IL-5 and 54% IFNγ).

To identify specific sets of T cells that respond to α-syn epitopes, the response to a pool of the 11 α-syn antigenic peptides by nine participants with Parkinson's disease was measured (FIG. 9). Approximately 0.2% of CD3⁺ T cells responded to the α-syn peptides. Of the responsive T cells, approximately 50% produced IL-4 and 50% produced IFNγ, with no detectable IL-10 or IL-17 production. In most cases, responses were mediated by CD4⁺ T cells, but response by one patient with Parkinson's disease was mostly mediated by IFNγ-producing CD8⁺ T cells. Therefore, the T cell response to α-syn antigenic peptides was largely mediated by IL-4 or IFNγ-producing CD4⁺ T cells, with potential contributions from IFNγ-producing CD8⁺ T cells.

To test whether the α-syn epitopes arise from processing of native and/or fibrilized α-syn, PBMCs were stimulated with α-syn epitopes for 14 days. The cultures were then assayed following exposure to α-syn peptides, 25 μg ml⁻¹ fibrilized (pre-formed fibrils, PFF) α-syn, 25 μg ml⁻¹ native α-syn or medium alone. FIG. 10 shows that T cell lines specific for the α-syn epitopes were activated by antigen-presenting cells pulsed with native or PFF protein in 7 out of 12 or 11 out of 12 cases, respectively. There was a significantly higher response to native α-syn (P=0.004) and to PFF α-syn (P=0.0005) than to medium alone. Therefore, T cells can respond to α-syn epitopes arising from natural processing of extracellular native α-syn, which is present in blood, and the fibrilized α-syn associated with Parkinson's disease.

Next, the HLA alleles that present α-syn peptides by in vitro binding to a panel of HLAs representing the common alleles expressed in worldwide populations were identified. A threshold of 1,000 nM binding affinity is associated with immunogenicity of HLA class II T cell epitopes, and most epitopes bind in the 1-100 nM range, with affinities in the 1-10 nM considered to be of high affinity. Of 26 common HLA class II alleles tested, five bound to a.a.32KTKEGVLYVGSKTKEa.a.46 (Table 5). The HLA class II variants DRB1*15:01 and DRB5*01:01 bound to the epitope with high affinity (2.8 nM and 8.1 nM, respectively), while DRB1*07:01, DRB1*09:01 and DQB1*03:01 bound in the 80-250 nM range. The a.a.32KTKEGVLYVGSKTKEa.a.46 epitope phosphorylated at Y39 also bound DRB1*15:01 and DRB5*01:01 with high affinity. Comparison of patients with Parkinson's disease with and without DRB1*15:01 alleles showed that there was no difference in levels of HLA class I or class II protein expression (FIG. 7 & FIG. 9). Thus, epitopes in the Y39 region of α-syn strongly bind HLA heterodimers including two HLA class II α chain alleles associated with Parkinson's disease.

By contrast, the C terminus peptides spanning S129 and its post-translational forms bound HLA class II alleles weakly, with the exception of a.a.121DNEAYEMPSEEGYQDa.a.135, which in both native and phosphorylated S129 forms strongly bound to DQBl*05:01. The a.a.116MPVDPDNEAYEMPSEa.a.130 epitope bound to several alleles with lower affinity, and the a.a.126EMPSEEGYQDYEPEAa.a.140 epitope bound to DQB1*04:02 and DQB1*05:01 with low affinity. Thus, antigenic peptides in the C terminus S129 antigenic region demonstrated relatively little clear restriction, suggesting that they are recognized promiscuously. DRB1*15:01 and DRB5*01:01 alleles are in linkage disequilibrium, and participants expressing one allele are likely to express both. Of participants with Parkinson's disease, 8 out of 13 responders to the a.a.32KTKEGVLYVGSKTKEa.a.46 epitope expressed both 1*15:01 and DRB5*01:01, while only 12 out of 45 (DRB1*15:01) and 13 out of 43 (DRB5*01:01) non-responders expressed the alleles, indicating an association between the alleles and antigenic response (odd ratios of 4.4 and 3.7, P values of 0.04 and 0.05, respectively) (Table 14). This analysis detected additional associations, with 2 out of 13 responders expressing DQBl*03:04 (P=0.05) compared to 0 out of 45 non-responders, as well as the HLA class I allele A*11:01, with 8 out of 13 responders expressing A*11:01 compared to 9 out of 45 non-responders (P=0.012). While A*11:01 is in relatively mild linkage disequilibrium with DRB1*15:01 and DRB1*01:01, the associations were largely independent (FIG. 13A). In general, participants with Parkinson's disease showed a trend towards higher expression of HLA molecules, particularly HLA class II. This is consistent with an inflammatory component of Parkinson's disease, and higher HLA class II expression and induction in PBMCs of patients with Parkinson's disease compared to healthy controls (Kannarkat, G. T. et al. Common genetic variant association with altered HLA Expression, synergy with pyrethroid exposure, and risk for Parkinson's Disease: an observational and case-control study. NPJ Parkinson's Dis. 1, 15002 (2015)). Little or no difference in HLA class II expression was found between participants expressing DRB1*15:01 versus other DRB1 alleles (FIG. 4). A similar but still less pronounced trend was noted for HLA class I (FIG. 5). This suggests that the association between DRB1*15:01 and Parkinson's disease is not based on differential expression of the protein. A negative association between recognition of a.a.32KTKEGVLYVGSKTKEa.a.46 and the DRB3*02:02 allele was detected, suggesting this allele might be protective. The four alleles DRB1*15:01, DRB5*01:01, DQB1*03:04 and A*11:01 accounted for every single individual responding to the a.a.39 epitope (P=0.00007 for Parkinson's disease, Table 14). This association was far more significant in Parkinson's disease than healthy controls (P=0.009). The combined association of the four alleles for Parkinson's disease versus healthy controls was significant (P=0.008 two-tailed Fisher's exact test compared to individual DRB1*15:01, P=0.05; and DRB5*01:01, P=0.03), with around half of the patients with Parkinson's disease (31 with alleles and 27 without) carrying one of the four alleles, whereas only around 20% of the healthy controls (8 with alleles and 26 without) expressed one of the four (Table 14).

Following detection of association of response to the Y39 region with the MHC class I allele HLA A*11:01, Parkinson's disease responses to shorter α-syn-derived peptide candidates for class I presentation were evaluated. 5 out of 19 Parkinson's disease responded to these short peptides, whereas 0 out of 12 healthy controls responded (FIG. 13B-13C) (two-tailed χ₁²=3.765,P=0.0523). Reactivity occurred mostly on peptides contained within the Y39 region, involving three peptides (a.a.36GVLYVGSKTKa.a.45, a.a.37VLYVGSKTKa.a.45, a.a.37VLYVGSKTKKa.a.46) predicted as potential A*11:01 binders (Vita, R. et al. The immune epitope database (IEDB) 3.0. Nucleic Acids Res. 43, D405-D412 (2015)). Each peptide was tested for binding to purified HLA A*11:01 molecules in vitro, and found that the 9-mer a.a.37VLYVGSKTKa.a.45, which is nested within the two 10-mers, bound with good 50% inhibitory concentration (IC₅₀)=161 nM), while the other two bound poorly, indicating that the 9-mer is responsible for T cell recognition. Reactivity to short peptides was mostly mediated by IFNy-producing cells and most pronounced for the All binding peptides. Therefore, immune responses to α-syn associated with Parkinson's disease have both MHC class I and II restricted components.

Alleles of over twenty genes are associated with familial Parkinson's disease, (Hernandez, D. G., Reed, X. & Singleton, A. B. Genetics in Parkinson disease: Mendelian versus non-Mendelian inheritance. J. Neurochem. 139, 59-74 (2016)) many of which encode proteins implicated in lysosomal degradation pathways including mitochondrial turnover. For example, mutations in α-syn or dopamine-modified α-syn, (Martinez-Vicente, M. et al. Dopamine-modified α-synuclein blocks chaperone-mediated autophagy. J. Clin. Invest. 118, 777-788 (2008); Cuervo, A. M., Stefanis, L., Fredenburg, R., Lansbury, P. T. & Sulzer, D. Impaired degradation of mutant α-synuclein by chaperone-mediated autophagy. Science 305, 1292-1295 (2004)), and LRRK2 (Orenstein, S. J. et al. Interplay of LRRK2 with chaperone-mediated autophagy. Nat. Neurosci. 16, 394-406 (2013)) interfere with protein degradation by chaperone-mediated autophagy, a process that becomes less efficient with age. Extracellular oligomeric α-syn may be acquired by brain cells during Parkinson's disease pathogenesis (Luk, K. C. et al. Pathological α-synuclein transmission initiates Parkinson-like neurodegeneration in nontransgenic mice. Science 338, 949-953 (2012)). Without being bound by theory, these reports suggest that altered degradation of proteins including α-syn could produce antigenic epitopes that trigger immune reactions during ageing and Parkinson's disease.

These results indicate that peptides derived from two regions of α-syn produce immune responses in patients with Parkinson's disease; their roles in additional synucleinopathies are untested. Epitopes derived from the Y39 region (approximately from a.a.31/32 to a.a.45/46) are specifically displayed by two MHC class II a-chain alleles, DRB5*01:01 and DRB1*15:01, associated with Parkinson's disease, as well as an additional MHC class II allele and an MHC class I allele not previously associated with Parkinson's disease. The response is enacted mostly by IL-5-secreting CD4⁺ T cells, as well as IFNγ-secreting CD8⁺ cytotoxic T cells. α-syn is, to our knowledge, not endogenously expressed by cells that express MHC class II, but is found from where it can be acquired by MHC class II-expressing cells. This situation is analogous to the experimental autoimmune encephalitis model of multiple sclerosis, as myelin proteins used to produce autoimmunity are not endogenous to MHC class II-expressing cells, but are accumulated and processed for MHC class II display by antigen-presenting cells and microglia. The Y39 antigenic region is strikingly close to the α-syn mutations that cause Parkinson's disease (A30P, E46K, H50Q, G51D, A53T). (Hernandez, D. G., Reed, X. & Singleton, A. B. Genetics in Parkinson disease: Mendelian versus non-Mendelian inheritance. J. Neurochem. 139, 59-74 (2016)). The second antigenic region encompasses S129 and requires 5129 phosphorylation, a form present in Lewy bodies (Fujiwara, H. et al. α-synuclein is phosphorylated in synucleinopathy lesions. Nat. Cell Biol. 4, 160-164 (2002)); antigenic epitopes from that region are not strongly restricted and can drive immune responses in patients who do not express HLA alleles that recognize the Y39 region.

Approximately 40% of the participants with Parkinson's disease in our cohort exhibited immune responses to α-syn epitopes, and responses may reflect variations in disease progression or environmental factors. The fraction of patients who display these responses in classic autoimmune disorders such as type-1 diabetes, rheumatoid arthritis and multiple sclerosis is often around 20-50% (Petrich de Marquesini, L. G. et al. IFN-γ and IL-10 islet-antigen-specific T cell responses in autoantibody-negative first-degree relatives of patients with type 1 diabetes. Diabetologia 53, 1451-1460 (2010); Arif, S. et al. Peripheral and islet interleukin-17 pathway activation characterizes human autoimmune diabetes and promotes cytokine-mediated)-cell death. Diabetes 60, 2112-2119 (2011)). As with type-1 diabetes, which features epitopes that are derived from both preproinsulin and additional proteins, it may be that epitopes related to Parkinson's disease are derived from α-syn and additional proteins. In classic autoimmune disorders, the MHC class II response may precede MHC class I, (Marrack, P. & Kappler, J. W. Do MHCII-presented neoantigens drive type 1 diabetes and other autoimmune diseases? Cold Spring Harb. Perspect. Med. 2, a007765 (2012)) and that exposing microglia to α-syn triggers MHC class I expression by dopamine neurons (Cebrián, C. et al. MHC-I expression renders catecholaminergic neurons susceptible to T-cell-mediated degeneration. Nat. Commun. 5, 3633 (2014)). The Parkinson's disease-associated proteins parkin and PINK1 may regulate antigenic presentation of mitochondrial peptides (Matheoud, D. et al. Parkinson's disease-related proteins PINK1 and Parkin repress mitochondrial antigen presentation. Cell 166, 314-327 (2016)) and it is possible that an autoimmune presentation of antigenic epitopes unites lysosomal and mitochondrial mechanisms of Parkinson's disease pathogenesis.

Methods

A) Study Subjects

All participants provided written informed consent for participation in the study. Ethical approval was obtained from the LJI and Columbia University. 67 participants with Parkinson's disease and 36 age-matched healthy controls from the greater San Diego (Parkinson's disease, n=9; healthy controls, n=13) and New York City (Parkinson's disease, n=58; healthy controls, n=23) areas were recruited. The New York cohort was recruited from the Center for Parkinson's Disease at Columbia University Medical Center through the Spot study (Alcalay, R. N. et al. Glucocerebrosidase activity in Parkinson's disease with and without GBA mutations. Brain 138, 2648-2658 (2015). Blood samples were collected by Dr. Sean Campbell and Suxiao Yang of the Columbia Center for Translational Immunology (CCTI) Human Studies Core and approved by the CUMC Institutional Review Board. Parkinson's disease was defined based on the UK Parkinson's Disease Brain Bank criteria, without excluding cases with a family history of Parkinson's disease (Hughes, A. J., Ben-Shlomo, Y., Daniel, S. E. & Lees, A. J. What features improve the accuracy of clinical diagnosis in Parkinson's disease: a clinicopathologic study. 1992. Neurology 57, S34-S38 (2001)). We collected demographics and disease characteristics including age, age of onset, sex, medications, comorbidities and motor disease severity as measured by the Unified Parkinson's Disease Rating Scale (UPDRS) motor score (UPDRS-III). We also collected family history of Parkinson's disease in first-degree relatives. The data are reported in Tables 11A and 11B. In the San Diego cohort, demographic data was recorded and Parkinson's disease was self-reported. Samples used for additional assays in FIG. 13 and FIG. 10 were collected from consecutive individuals based on the schedule of their appointment; the demographics and Parkinson's disease characteristics of these participants are shown in Tables 13 and 5. Healthy controls were recruited through a convenience sample of consecutive non-blood related individuals, and were mostly spouses of participants with Parkinson's disease. At Columbia University, Parkinson's disease and healthy controls were recruited only if there was no history of immune modulatory medications (for example, steroids) or overt autoimmune disorder (for example, lupus). No significant difference was detected in response rates as a function of sex or geographical location. Three participants with Parkinson's disease had a history of Crohn's disease and one patient had a history of Hashimoto's thyroiditis. Two of the three participants with Crohn's disease showed antigenic response to α-syn and the participant with Hashimoto's thyroiditis did not. Experimental blinding was accomplished by labelling the blood samples in a coded fashion without information on age/gender or Parkinson's disease status. The cohort was predominantly Caucasian (88.3%) and no firm conclusions between Crohn's disease and Parkinson's disease could be drawn because of the limited number of Crohn's disease patients studied.

B) Peptides

Peptides were synthesized as crude material on a small (1.mg) scale by A and A, LLC (San Diego). Peptides were forty 15-mers overlapping by 10-14 residues and seventy 9- or 10-mers predicted to bind common HLA class I alleles. In brief, each possible 9- and 10-mer from α-syn was scored for their capacity to bind a panel of 27 common HLA class I A and B molecules (Paul, S. et al. HLA class I alleles are associated with peptide-binding repertoires of different size, affinity, and immunogenicity. J. Immunol. 191, 5831-5839 (2013)). For each allele four peptides were synthesized (two 9-mers and two 10-mers, n=61 after removing redundant sequences that were selected for 2 or more alleles). In addition, any peptide that scored at the 2 percentile level or better for predicted binding, but were not within the four selected per allele were synthesized (n=9). Post-translationally modified peptides (n=7) were synthesized as purified material (>95% by reversed phase HPLC) by A and A, LLC (San Diego). Peptides were combined into pools of 14 peptides (range 11-16).

An alternative mode of stimulation is to use whole α-syn protein. However, synthetic peptides are preferred owing to their well-characterized and uniform chemical species, in contrast to α-syn preparations that contain varying amounts of different post-translational modifications, and as it is unclear which form(s) are processed by antigen presenting cells Parkinson's disease. In addition to a lower cost, synthetic peptides provide better mapping of specific epitopes and measurement of HLA binding.

C) PBMC Isolation and Culture

Venous blood was collected in heparin-containing blood bags or tubes. PBMCs were purified from whole blood by density-gradient centrifugation, according to the manufacturer's instructions. Cells were cryopreserved in liquid nitrogen suspended in FBS containing 10% (vol/vol) DMSO. Culturing of PBMCs for in vitro expansion was performed by incubating in RPMI (Omega Scientific) supplemented with 5% human AB serum (Gemini Bioscience), GlutaMAX (Gibco), and penicillin and streptomycin (Omega Scientific) at 2×10⁶ per ml in the presence of individual peptide pools at 5 μg ml⁻¹. Every three days, 10 U ml⁻¹ IL-2 in medium was added to the cultures.

D) ELISPOT Assays

After 14 days of culture with individual peptide pools (5 μg ml⁻¹), the response to pools and individual peptides (5 μg ml⁻¹) was measured by IFNγ and IL-5 dual ELISPOT (Oseroff, C. et al. Molecular determinants of T cell epitope recognition to the common Timothy grass allergen. J. Immunol. 185, 943-955 (2010)). ELISPOT antibodies, mouse anti-human IFNγ (clone 1-D1K), mouse anti-human IL-5 (clone TRFK5), mouse anti-human IFNγ-HRP (clone 7-B6-1), mouse anti-human IL-5 biotinylated (clone 5A10) were all from Mabtech. To be considered positive, a response had to match three criteria: (1) elicit at least 100 spot-forming cells (SFC) per 10⁶ PBMC; (2) P≤0.05 by Student's t-test or by a Poisson distribution test; (3) stimulation index≥2.

For the experiments with fibrilized or native α-syn, PBMCs were stimulated with epitopes derived from α-syn for 14 days. These cultures were then stimulated with α-syn peptides, 25 μg ml⁻¹ fibrilized α-syn or 25 μg ml⁻¹ native α-syn.

E) HLA Typing, Restriction, Binding Predictions and Assays

Participants were HLA-typed at the La Jolla Institute or by an American Society for Histocompatibility and Immunogenetics (ASHI)-accredited laboratory at Murdoch University (Western Australia). Typing at LJI was performed by next-generation sequencing (McKinney, D. M. et al. Development and validation of a sample sparing strategy for HLA typing utilizing next generation sequencing. Hum. Immunol. 76, 917-922 (2015)). Specifically, amplicons were generated from the appropriate class II locus for exons 2 through 4 by PCR amplification. From these amplicons, sequencing libraries were generated (Illumina Nextera XT) and sequenced with MiSeq Reagent Kit v3 as per the manufacturer's instructions (Illumina). Sequence reads were matched to HLA alleles and participant genotypes were assigned. HLA typing in Australia for class I (HLA A; B; C) and class II (DQA1; DQB1, DRB1 3,4,5; DPB1) was performed using locus-specific PCR amplification on genomic DNA. Primers used for amplification employed patient-specific barcoded primers. Amplified products were quantified and pooled by subject and up to 48 subjects were pooled. An unindexed (454 eight-lane runs) or indexed (8 indexed MiSeq runs) library was then quantified using Kappa universal QPCR library quantification kits. Sequencing was performed using either a Roche 454 FLX+ sequencer with titanium chemistry or an Illumina MiSeq using 2×300 paired-end chemistry. Reads were quality-filtered and passed through a proprietary allele-calling algorithm and analysis pipeline using the latest IMGT HLA allele database as a reference. The algorithm was developed by E. P. and S. M. and relies on periodically updated versions of the freely available international immunogenetics information system (http://www.imgt.org) and an ASHI-accredited HLA allele caller software pipeline, IIID HLA Analysis Suite (http://www.iiid.com.au/laboratory-testing/).

Potential HLA-epitope restrictions were inferred using the RATE program (Paul, S. et al. A population response analysis approach to assign class II HLA-epitope restrictions. J. Immunol. 194, 6164-6176 (2015)). HLA A*11:01 binding predictions were performed using the consensus prediction method publicly available through the Immune Epitope Database (IEDB) Analysis Resource (available at http://www.iedb.org) (Vita, R. et al. The immune epitope database (IEDB) 3.0. Nucleic Acids Res. 43, D405-D412 (2015)).

Classical competition assays to quantitatively measure peptide-binding affinities for HLA class I and II MHC molecules, based on inhibition of binding of high affinity radiolabelled peptides to purified MHC molecules, were performed as detailed elsewhere (Sidney, J. et al. Measurement of MHC/peptide interactions by gel filtration or monoclonal antibody capture. Current Protoc. Immunol. 18, 18.13 (2013)). In brief, 0.1-1 nM of radiolabelled peptide was co-incubated at room temperature or 37° C. with purified MHC in the presence of a cocktail of protease inhibitors (and, for class I, exogenous human α2-microglobulin). Following a two to four day incubation, MHC-bound radioactivity (c.p.m.) was determined by capturing MHC-peptide complexes on Lumitrac 600 plates (Greiner Bio-one, Frickenhausen, Germany) coated with either HLA DR (L243), DQ (HB180), DP (B7/21) or class I (W6/32) specific monoclonal antibodies. Bound c.p.m. was measured using the TopCount microscintillation counter (Packard Instrument Co.). The concentration of peptide yielding 50% inhibition of binding of the radiolabelled peptide was calculated. Under the conditions used, where [label]<[MHC] and IC₅₀≥[MHC], measured IC₅₀ values are reasonable approximations of true K_{d} (Cheng, Y. & Prusoff, W. H. Relationship between the inhibition constant (K1) and the concentration of inhibitor which causes 50 per cent inhibition (I50) of an enzymatic reaction. Biochem. Pharmacol. 22, 3099-3108 (1973); Gulukota, K., Sidney, J., Sette, A. & DeLisi, C. Two complementary methods for predicting peptides binding major histocompatibility complex molecules. J. Mol. Biol. 267, 1258-1267 (1997)). Each competitor peptide was tested at six different concentrations covering a 100,000-fold range, and in three or more independent experiments. As a positive control, the unlabelled version of the radiolabelled probe was also tested in each experiment. A threshold of 1,000 nM binding affinity is associated with immunogenicity of HLA class II T cell epitopes, and most epitopes bind in the 1-100 nm range, with affinities in the 1-10 nM considered to be of high affinity (Sidney, J. et al. Divergent motifs but overlapping binding repertoires of six HLA-DQ molecules frequently expressed in the worldwide human population. J. Immunol. 185, 4189-4198 (2010)).

F) Intracellular Cytokine Staining

After 14 days of culture, PBMCs were stimulated in the presence of 5 μg ml⁻¹ α-syn peptide pool for 2 h in complete RPMI medium at 37° C. with 5% CO₂. After 2 h, 2.5 μg ml⁻¹ each of BFA and monensin was added for an additional 4 h at 37° C. Unstimulated PBMCs were used to assess nonspecific/background cytokine production and PHA stimulation at 5 μg ml⁻¹ was used as a positive control. After a total of 6 h, cells were collected and stained for cell surface antigens CD4 (anti-CD4-APCeF780, RPA-T4, eBioscience), CD3 (anti-CD3-AF700, UCHT1, BD Pharmingen), CD8 (anti-CD8-BV650, RPA-T8, BioLegend), CD14 (anti-CD14-V500, M5E2, BD Pharmingen), CD19 (anti-CD19-V500, HIB19, BD Pharmingen), and fixable viability dye eFluor 506 (eBioscience). After washing, cells were fixed using 4% paraformaldehyde and permeabilized using saponin buffer. Cells were stained for IFNγ (anti-IFNγ-APC, 4S.B3, eBioscience), IL-17 (anti-IL-17-PECy7, eBio64DEC17, eBioscience), IL-4 (anti-IL-4-PE/Dazzle594, MP4-25D2, BioLegend), and IL-10 (anti-IL-10-AF488, JES3-907, eBioscience) in saponin buffer containing 10% FBS. Samples were acquired on a BD LSR II flow cytometer. Frequencies of CD3⁺ T cells responding to α-syn peptide pool were quantified by determining the total number of gated CD3⁺ and cytokine⁺ cells and background values were subtracted (as determined from the medium alone control) using FlowJo X Software (FlowJo). Combinations of cytokine producing cells were determined using Boolean gating.

G) HLA-DR and HLA-ABC Epression

PBMCs from DRB1*15:01⁺ or DRB1*15:01⁻ patients with Parkinson's disease (n=5 for both) and healthy controls (n=3 DRB1*15:01⁺ and n=5 DRB1*15:01⁻) were assessed for HLA-DR and HLA-ABC (as a control) expression. 721.221 and RM3 cells (both sourced from ATCC, mycoplasma free) were used as controls for HLA-DR and HLA-ABC expression. 721.221 cells lack HLA-ABC and express HLA-DR, whereas RM3 cells lack HLA-DR and express HLA-ABC. All cells were stained for cell-surface antigens CD14 (anti-CD14-APC, 61D3, Tonbo biosciences), CD3 (anti-CD3-AF700, UCHT1, BD Pharmingen), HLA-ABC (anti-HLA-ABC-AF488, W6/32; pan-HLA class I, BioLegend), HLA-DR (anti-HLA-DR-PE, L243; pan-HLA-DR, eBioscience), and fixable viability dye eFluor 506 (eBioscience) or isotype controls for HLA-ABC (AF488 mouse IgG2a, κ, catalogue number 400233, BioLegend) or HLA-DR (PE mouse IgG2a, κ, catalogue number 12-4724, eBioscience). After washing, cells were fixed using 4% paraformaldehyde. Samples were acquired on a BD LSR II flow cytometer. The fraction of living cells expressing HLA-ABC or HLA-DR was determined using FlowJo X Software.

H) α-syn Purification and α-syn PFF Preparation

The recombinant α-syn monomer was purified as previously described (Mao, X. et al. Pathological α-synuclein transmission initiated by binding lymphocyte-activation gene 3. Science 353, aah3374 (2016)). α-syn pre-formed fibrils (PFF) were prepared by agitating α-syn monomer in a transparent glass vial with a magnetic stirrer (350 r.p.m. at 37° C.). After 5-7 days of agitation, the clear α-syn monomer solution became turbid, indicative that α-syn fibrils were generated. The α-syn fibrils were then sonicated for 30 s at 10% amplitude to generate α-syn PFF (Branson Digital Sonifier). α-syn monomer and PFF were aliquoted and kept at −80° C.

Statistics and Reproducibility

A power analysis was not conducted a priori as there was no means to estimate effect size. Future validation studies will test whether the Y39 antigenic region is recognized significantly higher in patients with Parkinson's disease compared to healthy controls. The recognition frequency of this peptide was 17% in patients with Parkinson's disease and 3% in healthy controls, which achieves 61% power to detect a response difference between response rates of 14 percentage points. To achieve 80% power in a repeat study to detect a similar effect size, a total of 62 patients with Parkinson's disease and 62 healthy controls should be included. Additionally validation studies will test whether the overall recognition of the 11 peptides is significantly higher in patients with Parkinson's disease compared to healthy controls. On the basis of the combined cohort data, the recognition frequency of a pool of peptides was 37% in patients with Parkinson's disease and 8% in healthy controls. To obtain 80% power in a validation study a cohort size of 43 in both patients with Parkinson's disease and healthy controls will be required to detect the same effect.

The Fisher's exact (two-tailed) test was used to evaluate the contingency between carriers and non-carriers of the DRB1*15:01 and DRB5*01:01 alleles in the patients with Parkinson's disease and healthy control donors (Table 5), between the responses to phosphorylated S129 epitopes of patients with Parkinson's disease and healthy control donors (FIG. 6E-6G), and between DRB1*01/DRB5*01:01/DQB1*03:04/A*11:01 carriers and non-carriers in patients with Parkinson's disease and healthy controls (Table 14). A non-parametric test was used because the data are not normally distributed. A Fisher's exact test that provides exact P values for the analysis of contingency tables is available in most professional statistical analysis packages.

The Mann-Whitney test (two-tailed) was used to assess whether the number of SFC of healthy control donors would be less or greater than those of donors with Parkinson's disease (FIGS. 6B-6G, and FIG. 7). The Mann-Whitney U-test (two-tailed) was used to determine whether the number of IFNγ SFC was different from the number of IL-5 SFC in patients with Parkinson's disease (FIG. 8D). A non-parametric test was used because the data are not normally distributed. Student's t-tests were used to analyse parametric differences in demographics between patients with Parkinson's disease and healthy control donors (Tables 12A, 12B, and 13). The Wilcoxon signed-rank test was used to analyse differences in population means of the repeated measurements of number of SFC induced by medium and different isoforms of α-syn (FIG. 10). A non-parametric test was used because the data are not normally distributed. We hypothesized that responses to proteins and peptides would be higher than medium alone, therefore a one-tailed test was used for those comparisons. Comparison between PFF and native α-syn was two-tailed.

### Example 19

### Determining HLA and TCRs as Biomarkers

To develop diagnostic biomarkers for neurodegenerative disease, the following is performed:
a) Patients are HLA typed to identify those with an HLA capable of presenting certain disease associated epitopes (e.g. those HLA and epitopes identified using methods as described in Example 18).
b) in parallel cells from these patients expressing those HLA are expanded with the epitope in vitro, the and TCR is determined

Thus individuals are screened based on HLA plus TCR presence and identified as persons with the disease or at risk for it. In addition, this method can be used to determine TCRs that should be used in therapeutics.

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art in light of the present description that numerous modifications and variations can be made. The scope of the invention is defined more particularly in the appended claims

### REFERENCES

Alcalay, R. N. et al. Glucocerebrosidase activity in Parkinson's disease with and without GBA mutations. Brain 138, 2648-2658 (2015).
Anderson, J. P. et al. Phosphorylation of Ser-129 is the dominant pathological modification of alpha-synuclein in familial and sporadic Lewy body disease. J Biol Chem 281, 29739-29752 (2006).
Appel, S. H., Stockton-Appel, V., Stewart, S. S. & Kerman, R. H. Amyotrophic lateral sclerosis. Associated clinical disorders and immunological evaluations. Arch Neurol 43, 234-238 (1986).
Arai, T., et al. TDP-43 is a component of ubiquitin-positive tau-negative inclusions in frontotemporal lobar degeneration and amyotrophic lateral sclerosis. Biochem Biophys Res Commun 351, 602-611 (2006).
Arif, S. et al. Peripheral and islet interleukin-17 pathway activation characterizes human autoimmune diabetes and promotes cytokine-mediated beta-cell death. Diabetes 60, 2112-2119 (2011).
Arlehamn et al. (2014) Transcriptional profile of tuberculosis antigen-specific T cells reveals novel multifunctional features. Journal of immunology (Baltimore, Md : 1950) 193:2931-2940.
Arnold et al. (2013) Comparative survey of the topographical distribution of signature molecular lesions in major neurodegenerative diseases. J Comp Neurol 521:4339-4355.Pmc3872132.
Atik, A., Stewart, T. & Zhang, J. Alphα-Synuclein as a Biomarker for Parkinson's Disease. Brain Pathol 26, 410-418 (2016).
Bartos et al. (2012) Patients with Alzheimer disease have elevated intrathecal synthesis of antibodies against Tau protein and heavy neurofilament. J Neuroimmunol 252:100-105.
Benner, E. J. et al. Nitrated alpha-synuclein immunity accelerates degeneration of nigral dopaminergic neurons. PLoS ONE 3, e1376 (2008).
Blokhuis, A. M., Groen, E. J., Koppers, M., van den Berg, L. H. & Pasterkamp, R. J. Protein aggregation in amyotrophic lateral sclerosis. Acta Neuropathol 125, 777-794 (2013).
Brahmachari, S. et al. Activation of tyrosine kinase c-Ab1 contributes to alpha-synuclein-induced neurodegeneration. J Clin Invest 126, 2970-2988 (2016).
Brochard, V. et al. Infiltration of CD4+ lymphocytes into the brain contributes to neurodegeneration in a mouse model of Parkinson disease. J Clin Invest 119, 182-192 (2009).
Butovsky, O., et al. Modulating inflammatory monocytes with a unique microRNA gene signature ameliorates murine ALS. J Clin Invest 122, 3063-3087 (2012).
Cebrian, C. et al. MHC-I expression renders catecholaminergic neurons susceptible to T-cellmediated degeneration. Nat Commun 5, 3633 (2014).
Cebrian, C., Loike, J. D. & Sulzer, D. Neuroinflammation in Parkinson's disease animal models: a cell stress response or a step in neurodegeneration? Curr Top Behav Neurosci 22, 237-270 (2015).
Cecconi et al. (2010) The CD4+ T-cell epitope-binding register is a critical parameter when generating functional HLA-DR tetramers with promiscuous peptides. European Journal of Immunology 40:1603-1616.
Cheng, Y. & Prusoff, W. H. Relationship between the inhibition constant (K1) and the concentration of inhibitor which causes 50 per cent inhibition (I50) of an enzymatic reaction. Biochem Pharmacol 22, 3099-3108 (1973).
Chiu, I. M., et al. A neurodegeneration-specific gene-expression signature of acutely isolated microglia from an amyotrophic lateral sclerosis mouse model. Cell reports 4, 385-401 (2013).
Chiu, I. M., et al. Activation of innate and humoral immunity in the peripheral nervous system of ALS transgenic mice. Proc Natl Acad Sci U S A 106, 20960-20965 (2009).
Chiu, I. M., et al. T lymphocytes potentiate endogenous neuroprotective inflammation in a mouse model of ALS. Proc Natl Acad Sci U S A 105, 17913-17918 (2008).
Cuervo, A. M., et al. Autophagy and aging: the importance of maintaining “clean” cells. Autophagy 1, 131-140 (2005).
Cuervo, A. M., Stefanis, L., Fredenburg, R., Lansbury, P. T. & Sulzer, D. Impaired degradation of mutant alpha-synuclein by chaperone-mediated autophagy. Science 305, 1292-1295 (2004).
Dan et al. (2016) A Cytokine-Independent Approach To Identify Antigen-Specific Human Germinal Center T Follicular Helper Cells and Rare Antigen-Specific CD4+ T Cells in Blood. Journal of Immunology, 197:983-993.
Dufty, B. M. et al. Calpain-cleavage of alpha-synuclein: connecting proteolytic processing to disease-linked aggregation. Am J Pathol 170, 1725-1738 (2007).
Duka et al. (2013) Identification of the sites of Tau hyperphosphorylation and activation of Tau kinases in synucleinopathies and Alzheimer's diseases. PLoS One 8:e75025.Pmc3779212.
Edstrom, E., Kullberg, S., Ming, Y., Zheng, H. & Ulfhake, B. MHC class I, beta2 microglobulin, and the INF-gamma receptor are upregulated in aged motoneurons. J Neurosci Res 78, 892-900 (2004).
Foix, C. & Nicolesco, J. Cérébrale: Les Noyauz Gris Centraux Et La Région Mésencephalo-Soue-Optique. SuiviD'Un Appendice Sur L'Anatomic Pathologique De La Maladie De Parkinson. (Masson et Cie., 1925).
Foulds et al. (2013) A longitudinal study on alpha-synuclein in blood plasma as a biomarker for Parkinson's disease. Scientific reports 3:2540.Pmc3756331.
Fujiwara, H. et al. alphα-Synuclein is phosphorylated in synucleinopathy lesions. Nature cell biology 4, 160-164 (2002).
Ghidoni et al. (2006) The H2 MAPT haplotype is associated with familial frontotemporal dementia. Neurobiology of Disease 22:357-362.
Glanville et al. (2017) Identifying specificity groups in the T cell receptor repertoire. Nature 547:94-98.
Greenbaum, J. et al. Functional classification of class II human leukocyte antigen (HLA) molecules reveals seven different supertypes and a surprising degree of repertoire sharing across supertypes. Immunogenetics 63, 325-335 (2011).
Gulukota, K., Sidney, J., Sette, A. & DeLisi, C. Two complementary methods for predicting peptides binding major histocompatibility complex molecules. J Mol Biol 267, 1258-1267 (1997).
Hampel et al. (2010) Total and phosphorylated Tau protein as biological markers of Alzheimer's disease. Experimental gerontology 45:30-40.Pmc2815003.
Hamza, T. H. et al. Common genetic variation in the HLA region is associated with late-onset sporadic Parkinson's disease. Nat Genet 42, 781-785 (2010).
Harms, A. S. et al. MHCII is required for alpha-synuclein-induced activation of microglia, CD4 T cell proliferation, and dopaminergic neurodegeneration. J Neurosci 33, 9592-9600 (2013).
Hernandez, D. G., Reed, X. & Singleton, A. B. Genetics in Parkinson disease: Mendelian versus non-Mendelian inheritance. J Neurochem (2016).
Hossain, S. et al. Limited proteolysis of NACP/alpha-synuclein. Alzheimers Dis 3, 577-584 (2001).
Hughes, A. J., Ben-Shlomo, Y., Daniel, S. E. & Lees, A. J. What features improve the accuracy of clinical diagnosis in Parkinson's disease: a clinicopathologic study. 1992. Neurology 57, S34-38 (2001).
Kametani, F., et al. Mass spectrometric analysis of accumulated TDP-43 in amyotrophic lateral sclerosis brains. Scientific reports 6, 23281 (2016).
Kannarkat, G. T. et al. Common Genetic Variant Association with Altered HLA Expression, Synergy with Pyrethroid Exposure, and Risk for Parkinson's Disease: An Observational and Case-Control Study. NPJ Parkinson's disease 1 (2015).
Kawamata, T., Akiyama, H., Yamada, T. & McGeer, P. L. Immunologic reactions in amyotrophic lateral sclerosis brain and spinal cord tissue. Am J Pathol 140, 691-707 (1992).
Kasai, T. et al. Cleavage of normal and pathological forms of alpha-synuclein by neurosin in vitro. Neurosci Lett 436, 52-56 (2008).
Kim et al. (2014) Dataset size and composition impact the reliability of performance benchmarks for peptide-MHC binding predictions. BMC bioinformatics 15:241.
Kim, K. S. et al. Proteolytic cleavage of extracellular alpha-synuclein by plasmin: implications for Parkinson disease. J Biol Chem 287, 24862-24872 (2012).
Kim et al. (2012) Immune epitope database analysis resource. Nucleic Acids Research 40:W525-530.
Klinger et al. (2015) Multiplex Identification of Antigen-Specific T Cell Receptors Using a Combination of Immune Assays and Immune Receptor Sequencing. PloS one 10:e0141561.
Koehler et al. (2013) Altered serum IgG levels to alpha-synuclein in dementia with Lewy bodies and Alzheimer's disease. PLoS One 8:e64649.Pmc3669378.
Kramer et al. (2014) Causal analysis approaches in Ingenuity Pathway Analysis. Bioinformatics 30:523-530.Langfelder and Horvath (2008) WGCNA: an R package for weighted correlation network analysis. BMC Bioinformatics 9:559.
Li, W. et al. Aggregation promoting C-terminal truncation of alpha-synuclein is a normal cellular process and is enhanced by the familial Parkinson's disease-linked mutations. Proc Natl Acad Sci U S A 102, 2162-2167 (2005).
Linda, H., Hammarberg, H., Piehl, F., Khademi, M. & Olsson, T. Expression of MHC class I heavy chain and beta2-microglobulin in rat brainstem motoneurons and nigral dopaminergic neurons. J Neuroimmunol 101, 76-86 (1999).
Ling, S. C., Polymenidou, M. & Cleveland, D. W. Converging mechanisms in ALS and FTD: disrupted RNA and protein homeostasis. Neuron 79, 416-438 (2013).
Luk, K. C. et al. Pathological alpha-synuclein transmission initiates Parkinson-like neurodegeneration in nontransgenic mice. Science 338, 949-953 (2012):
Mao, X. et al. Pathological alpha-synuclein transmission initiated by binding lymphocyteactivation gene 3. Science 353 (2016).
Marrack, P. & Kappler, J. W. Do MHCII-presented neoantigens drive type 1 diabetes and other autoimmune diseases? Cold Spring Harbor perspectives in medicine 2, a007765 (2012).
Martinez-Vicente, M. et al. Dopamine-modified alpha-synuclein blocks chaperone-mediated autophagy. J Clin Invest 118, 777-788 (2008).
Matheoud, D. et al. Parkinson's Disease-Related Proteins PINK1 and Parkin Repress Mitochondrial Antigen Presentation. Cell 166, 314-327 (2016).
McGeer, P. L., et al. Microglia in degenerative neurological disease. Glia 7, 84-92 (1993).

McKinney, D. M. et al. Development and validation of a sample sparing strategy for HLA typing utilizing next generation sequencing. Hum Immunol (2015).
McKinney et al. (2013) A strategy to determine HLA class II restriction broadly covering the DR, DP, and DQ allelic variants most commonly expressed in the general population. Immunogenetics 65:357-370.
Mootha VK et al. (2003) PGC-lalpha-responsive genes involved in oxidative phosphorylation are coordinately downregulated in human diabetes. Nature Genetics 34:267-273.
Mor, F., Quintana, F., Mimran, A. & Cohen, I. R. Autoimmune encephalomyelitis and uveitis induced by T cell immunity to self betα-synuclein. J Immunol 170, 628-634 (2003).
Nardo, G., Trolese, M. C. & Bendotti, C. Major Histocompatibility Complex I Expression by Motor Neurons and Its Implication in Amyotrophic Lateral Sclerosis. Frontiers in neurology 7, 89 (2016).
Neumann, M., et al. Ubiquitinated TDP-43 in frontotemporal lobar degeneration and amyotrophic lateral sclerosis. Science 314, 130-133 (2006).
Orenstein, S. J. et al. Interplay of LRRK2 with chaperone-mediated autophagy. Nat Neurosci 16, 394-406 (2013).
Oseroff, C. et al. Molecular determinants of T cell epitope recognition to the common Timothy grass allergen. J Immunol 185, 943-955 (2010).
Paul et al. (2013) HLA class I alleles are associated with peptide-binding repertoires of different size, affinity, and immunogenicity. Journal of Immunology 191:5831-5839.
Paul et al. (2015) A population response analysis approach to assign class II HLA-epitope restrictions. Journal of Immunology 194:6164-176.
Petrich de Marquesini, L. G. et al. IFN-gamma and IL-10 islet-antigen-specific T cell responses in autoantibody-negative first-degree relatives of patients with type 1 diabetes. Diabetologia 53, 1451-1460 (2010).
Sharma M et al. (2012) Large-scale replication and heterogeneity in Parkinson disease genetic loci. Neurology 79:659-667.Pmc3414661.
Sidney, J. et al. Measurement of MHC/peptide interactions by gel filtration or monoclonal antibody capture. Current protocols in immunology Chapter 18, Unit 18.13, (2013).
Sidney, J. et al. Divergent motifs but overlapping binding repertoires of six HLA-DQ molecules frequently expressed in the worldwide human population. J Immunol 185, 4189-4198 (2010).
Spillantini, M. G., Crowther, R. A., Jakes, R., Hasegawa, M. & Goedert, M. Alphα-synuclein in filamentous inclusions of Lewy bodies from Parkinson's disease and dementia with Lewy bodies. Proc. Natl. Acad. Sci. USA 95, 6469-6473 (1998).
Smith, B. N., et al. Mutations in the vesicular trafficking protein annexin All are associated with amyotrophic lateral sclerosis. Science translational medicine 9(2017).
Subramanian et al. (2005) Gene set enrichment analysis: a knowledge-based approach for interpreting genome-wide expression profiles. Proceedings of the National Academy of Sciences of the United States of America 102:15545-15550.
Sulzer, D., et al. T cells of Parkinson's disease patients recognize alpha-synuclein peptides. Nature in press(2017).
Tacik et al. (2015) Genetic Disorders with Tau Pathology: A Review of the Literature and Report of Two Patients with Tauopathy and Positive Family Histories. Neuro-degenerative Diseases 16:12-21.
Thams, S., et al. Classical major histocompatibility complex class I molecules in motoneurons: new actors at the neuromuscular junction. J Neurosci 29, 13503-13515 (2009).
Theodore, S., Cao, S., McLean, P. J. & Standaert, D. G. Targeted overexpression of human alphasynuclein triggers microglial activation and an adaptive immune response in a mouse model of Parkinson disease. J Neuropathol Exp Neurol 67, 1149-1158 (2008).
Vita, R. et al. The immune epitope database (IEDB) 3.0. Nucleic Acids Res 43, D405-412 (2015).
Wang, W. et al. Caspase-1 causes truncation and aggregation of the Parkinson's diseaseassociated protein alpha-synuclein. Proc Nati Acad Sci U S A 113, 9587-9592 (2016).
Wang et al. (2009) Tau fragmentation, aggregation and clearance: the dual role of lysosomal processing. Hum Mol Genet 18:4153-4170.2758146.
Weiskopf et al. (2013) Comprehensive analysis of dengue virus- specific responses supports an HLA-linked protective role for CD8+ T, cells. Proceedings of the National Academy of Sciences of the United States of America 110:E2046-2053.
Wissemann, W. T. et al. Association of Parkinson disease with structural and regulatory variants in the HLA region. Am J Hum Genet 93, 984-993 (2013).
Yin et al. (2013) Recognition of self and altered self by T cells in autoimmunity and allergy. Protein Cell 4:8-16.Pmc3951410.
Zetterberg et al. (2013) Plasma Tau levels in Alzheimer's disease. Alzheimer's research & therapy 5:9.Pmc3707015.
Zhao, W., et al. Characterization of Gene Expression Phenotype in Amyotrophic Lateral Sclerosis Monocytes. JAMA neurology (2017).

1. A method for assessing whether:
A) a subject is at risk of developing, or for diagnosing or confirming whether a subject is afflicted with an α-synucleinopathy, a Tauopathy, Parkinson's disease (PD), amyotrophic lateral sclerosis (ALS), Lewy Body dementia (LBD), or Alzheimer's disease (AD);
B) a subject afflicted with an α-synucleinopathy, a Tauopathy, Parkinson's disease (PD), amyotrophic lateral sclerosis (ALS), Lewy Body dementia (LBD), or Alzheimer's disease (AD) is likely to benefit from a therapy or has benefitted from a therapy, wherein the therapy is directed to leukocytes that are activated by an epitope peptide;
C) leukocytes of a subject afflicted with an α-synucleinopathy, a Tauopathy, Parkinson's disease (PD), amyotrophic lateral sclerosis (ALS), Lewy Body dementia (LBD), or Alzheimer's disease (AD) are activated by an epitope peptide; or
D) a test compound comprises an epitope peptide to which leukocytes of a subject suffering from a neurological disorder are responsive, comprising
a)
i) obtaining leukocytes from the subject;
ii). contacting the leukocytes with an epitope peptide;
iii) determining whether the leukocytes have increased activation after contact with the epitope peptide; and
iv) A) if the method is for assessing whether a subject is at risk of developing, or for diagnosing or confirming whether a subject is afflicted with an α-synucleinopathy, a Tauopathy, Parkinson's disease (PD), amyotrophic lateral sclerosis (ALS), Lewy Body dementia (LBD), or Alzheimer's disease (AD), identifying the subject as at risk of developing, or as afflicted with the α-synucleinopathy, PD, ALS, LBD or AD if in step iii) the leukocytes are determined to have increased activation after contact with the epitope peptide, and identifying the subject as not at risk of developing, or as not afflicted. with the α-synucleinopathy, PD, ALS, LBD or AD if in step iii) the leukocytes are determined to not have increased activation after contact with the epitope peptide;
B) if the method is assessing whether a subject afflicted with an α-synucleinopathy, a Tauopathy, Parkinson's disease (PD), amyotrophic lateral sclerosis (ALS), Lewy Body dementia (LBD), or Alzheimer's disease (AD) is likely to benefit from a therapy or has benefitted from a therapy, wherein the therapy is directed to leukocytes that are activated by an epitope peptide, identifying the subject as likely to benefit from the therapy if in step iii) the leukocytes are determined to have increased activation after contact with the epitope peptide, and identifying the subject as unlikely to benefit from the therapy if in step iii) the leukocytes are determined to not have increased activation after contact with the epitope peptide;
C) if the method is assessing whether leukocytes of a subject afflicted with an α-synucleinopathy, a Tauopathy, Parkinson's disease (PD), amyotrophic lateral sclerosis (ALS), Lewy Body dementia (LBD), or Alzheimer's disease (AD) are activated by an epitope peptide, identifying the leukocytes of the subject as activated by the epitope peptide if in step iii) the leukocytes are determined to have increased activation after contact with the epitope peptide, and identifying the leukocytes of the subject as not activated by the epitope peptide if in step iii) the leukocytes are determined to not have increased activation after contact with the epitope peptide; or
D) if the method is for assessing whether a test compound comprises an epitope peptide to which leukocytes of a subject suffering from a neurological disorder are responsive, identifying the test compound as comprising an epitope peptide to which the leukocytes are responsive if in step iii) the leukocytes are determined to have increased activation after contact with the test compound, and identifying the test compound as not comprising an epitope to which the leukocytes are responsive if in step iii) the leukocytes are determined to not have increased activation after contact with the test compound, or
b)
i) obtaining leukocytes from the subject;
ii) separating the leukocytes into 2 or more pools of leukocytes and contacting each pool with an epitope peptide, wherein each pool is contacted with a different epitope;
iii) determining whether each pool has increased activation after contact with the epitope peptide; and
iv) A) if the method is for assessing whether a subject is at risk of developing, or for diagnosing or confirming whether a subject is afflicted with an α-synucleinopathy, a Tauopathy, Parkinson's disease (PD), amyotrophic lateral sclerosis (ALS), Lewy Body dementia (LBD), or Alzheimer's disease (AD), identifying the subject as at risk of developing, or as afflicted with the α-synucleinopathy, Tauopathy, PD, ALS, LBD, or AD if and only if in step iii) 1 or more pools is determined to have increased activation after contact with the epitope peptide;
B) if the method is assessing whether a subject afflicted with an α-synucleinopathy, a Tauopathy, Parkinson's disease (PD), amyotrophic lateral sclerosis (ALS), Lewy Body dementia (LBD), or Alzheimer's disease (AD) is likely to benefit from a therapy or has benefitted from a therapy, identifying the subject as having benefited from therapy if in step iii) 1 or more pools is determined to have increased activation after contact with the epitope peptide, and identifying the subject as not having benefitted from the therapy if in step iii) the leukocytes are determined to not have increased activation after contact with the epitope peptide;
C) if the method is assessing whether leukocytes of a subject afflicted with an α-synucleinopathy, a Tauopathy, Parkinson's disease (PD), amyotrophic lateral sclerosis (ALS), Lewy Body dementia (LBD), or Alzheimer's disease (AD) are activated by an epitope peptide, identifying the leukocytes of the subject as activated by the epitope peptide if in step iii) 1 or more pools of leukocytes are determined to have increased activation after contact with the epitope peptide, and identifying the leukocytes of the subject as not activated by the epitope peptide if in step iii) the leukocytes are determined to not have increased activation after contact with the epitope peptide; or
D) if the method is for assessing whether a test compound comprises an epitope peptide to which leukocytes of a subject suffering from a neurological disorder are responsive, identifying the test compound as comprising an epitope peptide to which the leukocytes are responsive if in step iii) 1 or more pools of leukocytes are determined to have increased activation after contact with the test compound, and identifying the test compound as not comprising an epitope to which the leukocytes are responsive if in step iii) the leukocytes are determined to not have increased activation after contact with the test compound.

2. A method for assessing whether an α-synucleinopathy, a Tauopathy, Parkinson's disease (PD), amyotrophic lateral sclerosis (ALS), Lewy Body dementia (LBD), or Alzheimer's disease (AD) has progressed or is developing in a subject afflicted with or who has been identified as being at risk of developing the α-synucleinopathy, PD, ALS, LBD or AD comprising
a) performing each of the following steps i) to iiiv):
i) obtaining leukocytes from the subject;
ii) contacting the leukocytes with an epitope peptide that was previously identified to increase activation of the leukocytes; and
iii) determining the level of activation of the leukocytes after contact with the epitope peptide at a first and a second point in time, and then
iv) concluding that the α-synucleinopathy, PD, ALS, LBD or AD has progressed or is developing in the subject if the leukocytes are determined to be more activated in step iii) performed at the second point in time compared to the level of activation in step iii) performed at the first point in time, or
b) performing each of the following steps i) to iiiv):
i) obtaining leukocytes from the subject;
ii) separating the leukocytes into two or more pools of leukocytes and contacting each pool with an epitope peptide, wherein each pool is contacted with a different epitope;
iii) determining whether each pool has increased activation after contact with the epitope peptide at a first and a second point in time, and then
iv) concluding that the α-synucleinopathy, PD, ALS, LBD or AD has progressed or is developing in the subject if more pools of leukocytes are determined to be activated in step iii) performed at the second point in time compared to the number of pools that are determined to be activated in step iii) performed at the first point in time.

3. (canceled)

4. A method for assessing whether a subject afflicted with a disease or condition involving an inflammatory response or related to inflammation, or a neurodegenerative disease or disorder is likely to benefit or has benefitted from a therapy, wherein the therapy comprises administration of an effective amount of a T cell receptor for a particular antigen:MHC complex, the method comprising:
a)
(i) obtaining leukocytes from the subject;
(ii) contacting the leukocytes with the antigen bound to an MHC molecule;
(iii) determining whether the leukocytes have increased activation after contact with the antigen bound to an MHC molecule; and
(iv) identifying the subject as likely to benefit from the therapy if in step (iii) the leukocytes are determined to have increased activation after contact with the antigen bound to an MHC molecule, and identifying the subject as unlikely to benefit from the therapy if in step (iii) the leukocytes are determined to not have increased activation after contact with the antigen bound to an MHC molecule; or
b)
(i) obtaining leukocytes from the subject;
(ii) contacting the leukocytes with the antigen bound to an MHC molecule;
(iii) determining whether the leukocytes have increased activation after contact with the antigen bound to an MHC molecule; and
(iv) identifying the subject as having benefited from the therapy if in step (iii) the leukocytes are determined to have increased activation after contact with the antigen bound to an MHC molecule, and identifying the subject as not having benefitted from the therapy if in step (iii) the leukocytes are determined to not have increased activation after contact with the antigen bound to an MHC molecule.

5-6. (canceled)

7. The method of claim 1, wherein the subject
a) is at least about 35, 40, 45, 50, 55, 60, 65, 70, 75 or 80 years of age;
b) is less than about 35, 40, 45, 50, 55, 60, 65, 70, 75 or 80 years of age;
c) has a symptom that has preceded the onset of the α-synucleinopathy, Tauopathy, PD, ALS, LBD or AD in subjects who have developed α-synucleinopathy, Tauopathy, PD, ALS, LBD or AD;
d) has a symptom that has preceded the onset of the α-synucleinopathy, Tauopathy, PD, ALS, LBD or AD in subjects who have developed the α-synucleinopathy, Tauopathy, PD, ALS, LBD or AD, wherein the symptom has preceded the onset of the α-synucleinopathy, Tauopathy, PD, ALS, LBD or AD in the subjects by at least about 5, 10, 15, 20, 25, 30 or 5-30 years;
e) is afflicted with cognitive decline, constipation or orthostatic hypotension
f) is afflicted with cognitive decline, and the cognitive decline is reduced spatial reasoning ability and/or reduced memory ability.
g) is afflicted with fasciculations or muscle twitches in the arm leg, shoulder, or tongue, muscle cramps, spasticity or tight and stiff muscles, muscle weakness affecting an arm, a leg, neck or diaphragm, slurred and nasal speech, and/or difficulty chewing or swallowing; or
h) is afflicted with cognitive decline, and the cognitive decline is reduced language or decision-making.

8. The method of claim 1, further comprising directing the subject to
a) be monitored more frequently for the α-synucleinopathy, Tauopathy, PD, ALS, LBD or AD; or
b) receive additional diagnostic testing for the α-synucleinopathy, Tauopathy, PD, ALS, LBD or AD,
if the subject is identified as at risk of developing the α-synucleinopathy, Tauopathy, PD, ALS, LBD or AD.

9. The method of claim 1, further comprising determining the presence of at least one human leukocyte antigen (HLA) allele, one T cell receptor (TCR) allele, or one MAPT allele in the subject.

10-13. (canceled)

14. A method for treating a subject afflicted with an α-synucleinopathy, a Tauopathy, Parkinson's disease (PD), amyotrophic lateral sclerosis (ALS), Lewy Body dementia (LBD), or Alzheimer's disease (AD)comprising
a) administering to the subject a compound that is approved for use in treating subjects afflicted with the α-synucleinopathy, PD, ALS, LBD or AD, wherein the subject has been diagnosed or confirmed to be afflicted with α-synucleinopathy, PD, ALS, LBD or AD according to the method of claim 1;
b) diagnosing or confirming the subject to be afflicted with the α-synucleinopathy, PD, ALS, LBD or AD according to the method of claim 1, and administering to the subject a compound that is approved for use in treating subjects afflicted with α-synucleinopathy, PD, ALS, LBD or AD;
c) administering to the subject a therapy that is directed to leukocytes that are activated by an epitope peptide, wherein leukocytes of the subject have been determined to have increased activation after contact with the epitope peptide;
d) administering an immunosuppressant therapy to the subject, wherein the subject has been identified as being likely to benefit therefrom by the method of claim 1; or
e) administering an immunosuppressant therapy to the subject, wherein the subject has been identified as being likely to benefit from a therapy directed to leukocytes that are activated by an epitope peptide according to the method of claim 1.

15-19. (canceled)

20. The method of claim 1, wherein the epitope peptide:
a) is or comprises part of a compound that is produced by neurons in subjects afflicted with the α-synucleinopathy, PD, ALS, LBD or AD;
b) comprises consecutive amino acids that are identical to a stretch of consecutive amino acids in a protein that is produced by the neurons;
c) comprises consecutive amino acids that are identical to a stretch of consecutive amino acids in a Tau mutant;
d) comprises about 16, at least 15, 5-50, 8-11, or 8-14 amino acids;
e) is phosphorylated, acetylated, nitrated, or dopamine modified;
f) comprises a phosphorylated serine or a phosphorylated tyrosine;
g) comprises a phosphorylated serine or a phosphorylated tyrosine, wherein the phosphorylated serine or phosphorylated tyrosine is within a stretch of consecutive amino acids that is identical to a stretch of consecutive amino acids comprising the serine at position 199, 202, 214, 262, 356, or 422 of Tau or the tyrosine at position 181, 205, 212, 231, or 262 of Tau.
h) is or comprises part of a compound that is produced by neurons in subjects afflicted with the α-synucleinopathy, PD, ALS, LBD or AD, wherein the neurons are in the ventral midbrain, the substantia nigra, the locus coeruleus, or the ventral tegmental area;
i) is or comprises part of a compound that is produced by neurons in subjects afflicted with the α-synucleinopathy, PD, ALS, LBD or AD, wherein the neurons are catecholamine neurons;
j) comprises consecutive amino acids that are identical to a stretch of consecutive amino acids in an α-syn mutant;
k) comprises consecutive amino acids that are identical to a stretch of consecutive amino acids in an α-syn mutant, wherein the α-syn mutant is an α-syn A53T or A30P mutant;
l) comprises a phosphorylated serine or a phosphorylated tyrosine, wherein the phosphorylated serine or phosphorylated tyrosine is within a stretch of consecutive amino acids that is identical to a stretch of consecutive amino acids comprising the serine at position 129 of α-syn or the tyrosine at position 39 of α-syn;
m) is or comprises part of a compound that is produced by neurons in ‘subjects afflicted with the ALS, wherein the neurons are in the motor area;
n) is or comprises part of a compound that is produced by neurons in subjects afflicted with ALS, wherein the neurons are motor neurons;
o) comprises consecutive amino acids that are identical to a stretch of consecutive amino acids in TDP43, FUS, or SOD-1;
p) comprises consecutive amino acids that are identical to a stretch of consecutive amino acids in TDP43 mutant, FUS mutant, or SOD-1 mutant;
q) comprises a deamidated asparagine, an oxidized threonine, or a phosphorylated tyrosine.

21. (canceled)

22. The method of claim 1, wherein in step iii) the leukocytes are determined to have increased activation after contact with the epitope peptide,
a) if the leukocytes express or release more of at least one cytokine compared to corresponding leukocytes not contacted with the epitope peptide;
b) if the leukocytes release at least one cytokine;
c) if the leukocytes release at least one cytokine, wherein in step iii) the leukocytes are determined to have released the at least one cytokine if there are over 20 spot-forming cells (SFC) per million cells as measured by an ELISpot assay comprising the colorimetric detection of the at least one cytokine.

23-27. (canceled)

28. The method of claim 1, wherein the test compound is or comprises part of a compound that is produced by neurons in subjects afflicted with an α-synucleinopathy, a Tauopathy, Parkinson's disease (PD), amyotrophic lateral sclerosis (ALS), Lewy Body dementia (LBD), or Alzheimer's disease (AD).

29-30. (canceled)

31. A kit comprising an epitope peptide as in claim 20.

32. A compound for treating an α-synucleinopathy, a Tauopathy, Parkinson's disease (PD), amyotrophic lateral sclerosis (ALS), Lewy Body dementia (LBD), or Alzheimer's disease (AD), comprising i) a major histocompatibility complex (MHC) Tetramer having four MHC molecules, wherein each MHC molecule is associated with an epitope peptide, and ii) a toxin, wherein
a. the epitope peptides is represented by an amino acid sequence selected from the group of Tau derived sequences represented by SEQ ID NO: 1-55 or 240-376,
b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or,
c. wherein the epitope peptide is represented by the amino acid sequence selected from the group of TDP43 derived sequences represented by SEQ ID NO: 56-239.

33. In a process for assessing whether a subject is at risk of developing an α-synucleinopathy, a Tauopathy, Parkinson's disease (PD), amyotrophic lateral sclerosis (ALS), Lewy Body dementia (LBD), or Alzheimer's disease (AD), which involves an array of testing, the improvement comprising including in the array of testing the steps of:
a)
i) obtaining leukocytes from the subject;
ii) contacting the leukocytes with an epitope peptide;
iii) determining whether the leukocytes have increased activation after contact with the epitope peptide; and
iv) identifying the subject as at risk of developing α-synucleinopathy, PD, ALS, LBD or AD if in step iii) the leukocytes are determined to have increased activation after contact with the epitope peptide, and identifying the subject as not at risk of developing the α-synucleinopathy, PD, ALS, LBD or AD if in step iii) the leukocytes are determined to not have increased activation after contact with the epitope peptide, wherein
a. the epitope peptides is represented by an amino acid sequence selected from the group of Tau derived sequences represented by SEQ ID NO: 1-55 or 240-376,
b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or.
c. wherein the epitope peptide is represented by the amino acid sequence selected from the group of TDP43 derived sequences represented by SEQ ID NO:
56-239 or
b)
i) obtaining leukocytes from the subject;
ii) separating the leukocytes into 2 or more pools of leukocytes and contacting each pool with an epitope peptide, wherein each pool is contacted with a different epitope;
iii) determining whether each pool has increased activation after contact with the epitope peptide; and
iv) identifying the subject as at risk of developing the α-synucleinopathy, PD, ALS, LBD or AD if in step iii) 1 or more pools is determined to have increased activation after contact with the epitope peptide, wherein
a. the epitope peptides is represented by an amino acid sequence selected from the group of Tau derived sequences represented by SEQ ID NO: 1-55 or 240-376,
b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or[[.]]
c. wherein the epitope peptide is represented by the amino acid sequence selected from the group of TDP43 derived sequences represented by SEQ ID NO: 56-239.

34. In a process for diagnosing or confirming whether a subject is afflicted with an α-synucleinopathy, a Tauopathy, Parkinson's disease (PD), amyotrophic lateral sclerosis (ALS), Lewy Body dementia (LBD), or Alzheimer's disease (AD), which involves an array of testing, the improvement comprising including in the array of testing the steps of:
a)
i) obtaining leukocytes from the subject;
ii) separating the leukocytes into 1 or more pools of leukocytes and contacting each pool with an epitope peptide, wherein each pool is contacted with a different epitope peptide;
iii) determining whether each pool has increased activation after contact with the epitope peptide; and
iv) identifying the subject as afflicted with the α-synucleinopathy, PD, ALS, LBD or AD if and only if in step
iii) 1 or more pools is determined to have increased activation after contact with an epitope peptide, or
b)
i) obtaining leukocytes from the subject;
ii) separating the leukocytes into 1 or more pools of leukocytes and contacting each pool with an epitope peptide, wherein each pool is contacted with a different epitope peptide;
iii) determining whether each pool has increased activation after contact with the epitope peptide; and
iv) identifying the subject as afflicted with the α-synucleinopathy, PD, ALS, LBD or AD if and only if in step iii) 1 or more pools is determined to have increased activation after contact with an epitope peptide, wherein
a. the epitope peptides is represented by an amino acid sequence selected from the group of Tau derived sequences represented by SEQ ID NO: 1-55 or 240-376,
b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or
c. wherein the epitope peptide is represented by the amino acid sequence selected from the group of TDP43 derived sequences represented by SEQ ID NO: 56-239.

35. (canceled)

36. A pharmaceutical composition for treating an α-synucleinopathy, a Tauopathy, Parkinson's disease (PD), amyotrophic lateral sclerosis (ALS), Lewy Body dementia (LBD), or Alzheimer's disease (AD), comprising
i) a protein comprising an amino acid sequence selected from the group of
a. the epitope peptides is represented by an amino acid sequence selected from the group of Tau derived sequences represented by SEQ ID NO: 1-55 or 240-376,
b. wherein the epitope peptide is represented by the amino acid sequence selected from the group of α-synuclein derived sequences GKTKEGVLYVGSKTK (SEQ ID NO: 487), KTKEGVLYVGSKTKE (SEQ ID NO: 488), MPVDPDNEAYEMPSE (SEQ ID NO: 489), DNEAYEMPSEEGYQD (SEQ ID NO: 490), EMPSEEGYQDYEPEA (SEQ ID NO: 491), SEEGYQDYEPEA (SEQ ID NO: 492), GVLYVGSKTK (SEQ ID NO: 493), VLYVGSKTK (SEQ ID NO: 494), or VLYVGSKTKK (SEQ ID NO: 495), or.
c. wherein the epitope peptide is represented by the amino acid sequence selected from the group of TDP43 derived sequences represented by SEQ ID NO: 56-239, and
ii) a pharmaceutically acceptable carrier.

37. (canceled)

38. A method comprising:
a. providing a biological sample from a subject;
b. processing the biological sample to determine presence of a T cell receptor (TCR) specific to a peptide, wherein the peptide is a fragment from a protein associated with said neurodegenerative disease.

39. The method of claim 38, wherein the processing step includes:
a) contacting T cells from said sample with said peptide, and detecting activation of a T cell having said TCR or
b) performing gene sequencing on at least a cellular fraction of said biological sample to amplify a gene encoding the TCR specific to said peptide, and detecting presence of said gene encoding said TCR, preferably wherein said at least a cellular fraction of said biological sample includes peripheral blood mononuclear cells (PBMC), preferably leukocytes.

40-42. (canceled)

43. A method comprising:
a) providing a biological sample from a subject;
b) processing the biological sample to determine presence of a human leukocyte antigen (HLA) capable of presenting a peptide, wherein the peptide is a fragment from a protein associated with said neurodegenerative disease; and
c) processing the biological sample to determine presence of a T cell receptor (TCR) specific to said peptide.

44. The method of claim 43, wherein the peptide is a fragment from a protein that forms aggregates in a patient having the neurodegenerative disease.

45. The method of claim 44, wherein:
a) step c) includes contacting T cells present in said sample with said peptide, and detecting activation of a T cell having said TCR;
b) step b) includes performing gene sequencing on at least a cellular fraction of said biological sample to amplify a gene encoding the HLA capable of presenting said peptide, and detecting presence of said gene encoding said HLA and c) includes performing gene sequencing on at least a cellular fraction of said biological sample to amplify a gene encoding the TCR specific to said peptide, and detecting presence of said gene encoding said TCR;
c) step b) includes performing gene sequencing on at least a cellular fraction of said biological sample to amplify a gene encoding the HLA capable of presenting said peptide, and detecting presence of said gene encoding said HLA and c) includes performing gene sequencing on at least a cellular fraction of said biological sample to amplify a gene encoding the TCR specific to said peptide, and detecting presence of said gene encoding said TCR, wherein said at least a cellular fraction of said biological sample includes peripheral blood mononuclear cells (PBMC), preferably leukocytes; or
d) the protein that forms aggregates in a patient having a neurodegenerative disease is tau, alpha-synuclein, or transactive response DNA binding protein 43 kDa (TDP-43).

46-54. (canceled)
